(19) United States
(12) Patent Application Publication
(10) Pub. No.: US 2017/0198006 A1
(43) Pub. Date: Jul. 13, 2017

(54) PRMT5 INHIBITORS AND USES THEREOF

(71) Applicant: Epizyme, Inc., Cambridge, MA (US)

(72) Inventors: Kenneth W. Duncan, Westwood, MA (US); Richard Chesworth, Concord, MA (US); Gideon Shapiro, Gainesvolle, FL (US); Edward James Olhava, Newton, MA (US); Michael A. Patane, Andover, MA (US); Michael John Munchhof, Salem, CT (US); Kevin Wayne Kuntz, Woburn, MA (US); Lei Jin, Wellesley, MA (US)

(73) Assignee: Epizyme, Inc., Cambridge, MA (US)

(21) Appl. No.: 15/321,280

(22) PCT Filed: Jun. 25, 2015
(86) PCT No.: PCT/US15/37768

§ 371 Date: Dec. 22, 2016

Related U.S. Application Data
(60) Provisional application No. 62/064,357, filed on Oct. 15, 2014.
(60) Provisional application No. 62/051,751, filed on Sep. 17, 2014.
(60) Provisional application No. 62/017,055, filed on Jun. 25, 2014.

(51) Int. Cl. C07H 19/16; C07D 405/06; C07D 401/12; C07D 403/04; C07C 311/41; C07D 233/64; C07D 207/14

(52) U.S. Cl. CPC C07H 19/16; C07D 233/64; C07D 405/06; C07D 207/14; C07D 403/04; C07C 311/41; C07D 401/12

(57) ABSTRACT

Described herein are compounds of Formula (I)-(XIII), pharmaceutically acceptable salts thereof, and pharmaceutical compositions thereof. Compounds of the present invention are useful for inhibiting PRMT5 activity. Methods of using the compounds for treating PRMT5-mediated disorders are also described.

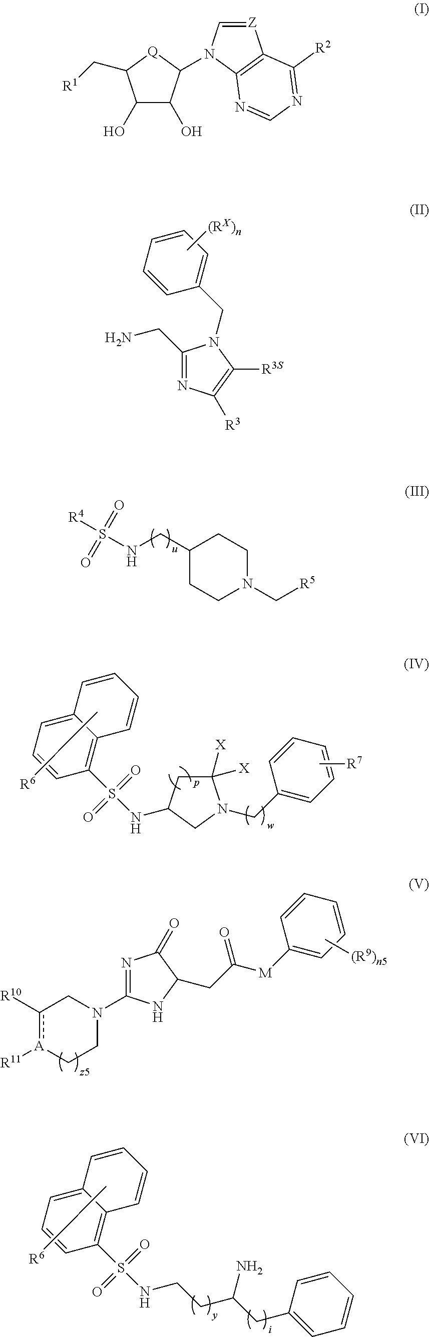

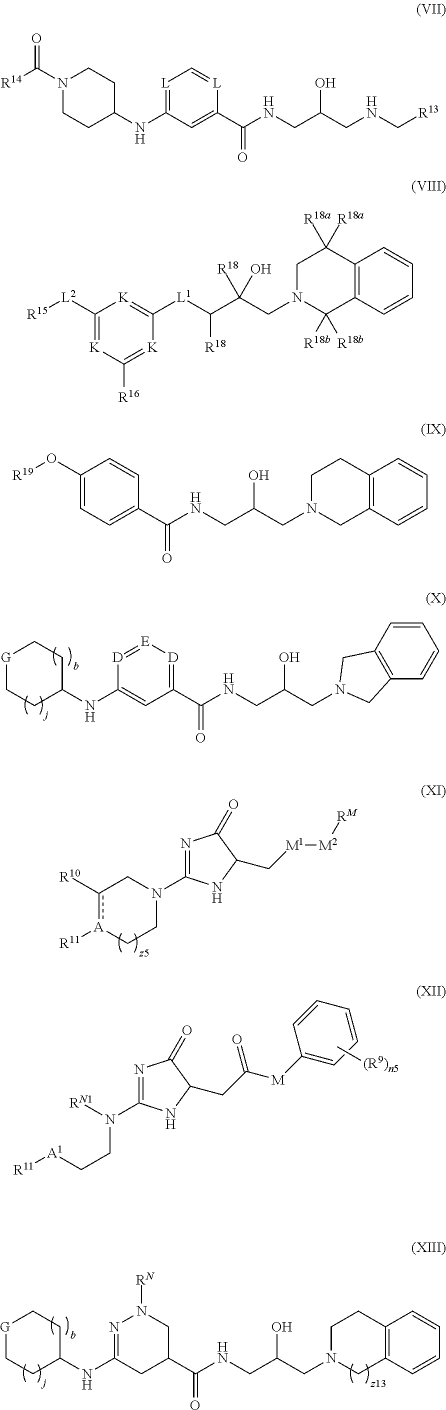

The present application claims priority under 35 U.S.C. §119(e) to U.S. provisional patent applications, U.S. Ser. No. 62/017,055, filed Jun. 25, 2014, U.S. Ser. No. 62/051,751, filed Sep. 17, 2014, and U.S. Ser. No. 62/064,357, filed Oct. 15, 2014, the entire contents of each of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease.

Disease-associated chromatin-modifying enzymes (e.g., PRMT5) play a role in diseases such as proliferative disorders, metabolic disorders, and blood disorders. Thus, there is a need for the development of small molecules that are capable of inhibiting the activity of PRMT5.

### DETAILED DESCRIPTION OF CERTAIN EMBODIMENTS

Protein arginine methyltransferase 5 (PRMT5) catalyzes the addition of two methyl groups to the two ω-guanidino nitrogen atoms of arginine, resulting in ω-NG, N′G symmetric dimethylation of arginine (sDMA) of the target protein. PRMT5 functions in the nucleus as well as in the cytoplasm, and its substrates include histones, spliceosomal proteins, transcription factors (See e.g., Sun et al., PNAS (2011), 108: 20538-20543). PRMT5 generally functions as part of a molecule weight protein complex. While the protein complexes of PRMT5 can have a variety of components, they generally include the protein MEP50 (methylosome protein 50). In addition, PRMT5 acts in conjunction with cofactor SAM (S-adenosyl methionine).

PRMT5 is an attractive target for modulation given its role in the regulation of diverse biological processes. It has now been found that compounds described herein, and pharmaceutically acceptable salts and compositions thereof, are effective as inhibitors of PRMT5.

Such compounds have the general Formula (I)-(XIII):

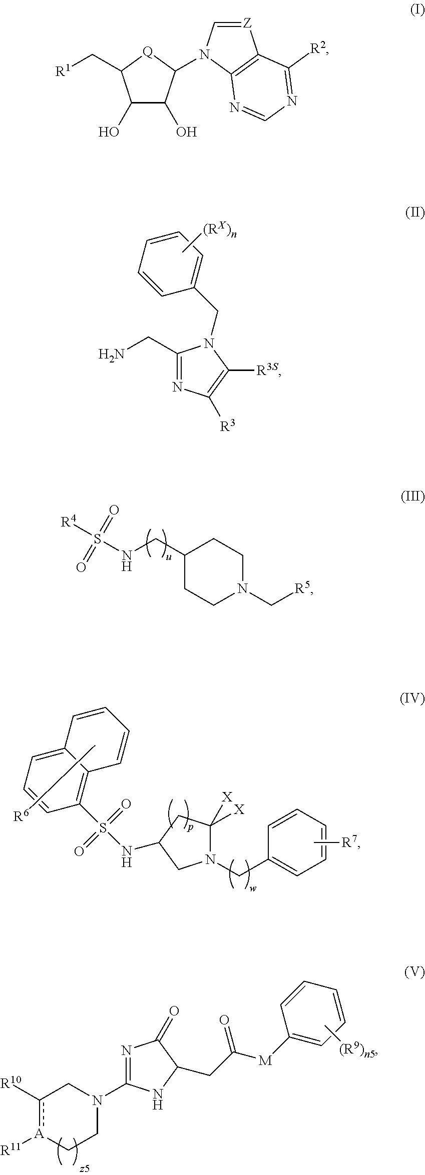

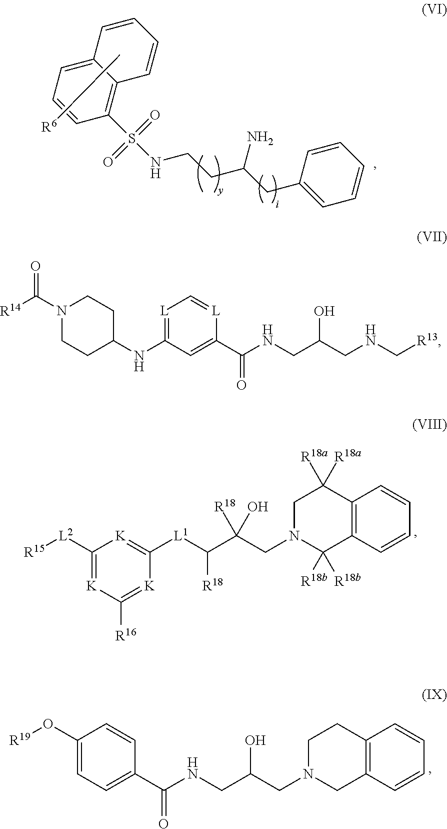

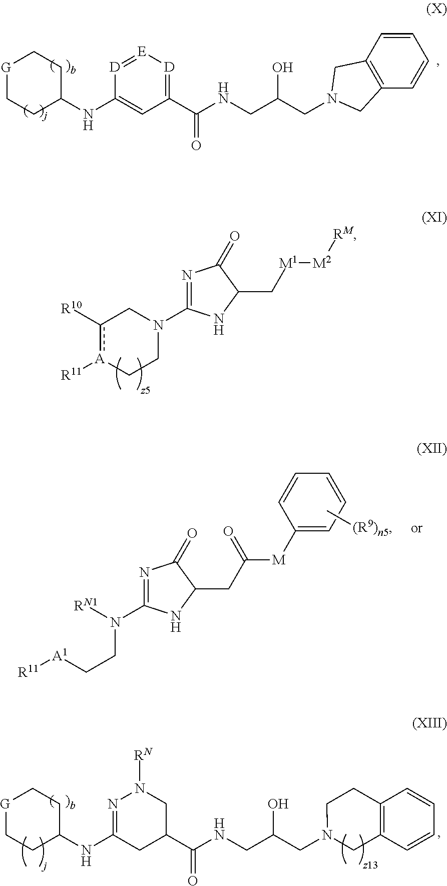

or a pharmaceutically acceptable salt thereof, wherein [symbol] , R¹, R², R³, R^{3S}, R⁴, R⁵, R⁶, R⁷, R⁹, R¹⁰, R¹¹, R¹³, R¹⁴, R¹⁵, R¹⁶, R¹⁸, R^{18a}, R^{18b}, R¹⁹, R^{X}, R^{M}, R^{N1}, A, A¹, D, E, G, L¹, L², M, M¹ M², Q, X, Z, z5, n5, z13, b, i, j, p, u, w, and y, are as defined herein.

In certain embodiments, pharmaceutical compositions are provided which comprise a compound described herein (e.g., a compound of Formula (I)-(XIII)), or a pharmaceutically acceptable salt thereof, and optionally a pharmaceutically acceptable excipient.

In certain embodiments, compounds described herein inhibit activity of PRMT5. In certain embodiments, methods of inhibiting PRMT5 are provided which comprise contacting PRMT5 with an effective amount of a compound of Formula (I)-(XIII), or a pharmaceutically acceptable salt thereof. The PRMT5 may be purified or crude, and may be present in a cell, tissue, or a subject. Thus, such methods encompass inhibition of PRMT5 activity both in vitro and in vivo. In certain embodiments, the PRMT5 is wild-type PRMT5. In certain embodiments, the PRMT5 is overexpressed. In certain embodiments, the PRMT5 is a mutant. In certain embodiments, the PRMT5 is in a cell. In certain embodiments, the PRMT5 is in an animal, e.g., a human. In certain embodiments, the PRMT5 is in a subject that is susceptible to normal levels of PRMT5 activity due to one or more mutations associated with a PRMT5 substrate. In certain embodiments, the PRMT5 is in a subject known or identified as having abnormal PRMT5 activity (e.g., overexpression). In certain embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective relative to one or more other methyltransferases.

In certain embodiments, methods of altering gene expression in a cell are provided which comprise contacting a cell with an effective amount of a compound of Formula (I)-(XIII), or a pharmaceutically acceptable salt thereof, or a pharmaceutical composition thereof. In certain embodiments, the cell in culture in vitro. In certain embodiments, cell is in an animal, e.g., a human.

In certain embodiments, methods of altering transcription in a cell are provided which comprise contacting a cell with an effective amount of a compound of Formula (I)-(XIII), or a pharmaceutically acceptable salt thereof, or a pharmaceutical composition thereof. In certain embodiments, the cell in culture in vitro. In certain embodiments, the cell is in an animal, e.g., a human.

In certain embodiments, methods of treating a PRMT5-mediated disorder are provided which comprise administering to a subject suffering from a PRMT5-mediated disorder an effective amount of a compound described herein (e.g., a compound of Formula (I)-(XIII)), or a pharmaceutically acceptable salt thereof, or a pharmaceutical composition thereof. In certain embodiments, the PRMT5-mediated disorder is a proliferative disorder, a metabolic disorder, or a blood disorder. In certain embodiments, compounds described herein are useful for treating cancer. In certain embodiments, compounds described herein are useful for treating hematopoietic cancer, lung cancer, prostate cancer, melanoma, or pancreatic cancer. In certain embodiments, compounds described herein are useful for treating a hemoglobinopathy. In certain embodiments, compounds described herein are useful for treating sickle cell anemia. In certain embodiments, compounds described herein are useful for treating diabetes or obesity. In certain embodiments, a provided compound is useful in treating inflammatory and autoimmune disease.

Compounds described herein are also useful for the study of PRMT5 in biological and pathological phenomena, the study of intracellular signal transduction pathways mediated by PRMT5, and the comparative evaluation of new PRMT5 inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

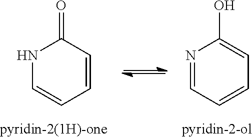 [SMILES: O=c1cccc[nH]1 ; Oc1ccccn1]

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “aliphatic,” as used herein, includes both saturated and unsaturated, nonaromatic, straight chain (i.e., unbranched), branched, acyclic, and cyclic (i.e., carbocyclic) hydrocarbons. In certain embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). In certain embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In certain embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In certain embodiments, the alkyl moiety has 1 to 8 carbon atoms (“C₁₋₈ perhaloalkyl”). In certain embodiments, the alkyl moiety has 1 to 6 carbon atoms (“C₁₋₆ perhaloalkyl”). In certain embodiments, the alkyl moiety has 1 to 4 carbon atoms (“C₁₋₄ perhaloalkyl”). In certain embodiments, the alkyl moiety has 1 to 3 carbon atoms (“C₁₋₃ perhaloalkyl”). In certain embodiments, the alkyl moiety has 1 to 2 carbon atoms (“C₁₋₂ perhaloalkyl”). In certain embodiments, all of the hydrogen atoms are replaced with fluoro. In certain embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 7 ring carbon atoms (“C₃₋₇ carbocyclyl”). In certain embodiments, a carbocyclyl group has 3 to 6 ring carbon atoms (“C₃₋₆ carbocyclyl”). In certain embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃ 8 carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In certain embodiments, “carbocyclyl” is a monocyclic, saturated carbocyclyl group having from 3 to 14 ring carbon atoms (“C₃₋₁₄ cycloalkyl”). In certain embodiments, a cycloalkyl group has 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In certain embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In certain embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In certain embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In certain embodiments, the 5-6 membered heterocyclyl has 1-3 ring heteroatoms independently selected from nitrogen, oxygen, and sulfur. In certain embodiments, the 5-6 membered heterocyclyl has 1-2 ring heteroatoms independently selected from nitrogen, oxygen, and sulfur. In certain embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl, oxadiazinanyl, thiadiazinanyl, oxathiazinanyl, and dioxazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In certain embodiments, an aryl group has six ring carbon atoms (“C₆ aryl”; e.g., phenyl). In certain embodiments, an aryl group has ten ring carbon atoms (“C₁₀ aryl”; e.g., naphthyl such as 1-naphthyl and 2-naphthyl). In certain embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In certain embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In certain embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In certain embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In certain embodiments, the 5-6 membered heteroaryl has 1-3 ring heteroatoms independently selected from nitrogen, oxygen, and sulfur. In certain embodiments, the 5-6 membered heteroaryl has 1-2 ring heteroatoms independently selected from nitrogen, oxygen, and sulfur. In certain embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Fused” or “ortho-fused” are used interchangeably herein, and refer to two rings that have two atoms and one bond in common, e.g.,

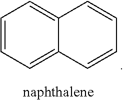

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

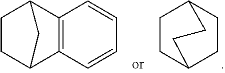

“Spiro” or “Spiro-fused” refers to a group of atoms which connect to the same atom of a carbocyclic or heterocyclic ring system (geminal attachment), thereby forming a ring, e.g.,

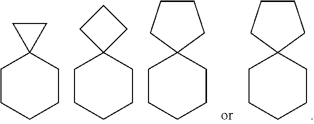

Spiro-fusion at a bridgehead atom is also contemplated.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In certain embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}C₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{a}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5, R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₄ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group”, or “LG”, is a term understood in the art to refere to a molecular fragment that departs with a pair of electrons upon heterolytic bond cleavage, wherein the molecular fragment is an anion or neutral molecule. See, for example, Smith, March Advanced Organic Chemistry 6th ed. (501-502). Examples of suitable leaving groups include, but are not limited to, halides (such as chloride, bromide, or iodide), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, haloformates, —NO₂, trialkylammonium, and aryliodonium salts. In certain embodiments, the leaving group is a sulfonic acid ester. In certain embodiments, the sulfonic acid ester comprises the formula —OSO₂R^{LG1} wherein R^{LG1} is selected from the group consisting alkyl optionally, alkenyl optionally substituted, heteroalkyl optionally substituted, aryl optionally substituted, heteroaryl optionally substituted, arylalkyl optionally substituted, and heterarylalkyl optionally substituted. In certain embodiments, R^{LG1} is substituted or unsubstituted C₁-C₆ alkyl. In certain embodiments, R^{LG1} is methyl. In certain embodiments, R^{LG1} is —CF₃. In certain embodiments, R^{LG1} is substituted or unsubstituted aryl. In certain embodiments, R^{LG1} is substituted or unsubstituted phenyl. In certain embodiments R^{LG1} is:

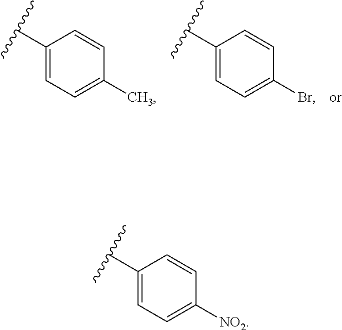 [SMILES: *c1ccc(C)cc1 ; *c1ccc(Br)cc1 ; *c1ccc([N+](=O)[O-])cc1]

In some cases, the leaving group is toluenesulfonate (tosylate, Ts), methanesulfonate (mesylate, Ms), p-bromobenzenesulfonyl (brosylate, Bs), or trifluoromethanesulfonate (triflate, Tf). In some cases, the leaving group is a brosylate (p-bromobenzenesulfonyl). In some cases, the leaving group is a nosylate (2-nitrobenzenesulfonyl). In certain embodiments, the leaving group is a sulfonate-containing group. In certain embodiments, the leaving group is a tosylate group. The leaving group may also be a phosphineoxide (e.g., formed during a Mitsunobu reaction) or an internal leaving group such as an epoxide or cyclic sulfate.

These and other exemplary substituents are described in more detail in the Detailed Description, Examples, and claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In certain embodiments, a methyltransferase described herein is a protein methyltransferase. In certain embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

### Compounds of Formula (I)

As generally described above, provided herein are compounds useful as PRMT5 inhibitors. In certain embodiments, the present disclosure provides a compound of Formula (I):

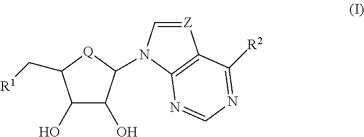

or a pharmaceutically acceptable salt thereof,
wherein:
R¹ is —SR^{1a} or —N(R^{1a})₂;
each R^{1a} is independently hydrogen, substituted or unsubstituted alkyl, substituted or unsubstituted cycloalkyl, substituted or unsubstituted heterocyclyl, —C(═O)R^{Z}, —C(═NH)R^{Z}, a nitrogen protecting group when attached to nitrogen, a sulfur protecting group when attached to sulfur, or two instances of R^{1a} can be taken together with their intervening atoms to form a substituted or unsubstituted heterocyclic ring;
R^{Z} is substituted or unsubstituted alkyl or substituted or unsubstituted aryl;
R² is hydrogen or —NH₂;
Z is CH or N; and
Q is CH₂ or O.

In certain embodiments, the provided compound is of a free base form. In certain embodiments, the provided compound is in the form of a pharmaceutically acceptable salt as generally defined herein.

As is generally described herein, R² is hydrogen or —NH₂, Z is CH or N, and Q is CH₂ or O. In certain embodiments, R² is —NH₂, Z is N, and Q is O. In certain embodiments, R² is —NH₂, Z is CH, and Q is CH₂. In certain embodiments, R² is hydrogen, Z is CH, and Q is CH₂.

In certain embodiments, R¹ is —N(R^{1a})₂; and at least one of R^{1a} is substituted alkyl. In certain embodiments, R¹ is —N(R^{1a})₂; and at least one of R^{1a} is substituted C₁₋₆alkyl. In certain embodiments, R¹ is —N(R^{1a})₂; and at least one of R^{1a} is substituted C₁₋₃alkyl. In certain embodiments, R¹ is —N(R^{1a})₂; and at least one of R^{1a} is substituted methyl. In certain embodiments, R¹ is —N(R^{1a})₂; and at least one of R^{1a} is substituted ethyl. In certain embodiments, R¹ is —N(R^{1a})₂; and at least one of R^{1a} is substituted propyl. In certain embodiments, R¹ is —N(R^{1a})₂; and at least one of R^{1a} is substituted isopropyl.

In certain embodiments, R¹ is —N(R^{1a})₂; and at least one of R^{1a} is C₁₋₆alkyl substituted with a substituted or unsubstituted phenyl ring. In certain embodiments, R¹ is —N(R^{1a})₂; and at least one of R^{1a} is —CH₂-Ph (benzyl, Bn). In certain embodiments, R¹ is —N(R^{1a})₂; and at least one of R^{1a} is C₁₋₆alkyl substituted with a substituted or unsubstituted cycloalkyl ring. In certain embodiments, R¹ is —N(R^{1a})₂; and at least one of R^{1a} is C₁₋₆alkyl substituted with a substituted or unsubstituted heteroaryl ring. In certain embodiments, R¹ is —N(R^{1a})₂; and at least one of R^{1a} is C₁₋₆alkyl substituted with a substituted or unsubstituted heterocyclyl ring.

In certain embodiments, R¹ is —N(R^{1a})₂; and at least one of R^{1a} is C₁₋₆alkyl substituted with an —NH₂ group.

In certain embodiments, R¹ is —N(R^{1a})₂; and at least one of R^{1a} is C₁₋₆alkyl substituted with —CO₂H.

In certain embodiments, R¹ is —N(R^{1a})₂; and at least one of R^{1a} is C₁₋₆alkyl substituted with an alkoxy group. In certain embodiments, R¹ is —N(R^{1a})₂; and at least one of R^{1a} is C₁₋₆alkyl substituted with a methoxy group. In certain embodiments, R¹ is —N(R^{1a})₂; and at least one of R^{1a} is C₁₋₆alkyl substituted with —OH.

In certain embodiments, R¹ is —N(R^{1a})₂; and at least one of R^{1a} is unsubstituted alkyl. In certain embodiments, R¹ is —N(R^{1a})₂; and at least one of R^{1a} is unsubstituted C₁₋₆alkyl. In certain embodiments, R¹ is —N(R^{1a})₂; and at least one of R^{1a} is unsubstituted C₁₋₃alkyl. In certain embodiments, R¹ is —N(R^{1a})₂; and at least one of R^{1a} is unsubstituted methyl. In certain embodiments, R¹ is —N(R^{1a})₂; and at least one of R^{1a} is unsubstituted ethyl. In certain embodiments, R¹ is —N(R^{1a})₂; and at least one of R^{1a} is unsubstituted propyl. In certain embodiments, R¹ is —N(R^{1a})₂; and at least one of R^{1a} is unsubstituted isopropyl.

In certain embodiments, R¹ is —N(R^{1a})₂; and at least one of R^{1a} is substituted cycloalkyl. In certain embodiments, R¹ is —N(R^{1a})₂; and at least one of R^{1a} is substituted C₃₋₆ cycloalkyl. In certain embodiments, R¹ is —N(R^{1a})₂; and at least one of R^{1a} is substituted cyclopropyl. In certain embodiments, R¹ is —N(R^{1a})₂; and at least one of R^{1a} is substituted cyclobutyl. In certain embodiments, R¹ is —N(R^{1a})₂; and at least one of R^{1a} is substituted cyclopentyl. In certain embodiments, R¹ is —N(R^{1a})₂; and at least one of R^{1a} is substituted cyclohexyl. In certain embodiments, R¹ is —N(R^{1a})₂; and at least one of R^{1a} is unsubstituted cycloalkyl.

In certain embodiments, R¹ is —N(R^{1a})₂; and at least one of R^{1a} is unsubstituted C₃₋₆ cycloalkyl. In certain embodiments, R¹ is —N(R^{1a})₂; and at least one of R^{1a} is unsubstituted cyclopropyl. In certain embodiments, R¹ is —N(R^{1a})₂; and at least one of R^{1a} is unsubstituted cyclobutyl. In certain embodiments, R¹ is —N(R^{1a})₂; and at least one of R^{1a} is unsubstituted cyclopentyl. In certain embodiments, R¹ is —N(R^{1a})₂; and at least one of R^{1a} is unsubstituted cyclohexyl.

In certain embodiments, R¹ is —N(R^{1a})₂; and at least one of R^{1a} is substituted heterocyclyl. In certain embodiments, R¹ is —N(R^{1a})₂; and at least one of R^{1a} is substituted 4-6 membered heterocyclyl. In certain embodiments, R¹ is —N(R^{1a})₂; and at least one of R^{1a} is substituted oxetanyl. In certain embodiments, R¹ is —N(R^{1a})₂; and at least one of R^{1a} is substituted azetidinyl. In certain embodiments, R¹ is —N(R^{1a})₂; and at least one of R^{1a} is substituted tetrahydrofuryl. In certain embodiments, R¹ is —N(R^{1a})₂; and at least one of R^{1a} is substituted pyrrolidinyl. In certain embodiments, R¹ is —N(R^{1a})₂; and at least one of R^{1a} is substituted tetrahydropyranyl. In certain embodiments, R¹ is —N(R^{1a})₂; and at least one of R^{1a} is substituted piperidinyl.

In certain embodiments, R¹ is —N(R^{1a})₂; and at least one of R^{1a} is unsubstituted heterocyclyl. In certain embodiments, R¹ is —N(R^{1a})₂; and at least one of R^{1a} is unsubstituted 4-6 membered heterocyclyl. In certain embodiments, R¹ is —N(R^{1a})₂; and at least one of R^{1a} is unsubstituted oxetanyl. In certain embodiments, R¹ is —N(R^{1a})₂; and at least one of R^{1a} is unsubstituted azetidinyl. In certain embodiments, R¹ is —N(R^{1a})₂; and at least one of R^{1a} is unsubstituted tetrahydrofuryl. In certain embodiments, R¹ is —N(R^{1a})₂; and at least one of R^{1a} is unsubstituted pyrrolidinyl. In certain embodiments, R¹ is —N(R^{1a})₂; and at least one of R^{1a} is unsubstituted tetrahydropyranyl. In certain embodiments, R¹ is —N(R^{1a})₂; and at least one of R^{1a} is unsubstituted piperidinyl.

In certain embodiments, R¹ is —N(R^{1a})₂; and at least one of R^{1a} is —C(═O)R^{Z}. In certain embodiments, R^{Z} is substituted alkyl. In certain embodiments, R^{Z} is substituted C₁₋₆alkyl. In certain embodiments, R^{Z} is substituted C₁₋₃alkyl. In certain embodiments, R^{Z} is substituted methyl. In certain embodiments, R^{Z} is substituted ethyl. In certain embodiments, R^{Z} is substituted propyl. In certain embodiments, R^{Z} is substituted isopropyl. In certain embodiments, R^{Z} is unsubstituted alkyl. In certain embodiments, R^{Z} is unsubstituted C₁₋₆alkyl. In certain embodiments, R^{Z} is unsubstituted C₁₋₃alkyl. In certain embodiments, R^{Z} is unsubstituted methyl. In certain embodiments, R^{Z} is unsubstituted ethyl. In certain embodiments, R^{Z} is unsubstituted propyl. In certain embodiments, R^{Z} is unsubstituted isopropyl. In certain embodiments, R^{Z} is substituted aryl. In certain embodiments, R^{Z} is substituted phenyl. In certain embodiments, R^{Z} is unsubstituted aryl. In certain embodiments, R^{Z} is unsubstituted phenyl. In certain embodiments, R¹ is —N(R^{1a})₂; and at least one of R^{1a} is —C(═O)CH₃ (acetyl).

In certain embodiments, R¹ is —N(R^{1a})₂; and at least one of R^{1a} is —C(═NH)R^{Z}. In certain embodiments, R^{Z} is substituted alkyl. In certain embodiments, R^{Z} is substituted C₁₋₆alkyl. In certain embodiments, R^{Z} is substituted C₁₋₃alkyl. In certain embodiments, R^{Z} is substituted methyl. In certain embodiments, R^{Z} is substituted ethyl. In certain embodiments, R^{Z} is substituted propyl. In certain embodiments, R^{Z} is substituted isopropyl. In certain embodiments, R^{Z} is unsubstituted alkyl. In certain embodiments, R^{Z} is unsubstituted C₁₋₆alkyl. In certain embodiments, R^{Z} is unsubstituted C₁₋₃alkyl. In certain embodiments, R^{Z} is unsubstituted methyl. In certain embodiments, R^{Z} is unsubstituted ethyl. In certain embodiments, R^{Z} is unsubstituted propyl. In certain embodiments, R^{Z} is unsubstituted isopropyl. In certain embodiments, R^{Z} is substituted aryl. In certain embodiments, R^{Z} is substituted phenyl. In certain embodiments, R^{Z} is unsubstituted aryl. In certain embodiments, R^{Z} is unsubstituted phenyl (Ph). In certain embodiments, R¹ is —N(R^{1a})₂; and at least one of R^{1a} is —C(═NH)Ph.

In certain embodiments, R¹ is —N(R^{1a})₂; and two instances of R^{1a} are taken together to form a substituted heterocyclic ring. In certain embodiments, R¹ is —N(R^{1a})₂; and two instances of R^{1a} are taken together to form a substituted 4-7 membered heterocyclic ring. In certain embodiments, R¹ is —N(R^{1a})₂; and two instances of R^{1a} are taken together to form a substituted azetidine. In certain embodiments, R¹ is —N(R^{1a})₂; and two instances of R^{1a} are taken together to form a substituted pyrrolidine. In certain embodiments, R¹ is —N(R^{1a})₂; and two instances of R^{1a} are taken together to form a substituted piperidine. In certain embodiments, R¹ is —N(R^{1a})₂; and two instances of R^{1a} are taken together to form a substituted piperazine. In certain embodiments, R¹ is —N(R^{1a})₂; and two instances of R^{1a} are taken together to form a substituted azepane. In certain embodiments, R¹ is —N(R^{1a})₂; and two instances of R^{1a} are taken together to form a substituted diazepane. In certain embodiments, R¹ is —N(R^{1a})₂; and two instances of R^{1a} are taken together to form an unsubstituted heterocyclic ring. In certain embodiments, R¹ is —N(R^{1a})₂; and two instances of R^{1a} are taken together to form an unsubstituted 4-7 membered heterocyclic ring. In certain embodiments, R¹ is —N(R^{1a})₂; and two instances of R^{1a} are taken together to form an unsubstituted azetidine. In certain embodiments, R¹ is —N(R^{1a})₂; and two instances of R^{1a} are taken together to form an unsubstituted pyrrolidine. In certain embodiments, R¹ is —N(R^{1a})₂; and two instances of R^{1a} are taken together to form an unsubstituted piperidine. In certain embodiments, R¹ is —N(R^{1a})₂; and two instances of R^{1a} are taken together to form an unsubstituted piperazine. In certain embodiments, R¹ is —N(R^{1a})₂; and two instances of R^{1a} are taken together to form an unsubstituted azepane. In certain embodiments, R¹ is —N(R^{1a})₂; and two instances of R^{1a} are taken together to form an unsubstituted diazepane.

In certain embodiments, R¹ is —N(R^{1a})₂; and neither instance of R^{1a} is hydrogen. In certain embodiments, R¹ is —N(R^{1a})₂; and neither instance of R^{1a} is methyl. In certain embodiments, R¹ is —N(R^{1a})₂; and neither instance of R^{1a} is hydrogen. In certain embodiments, R¹ is —N(R^{1a})₂; and neither instance of R^{1a} is ethyl. In certain embodiments, R¹ is —N(R^{1a})₂; and neither instance of R^{1a} is isopropyl. In certain embodiments, R¹ is —N(R^{1a})₂; and neither instance of R^{1a} is unsubstituted benzyl. In certain embodiments, R¹ is —N(R^{1a})₂; and neither instance of R^{1a} is methyl. In certain embodiments, when R¹ is —N(R^{1a})₂ and one instance of R^{1a} is a nitrogen protecting group, the second instance of R^{1a} is not a nitrogen protecting group. In certain embodiments, when R¹ is —N(R^{1a})₂ and one instance of R^{1a} is a nitrogen protecting group, the second instance of R^{1a} is not hydrogen.

In certain embodiments, R¹ is —SR^{1a}; and R^{1a} is substituted alkyl. In certain embodiments, R¹ is —SR^{1a}; and R^{1a} is substituted C₁₋₆alkyl. In certain embodiments, R¹ is —SR^{1a}; and R^{1a} is substituted C₁₋₃alkyl. In certain embodiments, R¹ is —SR^{1a}; and R^{1a} is substituted methyl. In certain embodiments, R¹ is —SR^{1a}; and R^{1a} is substituted ethyl. In certain embodiments, R¹ is —SR^{1a}; and R^{1a} is substituted propyl. In certain embodiments, R¹ is —SR^{1a}; and R^{1a} is substituted isopropyl.

In certain embodiments, R¹ is —SR^{1a}; and R^{1a} is C₁₋₆alkyl substituted with a substituted or unsubstituted phenyl ring. In certain embodiments, R¹ is —SR^{1a}; and R^{1a} is —CH₂Ph (Bn, benzyl). In certain embodiments, R¹ is —SR^{1a}; and R^{1a} is C₁₋₆alkyl substituted with a substituted or unsubstituted cycloalkyl ring. In certain embodiments, R¹ is —SR^{1a}; and R^{1a} is C₁₋₆alkyl substituted with a substituted or unsubstituted heteroaryl ring. In certain embodiments, R¹ is —SR^{1a}; and R^{1a} is C₁₋₆alkyl substituted with a substituted or unsubstituted heterocyclyl ring.

In certain embodiments, R¹ is —SR^{1a}; and R^{1a} is C₁₋₆alkyl substituted with an —NH₂ group.

In certain embodiments, R¹ is —SR^{1a}; and R^{1a} is C₁₋₆alkyl substituted with —CO₂H.

In certain embodiments, R¹ is —SR^{1a}; and R^{1a} is C₁₋₆alkyl substituted with an alkoxy group. In certain embodiments, R¹ is —SR^{1a}; and R^{1a} is C₁₋₆alkyl substituted with a methoxy group. In certain embodiments, R¹ is —SR^{1a}; and R^{1a} is C₁₋₆alkyl substituted with —OH.

In certain embodiments, R¹ is —SR^{1a}; and R^{1a} is unsubstituted alkyl. In certain embodiments, R¹ is —SR^{1a}; and R^{1a} is unsubstituted C₁₋₆alkyl. In certain embodiments, R¹ is —SR^{1a}; and R^{1a} is unsubstituted C₁₋₃alkyl. In certain embodiments, R¹ is —SR^{1a}; and R^{1a} is unsubstituted methyl. In certain embodiments, R¹ is —SR^{1a}; and R^{1a} is unsubstituted ethyl. In certain embodiments, R¹ is —SR^{1a}; and R^{1a} is unsubstituted propyl. In certain embodiments, R¹ is —SR^{1a}; and R^{1a} is unsubstituted isopropyl. In certain embodiments, R¹ is —SR^{1a}; and R^{1a} is unsubstituted isopropyl.

In certain embodiments, R¹ is —SR^{1a}; and R^{1a} is substituted cycloalkyl. In certain embodiments, R¹ is —SR^{1a}; and R^{1a} is substituted C₃₋₆cycloalkyl. In certain embodiments, R¹ is —SR^{1a}; and R^{1a} is substituted cyclopropyl. In certain embodiments, R¹ is —SR^{1a}; and R^{1a} is substituted cyclobutyl. In certain embodiments, R¹ is —SR^{1a}; and R^{1a} is substituted cyclopentyl. In certain embodiments, R¹ is —SR^{1a}; and R^{1a} is substituted cyclohexyl.

In certain embodiments, R¹ is —SR^{1a}; and R^{1a} is unsubstituted cycloalkyl. In certain embodiments, R¹ is —SR^{1a}; and R^{1a} is unsubstituted C₃₋₆cycloalkyl. In certain embodiments, R¹ is —SR^{1a}; and R^{1a} is unsubstituted cyclopropyl. In certain embodiments, R¹ is —SR^{1a}; and R^{1a} is unsubstituted cyclobutyl. In certain embodiments, R¹ is —SR^{1a}; and R^{1a} is unsubstituted cyclopentyl. In certain embodiments, R¹ is —SR^{1a}; and R^{1a} is unsubstituted cyclohexyl.

In certain embodiments, R¹ is —SR^{1a}; and R^{1a} is substituted heterocyclyl. In certain embodiments, R¹ is —SR^{1a}; and R^{1a} is substituted 4-6 membered heterocyclyl. In certain embodiments, R¹ is —SR^{1a}; and R^{1a} is substituted oxetanyl. In certain embodiments, R¹ is —SR^{1a}; and R^{1a} is substituted azetidinyl. In certain embodiments, R¹ is —SR^{1a}; and R^{1a} is substituted tetrahydrofuryl. In certain embodiments, R¹ is —SR^{1a}; and R^{1a} is substituted pyrrolidinyl. In certain embodiments, R¹ is —SR^{1a}; and R^{1a} is substituted tetrahydropyranyl. In certain embodiments, R¹ is —SR^{1a}; and R^{1a} is substituted piperidinyl.

In certain embodiments, R¹ is —SR^{1a}; and R^{1a} is unsubstituted heterocyclyl. In certain embodiments, R¹ is —SR^{1a}; and R^{1a} is unsubstituted 4-6 membered heterocyclyl. In certain embodiments, R¹ is —SR^{1a}; and R^{1a} is unsubstituted oxetanyl. In certain embodiments, R¹ is —SR^{1a}; and R^{1a} is unsubstituted azetidinyl. In certain embodiments, R¹ is —SR^{1a}; and R^{1a} is unsubstituted tetrahydrofuryl. In certain embodiments, R¹ is —SR^{1a}; and R^{1a} is unsubstituted pyrrolidinyl. In certain embodiments, R¹ is —SR^{1a}; and R^{1a} is unsubstituted tetrahydropyranyl. In certain embodiments, R¹ is —SR^{1a}; and R^{1a} is unsubstituted piperidinyl.

In certain embodiments, R¹ is —SR^{1a}; and R^{1a} is not hydrogen. In certain embodiments, R¹ is —SR^{1a}; and R^{1a} is not methyl. In certain embodiments, R¹ is —SR^{1a}; and R^{1a} is not hydrogen. In certain embodiments, R¹ is —SR^{1a}; and R^{1a} is not ethyl. In certain embodiments, R¹ is —SR^{1a}; and R^{1a} is not isopropyl. In certain embodiments, R¹ is —SR^{1a}; and R^{1a} is not unsubstituted benzyl. In certain embodiments, R¹ is —SR^{1a}; and R^{1a} is not a sulfur protecting group.

In certain embodiments, a compound of Formula (I) is of Formula (I-a):

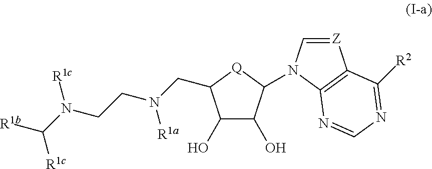

or a pharmaceutically acceptable salt thereof,
wherein:
R^{1a} is —CH₃;
R^{1b} is substituted or unsubstituted aryl or substituted or unsubstituted heteroaryl; and
each R¹ is independently hydrogen, —CH₃, or —CH₂CH₃.

In certain embodiments, a compound of Formula (I-a) is of Formula (I-a1):

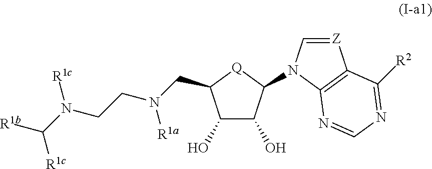

or a pharmaceutically acceptable salt thereof.

In certain embodiments, R^{1b} is substituted aryl. In certain embodiments, R^{1b} is unsubstituted aryl. In certain embodiments, R^{1b} is monosubstituted phenyl. In certain embodiments, R^{1b} is disubstituted phenyl. In certain embodiments, R^{1b} is trisubstituted phenyl. In certain embodiments, R^{1b} is tetrasubstituted phenyl. In certain embodiments, R^{1b} is pentasubstituted phenyl. In certain embodiments, R^{1b} is unsubstituted phenyl. In certain embodiments, R^{1b} is phenyl substituted with at least one alkoxy group. In certain embodiments, R^{1b} is phenyl substituted with at least one —O(C₁₋₆haloalkyl) group. In certain embodiments, R^{1b} is phenyl substituted with at least one —OCF₃ group. In certain embodiments, R^{1b} is phenyl substituted with at least one —OCF₂H group. In certain embodiments, R^{1b} is phenyl substituted with at least one —O(C₁₋₆alkyl) group. In certain embodiments, R^{1b} is phenyl substituted with at least one —OMe group. In certain embodiments, R^{1b} is phenyl substituted with at least two —O(C₁₋₆alkyl) groups. In certain embodiments, R^{1b} is phenyl substituted with at least two —OMe groups. In certain embodiments, R^{1b} is phenyl substituted with at least one substituted or unsubstituted alkyl group. In certain embodiments, R^{1b} is phenyl substituted with at least one substituted or unsubstituted C₁₋₆alkyl group. In certain embodiments, R^{1b} is phenyl substituted with at least one substituted or unsubstituted C₁₋₃alkyl group. In certain embodiments, R^{1b} is phenyl substituted with at least one substituted or unsubstituted methyl group. In certain embodiments, R^{1b} is phenyl substituted with at least one halogen (e.g., fluoro, chloro, bromo, or iodo). In certain embodiments, R^{1b} is phenyl substituted with at least two halogens (e.g., fluoro, chloro, bromo, or iodo).

In certain embodiments, R^{1b} is of formula:

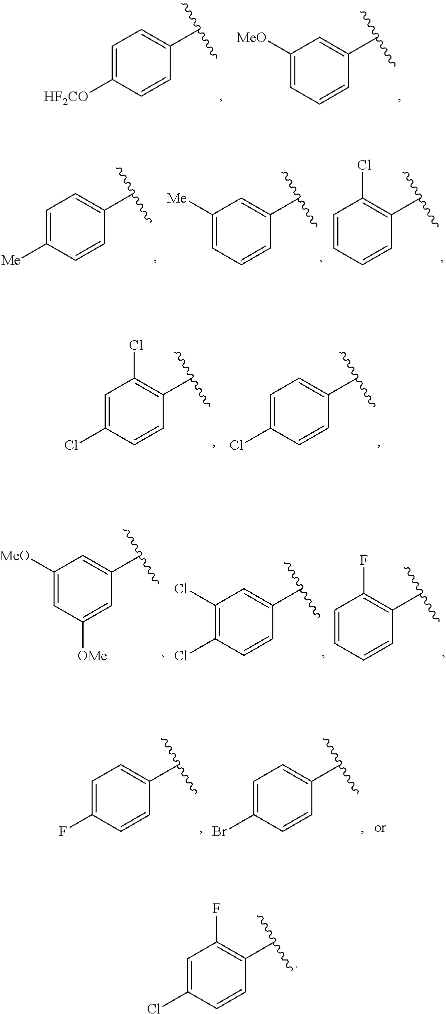

In certain embodiments, R^{1b} is substituted heteroaryl. In certain embodiments, R^{1b} is substituted 5-6 membered heteroaryl. In certain embodiments, R^{1b} is substituted imidazolyl. In certain embodiments, R^{1b} is substituted pyrazolyl. In certain embodiments, R^{1b} is substituted oxazolyl. In certain embodiments, R^{1b} is substituted thiazolyl. In certain embodiments, R^{1b} is substituted oxadiazolyl. In certain embodiments, R^{1b} is substituted thiadiazolyl. In certain embodiments, R^{1b} is substituted triazolyl. In certain embodiments, R^{1b} is substituted tetrazolyl. In certain embodiments, R^{1b} is substituted pyridyl. In certain embodiments, R^{1b} is substituted pyrazinyl. In certain embodiments, R^{1b} is substituted pyrimidinyl. In certain embodiments, R^{1b} is substituted pyridizinyl. In certain embodiments, R^{1b} is unsubstituted heteroaryl. In certain embodiments, R^{1b} is unsubstituted 5-6 membered heteroaryl. In certain embodiments, R^{1b} is unsubstituted imidazolyl. In certain embodiments, R^{1b} is unsubstituted pyrazolyl. In certain embodiments, R^{1b} is unsubstituted oxazolyl. In certain embodiments, R^{1b} is unsubstituted thiazolyl. In certain embodiments, R^{1b} is unsubstituted oxadiazolyl. In certain embodiments, R^{1b} is unsubstituted thiadiazolyl. In certain embodiments, R^{1b} is unsubstituted triazolyl. In certain embodiments, R^{1b} is unsubstituted tetrazolyl. In certain embodiments, R^{1b} is unsubstituted pyridyl. In certain embodiments, R^{1b} is unsubstituted pyrazinyl. In certain embodiments, R^{1b} is unsubstituted pyrimidinyl. In certain embodiments, R^{1b} is unsubstituted pyridizinyl.

In certain embodiments, R^{1b} is of formula:

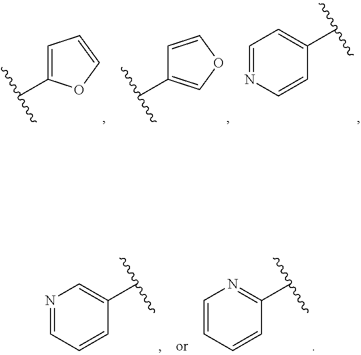

In certain embodiments, at least one instance of R^{1c} is hydrogen. In certain embodiments, both instances of R^{1c} are hydrogen. In certain embodiments, at least one instance of R^{1c} is —CH₃. In certain embodiments, both instances of R^{1c} are —CH₃. In certain embodiments, at least one instance of R^{1c} is —CH₂CH₃. In certain embodiments, both instances of R^{1c} are —CH₂CH₃. In certain embodiments, one instance of R^{1c} is hydrogen; and the second instance of R^{1c} is —CH₃. In certain embodiments, one instance of R^{1c} is hydrogen; and the second instance of R^{1c} is —CH₂CH₃. In certain embodiments, one instance of R^{1c} is —CH₃; and the second instance of R^{1c} is —CH₂CH₃.

In certain embodiments, a compound of Formula (I) is of Formula (I-b):

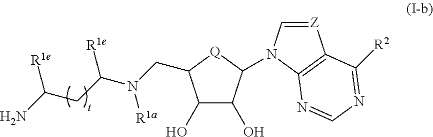

or a pharmaceutically acceptable salt thereof,
wherein:
R^{1a} is substituted or unsubstituted alkyl;
each R^{1e} is independently hydrogen, —CH₃, or —CO₂H; and
t is 0, 1, or 2.

In certain embodiments, a compound of Formula (I-b) is of Formula (I-b1):

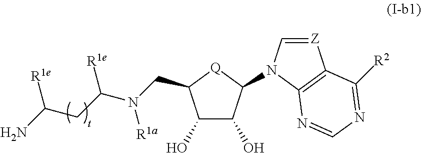

or a pharmaceutically acceptable salt thereof.

In certain embodiments, at least on instance of R^{1e} is hydrogen. In certain embodiments, both instances of R^{1e} are hydrogen. In certain embodiments, at least on instance of R^{1e} is —CH₃. In certain embodiments, both instances of R^{1e} are —CH₃. In certain embodiments, at least on instance of R^{1e} is —CO₂H. In certain embodiments, both instances of R^{1e} are —CO₂H. In certain embodiments, one instance of R^{1e} is hydrogen; and the second instance of R^{1e} is —CH₃. In certain embodiments, one instance of R^{1e} is hydrogen; and the second instance of R^{1e} is —CO₂H. In certain embodiments, one instance of R^{1e} is —CH₃; and the second instance of R^{1e} is —CO₂H.

In certain embodiments, t is 0. In certain embodiments, t is 1. In certain embodiments, t is 2.

In certain embodiments, a compound of Formula (I) is of Formula (I-c):

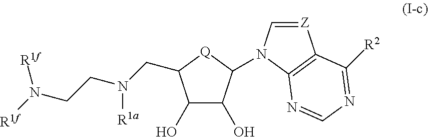

or a pharmaceutically acceptable salt thereof,
wherein:
each R^{1f} is independently substituted or unsubstituted alkyl, substituted or unsubstituted heterocyclyl, a nitrogen protecting group, or two of R^{1f} can be taken together with their intervening atoms to form a substituted or unsubstituted heterocyclic ring; and
R^{1a} is substituted or unsubstituted alkyl.

In certain embodiments, a compound of Formula (I-c) is of Formula (I-c1):

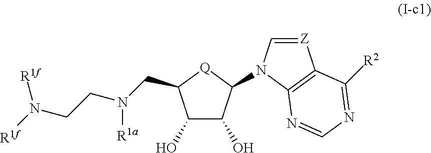

or a pharmaceutically acceptable salt thereof.

In certain embodiments, at least one R^{1f} is substituted alkyl. In certain embodiments, at least one R^{1f} is substituted C₁₋₆alkyl. In certain embodiments, at least one R^{1f} is substituted C₁₋₃alkyl. In certain embodiments, at least one R^{1f} is substituted methyl. In certain embodiments, at least one R^{1f} is substituted ethyl. In certain embodiments, at least one R^{1f} is substituted propyl. In certain embodiments, at least one R^{1f} is substituted isopropyl.

In certain embodiments, at least one R^{1f} is —CH₂(cycloalkyl), wherein the cycloalkyl is substituted or unsubstituted. In certain embodiments, at least one R^{1f} is —CH₂(cyclopropyl), wherein the cyclopropyl is substituted or unsubstituted.

In certain embodiments, at least one R^{1f} is —CH₂CH₂OH.

In certain embodiments, at least one R^{1f} is

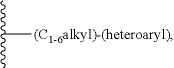

wherein the heteroaryl is substituted or unsubstituted. In certain embodiments, at least one R^{1f} is

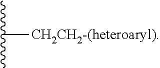

In certain embodiments, at least one R^{1f} is

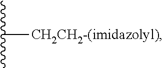

wherein the imidazolyl is substituted or unsubstituted. In certain embodiments, at least one R^{1f is}

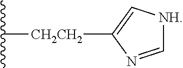

In certain embodiments, at least one R^{1f} is

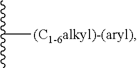

wherein the aryl is substituted or unsubstituted. In certain embodiments, at least one R^{1f} is

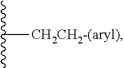

wherein the aryl is substituted or unsubstituted. In certain embodiments, at least one R^{1f} is

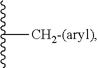

wherein the aryl is substituted or unsubstituted. In certain embodiments, at least one R^{1f} is

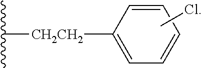

In certain embodiments, at leas one R^{1f} is

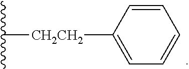

In certain embodiments, at least one R^{1f} is

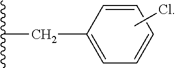

In certain embodiments, at least one R^{1f} is

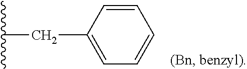 [SMILES: *Cc1ccccc1]

In certain embodiments, two instances of R^{1f} are unsubstituted methyl. In certain embodiments, two instances of R^{1f} are unsubstituted ethyl.

In certain embodiments, at least one R^{1f} is substituted heterocyclyl. In certain embodiments, at least one R^{1f} is substituted 4-6 membered heterocyclyl. In certain embodiments, at least one R^{1f} is substituted oxetanyl. In certain embodiments, at least one R^{1f} is substituted azetidinyl. In certain embodiments, at least one R^{1f} is substituted tetrahydrofuryl. In certain embodiments, at least one R^{1f} is substituted pyrrolidinyl. In certain embodiments, at least one R^{1f} is substituted tetrahydropyranyl. In certain embodiments, at least one R^{1f} is substituted piperidinyl. In certain embodiments, at least one R^{1f} is substituted piperazinyl.

In certain embodiments, at least one R^{1f} is unsubstituted heterocyclyl. In certain embodiments, at least one R^{1f} is unsubstituted 4-6 membered heterocyclyl. In certain embodiments, at least one R^{1f} is unsubstituted oxetanyl. In certain embodiments, at least one R^{1f} is unsubstituted azetidinyl. In certain embodiments, at least one R^{1f} is unsubstituted tetrahydrofuryl. In certain embodiments, at least one R^{1f} is unsubstituted pyrrolidinyl. In certain embodiments, at least one R^{1f} is unsubstituted tetrahydropyranyl. In certain embodiments, at least one R^{1f} is unsubstituted piperidinyl. In certain embodiments, at least one R^{1f} is unsubstituted piperazinyl.

In certain embodiments, R^{1f} is of formula

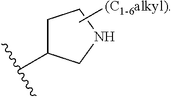

In certain embodiments, R^{1f} is of formula

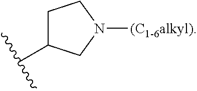

In certain embodiments, R^{1f} is of formula

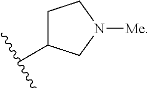 [SMILES: *C1CCN(C)C1]

In certain embodiments, one instance of R^{1f} is unsubstituted methyl; and a second instance of R^{1f} is substituted or unsubstituted pyrrolidinyl. In certain embodiments, one instance of R^{1f} is substituted or unsubstituted propyl; and a second instance of R^{1f} is substituted or unsubstituted —CH₂(cycloalkyl). In certain embodiments, one instance of R^{1f} is substituted or unsubstituted propyl; and a second instance of R^{1f} is substituted or unsubstituted —CH₂(cyclopropyl).

In certain embodiments, two instances of R^{1f} are taken together to form a substituted heterocyclic ring. In certain embodiments, two instances of R^{1f} are taken together to form a substituted 4-7 membered heterocyclic ring. In certain embodiments, two instances of R^{1f} are taken together to form a substituted azetidine. In certain embodiments, two instances of R^{1f} are taken together to form a substituted pyrrolidine. In certain embodiments, two instances of R^{1f} are taken together to form a substituted piperidine. In certain embodiments, two instances of R^{1f} are taken together to form a substituted piperazine. In certain embodiments, two instances of R^{1f} are taken together to form a substituted azepane. In certain embodiments, two instances of R^{1f} are taken together to form a substituted diazepane. In certain embodiments, two instances of R^{1f} are taken together to form an unsubstituted heterocyclic ring.

In certain embodiments, two instances of R^{1f} are taken together to form an unsubstituted 4-7 membered heterocyclic ring. In certain embodiments, two instances of R^{1f} are taken together to form an unsubstituted azetidine. In certain embodiments, two instances of R^{1f} are taken together to form an unsubstituted pyrrolidine. In certain embodiments, two instances of R^{1f} are taken together to form an unsubstituted piperidine. In certain embodiments, two instances of R^{1f} are taken together to form an unsubstituted piperazine. In certain embodiments, two instances of R^{1f} are taken together to form an unsubstituted azepane. In certain embodiments, two instances of R^{1f} are taken together to form an unsubstituted diazepane.

In certain embodiments, two instances of R^{1f} are taken together to form a ring of formula:

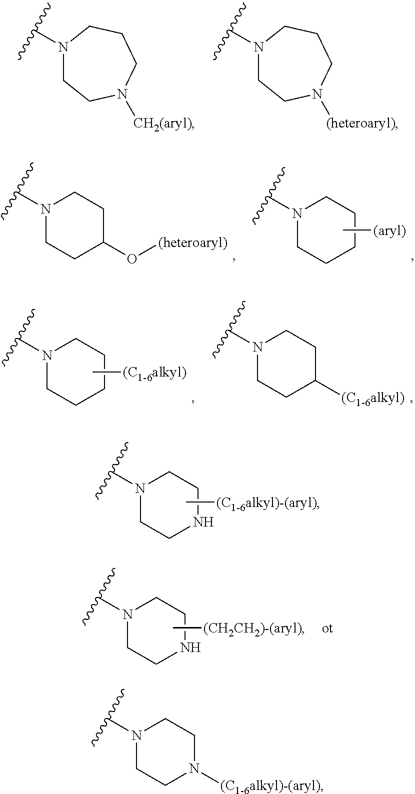

wherein aryl, heteroaryl, or C₁₋₆alkyl are substituted or unsubstituted.

In certain embodiments, two instances of R^{1f} are taken together to form a ring of formula:

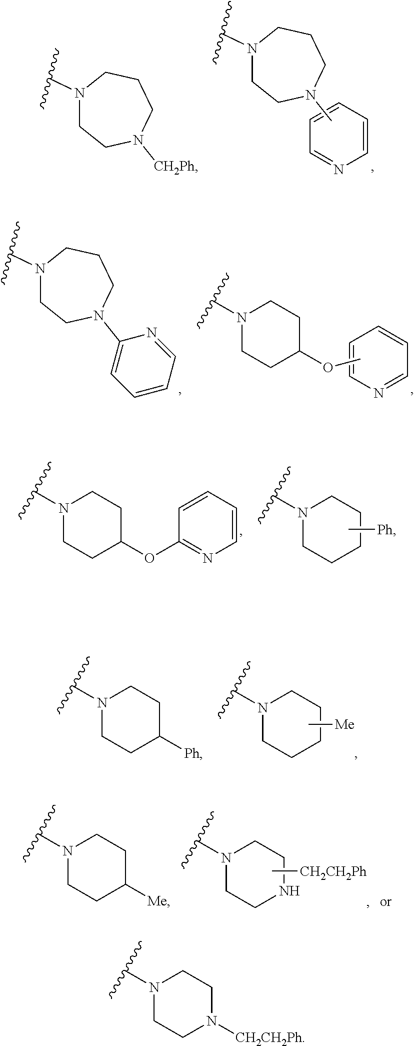

In certain embodiments, a compound of Formula (I) is of Formula (I-d):

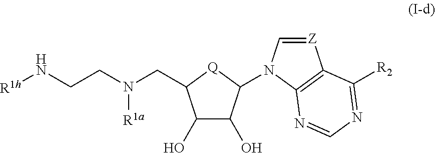

or a pharmaceutically acceptable salt thereof,
wherein:
R^{1h} is substituted or unsubstituted alkyl, substituted or unsubstituted carbocyclyl, substituted or unsubstituted heterocyclyl, substituted or unsubstituted aryl, substituted or unsubstituted heteroaryl, —C(═O)NHR^{1ha}, or a nitrogen protecting group;
R^{1ha} is substituted or unsubstituted aryl; and
R^{1a} is hydrogen, substituted or unsubstituted alkyl, or a nitrogen protecting group.

In certain embodiments, a compound of Formula (I-d) is of Formula (I-d1):

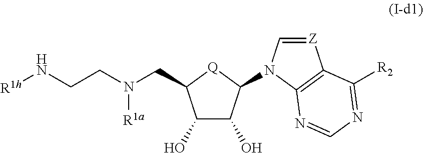

or a pharmaceutically acceptable salt thereof.

In certain embodiments, R^{1h} is substituted or unsubstituted alkyl. In certain embodiments, R^{1h} is substituted or unsubstituted C₁₋₆alkyl. In certain embodiments, R^{1h} is substituted or unsubstituted C₁₋₃alkyl. In certain embodiments, R^{1h} is substituted or unsubstituted methyl. In certain embodiments, R^{1h} is substituted or unsubstituted ethyl. In certain embodiments, R^{1h} is substituted or unsubstituted propyl. In certain embodiments, R^{1h} is substituted or unsubstituted ethyl. In certain embodiments, R^{1h} is substituted or unsubstituted butyl. In certain embodiments, R^{1h} is substituted or unsubstituted isopropyl.

In certain embodiments, R^{1h} is —CH₂(cycloalkyl), wherein cycloalkyl is substituted or unsubstituted. In certain embodiments, R^{1h} is —CH₂(cyclopropyl), wherein cyclopropyl is substituted or unsubstituted.

In certain embodiments, R^{1h} is —CH₂CH₂OH.

In certain embodiments, R^{1h} is —CH(C₁₋₆alkyl)CH₂OH, wherein the C₁₋₆alkyl is substituted or unsubstituted. In certain embodiments, R^{1h} is —CH(Me)CH₂OH.

In certain embodiments, R^{1h} is —CH₂CH₂O(C₁₋₆alkyl), wherein the C₁₋₆alkyl is substituted or unsubstituted. In certain embodiments, R^{1h} is —CH₂CH₂OMe.

In certain embodiments, R^{1h} is —CH(C₁₋₆alkyl)CH₂O(C₁₋₆alkyl), wherein each instance of C₁₋₆alkyl is independently substituted or unsubstituted. In certain embodiments, R^{1h} is —CH(C₁₋₆alkyl)CH₂OMe, wherein the C₁₋₆alkyl is substituted or unsubstituted. In certain embodiments, R^{1h} is substituted —CH(Et)CH₂O(C₁₋₆alkyl), wherein the C₁₋₆alkyl is substituted or unsubstituted. In certain embodiments, R^{1h} is —CH(Me)CH₂O(C₁₋₆alkyl), wherein the C₁₋₆alkyl is substituted or unsubstituted. In certain embodiments, R^{1h} is —CH(Et)CH₂OMe. In certain embodiments, R^{1h} is —CH(Me)CH₂OMe.

In certain embodiments, R^{1h} is —CH₂CH₂O(aryl), wherein the aryl is substituted or unsubstituted. In certain embodiments, R^{1h} is

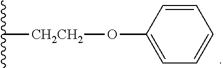

In certain embodiments, R^{1h} is

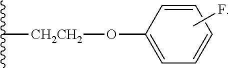

In certain embodiments, R^{1h} is

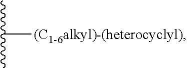

wherein the C₁₋₆alkyl and heterocyclycl are each independently substitutepi₉, substituted. In certain embodiments, R^{1h} is

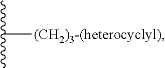

wherein the heterocyclycl is substituted or unsubstituted.

In certain embodiments, R^{1h} is

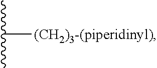

wherein the piperidinyl is substituted or unsubstituted. In certain embodiments, R^{1h} is

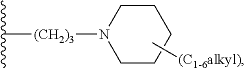

wherein the C₁₋₆alkyl is substituted or unsubstituted. In certain embodiments, R^{1h} is

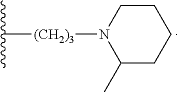

In certain embodiments, R^{1h} is

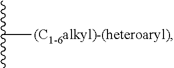

wherein the C₁₋₆alkyl and heteroaryl are each independently substituted or unsubstituted. In certain embodiments, R^{1h} is

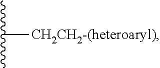

wherein the heteroaryl is substituted or unsubstituted.

In certain embodiments, R^{1h} is

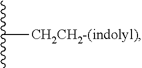

wherein the indolyl is substituted or unsubstituted. In certain embodiments, R^{1h} is

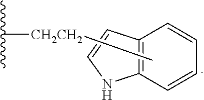

In certain embodiments, R^{1h} is

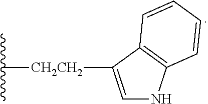

In certain embodiments, R^{1h} is

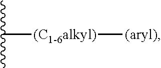

wherein the C₁₋₆alkyl and aryl are each independently substituted or unsubstituted. In certain embodiments, R^{1h} is

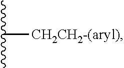

wherein aryl is substituted or unsubstituted. In certain embodiments, R^{1h} is

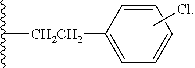

In certain embodiments, R^{1h} is substituted

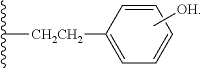

In certain embodiments, R^{1h} is

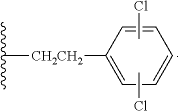

In certain embodiments, R^{1h} is

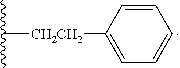

In certain embodiments, R^{1h} is

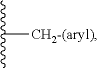

wherein aryl is substituted or unsubstituted. In certain embodiments, R^{1h} is

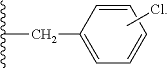

In certain embodiments, R^{1h} is

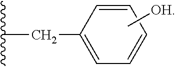

In certain embodiments, R^{1h} is

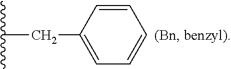 [SMILES: *Cc1ccccc1]

In certain embodiments, R^{1h} is substituted or unsubstituted cycloalkyl. In certain embodiments, R^{1h} is substituted or unsubstituted C₃₋₆ cycloalkyl. In certain embodiments, R^{1h} is substituted or unsubstituted cyclopropyl. In certain embodiments, R^{1h} is substituted or unsubstituted cyclobutyl. In certain embodiments, R^{1h} is substituted or unsubstituted cyclopentyl. In certain embodiments, R^{1h} is substituted or unsubstituted cyclohexyl. In certain embodiments, R^{1h} is of formula

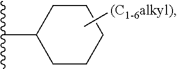

wherein the C₁₋₆alkyl is substituted or unsubstituted. In certain embodiments, R^{1h} is of formula

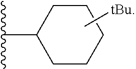

In certain embodiments, R^{1h} is of formula

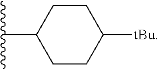 [SMILES: *C1CCC(C(C)(C)C)CC1]

In certain embodiments, R^{1h} is substituted or unsubstituted heterocyclyl. In certain embodiments, R^{1h} is substituted or unsubstituted 4-6 membered heterocyclyl. In certain embodiments, R^{1h} is substituted or unsubstituted oxetanyl. In certain embodiments, R^{1h} is substituted or unsubstituted azetidinyl. In certain embodiments, R^{1h} is substituted or unsubstituted tetrahydrofuryl. In certain embodiments, R^{1h} is substituted or unsubstituted pyrrolidinyl. In certain embodiments, R^{1h} is substituted or unsubstituted tetrahydropyranyl. In certain embodiments, R^{1h} is substituted or unsubstituted piperidinyl. In certain embodiments, R^{1h} is substituted or unsubstituted piperazinyl. In certain embodiments, R^{1h} is of formula

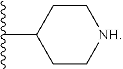 [SMILES: *C1CCNCC1]

In certain embodiments, R^{1h} is substituted or unsubstituted aryl. In certain embodiments, R^{1h} is monosubstituted phenyl. In certain embodiments, R^{1h} is disubstituted phenyl. In certain embodiments, R^{1h} is trisubstituted phenyl. In certain embodiments, R^{1h} is tetrasubstituted phenyl. In certain embodiments, R^{1h} is pentasubstituted phenyl. In certain embodiments, R^{1h} is unsubstituted phenyl.

In certain embodiments, R^{1h} is substituted or unsubstituted heteroaryl. In certain embodiments, R^{1h} is substituted or unsubstituted 5-6 membered heteroaryl. In certain embodiments, R^{1h} is substituted or unsubstituted imidazolyl. In certain embodiments, R^{1h} is substituted or unsubstituted pyrazolyl. In certain embodiments, R^{1h} is substituted or unsubstituted oxazolyl. In certain embodiments, R^{1h} is substituted or unsubstituted thiazolyl. In certain embodiments, R^{1h} is substituted or unsubstituted oxadiazolyl. In certain embodiments, R^{1h} is substituted or unsubstituted thiadiazolyl. In certain embodiments, R^{1h} is substituted or unsubstituted triazolyl. In certain embodiments, R^{1h} is substituted or unsubstituted tetrazolyl. In certain embodiments, R^{1h} is substituted or unsubstituted pyridyl. In certain embodiments, R^{1h} is substituted or unsubstituted pyrazinyl. In certain embodiments, R^{1h} is substituted or unsubstituted pyrimidinyl. In certain embodiments, R^{1h} is substituted or unsubstituted pyridizinyl.

In certain embodiments, R^{1h} is of formula:

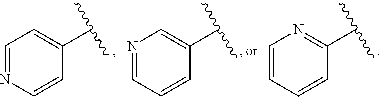

In certain embodiments, R^{1ha} is substituted aryl. In certain embodiments, R^{1ha} is unsubstituted aryl. In certain embodiments, R^{1ha} is monosubstituted phenyl. In certain embodiments, R^{1ha} is disubstituted phenyl. In certain embodiments, R^{1ha} is trisubstituted phenyl. In certain embodiments, R^{1ha} is tetrasubstituted phenyl. In certain embodiments, R^{1ha} is pentasubstituted phenyl. In certain embodiments, R^{1ha} is unsubstituted phenyl.

In certain embodiments, R^{1ha} is of formula

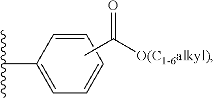

wherein the C₁₋₆alkyl is substituted or unsubstituted.

In certain embodiments, R^{1ha} is of formula

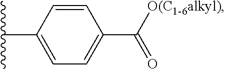

wherein the C₁₋₆alkyl is substituted or unsubstituted.

In certain embodiments, a compound of Formula (I) is of Formula (I-e):

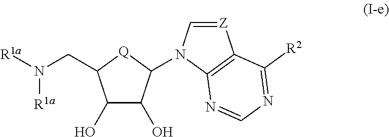

or a pharmaceutically acceptable salt thereof,
wherein:
each R^{1a} is independently —CH₃, —C(═O)R^{Z}, —C(═NH)R^{Z}, substituted or unsubstituted carbocyclyl, substituted or unsubstituted heterocyclyl, or two of R^{1a} can be taken together with their intervening atoms to form a substituted or unsubstituted heterocyclic ring; and R^{Z} is substituted or unsubstituted aryl.

In certain embodiments, a compound of Formula (I-e) is of Formula (I-e1):

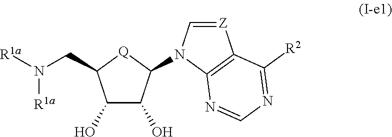

or a pharmaceutically acceptable salt thereof.

In certain embodiments, a compound of Formula (I) is of Formula (I-f):

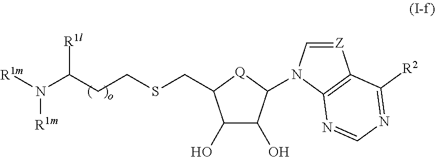

or a pharmaceutically acceptable salt thereof,
wherein:
R^{1l} is —H, —CO₂H, or —CH₂OH;
each R^{1m} is independently hydrogen or two of R^{1m} can be taken together with their intervening atoms to form a substituted or unsubstituted heterocyclic ring; and
o is 0, 1, or 2.

In certain embodiments, a compound of Formula (I-f) is of Formula (I-f1):

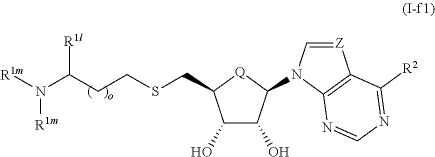

or a pharmaceutically acceptable salt thereof.

In certain embodiments, o is 0. In certain embodiments, o is 1. In certain embodiments, 0 is 2.

In certain embodiments, R^{1l} is hydrogen. In certain embodiments, R^{1l} is —CO₂H. In certain embodiments, R^{1l} is —CH₂OH.

In certain embodiments, two instances of R^{1l} are taken together to form a substituted or unsubstituted heterocyclic ring. In certain embodiments, two instances of R^{1m} are taken together to form a substituted or unsubstituted 4-7 membered heterocyclic ring. In certain embodiments, two instances of R^{1m} are taken together to form a substituted or unsubstituted azetidine. In certain embodiments, two instances of R^{1m} are taken together to form a substituted or unsubstituted pyrrolidine. In certain embodiments, two instances of R^{1m} are taken together to form a substituted or unsubstituted piperidine. In certain embodiments, two instances of R^{1m} are taken together to form a substituted or unsubstituted piperazine. In certain embodiments, two instances of R^{1m} are taken together to form a substituted or unsubstituted azepane. In certain embodiments, two instances of R^{1m} are taken together to form a substituted or unsubstituted diazepane. In certain embodiments, two instances of R^{1m} are taken together with the nitrogen atom to which they are attached to form a ring of formula:

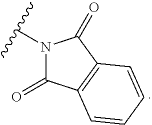 [SMILES: *N1C(=O)c2ccccc2C1=O]

In certain embodiments, a compound of Formula (I) is of Formula (I-h):

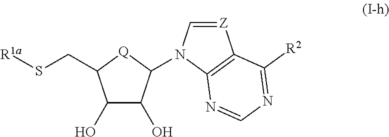

or a pharmaceutically acceptable salt thereof, wherein R^{1a} is C₁₋₃ alkyl optionally substituted with one instance of —CO₂H.

In certain embodiments, a compound of Formula (I-h) is of Formula (I-h1):

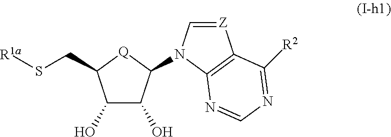

or a pharmaceutically acceptable salt thereof.

In certain embodiments, a provided compound is not a compound as disclosed in the PCT application WO2012/082436.

In certain embodiments, a provided compound is not any one of the following compounds:

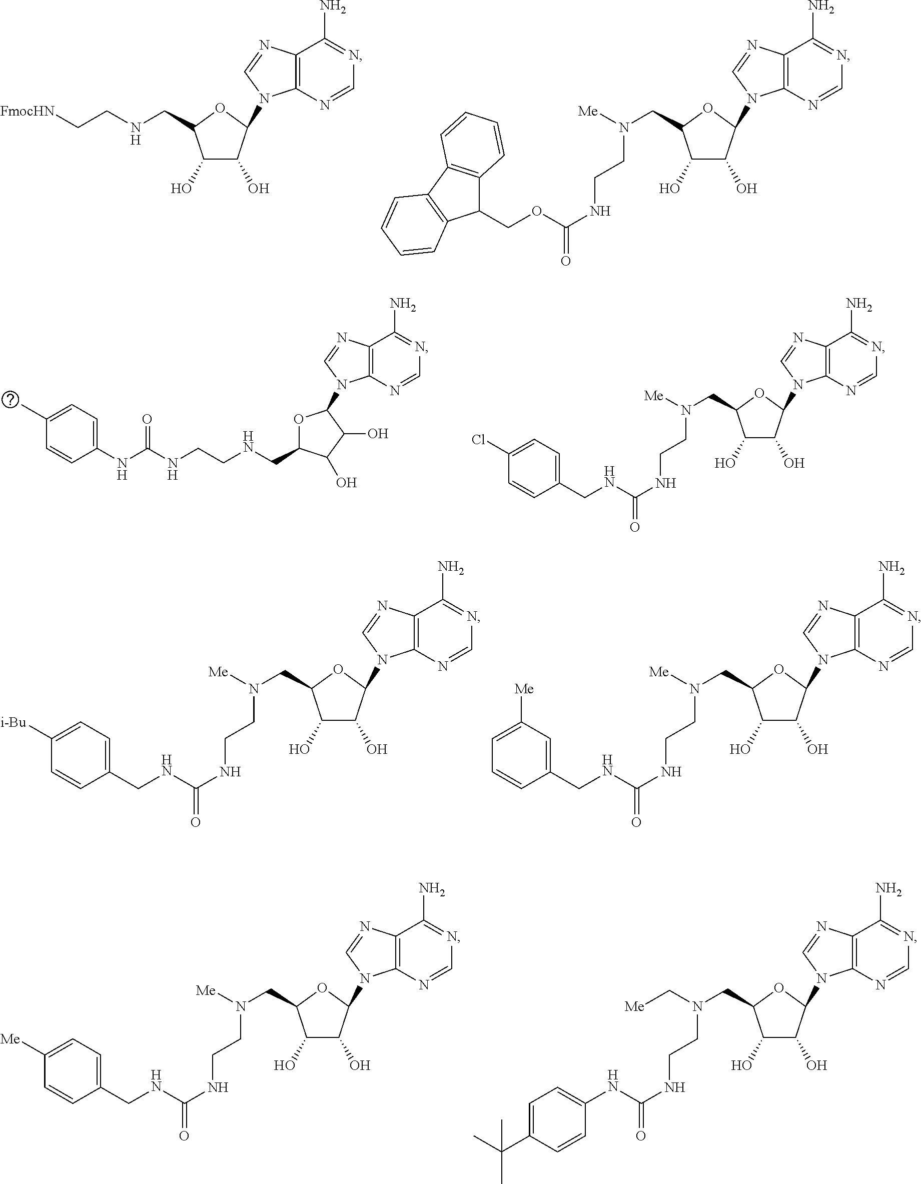

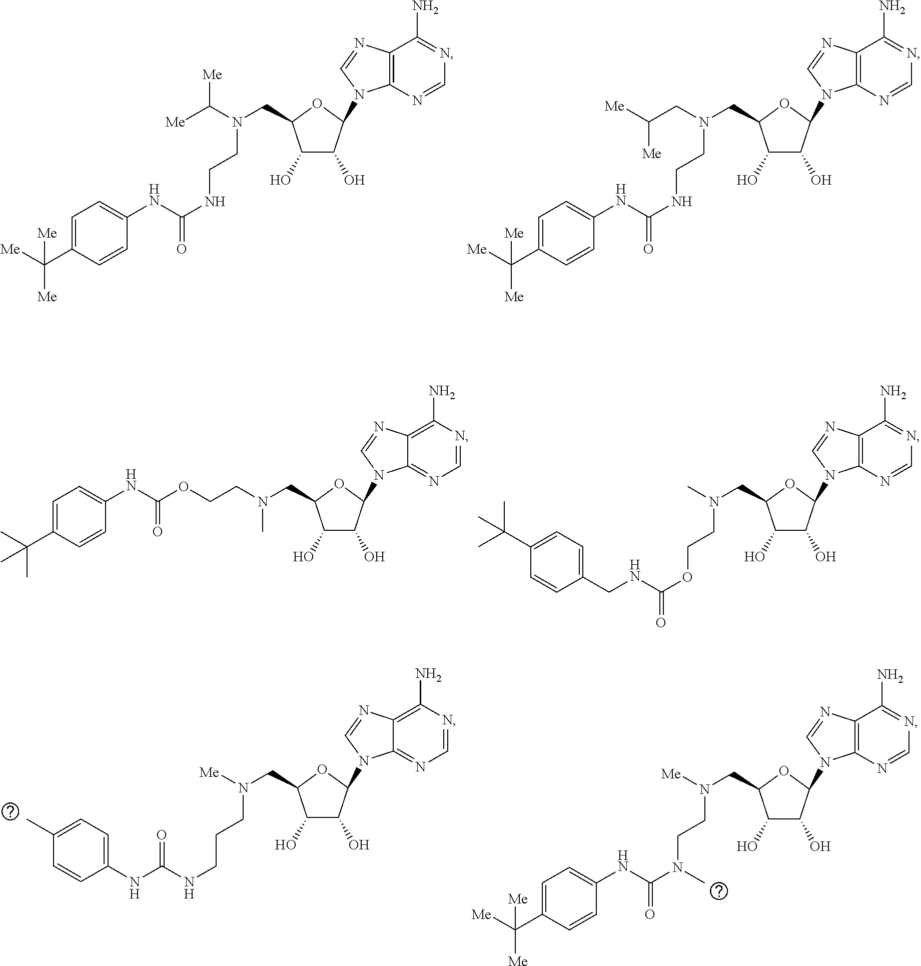 [SMILES: CC(C)N(CCNC(=O)Nc1ccc(C(C)(C)C)cc1)C[C@H]1O[C@@H](n2cnc3c(N)ncnc32)[C@H](O)[C@@H]1O ; CC(C)CN(CCNC(=O)Nc1ccc(C(C)(C)C)cc1)C[C@H]1O[C@@H](n2cnc3c(N)ncnc32)[C@H](O)[C@@H]1O ; CN(CCOC(=O)Nc1ccc(C(C)(C)C)cc1)C[C@H]1O[C@@H](n2cnc3c(N)ncnc32)[C@H](O)[C@@H]1O ; CN(CCOC(=O)NCc1ccc(C(C)(C)C)cc1)C[C@H]1O[C@@H](n2cnc3c(N)ncnc32)[C@H](O)[C@@H]1O ; Cc1ccc(NC(=O)NCCCN(C)C[C@H]2O[C@@H](n3cnc4c(N)ncnc43)[C@H](O)[C@@H]2O)cc1 ; CN(CCN(C)C(=O)Nc1ccc(C(C)(C)C)cc1)C[C@H]1O[C@@H](n2cnc3c(N)ncnc32)[C@H](O)[C@@H]1O]

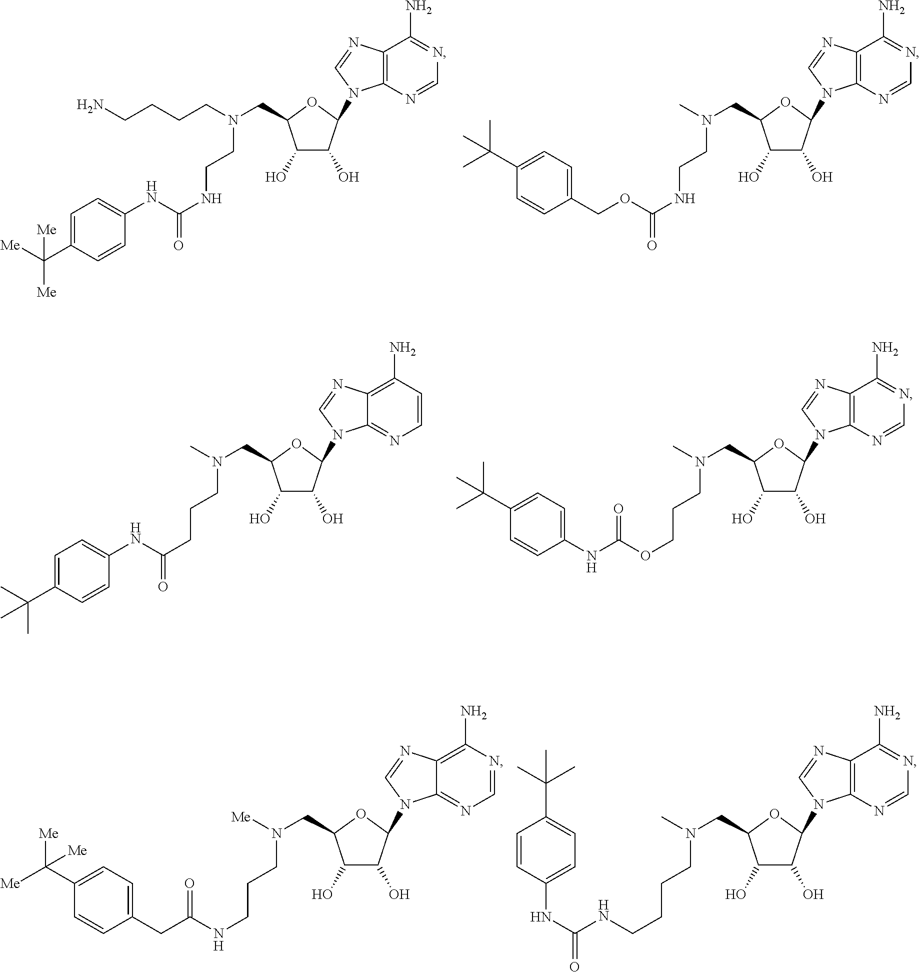

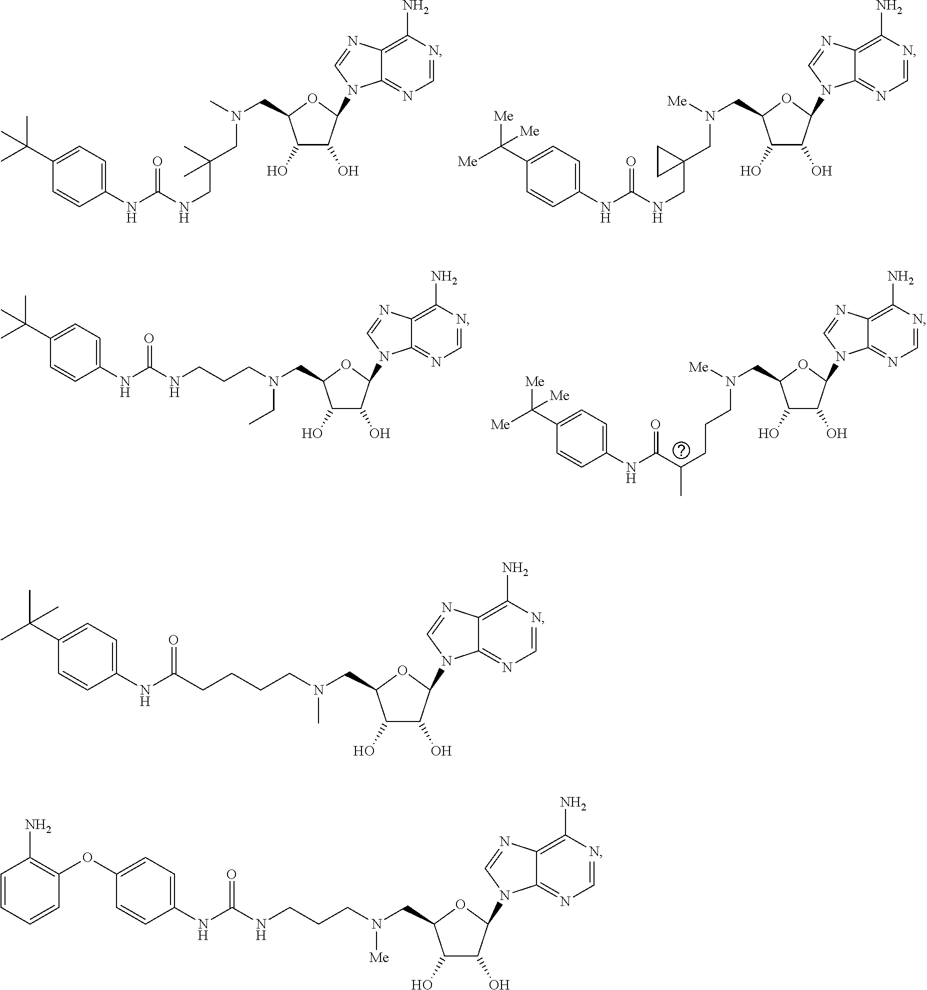

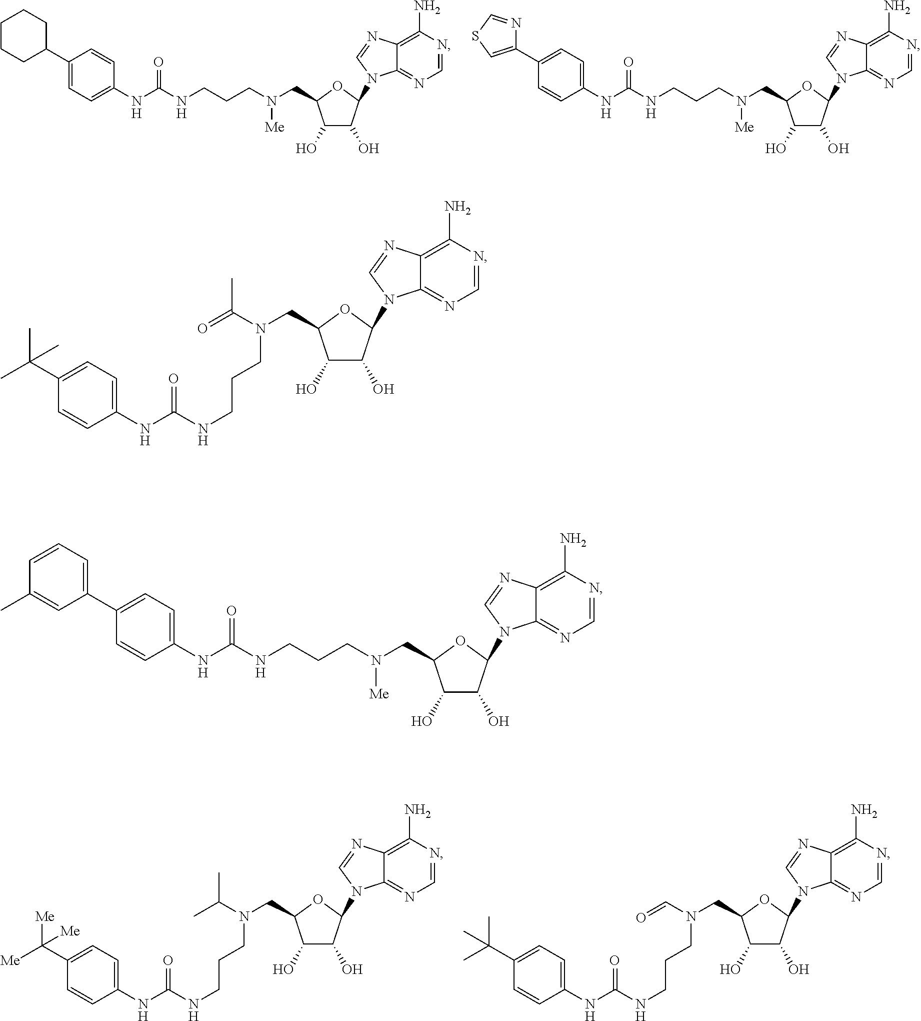

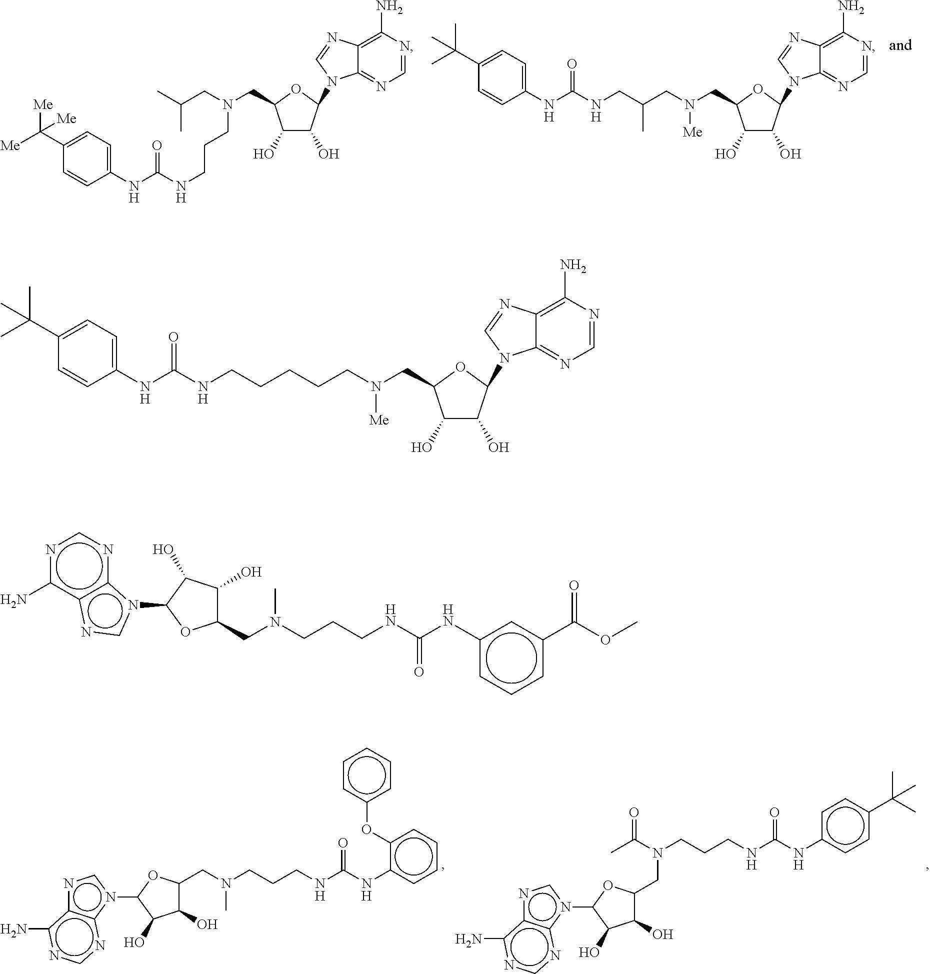

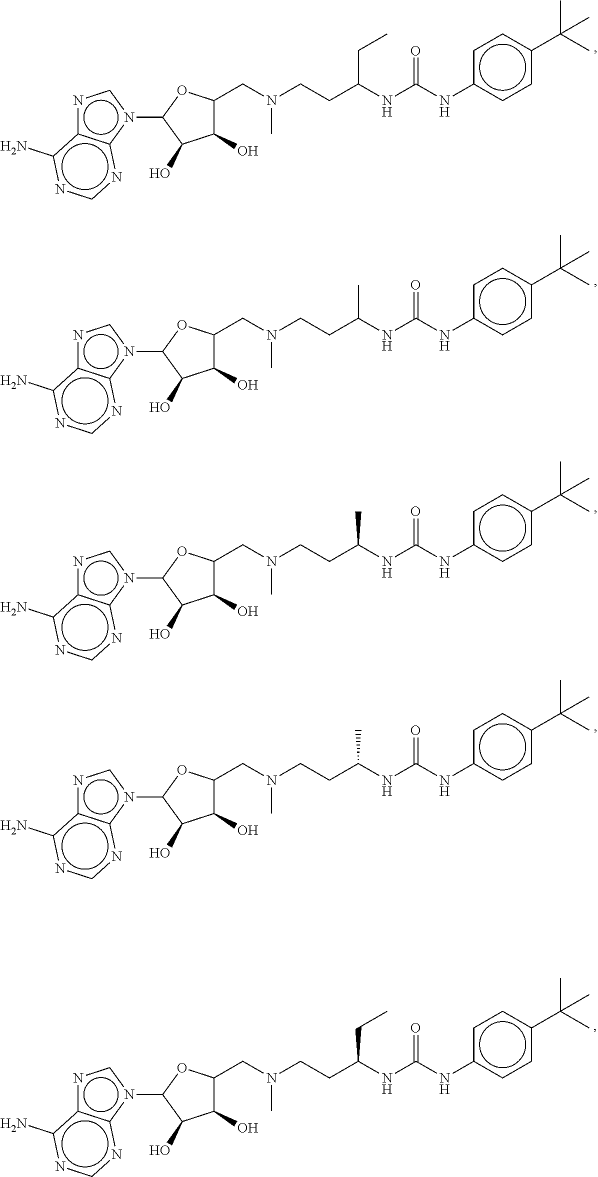

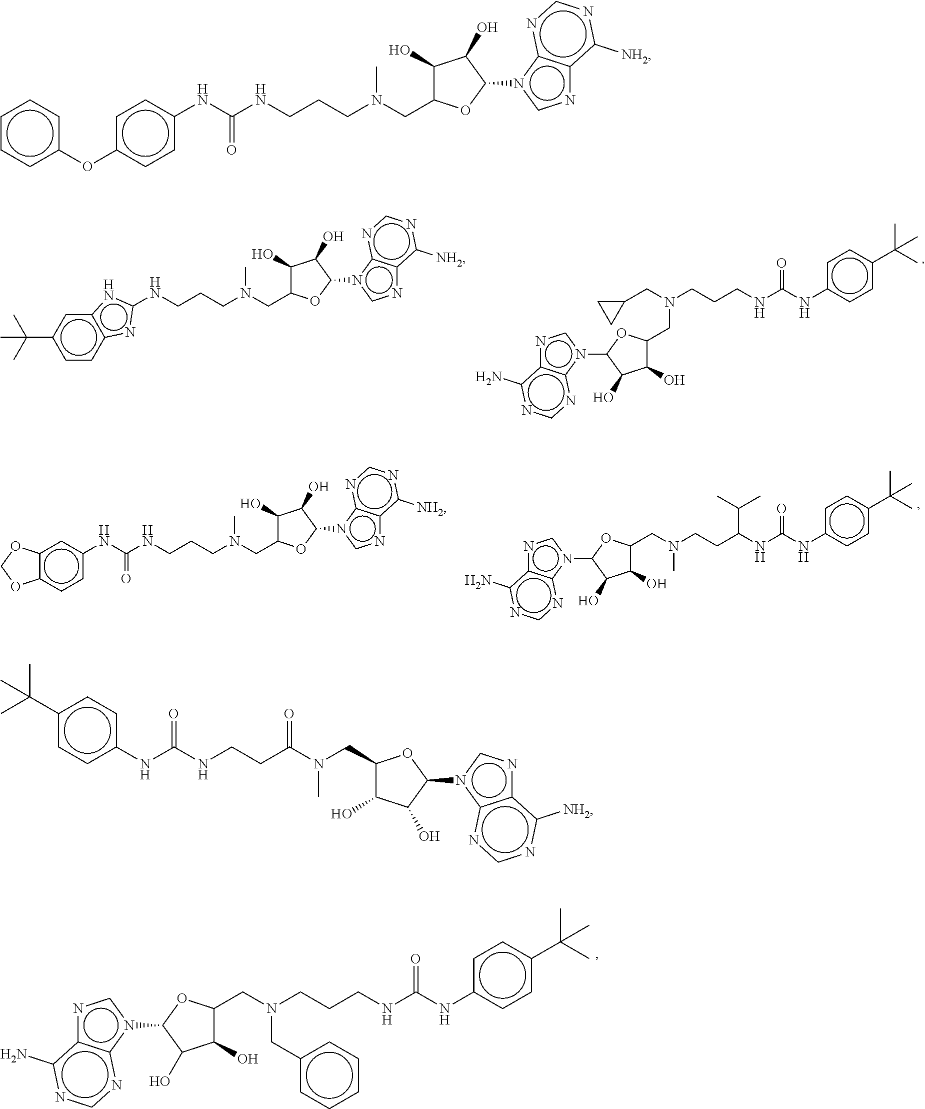

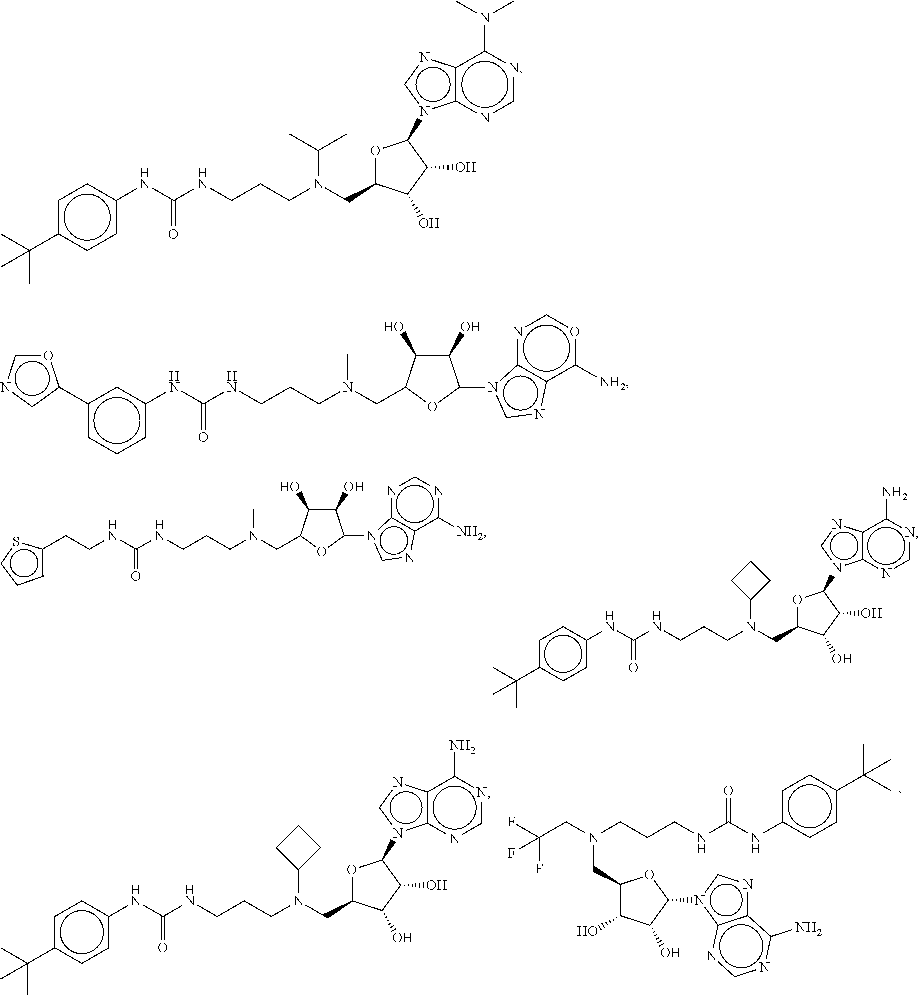

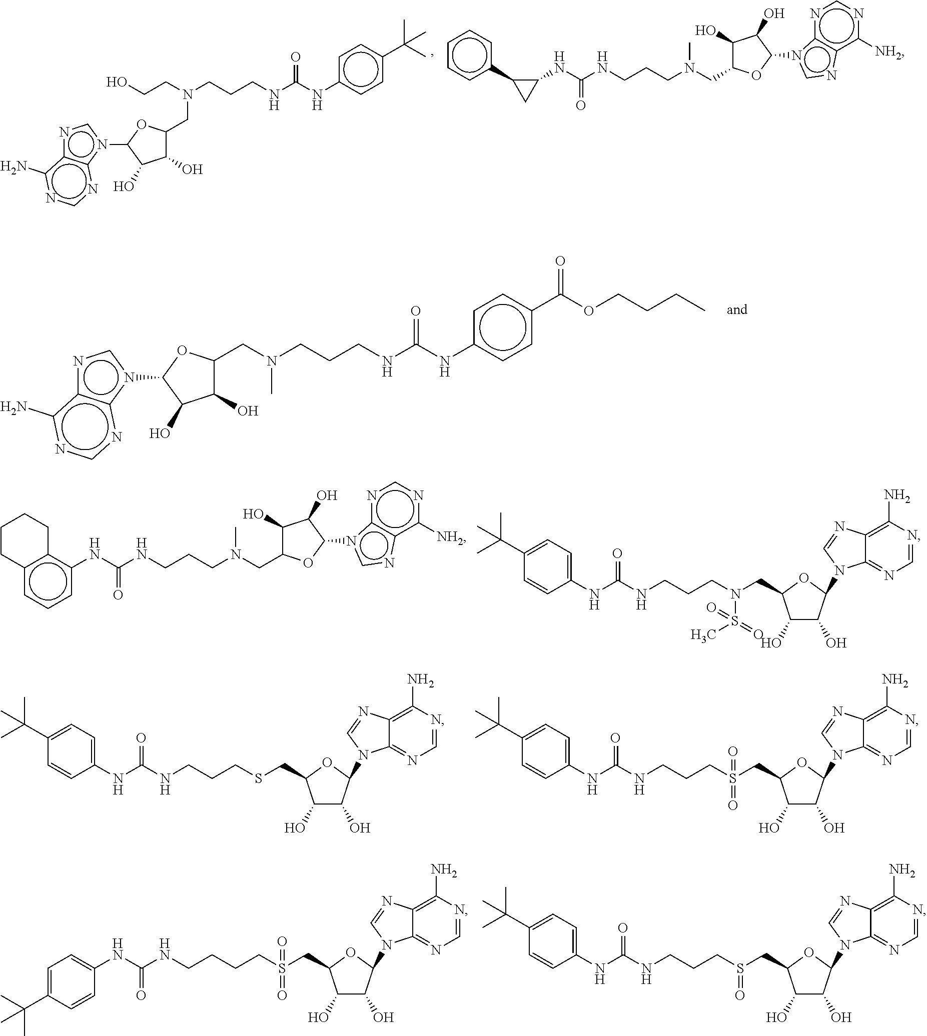

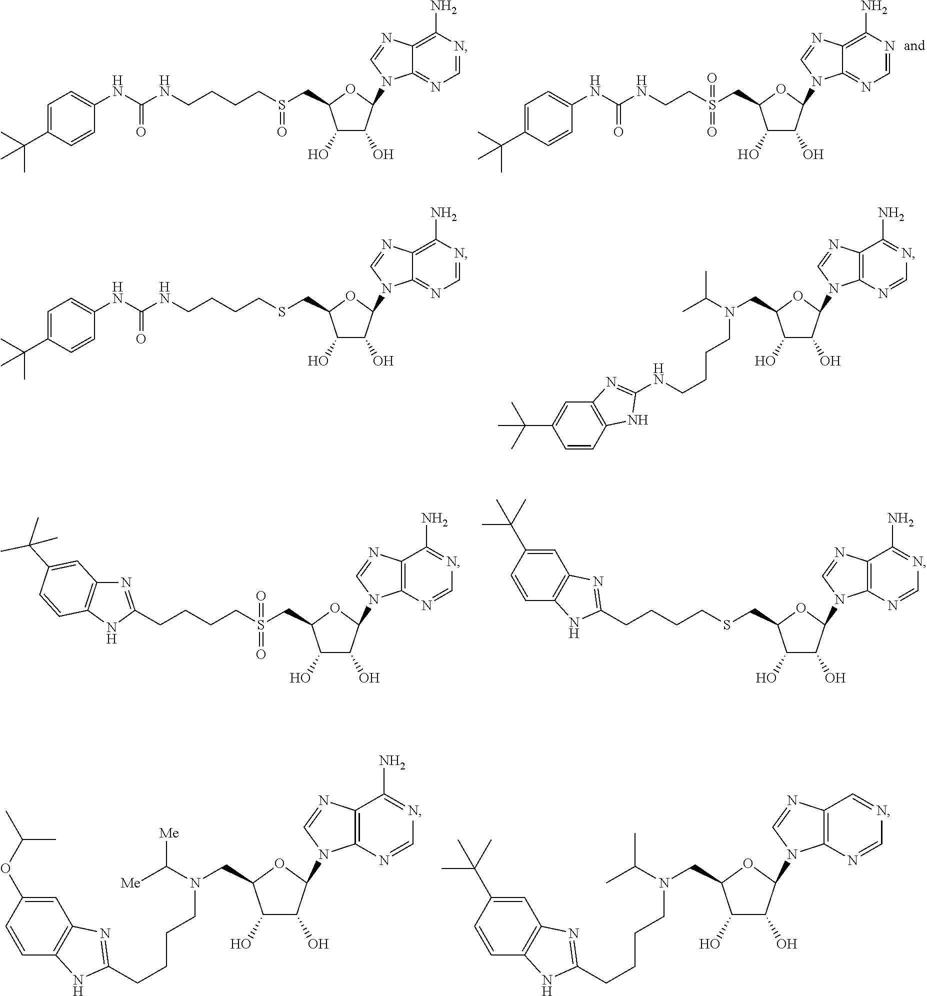

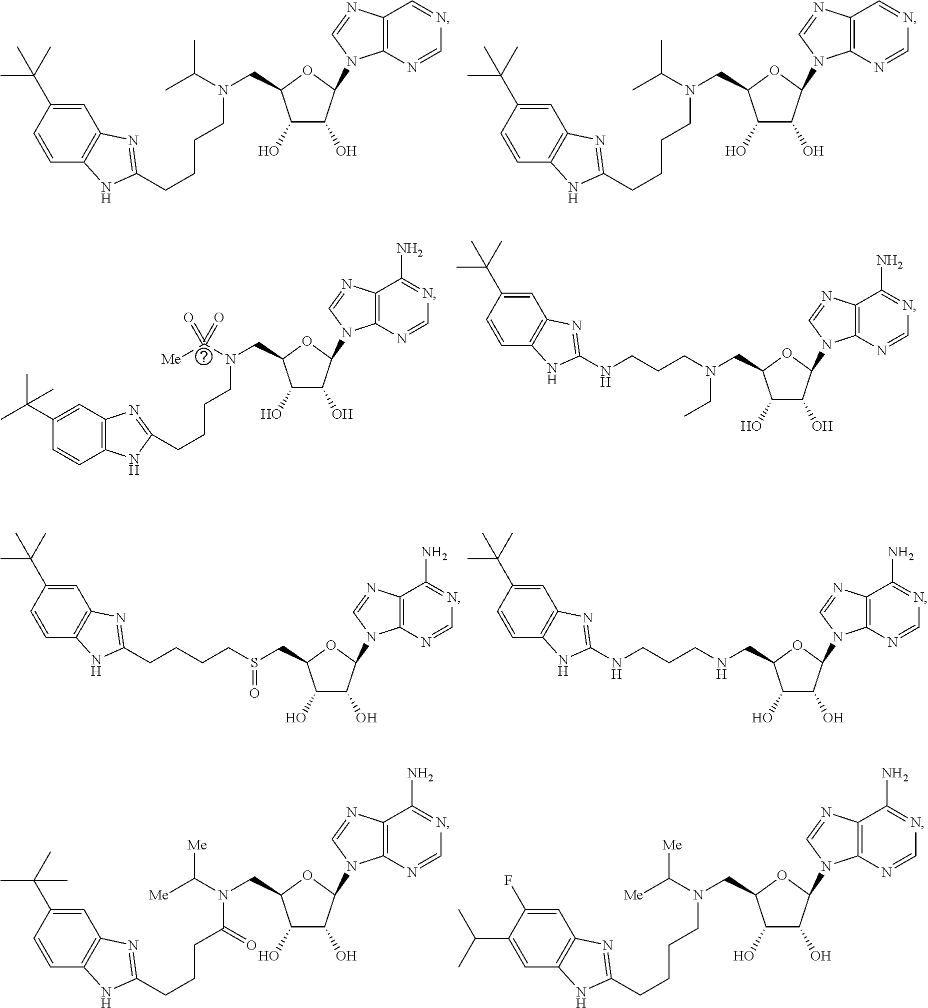

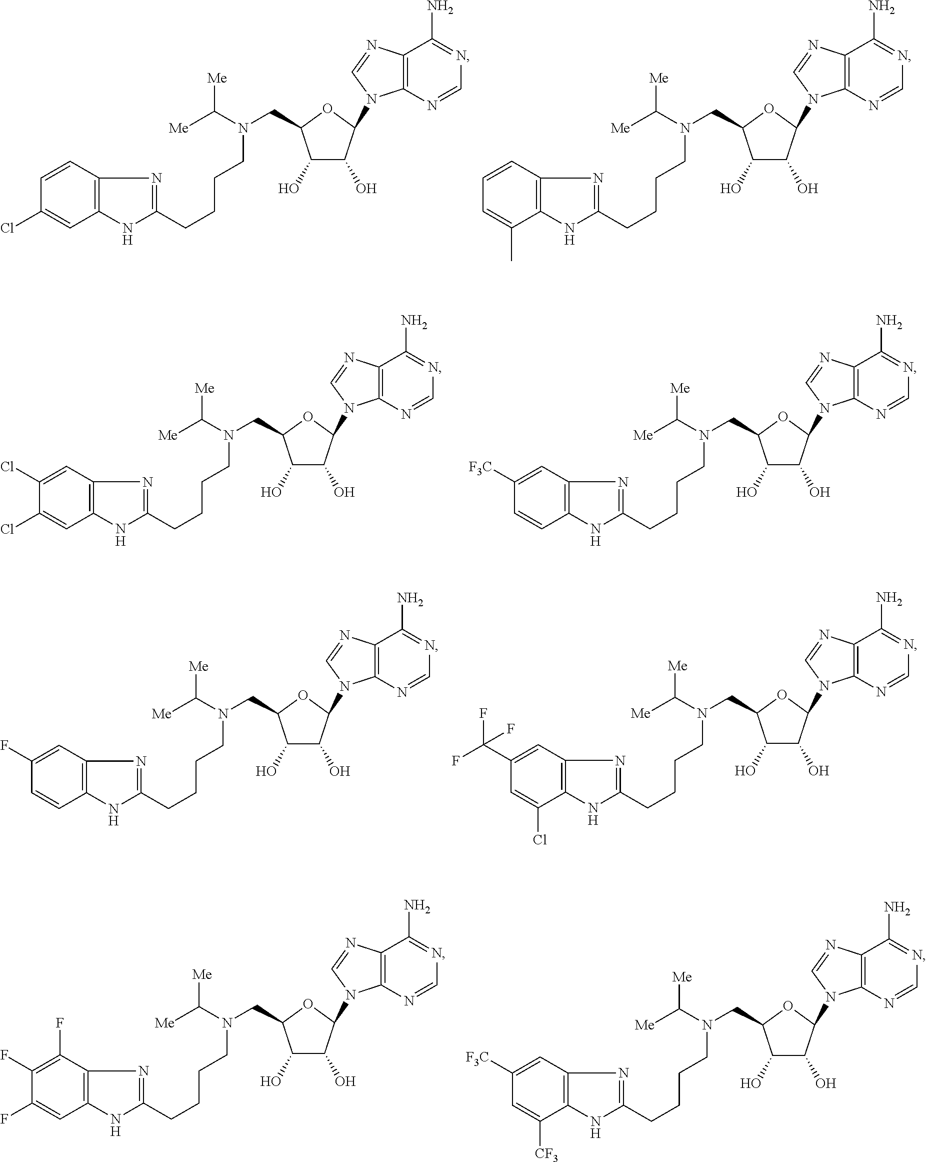

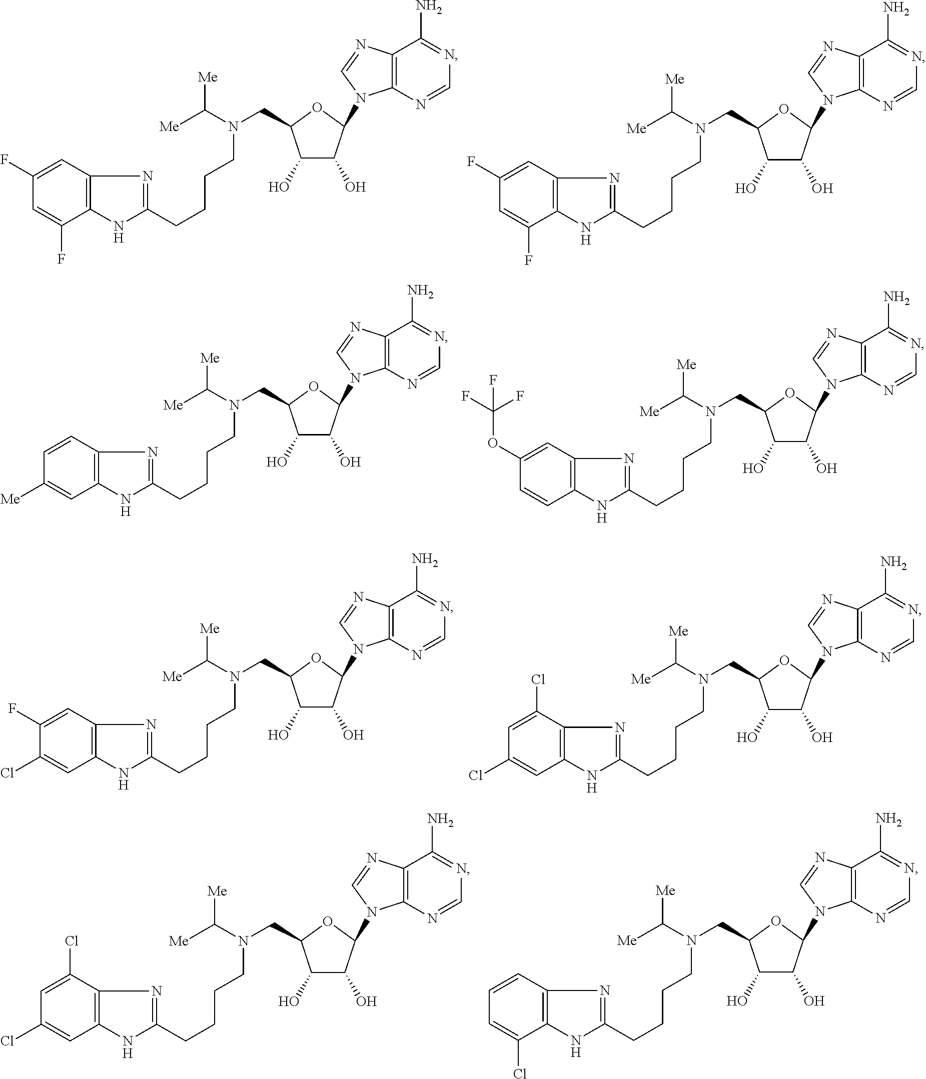

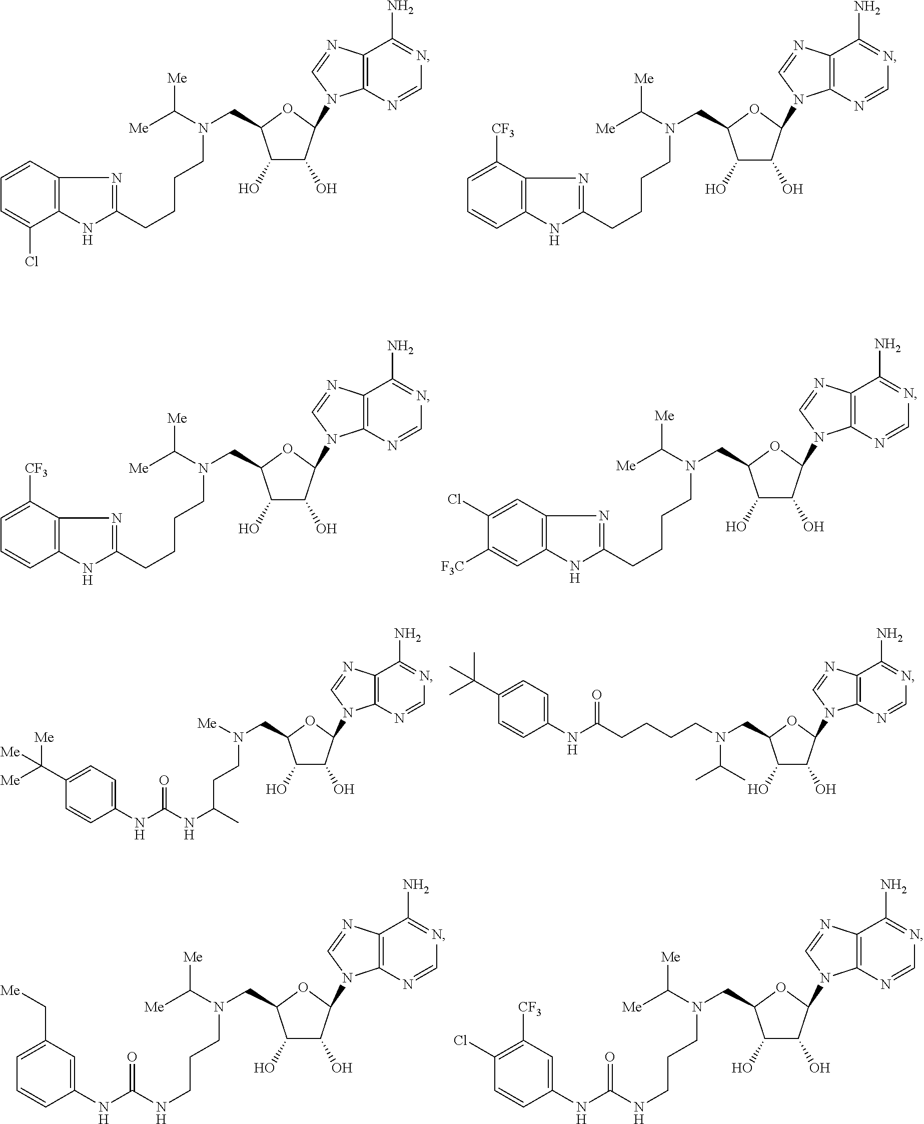

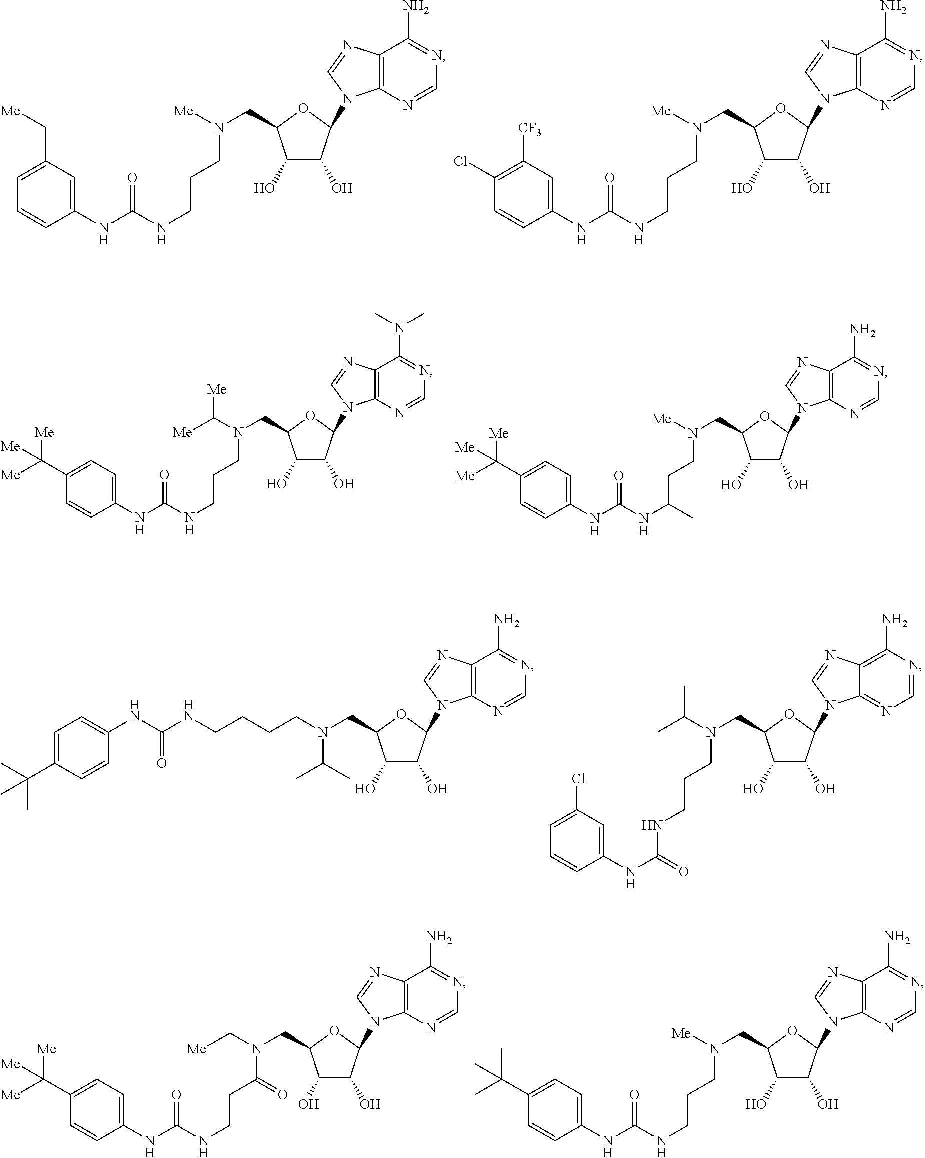

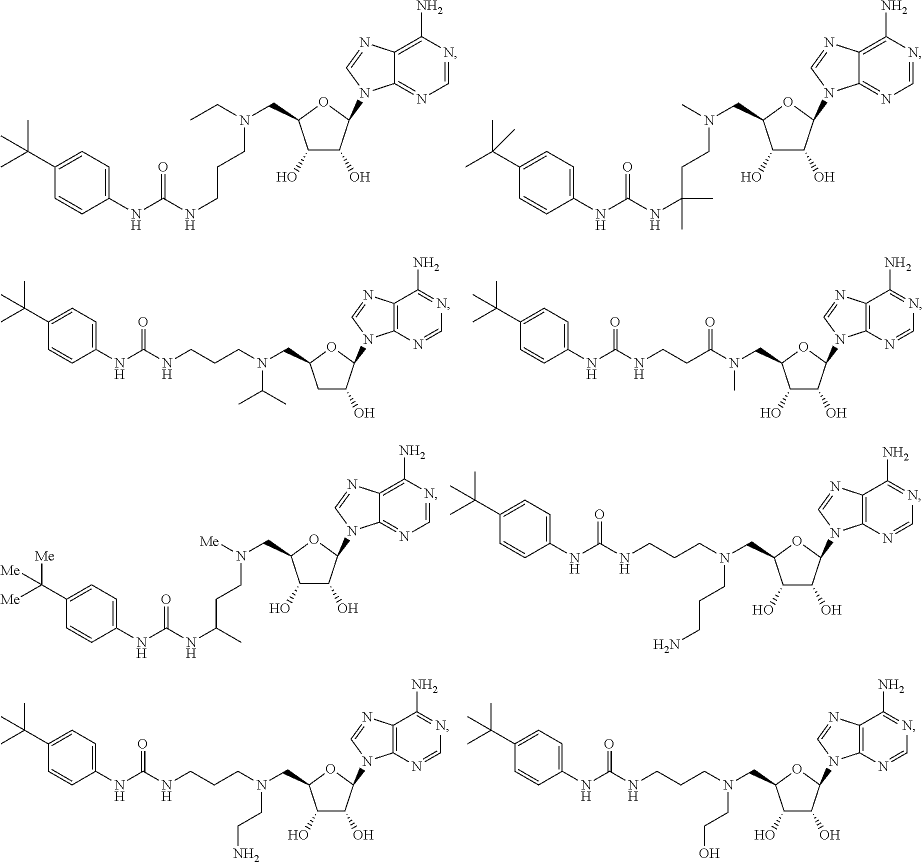

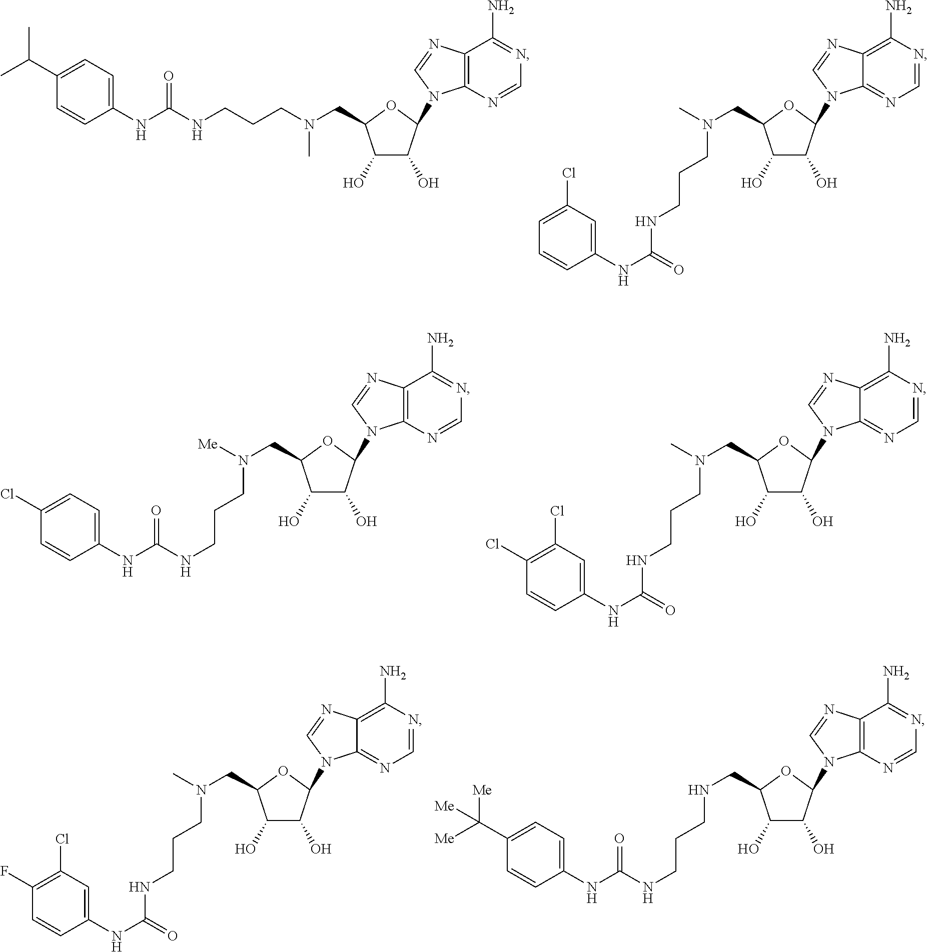 [SMILES: CC(C)c1ccc(NC(=O)NCCCN(C)C[C@H]2O[C@@H](n3cnc4c(N)ncnc43)[C@H](O)[C@@H]2O)cc1 ; CN(CCCNC(=O)Nc1cccc(Cl)c1)C[C@H]1O[C@@H](n2cnc3c(N)ncnc32)[C@H](O)[C@@H]1O ; CN(CCCNC(=O)Nc1ccc(Cl)cc1)C[C@H]1O[C@@H](n2cnc3c(N)ncnc32)[C@H](O)[C@@H]1O ; CN(CCCNC(=O)Nc1ccc(Cl)c(Cl)c1)C[C@H]1O[C@@H](n2cnc3c(N)ncnc32)[C@H](O)[C@@H]1O ; CN(CCCNC(=O)Nc1ccc(F)c(Cl)c1)C[C@H]1O[C@@H](n2cnc3c(N)ncnc32)[C@H](O)[C@@H]1O ; CC(C)(C)c1ccc(NC(=O)NCCCNC[C@H]2O[C@@H](n3cnc4c(N)ncnc43)[C@H](O)[C@@H]2O)cc1]

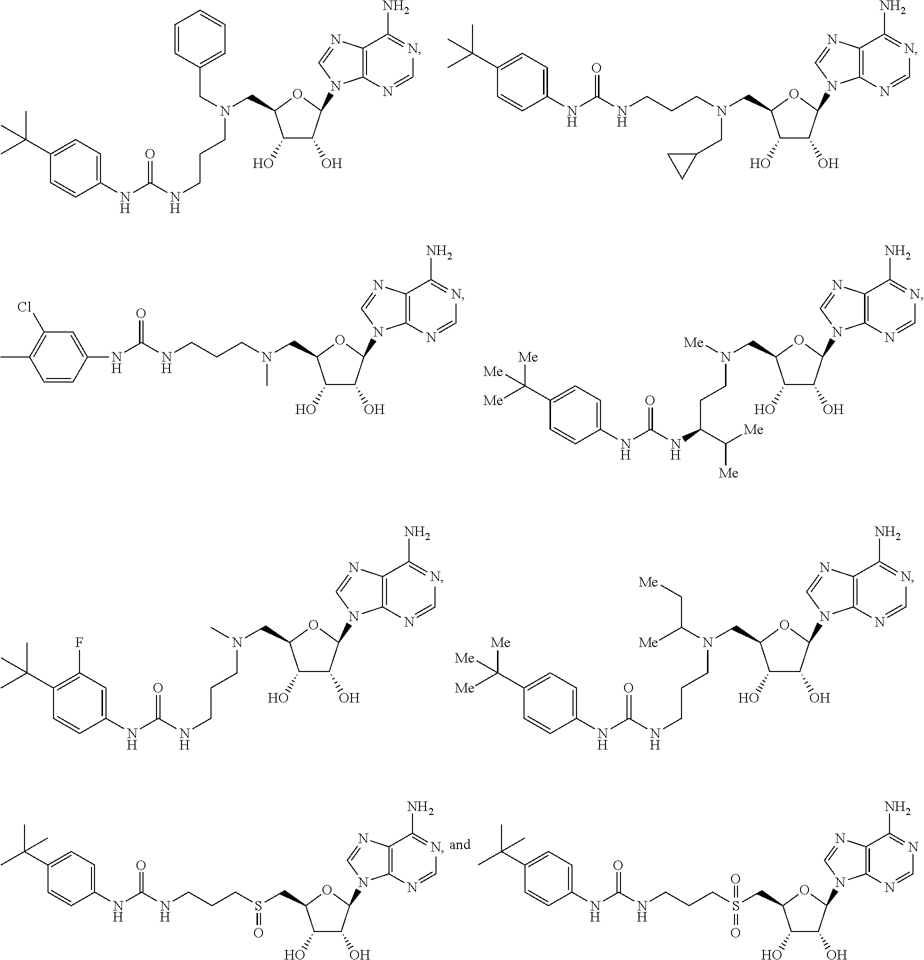

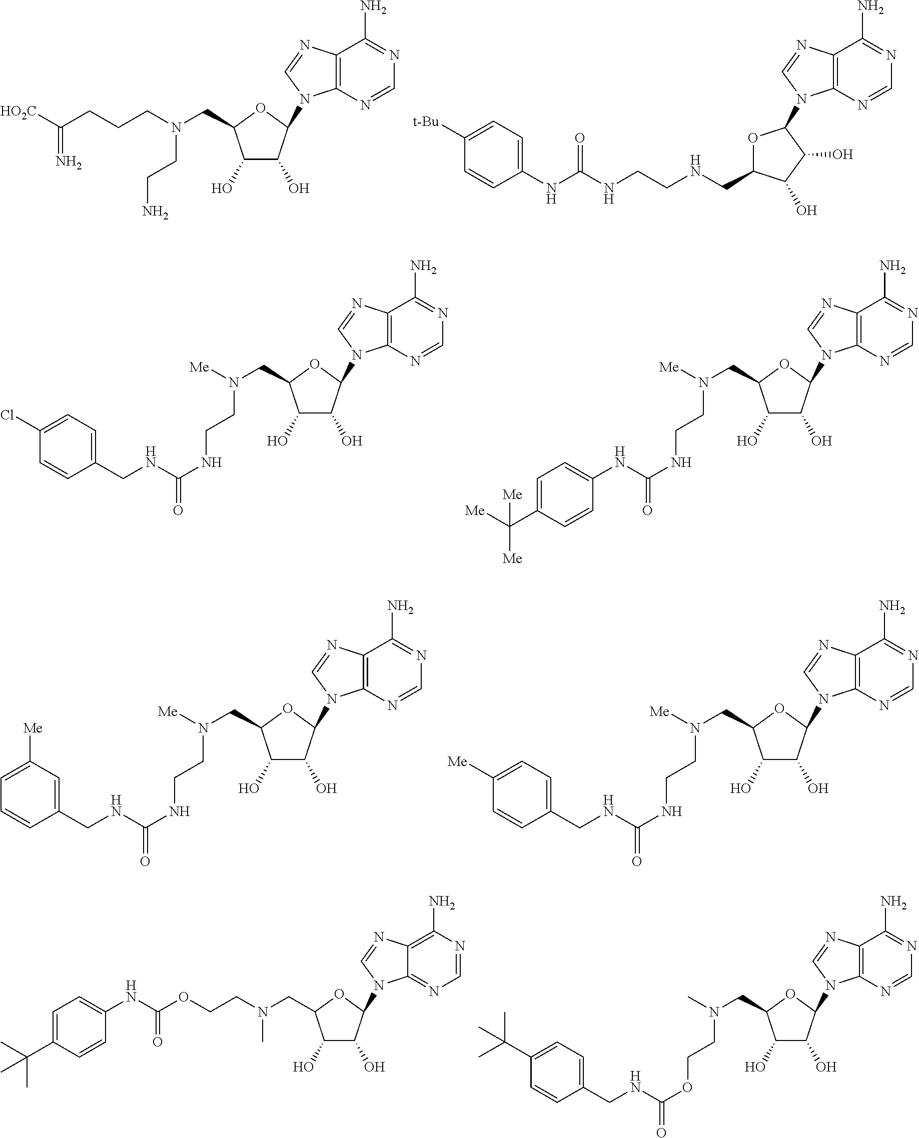

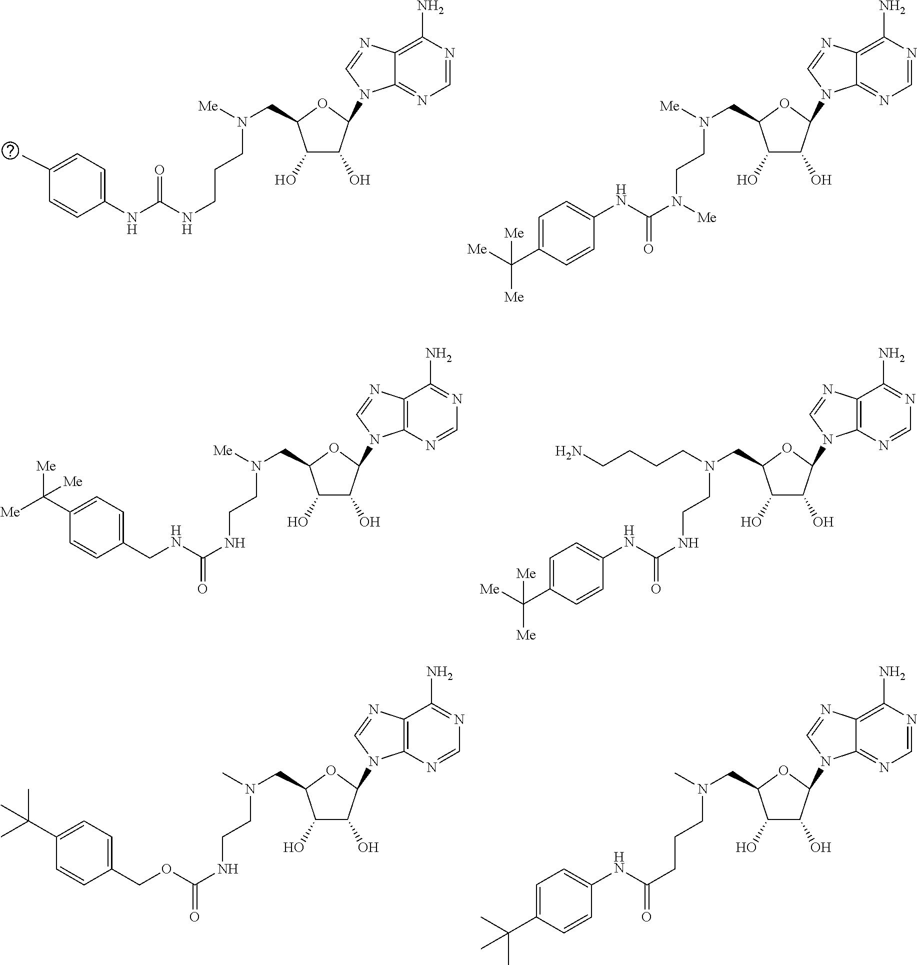 [SMILES: Cc1ccc(NC(=O)NCCCN(C)C[C@H]2O[C@@H](n3cnc4c(N)ncnc43)[C@H](O)[C@@H]2O)cc1 ; CN(CCN(C)C(=O)Nc1ccc(C(C)(C)C)cc1)C[C@H]1O[C@@H](n2cnc3c(N)ncnc32)[C@H](O)[C@@H]1O ; CN(CCNC(=O)NCc1ccc(C(C)(C)C)cc1)C[C@H]1O[C@@H](n2cnc3c(N)ncnc32)[C@H](O)[C@@H]1O ; CC(C)(C)c1ccc(NC(=O)NCCN(CCCCN)C[C@H]2O[C@@H](n3cnc4c(N)ncnc43)[C@H](O)[C@@H]2O)cc1 ; CN(CCNC(=O)OCc1ccc(C(C)(C)C)cc1)C[C@H]1O[C@@H](n2cnc3c(N)ncnc32)[C@H](O)[C@@H]1O ; CN(CCCC(=O)Nc1ccc(C(C)(C)C)cc1)C[C@H]1O[C@@H](n2cnc3c(N)ncnc32)[C@H](O)[C@@H]1O]

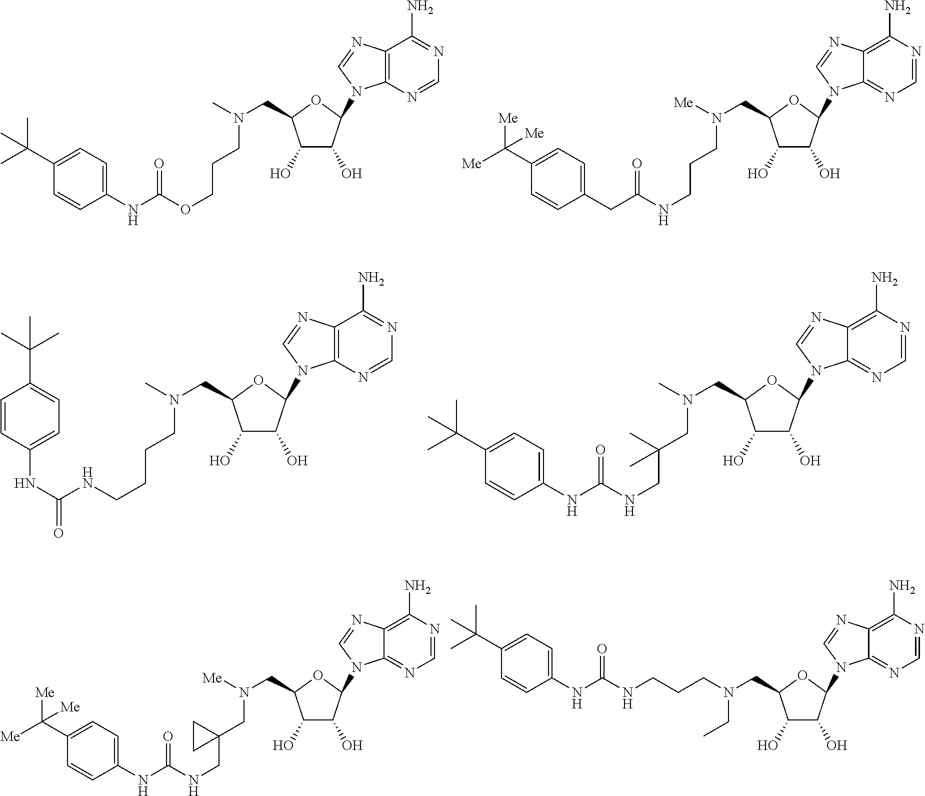

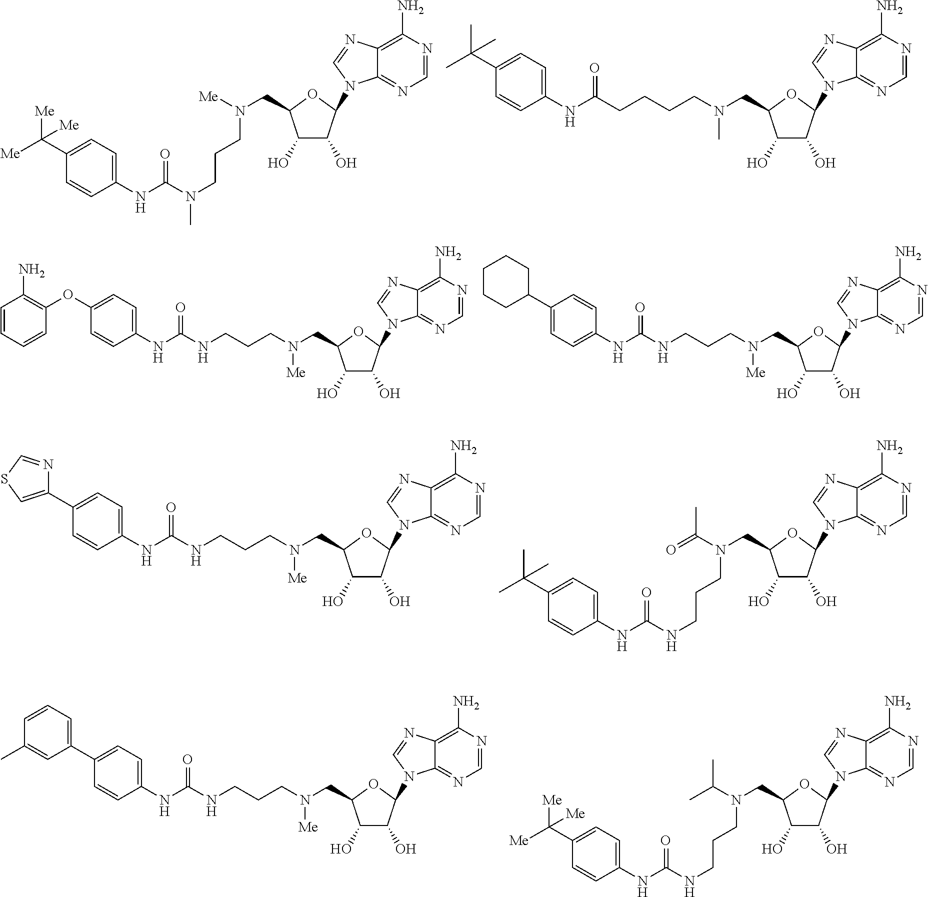

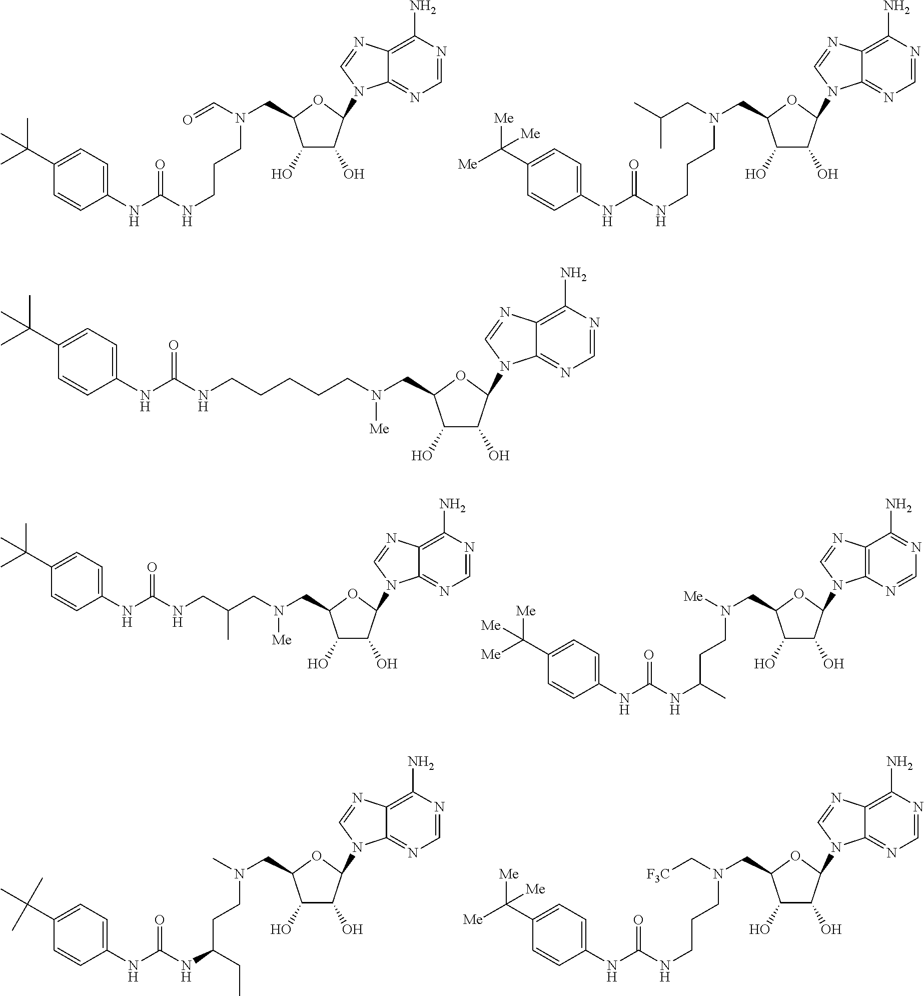

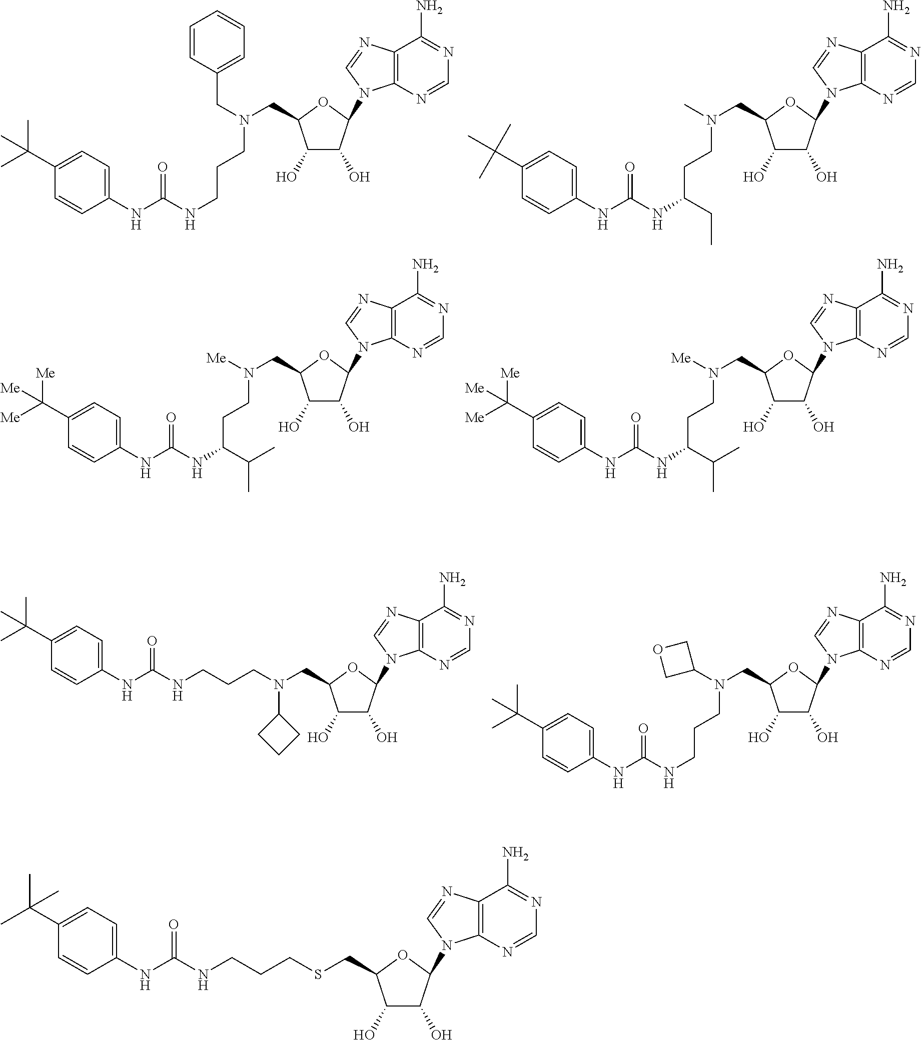

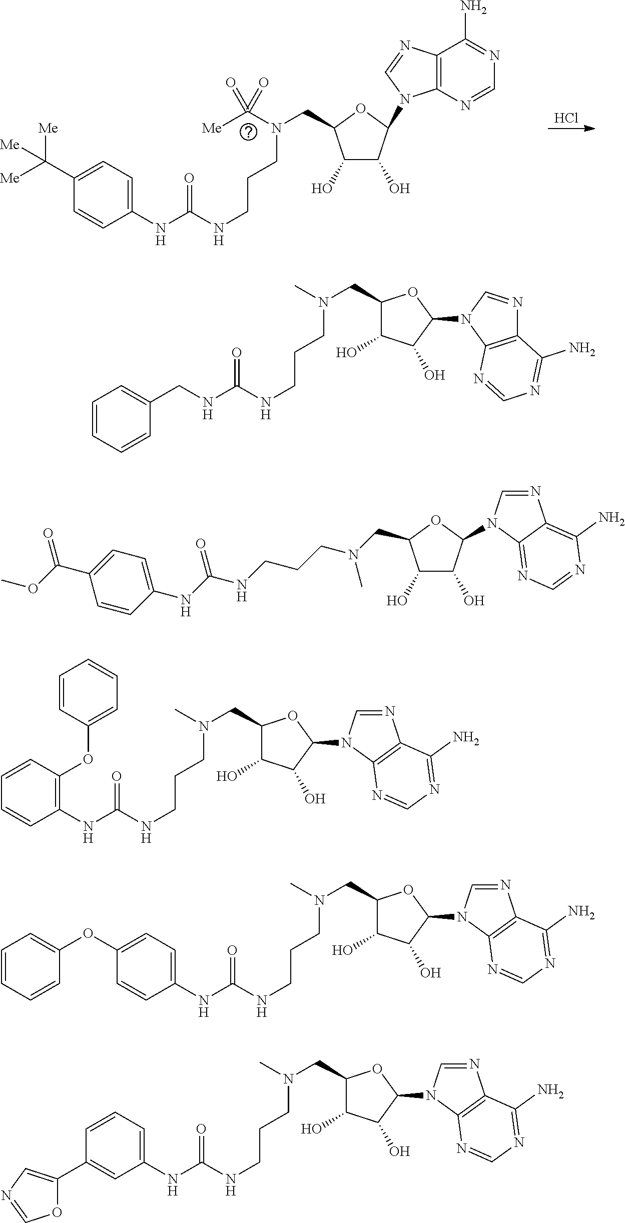

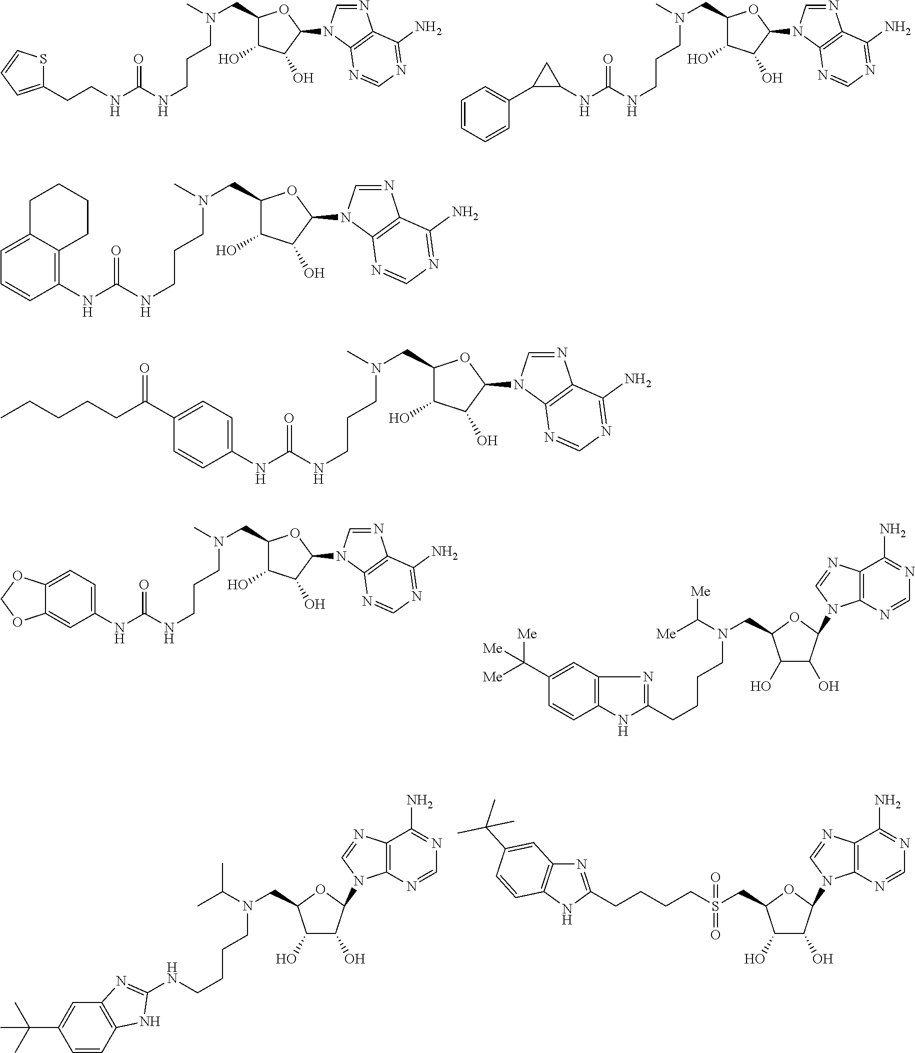

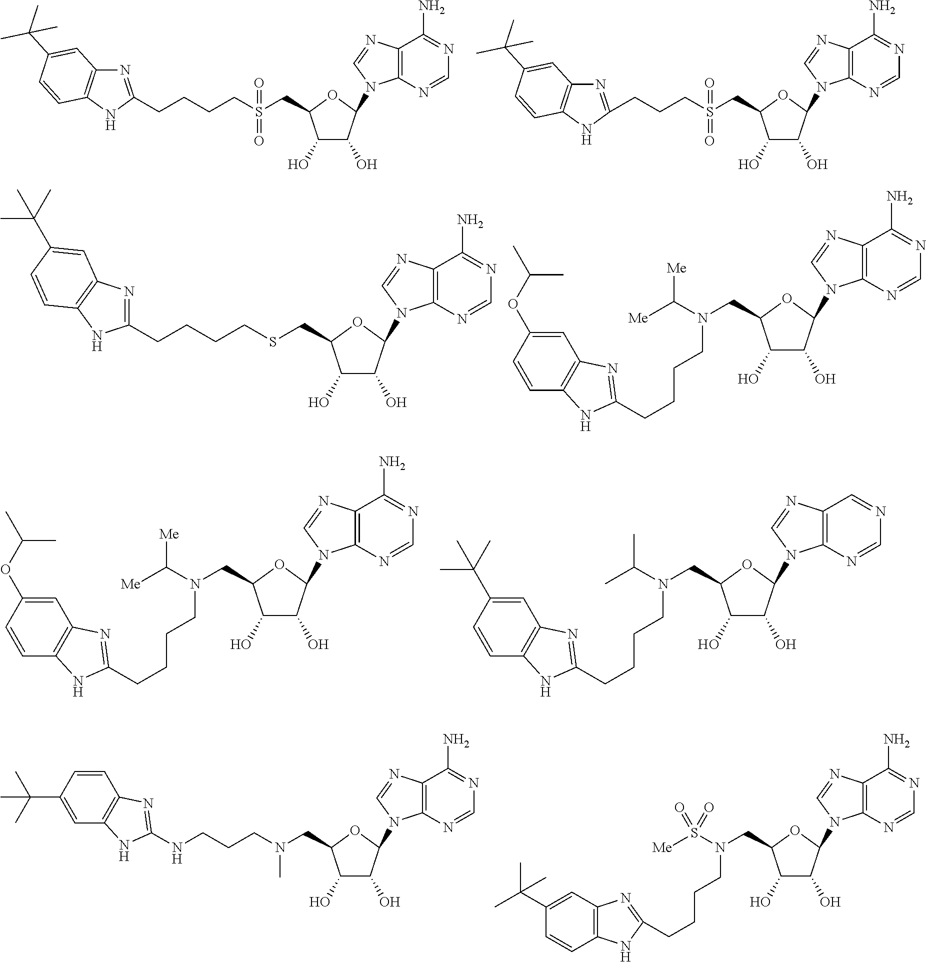

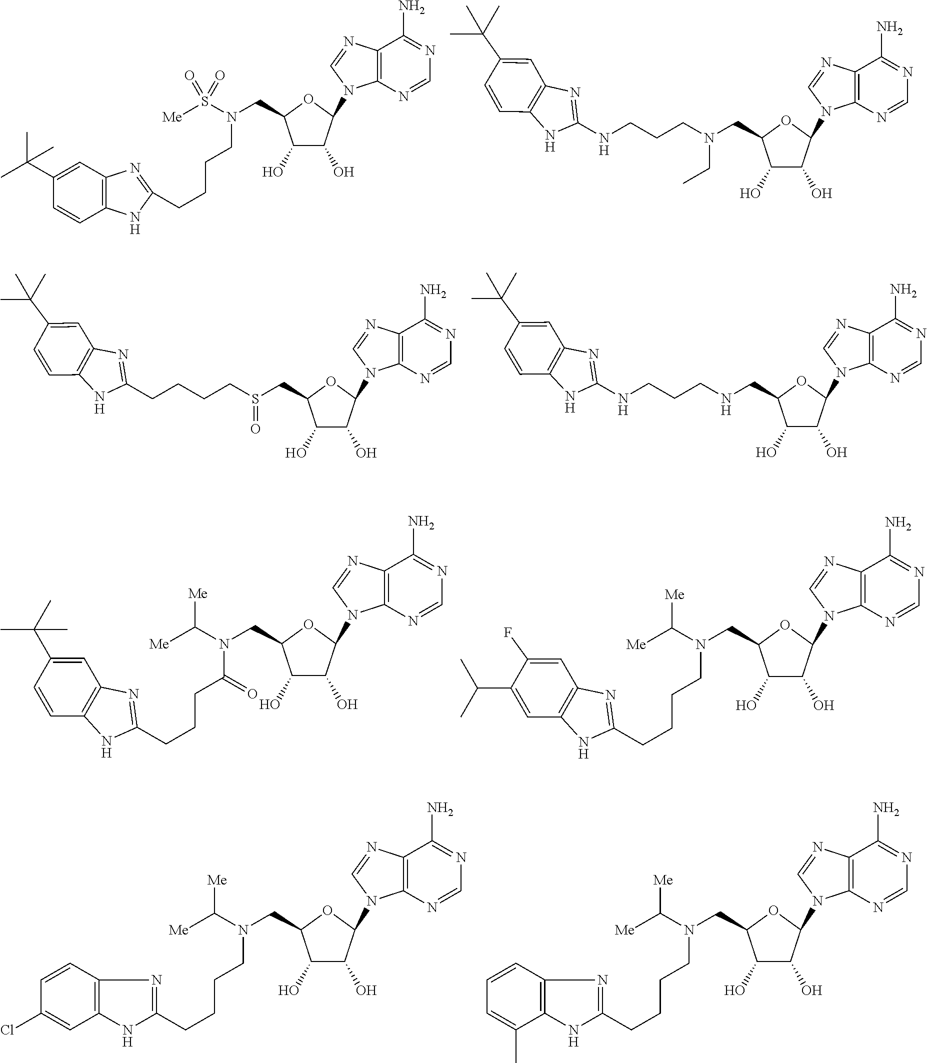

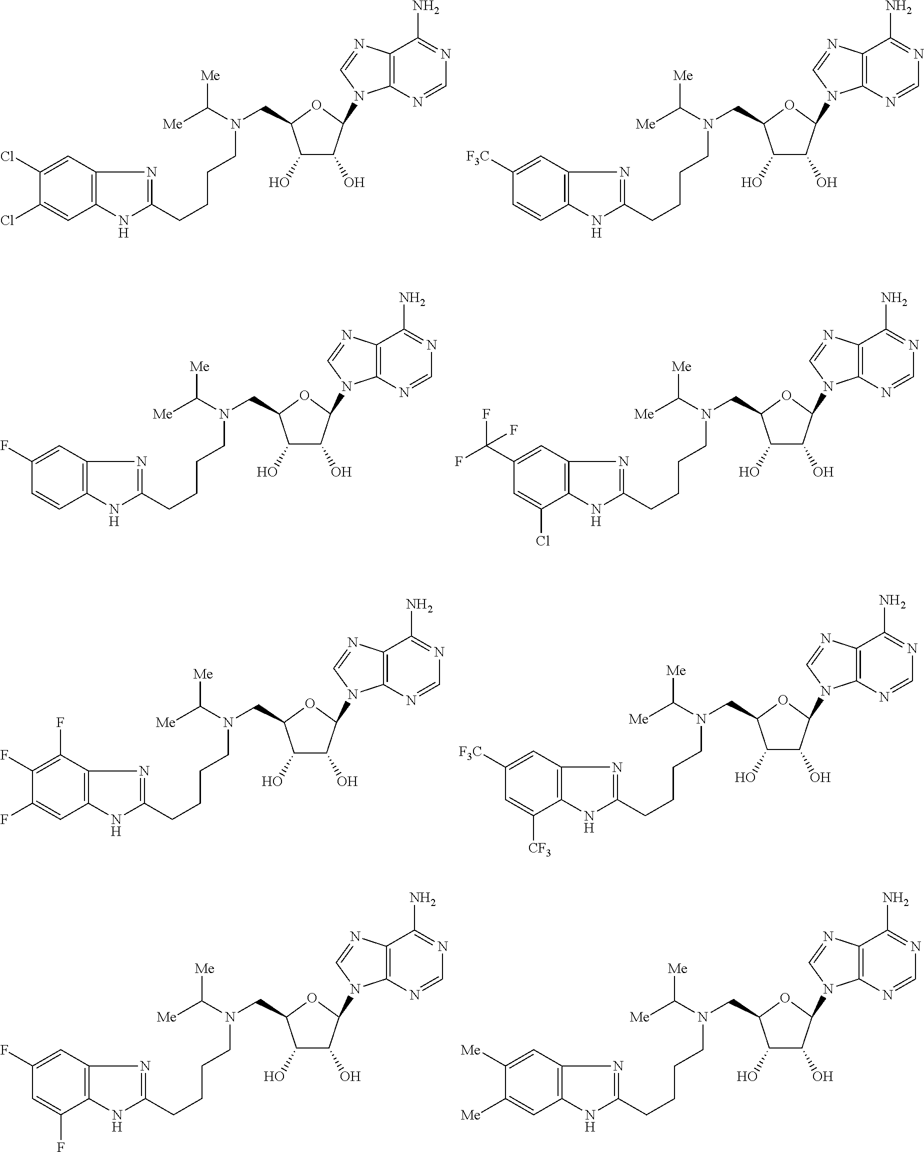

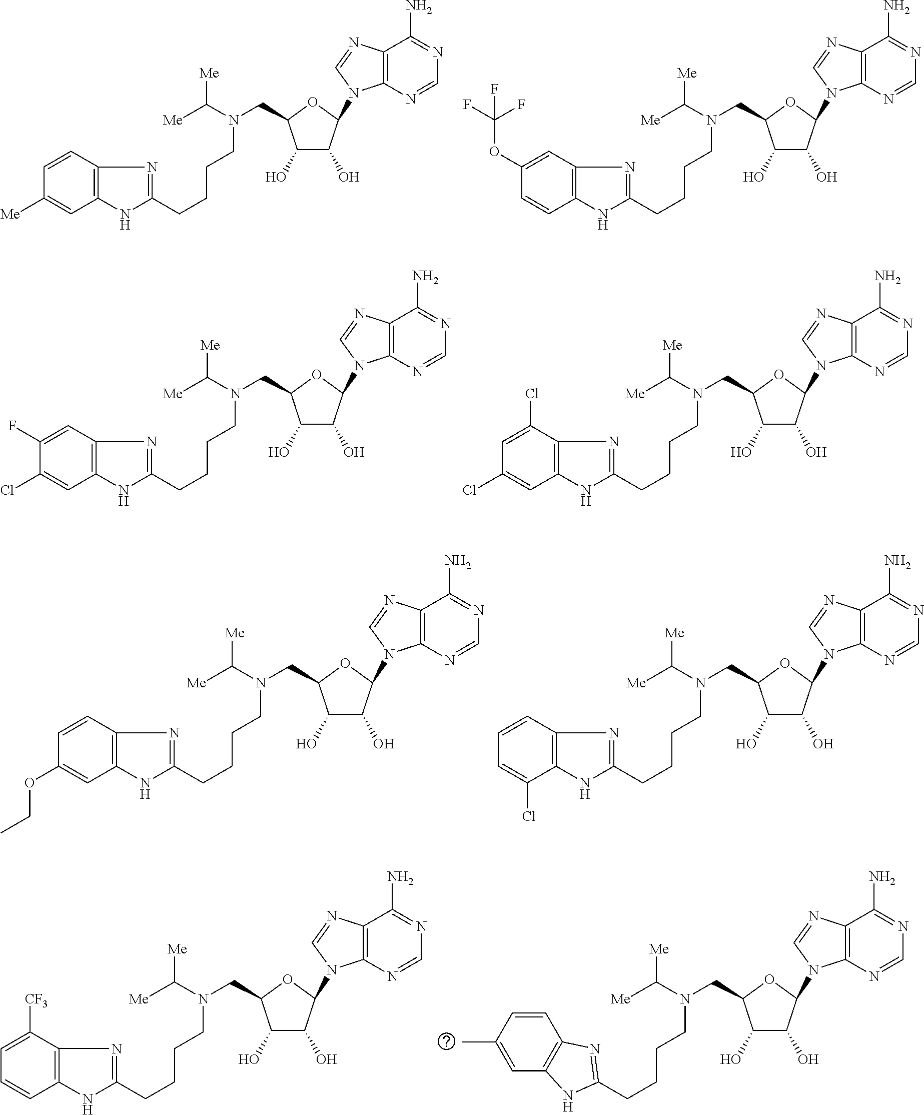

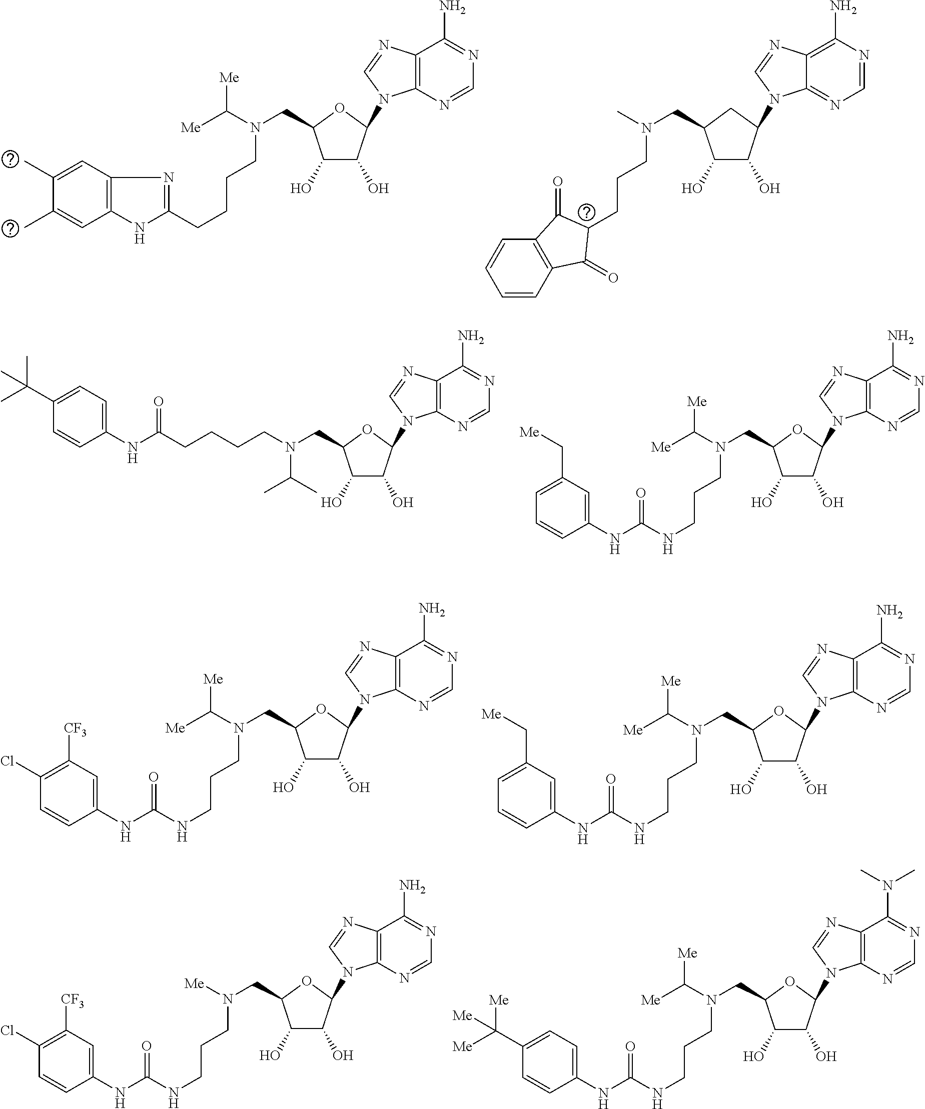

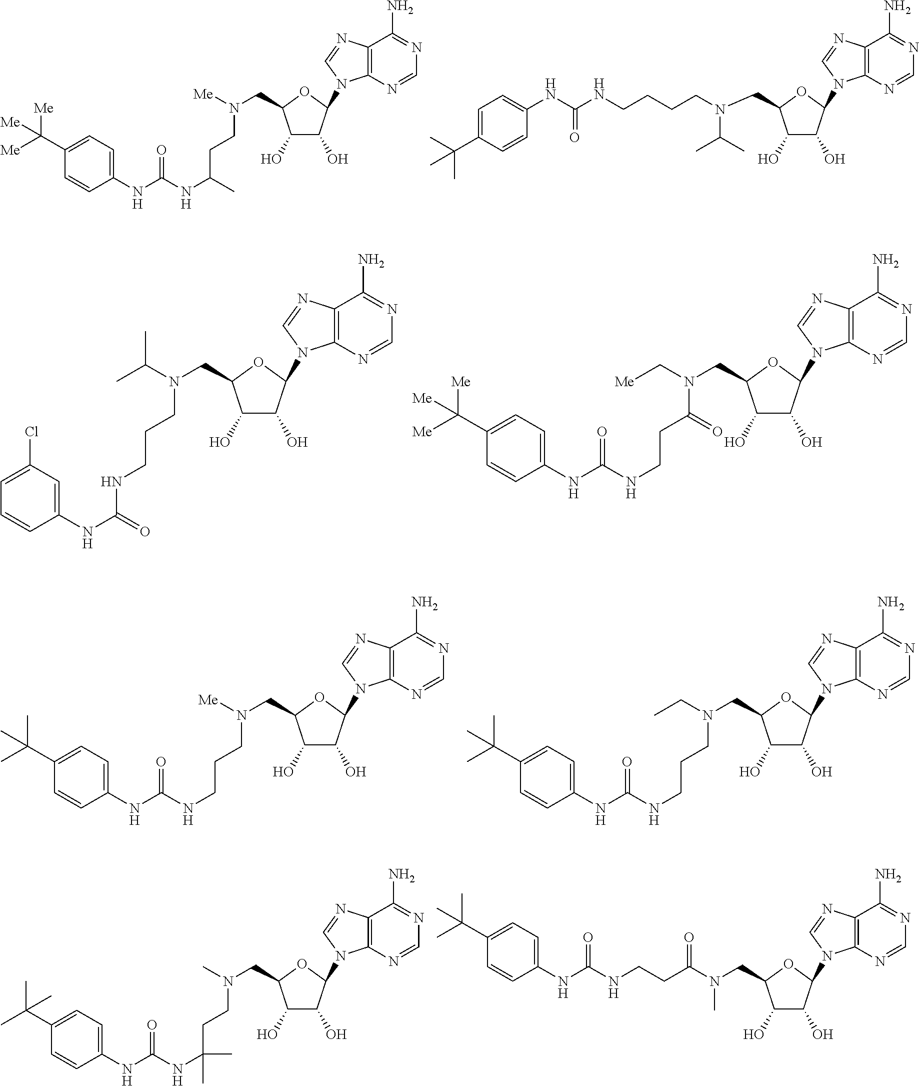

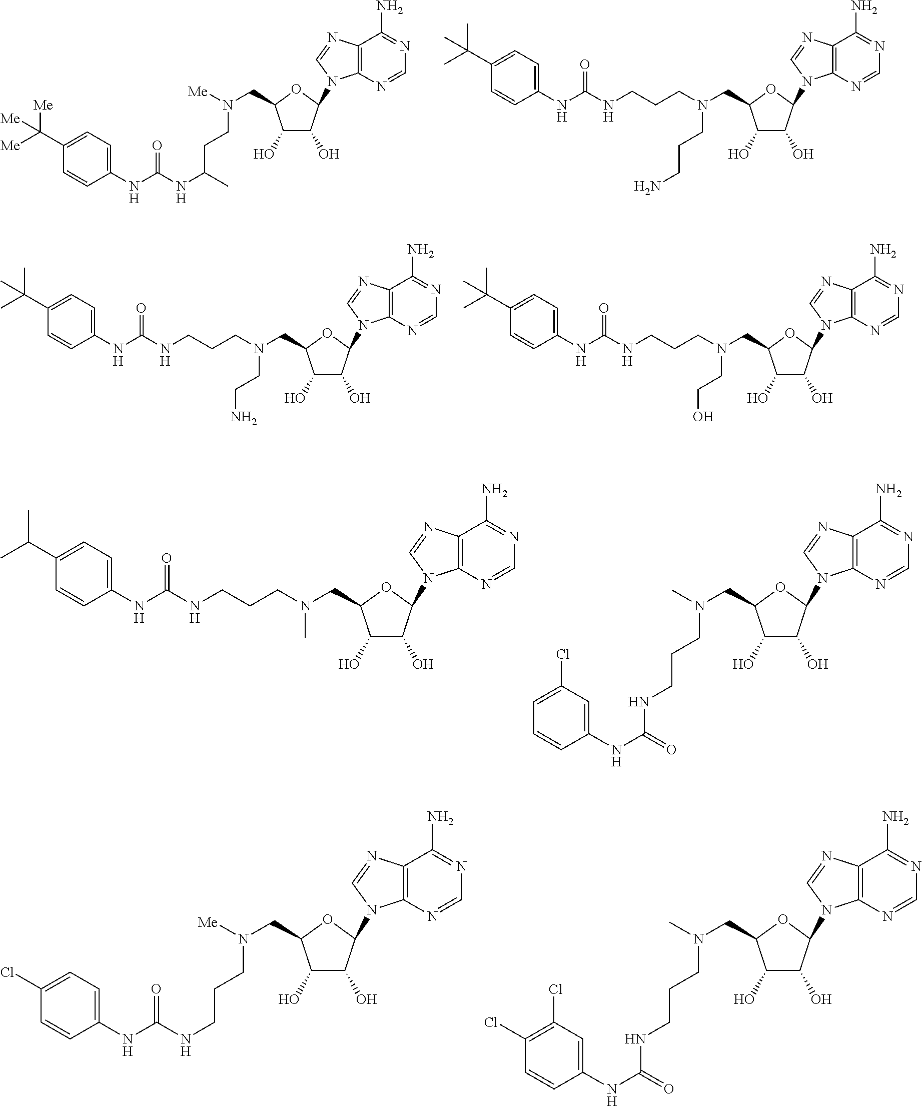

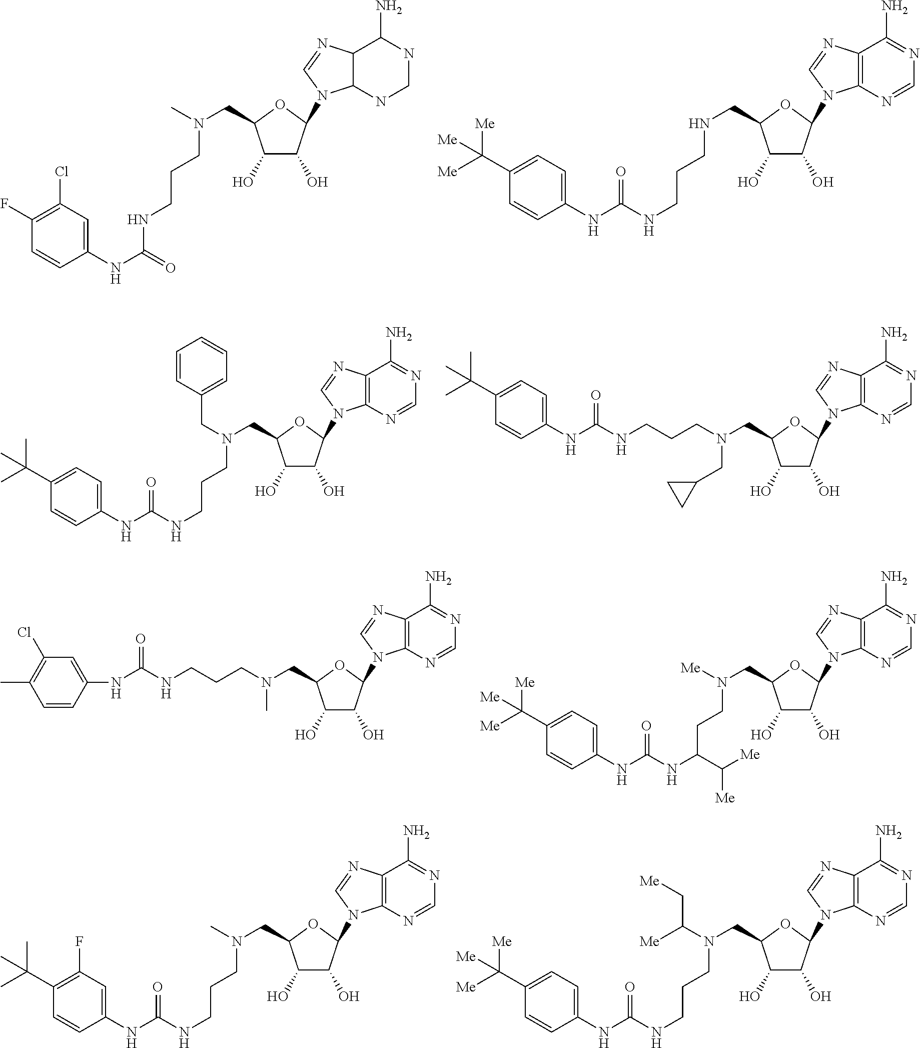

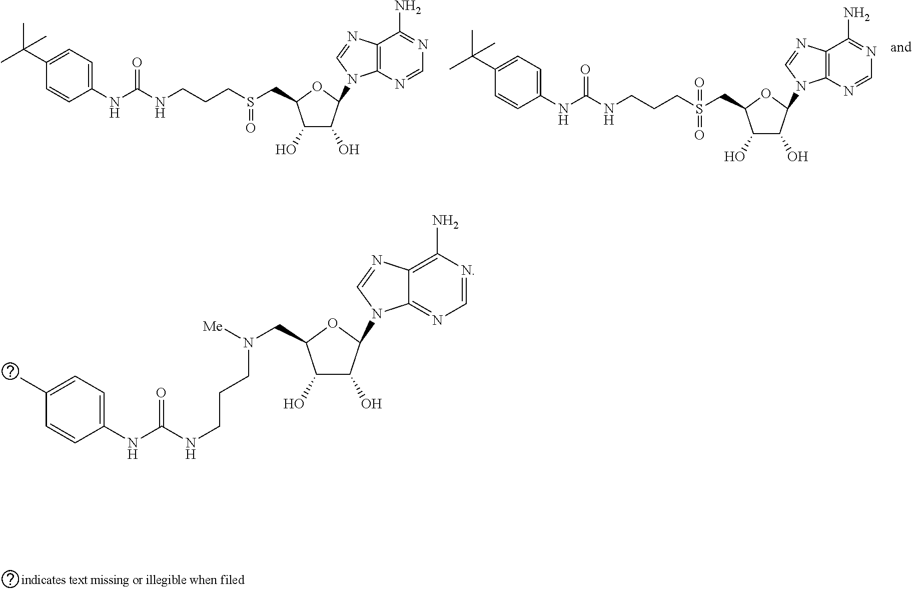

### Compounds of Formula (II)

In certain embodiments, the present disclosure provides a compound of Formula (II):

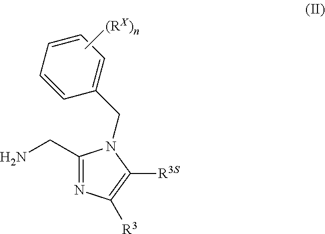

or a pharmaceutically acceptable salt thereof,
wherein:
R³ is hydrogen, methyl, ethyl, propyl, substituted or unsubstituted heteroarylalkyl, or —CH₂CH₂NHC(═O)R^{3a};
R^{3S} is hydrogen, methyl, ethyl, propyl, substituted or unsubstituted heteroarylalkyl, or —CH₂CH₂NHC(═O)R^{3a};
each occurrence of R^{3a} is independently substituted or unsubstituted alkyl, or substituted or unsubstituted aryl;
each R^{X} is independently selected from the group consisting of substituted or unsubstituted alkyl, substituted or unsubstituted cycloalkyl, substituted or unsubstituted aryl, substituted or unsubstituted heterocyclyl, substituted or unsubstituted heteroaryl, halogen, —CN, —NO₂, —OR^{X1}, —N(R^{X2})₂, —SR^{X1}, —C(═O)R^{X1}, —C(═O)OR^{X1}, —C(═O)SR^{X1}, —C(═O)N(R^{X2})₂, —C(═O)N(R^{X2})N(R^{X2})₂, —OC(═O)R^{X1}, —OC(═O)N(R^{X2})₂, —NR^{X2}C(═O)R^{X1}, —NR^{X2}C(═O)N(R^{X2})₂, —NR^{X2}C(═O)N(R^{X2})N(R^{X2})₂, —NR^{X2}C(═O)OR^{X1}, —SC(O)R^{X1}, —C(═NR^{X2})R^{X1}, —C(═NNR^{X2})R^{X1}, —C(═NOR^{X1})R^{X1}, —C(═NR²)N(R^{X2})₂, —NR^{X2}C(═NR^{X2})R^{X2}, —C(═S)R^{X1}, —C(═S)N(R^{X2})₂, —NR^{X2}C(═S)R^{X1}, —S(═O)R^{X1}, —OS(═O)₂R^{X1}, —SO₂R^{X1}, NR^{X2}SO₂R^{X1}, or —SO₂N(R^{X2})₂;
each R^{X1} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl;
each R^{X2} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl, or two R^{X2} groups are taken together with their intervening atoms to form a substituted or unsubstituted heterocyclic ring; and
n is 0, 1, 2, 3, 4, or 5.

In certain embodiments, the compound of Formula (II) is a compound of Formula (II′):

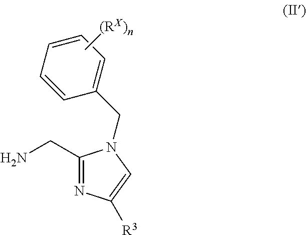

or a pharmaceutically acceptable salt thereof,
wherein:
R³ is hydrogen, methyl, ethyl, propyl, or —CH₂CH₂NHC(═O)R^{3a};
R^{3a} is substituted or unsubstituted alkyl, or substituted or unsubstituted aryl;
each R^{X} is independently selected from the group consisting of substituted or unsubstituted alkyl, substituted or unsubstituted cycloalkyl, substituted or unsubstituted aryl, substituted or unsubstituted heterocyclyl, substituted or unsubstituted heteroaryl, halogen, —CN, —NO₂, —OR^{X1}, —N(R^{X2})₂, —SR^{X1}, —C(═O)R^{X1}, —C(═O)OR^{X1}, —C(═O)SR^{X1}, —C(═O)N(R^{X2})₂, —C(═O)N(R^{X2})N(R^{X2})₂, —OC(═O)R^{X1}, —OC(═O)N(R^{X2})₂, —NRXC(═O)R^{X1}, —NR^{X2}C(═O)N(R^{X2})₂, —NR^{X2}C(═O)N(R^{X2})N(R^{X2})₂, —NRXC(═O)OR^{X1}, —SC(O)R^{X1}, —C(═NR^{X2})R^{X1}, —C(═NNR^{X2})R^{X1}, —C(═NOR^{X1})R^{X1}, —C(═NR^{X2})N(R^{X2})₂, —NR^{X2}C(═NR^{X2})R^{X2}, —C(═S)R^{X1}, —C(═S)N(R^{X2})₂, —NR^{X2}C(═S)R^{X1}, —S(═O)R^{X1}, —OS(═O)₂R^{X1}, —SO₂R^{X1}, —NR^{X2}SO₂R^{X1}, or —SO₂N(R^{X2})₂;
each R^{X1} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl;
each R^{X2} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl, or two R^{X2} groups are taken together with their intervening atoms to form a substituted or unsubstituted heterocyclic ring; and
n is 0, 1, 2, 3, 4, or 5.

In certain embodiments, the provided compound is of a free base form. In certain embodiments, the provided compound is in the form of a pharmaceutically acceptable salt as generally defined herein.

In certain embodiments, R³ is hydrogen.

In certain embodiments, R³ is methyl. In certain embodiments, R³ is ethyl. In certain embodiments, R³ is propyl.

In certain embodiments, R³ is —CH₂CH₂NHC(═O)R^{3a}; and R^{3a} is substituted or unsubstituted alkyl. In certain embodiments, R³ is —CH₂CH₂NHC(═O)R^{3a}; and R^{3a} is substituted or unsubstituted C₁₋₆alkyl. In certain embodiments, R³ is —CH₂CH₂NHC(═O)R^{3a}; and R^{3a} is substituted or unsubstituted C₁₋₃alkyl. In certain embodiments, R³ is —CH₂CH₂NHC(═O)R^{3a}; and R^{3a} is substituted or unsubstituted methyl. In certain embodiments, R³ is —CH₂CH₂NHC(═O)R^{3a}; and R^{3a} is substituted or unsubstituted ethyl. In certain embodiments, R³ is —CH₂CH₂NHC(═O)R^{3a}; and R^{3a} is substituted or unsubstituted propyl. In certain embodiments, R³ is —CH₂CH₂NHC(═O)R^{3a}; and R^{3a} is substituted or unsubstituted isopropyl. In certain embodiments, R³ is hydrogen. In certain embodiments, R³ is —CH₂CH₂NHC(═O)R^{3a}; and R^{3a} is substituted or unsubstituted aryl. In certain embodiments, R³ is —CH₂CH₂NHC(═O)R^{3a}; and R^{3a} is substituted or unsubstituted phenyl.

In certain embodiments, at least one instance of R^{X} is hydrogen. In certain embodiments, at least two instances of R^{X} are hydrogen. In certain embodiments, at least three instances of R^{X} are hydrogen. In certain embodiments, at least four instances of R^{X} are hydrogen. In certain embodiments, at least five instances of R^{X} are hydrogen. In certain embodiments, at least one instance of R^{X} is substituted or unsubstituted C₁₋₆alkyl. In certain embodiments, at least one instance of R^{X} is substituted or unsubstituted C₁₋₃alkyl. In certain embodiments, at least one instance of R^{X} is substituted or unsubstituted methyl. In certain embodiments, at least one instance of R^{X} is substituted or unsubstituted ethyl. In certain embodiments, at least one instance of R^{X} is substituted or unsubstituted propyl. In certain embodiments, at least one instance of R^{X} is substituted or unsubstituted isopropyl. In certain embodiments, at least one instance of R^{X} is substituted or unsubstituted cycloalkyl. In certain embodiments, at least one instance of R^{X} is substituted or unsubstituted 3-6 membered cycloalkyl. In certain embodiments, at least one instance of R^{X} is substituted or unsubstituted cyclopropyl. In certain embodiments, at least one instance of R^{X} is substituted or unsubstituted cyclobutyl. In certain embodiments, at least one instance of R^{X} is substituted or unsubstituted cyclopentyl. In certain embodiments, at least one instance of R^{X} is substituted or unsubstituted cyclohexyl.

In certain embodiments, at least one instance of R^{X} is substituted or unsubstituted heterocyclyl. In certain embodiments, at least one instance of R^{X} is substituted or unsubstituted 4-6 membered heterocyclyl. In certain embodiments, at least one instance of R^{X} is substituted or unsubstituted oxetanyl. In certain embodiments, at least one instance of R^{X} is substituted or unsubstituted azetidinyl. In certain embodiments, at least one instance of R^{X} is substituted or unsubstituted tetrahydrofuryl. In certain embodiments, at least one instance of R^{X} is substituted or unsubstituted pyrrolidinyl. In certain embodiments, at least one instance of R^{X} is substituted or unsubstituted tetrahydropyranyl. In certain embodiments, at least one instance of R^{X} is substituted or unsubstituted piperidinyl.

In certain embodiments, at least one instance of R^{X} is substituted or unsubstituted aryl. In certain embodiments, at least one instance of R^{X} is unsubstituted aryl. In certain embodiments, at least one instance of R^{X} is monosubstituted phenyl. In certain embodiments, at least one instance of R^{X} is disubstituted phenyl. In certain embodiments, at least one instance of R^{X} is trisubstituted phenyl. In certain embodiments, at least one instance of R^{X} is tetrasubstituted phenyl. In certain embodiments, at least one instance of R^{X} is pentasubstituted phenyl. In certain embodiments, at least one instance of R^{X} is unsubstituted phenyl.

In certain embodiments, at least one instance of R^{X} is substituted or unsubstituted heteroaryl. In certain embodiments, at least one instance of R^{X} is substituted or unsubstituted 5-6 membered heteroaryl. In certain embodiments, at least one instance of R^{X} is substituted or unsubstituted imidazolyl. In certain embodiments, at least one instance of R^{X} is substituted or unsubstituted pyrazolyl. In certain embodiments, at least one instance of R^{X} is substituted or unsubstituted oxazolyl. In certain embodiments, at least one instance of R^{X} is substituted or unsubstituted thiazolyl. In certain embodiments, at least one instance of R^{X} is substituted or unsubstituted oxadiazolyl. In certain embodiments, at least one instance of R^{X} is substituted or unsubstituted thiadiazolyl. In certain embodiments, at least one instance of R^{X} is substituted or unsubstituted triazolyl. In certain embodiments, at least one instance of R^{X} is substituted or unsubstituted tetrazolyl. In certain embodiments, at least one instance of R^{X} is substituted or unsubstituted pyridyl. In certain embodiments, at least one instance of R^{X} is substituted or unsubstituted pyrazinyl. In certain embodiments, at least one instance of R^{X} is substituted or unsubstituted pyrimidinyl. In certain embodiments, at least one instance of R^{X} is substituted or unsubstituted pyridizinyl.

In certain embodiments, at least one instance of R^{X} is —CN. In certain embodiments, at least one instance of R^{X} is —NO₂.

In certain embodiments, at least one instance of R^{X} is —OR^{X1}. In certain embodiments, at least one instance of R^{X} is —OMe.

In certain embodiments, at least one instance of R^{X} is —N(R^{X2})₂. In certain embodiments, at least one instance of R^{X} is —NMe₂. In certain embodiments, at least one instance of R^{X} is —NHMe. In certain embodiments, at least one instance of R^{X} is —NH₂.

In certain embodiments, at least one instance of R^{X} is —SR^{X1}. In certain embodiments, at least one instance of R^{X} is —SMe.

In certain embodiments, at least one instance of R^{X} is —C(═O)R^{X1}. In certain embodiments, at least one instance of R^{X} is —C(═O)Me.

In certain embodiments, at least one instance of R^{X} is —C(O)OR^{X1}. In certain embodiments, at least one instance of R^{X} is —C(═O)OMe. In certain embodiments, at least one instance of R^{X} is —C(═O)OEt. In certain embodiments, at least one instance of R^{X} is —C(═O)OtBu.

In certain embodiments, at least one instance of R^{X} is —C(═O)SR^{X1}. In certain embodiments, at least one instance of R^{X} is —C(═O)SMe.

In certain embodiments, at least one instance of R^{X} is —C(═O)N(R²)₂. In certain embodiments, at least one instance of R^{X} is —C(═O)NMe₂. In certain embodiments, at least one instance of R^{X} is —C(═O)NHMe. In certain embodiments, at least one instance of R^{X} is —C(═O)NH₂.

In certain embodiments, at least one instance of R^{X} is —C(═O)N(R^{X2})N(R^{X2})₂.

In certain embodiments, at least one instance of R^{X} is —OC(═O)R^{X1}. In certain embodiments, at least one instance of R^{X} is —OC(═O)Me.

In certain embodiments, at least one instance of R^{X} is —OC(═O)N(R^{X2})₂. In certain embodiments, at least one instance of R^{X} is —OC(═O)NMe₂. In certain embodiments, at least one instance of R^{X} is —OC(═O)NHMe. In certain embodiments, at least one instance of R^{X} is —OC(═O)NH₂. In certain embodiments, at least one instance of R^{X} is —OC(═O)NHtBu.

In certain embodiments, at least one instance of R^{X} is —NR^{X2}C(═O)R^{X1}. In certain embodiments, at least one instance of R^{X} is —NHC(O)R^{X1}. In certain embodiments, at least one instance of R^{X} is —NMeC(═O)R^{X1}. In certain embodiments, at least one instance of R^{X} is —NR^{X2}C(═O)Me. In certain embodiments, at least one instance of R^{X} is —NHC(═O)Me. In certain embodiments, at least one instance of R^{X} is —NMeC(═O)Me.

In certain embodiments, at least one instance of R^{X} is —NR^{X2}C(═O)N(R^{X2})₂. In certain embodiments, at least one instance of R^{X} is —NHC(═O)N(R^{X2})₂. In certain embodiments, at least one instance of R^{X} is —NR^{X2}C(═O)NH₂. In certain embodiments, at least one instance of R^{X} is —NMeC(═O)N(R^{X2})₂. In certain embodiments, at least one instance of R^{X} is —NR^{X2}C(═O)NMe₂. In certain embodiments, at least one instance of R^{X} is —NR^{X2}C(═O)NHMe. In certain embodiments, at least one instance of R^{X} is —NHC(═O)NMe₂. In certain embodiments, at least one instance of R^{X} is —NHC(═O)NMe₂. In certain embodiments, at least one instance of R^{X} is —NHC(═O)NHMe. In certain embodiments, at least one instance of R^{X} is —NMeC(═O)NMe₂. In certain embodiments, at least one instance of R^{X} is —NMeC(═O)NHMe. In certain embodiments, at least one instance of R^{X} is —NMeC(═O)NH₂. In certain embodiments, at least one instance of R^{X} is —NHC(═O)NH₂.

In certain embodiments, at least one instance of R^{X} is —NR^{X2}C(═O)N(R^{X2})N(R^{X2})₂.

In certain embodiments, at least one instance of R^{X} is —NR^{X2}C(═O)OR^{X1}. In certain embodiments, at least one instance of R^{X} is —NHC(═O)OR^{X1}. In certain embodiments, at least one instance of R^{X} is —NMeC(═O)OR^{X1}. In certain embodiments, at least one instance of R^{X} is —NHC(O)OtBu. In certain embodiments, at least one instance of R^{X} is —NMeC(═O)OtBu. In certain embodiments, at least one instance of R^{X} is —NR^{X2}C(═O)OtBu. In certain embodiments, at least one instance of R^{X} is —NR^{X2}C(═O)OBn.

In certain embodiments, at least one instance of R^{X} is —SC(═O)R^{X1}.

In certain embodiments, at least one instance of R^{X} is —C(═NR^{X2})R^{X1}. In certain embodiments, at least one instance of R^{X} is —C(═NNR^{X2})R^{X1}. In certain embodiments, at least one instance of R^{X} is —C(═NOR^{X1})R^{X1}. In certain embodiments, at least one instance of R^{X} is —C(═NR^{X2})N(R^{X2})₂. In certain embodiments, at least one instance of R^{X} is —NR^{X2}C(═NR^{X2})R^{X2}.

In certain embodiments, at least one instance of R^{X} is —C(═S)R^{X1}. In certain embodiments, at least one instance of R^{X} is —C(═S)N(R^{X2})₂. In certain embodiments, at least one instance of R^{X} is —NR^{X2}C(═S)R^{X1}.

In certain embodiments, at least one instance of R^{X} is —S(O)R^{X1}.

In certain embodiments, at least one instance of R^{X} is —OS(O)₂R^{X1}. In certain embodiments, at least one instance of R^{X} is —OS(O)₂Me. In certain embodiments, at least one instance of R^{X} is —OS(O)₂Ph. In certain embodiments, at least one instance of R^{X} is —OS(O)₂PhMe. In certain embodiments, at least one instance of R^{X} is —OS(O)₂CF₃.

In certain embodiments, at least one instance of R^{X} is —SO₂R^{X1}. In certain embodiments, at least one instance of R^{X} is —SO₂Me. In certain embodiments, at least one instance of R^{X} is —SO₂tBu.

In certain embodiments, at least one instance of R^{X} is —NR^{X2}SO₂R^{X1}. In certain embodiments, at least one instance of R^{X} is —NHSO₂R^{X1}. In certain embodiments, at least one instance of R^{X} is —NR^{X2}SO₂Me. In certain embodiments, at least one instance of R^{X} is —NR^{X2}SO₂tBu. In certain embodiments, at least one instance of R^{X} is —NR^{X2}SO₂Ph.

In certain embodiments, at least one instance of R^{X} is —SO₂N(R^{X2})₂. In certain embodiments, at least one instance of R^{X} is —SO₂NMe₂. In certain embodiments, at least one instance of R^{X} is —SO₂NHMe. In certain embodiments, at least one instance of R^{X} is —SO₂NH₂.

In certain embodiments, at least one instance of R^{X} is halogen (e.g., fluoro, chloro, bromo, or iodo).

In certain embodiments, at least one instance of R^{X1} is hydrogen. In certain embodiments, at least one instance of R^{X1} is substituted or unsubstituted alkyl. In certain embodiments, at least one instance of R^{X1} is substituted or unsubstituted C₁₋₆alkyl. In certain embodiments, at least one instance of R^{X1} is substituted or unsubstituted C₁₋₃alkyl. In certain embodiments, at least one instance of R^{X1} is substituted or unsubstituted methyl. In certain embodiments, at least one instance of R^{X1} is substituted or unsubstituted ethyl. In certain embodiments, at least one instance of R^{X1} is substituted or unsubstituted propyl. In certain embodiments, at least one instance of R^{X1} is substituted or unsubstituted isopropyl.

In certain embodiments, at least one instance of R^{X1} is substituted or unsubstituted cycloalkyl. In certain embodiments, at least one instance of R^{X1} is substituted or unsubstituted 3-6 membered cycloalkyl. In certain embodiments, at least one instance of R^{X1} is substituted or unsubstituted cyclopropyl. In certain embodiments, at least one instance of R^{X1} is substituted or unsubstituted cyclobutyl. In certain embodiments, at least one instance of R^{X1} is substituted or unsubstituted cyclopentyl. In certain embodiments, at least one instance of R^{X1} is substituted or unsubstituted cyclohexyl.

In certain embodiments, at least one instance of R^{X1} is substituted or unsubstituted heterocyclyl. In certain embodiments, at least one instance of R^{X1} is substituted or unsubstituted 4-6 membered heterocyclyl. In certain embodiments, at least one instance of R^{X1} is substituted or unsubstituted oxetanyl. In certain embodiments, at least one instance of R^{X1} is substituted or unsubstituted azetidinyl. In certain embodiments, at least one instance of R^{X1} is substituted or unsubstituted tetrahydrofuryl. In certain embodiments, at least one instance of R^{X1} is substituted or unsubstituted pyrrolidinyl. In certain embodiments, at least one instance of R^{X1} is substituted or unsubstituted tetrahydropyranyl. In certain embodiments, at least one instance of R^{X1} is substituted or unsubstituted piperidinyl.

In certain embodiments, at least one instance of R^{X1} is substituted or unsubstituted aryl. In certain embodiments, at least one instance of R^{X1} is unsubstituted aryl. In certain embodiments, at least one instance of R^{X1} is monosubstituted phenyl. In certain embodiments, at least one instance of R^{X1} is disubstituted phenyl. In certain embodiments, at least one instance of R^{X1} is trisubstituted phenyl. In certain embodiments, at least one instance of R^{X1} is tetrasubstituted phenyl. In certain embodiments, at least one instance of R^{X1} is pentasubstituted phenyl. In certain embodiments, at least one instance of R^{X1} is unsubstituted phenyl.

In certain embodiments, at least one instance of R^{X1} is substituted or unsubstituted heteroaryl. In certain embodiments, at least one instance of R^{X1} is substituted or unsubstituted 5-6 membered heteroaryl. In certain embodiments, at least one instance of R^{X1} is substituted or unsubstituted imidazolyl. In certain embodiments, at least one instance of R^{X1} is substituted or unsubstituted pyrazolyl. In certain embodiments, at least one instance of R^{X1} is substituted or unsubstituted oxazolyl. In certain embodiments, at least one instance of R^{X1} is substituted or unsubstituted thiazolyl. In certain embodiments, at least one instance of R^{X1} is substituted or unsubstituted oxadiazolyl. In certain embodiments, at least one instance of R^{X1} is substituted or unsubstituted thiadiazolyl. In certain embodiments, at least one instance of R^{X1} is substituted or unsubstituted triazolyl. In certain embodiments, at least one instance of R^{X1} is substituted or unsubstituted tetrazolyl. In certain embodiments, at least one instance of R^{X1} is substituted or unsubstituted pyridyl. In certain embodiments, at least one instance of R^{X1} is substituted or unsubstituted pyrazinyl. In certain embodiments, at least one instance of R^{X1} is substituted or unsubstituted pyrimidinyl. In certain embodiments, at least one instance of R^{X1} is substituted or unsubstituted pyridizinyl.

In certain embodiments, at least one instance of R^{X2} is hydrogen. In certain embodiments, at least one instance of R^{X2} is substituted or unsubstituted alkyl. In certain embodiments, at least one instance of R^{X2} is substituted or unsubstituted C₁₋₆alkyl. In certain embodiments, at least one instance of R^{X2} is substituted or unsubstituted C₁₋₃alkyl. In certain embodiments, at least one instance of R^{X2} is substituted or unsubstituted methyl. In certain embodiments, at least one instance of R^{X2} is substituted or unsubstituted ethyl. In certain embodiments, at least one instance of R^{X2} is substituted or unsubstituted propyl. In certain embodiments, at least one instance of R^{X2} is substituted or unsubstituted isopropyl.

In certain embodiments, at least one instance of R^{X2} is substituted or unsubstituted cycloalkyl. In certain embodiments, at least one instance of R^{X2} is substituted or unsubstituted 3-6 membered cycloalkyl. In certain embodiments, at least one instance of R^{X2} is substituted or unsubstituted cyclopropyl. In certain embodiments, at least one instance of R^{X2} is substituted or unsubstituted cyclobutyl. In certain embodiments, at least one instance of R^{X2} is substituted or unsubstituted cyclopentyl. In certain embodiments, at least one instance of R^{X2} is substituted or unsubstituted cyclohexyl.

In certain embodiments, at least one instance of R^{X2} is substituted or unsubstituted heterocyclyl. In certain embodiments, at least one instance of R^{X2} is substituted or unsubstituted 4-6 membered heterocyclyl. In certain embodiments, at least one instance of R^{X2} is substituted or unsubstituted oxetanyl. In certain embodiments, at least one instance of R^{X2} is substituted or unsubstituted azetidinyl. In certain embodiments, at least one instance of R^{X2} is substituted or unsubstituted tetrahydrofuryl. In certain embodiments, at least one instance of R^{X2} is substituted or unsubstituted pyrrolidinyl. In certain embodiments, at least one instance of R^{X2} is substituted or unsubstituted tetrahydropyranyl. In certain embodiments, at least one instance of R^{X2} is substituted or unsubstituted piperidinyl.

In certain embodiments, at least one instance of R^{X2} is substituted or unsubstituted aryl. In certain embodiments, at least one instance of R^{X2} is unsubstituted aryl. In certain embodiments, at least one instance of R^{X2} is monosubstituted phenyl. In certain embodiments, at least one instance of R^{X2} is disubstituted phenyl. In certain embodiments, at least one instance of R^{X2} is trisubstituted phenyl. In certain embodiments, at least one instance of R^{X2} is tetrasubstituted phenyl. In certain embodiments, at least one instance of R^{X2} n is pentasubstituted phenyl. In certain embodiments, at least one instance of R^{X2} is unsubstituted phenyl.

In certain embodiments, at least one instance of R^{X2} is substituted or unsubstituted heteroaryl. In certain embodiments, at least one instance of R^{X2} is substituted or unsubstituted 5-6 membered heteroaryl. In certain embodiments, at least one instance of R^{X2} is substituted or unsubstituted imidazolyl. In certain embodiments, at least one instance of R^{X2} is substituted or unsubstituted pyrazolyl. In certain embodiments, at least one instance of R^{X2} is substituted or unsubstituted oxazolyl. In certain embodiments, at least one instance of R^{X2} is substituted or unsubstituted thiazolyl. In certain embodiments, at least one instance of R^{X2} is substituted or unsubstituted oxadiazolyl. In certain embodiments, at least one instance of R^{X2} is substituted or unsubstituted thiadiazolyl. In certain embodiments, at least one instance of R^{X2} is substituted or unsubstituted triazolyl. In certain embodiments, at least one instance of R^{X2} is substituted or unsubstituted tetrazolyl. In certain embodiments, at least one instance of R^{X2} substituted or unsubstituted pyridyl. In certain embodiments, at least one instance of R^{X2} is substituted or unsubstituted pyrazinyl. In certain embodiments, at least one instance of R^{X2} is substituted or unsubstituted pyrimidinyl. In certain embodiments, at least one instance of R^{X2} is substituted or unsubstituted pyridizinyl.

In certain embodiments, two R^{X2} groups are taken together with their intervening atoms to form a substituted or unsubstituted heterocyclic ring. In certain embodiments, two R^{X2} groups are taken together with their intervening atoms to form a substituted or unsubstituted 4-6 membered heterocyclic ring. In certain embodiments, two R^{X2} groups are taken together with their intervening atoms to form a substituted or unsubstituted azetidine. In certain embodiments, two R^{X2} groups are taken together with their intervening atoms to form a substituted or unsubstituted pyrrolidine. In certain embodiments, two R^{X2} groups are taken together with their intervening atoms to form a substituted or unsubstituted piperidine. In certain embodiments, two R^{X2} groups are taken together with their intervening atoms to form a substituted or unsubstituted piperazine.

In certain embodiments, n is 0; and the phenyl ring to which R^{X} is attached is of formula:

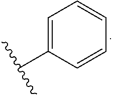 [SMILES: *c1ccccc1]

In certain embodiments, n is 1; and the phenyl ring to which R^{X} is attached is of formula:

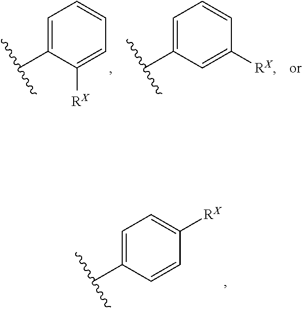

In certain embodiments, n is 2; and the phenyl ring to which R^{X} is attached is of formula:

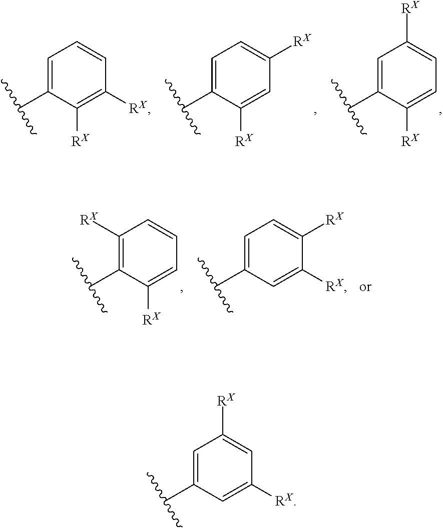

In certain embodiments, n is 3; and the phenyl ring to which R^{X} is attached is of formula:

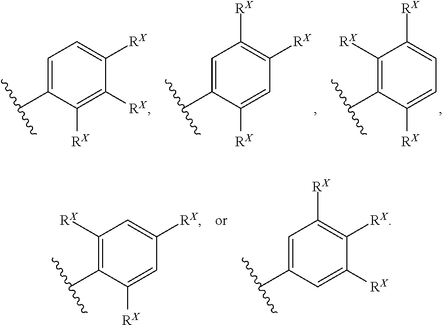

In certain embodiments, n is 4; and the phenyl ring to which R^{X} is attached is of formula:

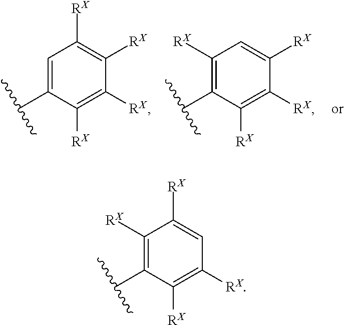

In certain embodiments, n is 5; and the phenyl ring to which R^{X} is attached is of formula:

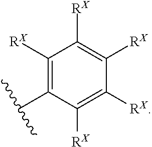

In certain embodiments, a compound of Formula (II) is not of formula:

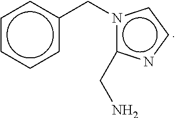

In certain embodiments, R³ is not hydrogen. In certain embodiments, n is not 0. In certain embodiments, n is at least 1. In certain embodiments, n is at least 2. In certain embodiments, n is at least 3. In certain embodiments, n is at least 4.

### Compounds of Formula (III)

In certain embodiments, the present disclosure provides a compound of Formula (III):

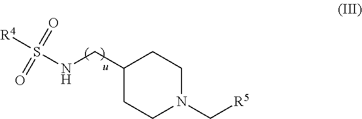

or a pharmaceutically acceptable salt thereof,
wherein:
R⁴ is substituted or unsubstituted aryl;
R⁵ is substituted or unsubstituted aryl or substituted or unsubstituted heteroaryl; and
u is 0 or 1.

In certain embodiments, the provided compound is of a free base form. In certain embodiments, the provided compound is in the form of a pharmaceutically acceptable salt as generally defined herein.

In certain embodiments, u is 0. In certain embodiments, u is 1.

In certain embodiments, R⁴ is substituted or unsubstituted aryl. In certain embodiments, R⁴ is monosubstituted phenyl. In certain embodiments, R⁴ is disubstituted phenyl. In certain embodiments, R⁴ is trisubstituted phenyl. In certain embodiments, R⁴ is tetrasubstituted phenyl. In certain embodiments, R⁴ is pentasubstituted phenyl. In certain embodiments, R⁴ is unsubstituted phenyl.

In certain embodiments, R⁴ is phenyl substituted with at least one substituted or unsubstituted alkyl group. In certain embodiments, R⁴ is phenyl substituted with at least one substituted or unsubstituted C₁₋₆alkyl group. In certain embodiments, R⁴ is phenyl substituted with at least one substituted or unsubstituted methyl group. In certain embodiments, R⁴ is phenyl substituted with at least one substituted or unsubstituted ethyl group. In certain embodiments, R⁴ is phenyl substituted with at least two substituted or unsubstituted alkyl groups. In certain embodiments, R⁴ is phenyl substituted with at least two substituted or unsubstituted C₁₋₆alkyl groups. In certain embodiments, R⁴ is phenyl substituted with at least two substituted or unsubstituted methyl groups. In certain embodiments, R⁴ is phenyl substituted with at least two substituted or unsubstituted ethyl groups.

In certain embodiments, R⁴ is phenyl substituted with at least one alkoxy group. In certain embodiments, R⁴ is phenyl substituted with at least one methoxy group. In certain embodiments, R⁴ is phenyl substituted with at least one ethoxy group. In certain embodiments, R⁴ is phenyl substituted with at least one substituted or unsubstituted —OPh group.

In certain embodiments, R⁴ is phenyl substituted with at least one halogen (e.g., fluoro, chloro, bromo, or iodo).

In certain embodiments, R⁴ is phenyl substituted with at least one —NH₂ group. In certain embodiments, R⁴ is phenyl substituted with at least one substituted or unsubstituted —NH(alkyl) group, wherein the alkyl is substituted or unsubstituted. In certain embodiments, R⁴ is phenyl substituted with at least one substituted or unsubstituted —N(alkyl)₂ group, wherein the alkyl is substituted or unsubstituted. In certain embodiments, R⁴ is phenyl substituted with at least one substituted or unsubstituted —NH(C₁₋₆alkyl) group, wherein the C₁₋₆alkyl alkyl is substituted or unsubstituted. In certain embodiments, R⁴ is phenyl substituted with at least one substituted or unsubstituted —N(C₁₋₆alkyl)₂ group, wherein the C₁₋₆alkyl alkyl is substituted or unsubstituted. In certain embodiments, R⁴ is phenyl substituted with at least one substituted or unsubstituted —NHMe group. In certain embodiments, R⁴ is phenyl substituted with at least one substituted or unsubstituted —NMe₂ group. In certain embodiments, R⁴ is phenyl substituted with at least one unsubstituted —NH(alkyl) group.

In certain embodiments, R⁴ is of formula:

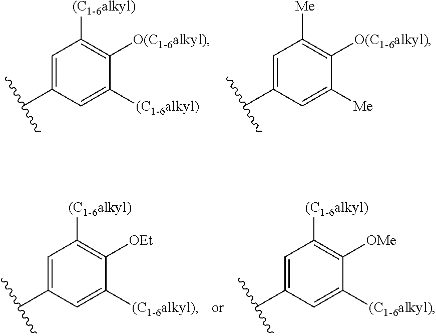

wherein the alkyl is substituted or unsubstituted.

In certain embodiments, R⁴ is of formula:

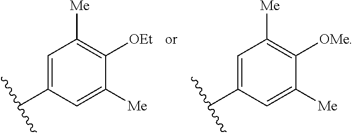 [SMILES: *c1cc(C)c(OCC)c(C)c1 ; *c1cc(C)c(OC)c(C)c1]

In certain embodiments, R⁴ is monosubstituted naphthyl. In certain embodiments, R⁴ is disubstituted naphthyl. In certain embodiments, R⁴ is trisubstituted naphthyl. In certain embodiments, R⁴ is tetrasubstituted naphthyl. In certain embodiments, R⁴ is pentasubstituted naphthyl. In certain embodiments, R⁴ is hexasubstituted naphthyl. In certain embodiments, R⁴ is heptasubstituted naphthyl. In certain embodiments, R⁴ is unsubstituted naphthyl.

In certain embodiments, R⁴ is naphthyl substituted with at least one substituted or unsubstituted alkyl group. In certain embodiments, R⁴ is naphthyl substituted with at least one substituted or unsubstituted C₁₋₆alkyl group. In certain embodiments, R⁴ is naphthyl substituted or unsubstituted with at least one substituted methyl group. In certain embodiments, R⁴ is naphthyl substituted with at least one substituted or unsubstituted ethyl group. In certain embodiments, R⁴ is naphthyl substituted with at least two substituted or unsubstituted alkyl groups. In certain embodiments, R⁴ is naphthyl substituted with at least two substituted or unsubstituted C₁₋₆alkyl groups. In certain embodiments, R⁴ is naphthyl substituted with at least two substituted or unsubstituted methyl groups. In certain embodiments, R⁴ is naphthyl substituted with at least two substituted or unsubstituted ethyl groups.

In certain embodiments, R⁴ is naphthyl substituted with at least one alkoxy group. In certain embodiments, R⁴ is naphthyl substituted with at least one methoxy group. In certain embodiments, R⁴ is naphthyl substituted with at least one ethoxy group. In certain embodiments, R⁴ is naphthyl substituted with at least one substituted or unsubstituted —OPh group.

In certain embodiments, R⁴ is naphthyl substituted with at least one halogen (e.g., fluoro, chloro, bromo, or iodo).

In certain embodiments, R⁴ is naphthyl substituted with at least one —NH₂ group. In certain embodiments, R⁴ is naphthyl substituted with at least one substituted or unsubstituted —NH(alkyl) group. In certain embodiments, R⁴ is naphthyl substituted with at least one substituted or unsubstituted —N(alkyl)₂ group. In certain embodiments, R⁴ is naphthyl substituted with at least one substituted or unsubstituted —NH(C₁₋₆alkyl) group. In certain embodiments, R⁴ is naphthyl substituted with at least one substituted or unsubstituted —N(C₁₋₆alkyl)₂ group. In certain embodiments, R⁴ is naphthyl substituted with at least one substituted or unsubstituted —NHMe group. In certain embodiments, R⁴ is naphthyl substituted with at least one substituted or unsubstituted —NMe₂ group.

In certain embodiments, R⁴ is of formula:

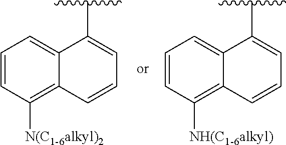

wherein the alkyl is substituted or unsubstituted.

In certain embodiments, R⁴ is of formula:

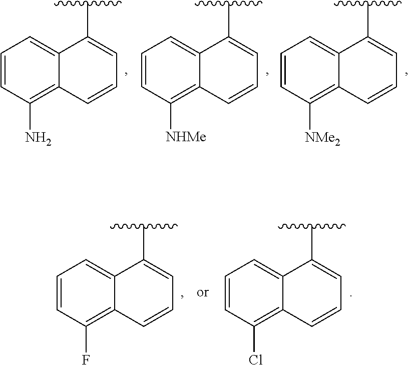

In certain embodiments, R⁵ is substituted or unsubstituted aryl. In certain embodiments, R⁵ is monosubstituted phenyl. In certain embodiments, R⁵ is disubstituted phenyl. In certain embodiments, R⁵ is trisubstituted phenyl. In certain embodiments, R⁵ is tetrasubstituted phenyl. In certain embodiments, R⁵ is pentasubstituted phenyl. In certain embodiments, R⁵ is unsubstituted phenyl. In certain embodiments, R⁵ is phenyl substituted with at least one substituted or unsubstituted alkyl group. In certain embodiments, R⁵ is phenyl substituted with at least one substituted or unsubstituted C₁₋₆alkyl group. In certain embodiments, R⁵ is phenyl substituted with at least one substituted or unsubstituted methyl group. In certain embodiments, R⁵ is phenyl substituted with at least one substituted or unsubstituted ethyl group. In certain embodiments, R⁵ is substituted phenyl ring fused with a substituted or unsubstituted 5-membered heterocyclic ring. In certain embodiments, R⁵ is substituted phenyl ring fused with a substituted or unsubstituted dioxolane ring. In certain embodiments, R⁵ is substituted phenyl ring fused with a substituted or unsubstituted 6-membered heterocyclic ring.

In certain embodiments, R⁵ is phenyl substituted with at least one alkoxy group. In certain embodiments, R⁵ is phenyl substituted with at least one methoxy group. In certain embodiments, R⁵ is phenyl substituted with at least one ethoxy group.

In certain embodiments, R⁵ is phenyl substituted with at least one halogen (e.g., fluoro, chloro, bromo, or iodo).

In certain embodiments, R⁵ is phenyl substituted with at least one substituted or unsubstituted phenyl ring.

In certain embodiments, R⁵ is of formula:

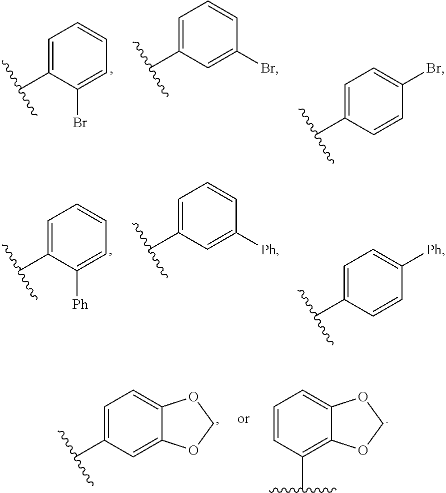

In certain embodiments, R⁵ is monosubstituted naphthyl. In certain embodiments, R⁵ is disubstituted naphthyl. In certain embodiments, R⁵ is trisubstituted naphthyl. In certain embodiments, R⁵ is tetrasubstituted naphthyl. In certain embodiments, R⁵ is pentasubstituted naphthyl. In certain embodiments, R⁵ is hexasubstituted naphthyl. In certain embodiments, R⁵ is heptasubstituted naphthyl. In certain embodiments, R⁵ is unsubstituted naphthyl. In certain embodiments, R⁵ is naphthyl substituted with at least one substituted or unsubstituted alkyl group. In certain embodiments, R⁵ is naphthyl substituted with at least one substituted or unsubstituted C₁₋₆alkyl group. In certain embodiments, R⁵ is naphthyl substituted with at least one substituted or unsubstituted methyl group. In certain embodiments, R⁵ is naphthyl substituted with at least one substituted or unsubstituted ethyl group.

In certain embodiments, R⁵ is naphthyl substituted with at least one alkoxy group. In certain embodiments, R⁵ is naphthyl substituted with at least one methoxy group. In certain embodiments, R⁵ is naphthyl substituted with at least one ethoxy group.

In certain embodiments, R⁵ is naphthyl substituted with at least one halogen (e.g., fluoro, chloro, bromo, or iodo).

In certain embodiments, R⁵ is of formula:

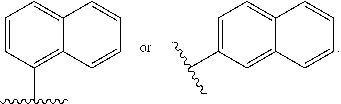 [SMILES: *c1cccc2ccccc12 ; *c1ccc2ccccc2c1]

In certain embodiments, at least one instance of R⁵ is substituted or unsubstituted heteroaryl. In certain embodiments, at least one instance of R⁵ is substituted or unsubstituted 5-6 membered heteroaryl. In certain embodiments, at least one instance of R⁵ is substituted or unsubstituted imidazolyl. In certain embodiments, at least one instance of R⁵ is substituted or unsubstituted pyrazolyl. In certain embodiments, at least one instance of R⁵ is substituted or unsubstituted oxazolyl. In certain embodiments, at least one instance of R⁵ is substituted or unsubstituted thiazolyl. In certain embodiments, at least one instance of R⁵ is substituted or unsubstituted oxadiazolyl. In certain embodiments, at least one instance of R⁵ is substituted or unsubstituted thiadiazolyl. In certain embodiments, at least one instance of R⁵ is substituted or unsubstituted triazolyl. In certain embodiments, at least one instance of R⁵ is substituted or unsubstituted tetrazolyl. In certain embodiments, at least one instance of R⁵ is substituted or unsubstituted pyridyl. In certain embodiments, at least one instance of R⁵ is substituted or unsubstituted pyrazinyl. In certain embodiments, at least one instance of R⁵ is substituted or unsubstituted pyrimidinyl. In certain embodiments, at least one instance of R⁵ is substituted or unsubstituted pyridizinyl.

In certain embodiments, R⁵ is of formula:

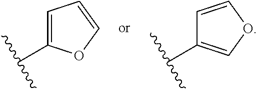 [SMILES: *c1ccco1 ; *c1ccoc1]

In certain embodiments, R⁵ is a substituted or unsubstituted 8- to 10-membered bicyclic heteroaryl having 1-4 heteroatoms independently selected from nitrogen, oxygen, and sulfur. In certain embodiments, R⁵ is a substituted or unsubstituted 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl). In certain embodiments, R⁵ is a substituted or unsubstituted 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl). In certain embodiments, R⁵ is substituted or unsubstituted quinoline. In certain embodiments, R⁵ is substituted or unsubstituted imidazo[2,1-b]thiazole. In certain embodiments, R⁵ is substituted or unsubstituted indole.

In certain embodiments, R⁵ is of formula

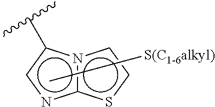

wherein the alkyl is substituted or unsubstituted. In certain embodiments, R⁵ is of formula

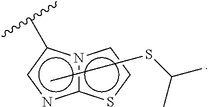

In certain embodiments, R⁵ is of formula

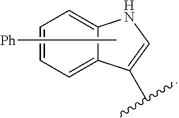

In certain embodiments, R⁵ is of formula

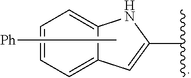

In certain embodiments, R⁵ is of formula:

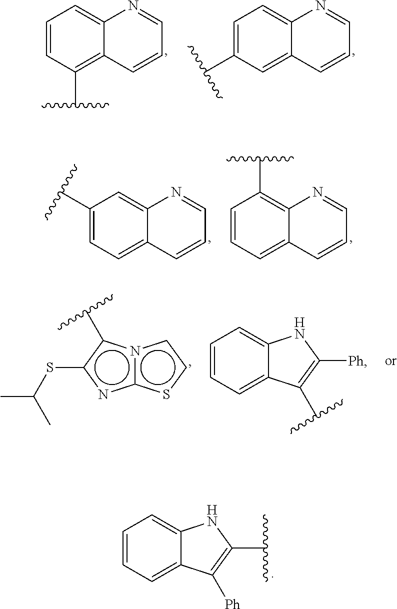

In certain embodiments, a compound of Formula (III) is not of formula:

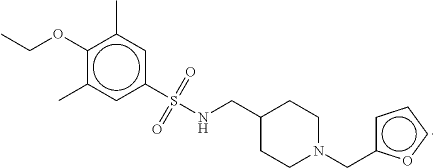

In certain embodiments, R⁵ is not unsubstituted heteroaryl. In certain embodiments, R⁵ is not unsubstituted 5-membered heteroaryl. In certain embodiments, R⁵ is not unsubstituted furyl. In certain embodiments, R⁵ is not of formula:

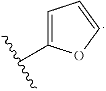 [SMILES: *c1ccco1]

In certain embodiments, R⁴ is not trisubstituted phenyl. In certain embodiments, R⁴ is not of formula:

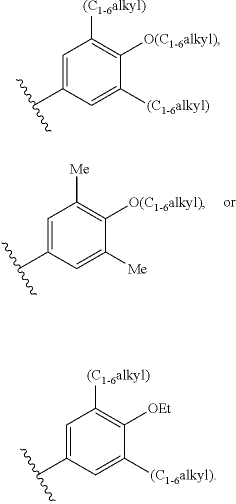

In certain embodiments, R⁴ is not of formula:

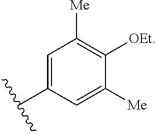 [SMILES: *c1cc(C)c(OCC)c(C)c1]

### Compounds of Formula (IV)

In certain embodiments, the present disclosure provides a compound of Formula (IV):

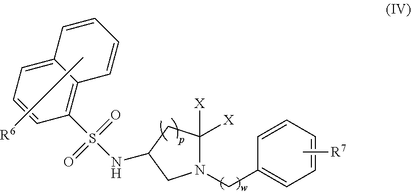

or a pharmaceutically acceptable salt thereof,
wherein:
R⁶ is hydrogen or —N(R^{6a})₂;
each R^{6a} is independently substituted or unsubstituted alkyl;
each X is hydrogen, or two X groups are joined to form ═O;
R⁷ is hydrogen, halogen, substituted or unsubstituted alkyl, or —N(R^{7a})₂;
R^{7a} is hydrogen or substituted or unsubstituted alkyl;
p is 0 or 1; and
w is 0 or 1,
   wherein if p is 0, then two X groups are not joined to form ═O.

In certain embodiments, a compound of Formula (IV) is of Formula (IV-a):

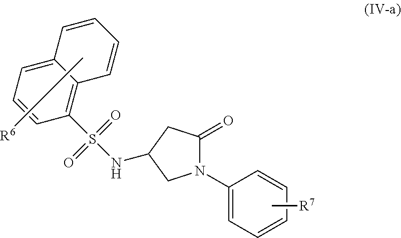

or a pharmaceutically acceptable salt thereof.

In certain embodiments, a compound of Formula (IV) is of Formula (IV-b):

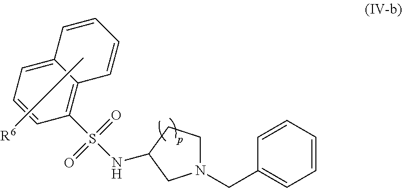

or a pharmaceutically acceptable salt thereof.

In certain embodiments, a compound of Formula (IV) is of Formula (IV-c):

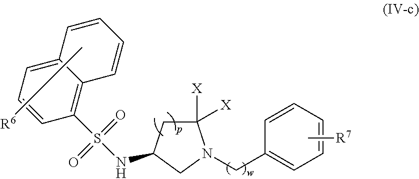

or a pharmaceutically acceptable salt thereof.

In certain embodiments, a compound of Formula (IV) is of Formula (IV-d):

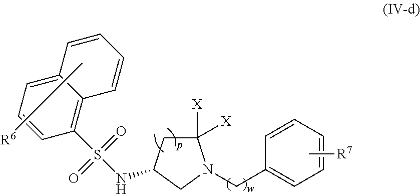

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, R⁶ is hydrogen.

In certain embodiments, R⁶ is —N(R^{6a})₂; and each R^{6a} is independently substituted or unsubstituted alkyl. In certain embodiments, R⁶ is —N(R^{6a})₂; and each R^{6a} is independently substituted or unsubstituted C₁₋₆alkyl. In certain embodiments, R⁶ is —N(R^{6a})₂; and each R^{6a} is independently substituted or unsubstituted C₁₋₃alkyl. In certain embodiments, R⁶ is —N(R^{6a})₂; and each R^{6a} is independently substituted or unsubstituted methyl. In certain embodiments, R⁶ is —N(R^{6a})₂; and each R^{6a} is independently substituted or unsubstituted ethyl. In certain embodiments, R⁶ is —N(R^{6a})₂; and each R^{6a} is independently substituted or unsubstituted propyl. In certain embodiments, R⁶ is —N(R^{6a})₂; and each R^{6a} is independently substituted or unsubstituted isopropyl.

In certain embodiments, the naphthyl ring to which —N(R^{6a})₂ is attached is of formula:

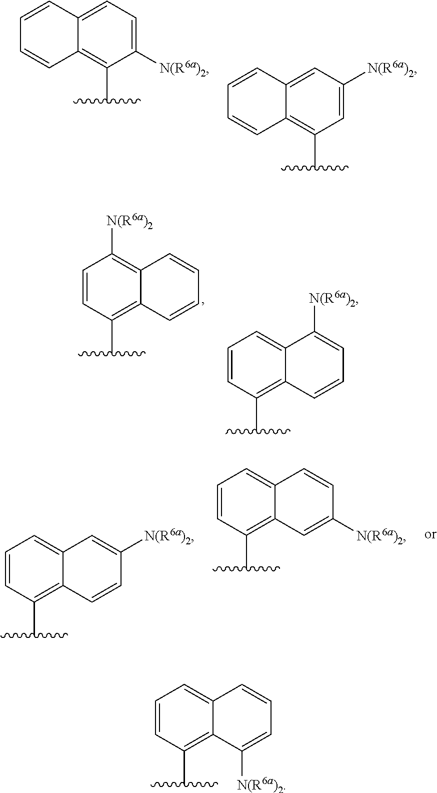

In certain embodiments, R⁶ is —N(R^{6a})₂; each R^{6a} is unsubstituted methyl; and the naphthyl ring to which —N(R^{6a})₂ is attached is of formula:

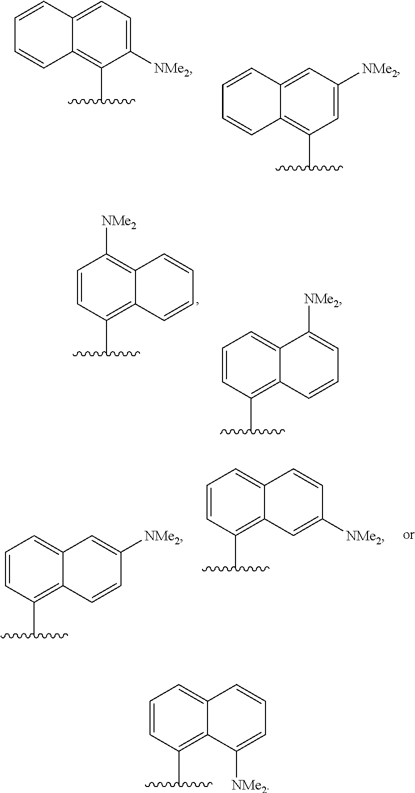

In certain embodiments, R⁷ is hydrogen.

In certain embodiments, R⁷ is halogen (e.g., fluoro, chloro, bromo, or iodo).

In certain embodiments, R⁷ is substituted or unsubstituted alkyl. In certain embodiments, R⁷ is substituted or unsubstituted C₁₋₆alkyl. In certain embodiments, R⁷ is substituted or unsubstituted C₁₋₃alkyl. In certain embodiments, R⁷ is substituted or unsubstituted methyl. In certain embodiments, R⁷ is substituted or unsubstituted ethyl. In certain embodiments, R⁷ is substituted or unsubstituted propyl. In certain embodiments, R⁷ is substituted or unsubstituted isopropyl.

In certain embodiments, R⁷ is —N(R^{7a})₂; and at least one R^{7a} is hydrogen. In certain embodiments, R⁷ is —NH₂. In certain embodiments, R⁷ is —N(R^{7a})₂; and each R^{7a} is independently substituted or unsubstituted alkyl. In certain embodiments, R⁷ is —N(R^{7a})₂; and each R^{7a} is independently substituted or unsubstituted C₁₋₆alkyl. In certain embodiments, R⁷ is —N(R^{7a})₂; and each R^{7a} is independently substituted or unsubstituted C₁₋₃alkyl. In certain embodiments, R⁷ is —N(R^{7a})₂; and each R^{7a} is independently substituted or unsubstituted methyl. In certain embodiments, R⁷ is —N(R^{7a})₂; and each R^{7a} is independently substituted or unsubstituted ethyl. In certain embodiments, R⁷ is —N(R^{7a})₂; and each R^{7a} is independently substituted or unsubstituted propyl. In certain embodiments, R⁷ is —N(R^{7a})₂; and each R^{7a} is independently substituted or unsubstituted isopropyl.

In certain embodiments, the phenyl ring to which R⁷ is attached is of formula:

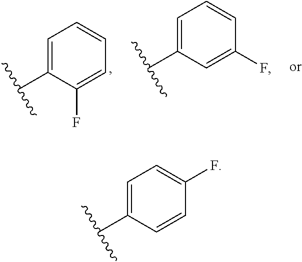

In certain embodiments, the phenyl ring to which R⁷ is attached is of formula:

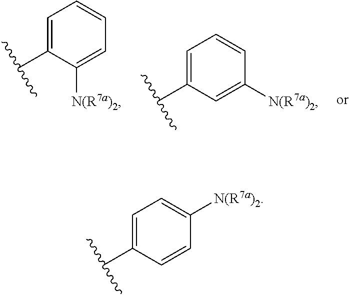

In certain embodiments, the phenyl ring to which R⁷ is attached is of formula:

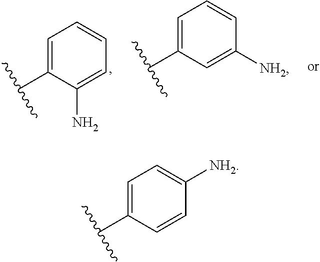

In certain embodiments, the phenyl ring to which R⁷ is attached is of formula:

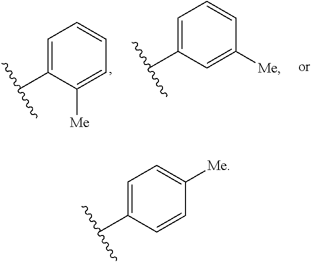

In certain embodiments, p is 0, w is 0, and each X is hydrogen. In certain embodiments, p is 0, w is 1, and each X is hydrogen. In certain embodiments, p is 1, w is 0, and each X is hydrogen. In certain embodiments, p is 1, w is 1, and each X is hydrogen. In certain embodiments, p is 0, w is 0, and two X groups are not joined to form ═O. In certain embodiments, p is 0, w is 1, and two X groups are not joined to form ═O. In certain embodiments, p is 1, w is 0, and two X groups are joined to form ═O. In certain embodiments, p is 1, w is 1, and two X groups are joined to form ═O.

### Compounds of Formula (V)

In certain embodiments, the present disclosure provides a compound of Formula (V):

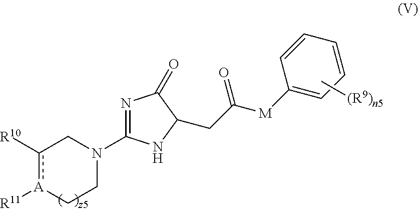

or a pharmaceutically acceptable salt thereof,
wherein:
each occurrence of R⁹ is independently substituted or unsubstituted alkyl, —OR^{9a}, —C(═O)R^{9a}, —C(═O)OR^{9a}, —(C═O)NHR^{9a}, —NH(C═O)R^{9a}, —CN, or halogen, or two vicinal R⁹ are taken together with their intervening atoms to form a substituted or unsubstituted heterocyclic ring;
each occurrence of R^{9a} is independently hydrogen, independently substituted or unsubstituted alkyl, or substituted or unsubstituted aryl;
M is a bond or NH;
when [symbol] is a single bond, then A is N and R¹¹ is present, or A is CH or C(OH) and R¹¹ is present;
when [symbol] is a double bond, then A is N and R¹¹ is absent, or A is C and R¹¹ is present; and
R¹⁰ is hydrogen;
R¹¹ is substituted or unsubstituted alkyl, substituted or unsubstituted aralkyl, substituted or unsubstituted heteroaralkyl, substituted or unsubstituted aryl, or substituted or unsubstituted heteroaryl; or
R¹⁰ and R¹¹ are taken together with their intervening atoms to form an unsubstituted fused phenyl ring; and
n5 is 0, 1, 2, 3, 4, or 5; and
z5 is 0 or 1.

In certain embodiments, R¹¹ is substituted or unsubstituted phenyl. In certain embodiments, R¹¹ is unsubstituted phenyl.

In certain embodiments, the compound of Formula (V) is a compound of Formula (V′):

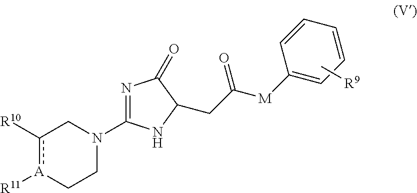

or a pharmaceutically acceptable salt thereof,
wherein:
R⁹ is —OR^{9a} or halogen;
R^{9a} is substituted or unsubstituted alkyl;
M is a bond or NH;
when [symbol] is a single bond, then A is N and R¹¹ is present, or A is CH and R¹¹ is present;
when [symbol] is a double bond, then A is N and R¹¹ is absent, or A is C and R¹¹ is present; and
R¹⁰ is hydrogen and R¹¹ is unsubstituted phenyl; or
R¹⁰ and R¹¹ are taken together with their intervening atoms to form an unsubstituted fused phenyl ring.

In certain embodiments, the provided compound is of a free base form. In certain embodiments, the provided compound is in the form of a pharmaceutically acceptable salt as generally defined herein.

In certain embodiments, R⁹ is a halogen (e.g., fluoro, chloro, bromo, or iodo).

In certain embodiments, R⁹ is —OR^{9a}; and R^{9a} is substituted or unsubstituted alkyl. In certain embodiments, R⁹ is —OR^{9a}; and R^{9a} is substituted or unsubstituted C₁₋₆alkyl. In certain embodiments, R⁹ is —OR^{9a}; and R^{9a} is substituted or unsubstituted C₁₋₃alkyl. In certain embodiments, R⁹ is —OR^{9a}; and R^{9a} is substituted or unsubstituted methyl. In certain embodiments, R⁹ is —OR^{9a}; and R^{9a} is substituted or unsubstituted ethyl. In certain embodiments, R⁹ is —OR^{9a}; and R^{9a} is substituted or unsubstituted propyl. In certain embodiments, R⁹ is —OR^{9a}; and R^{9a} is substituted or unsubstituted isopropyl.

In certain embodiments, the phenyl ring to which R⁹ is attached is of formula:

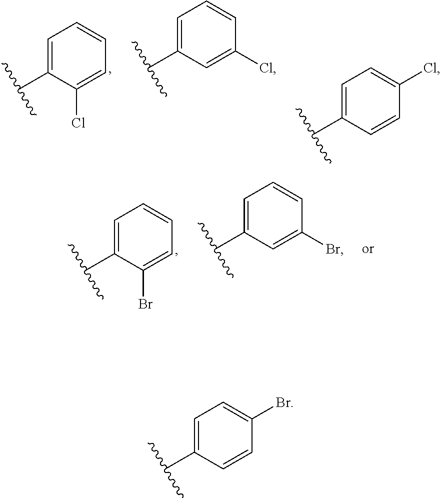

In certain embodiments, the phenyl ring to which R⁹ is attached is of formula:

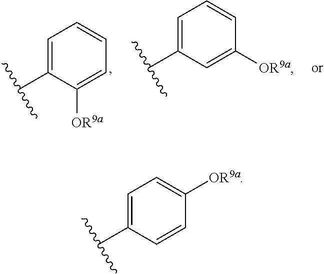

In certain embodiments, M is a bond. In certain embodiments, M is NH.

In certain embodiments, M is a bond and the phenyl ring to which R⁹ is attached is of formula:

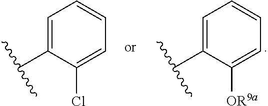

In certain embodiments, M is NH and the phenyl ring to which R⁹ is attached is of formula:

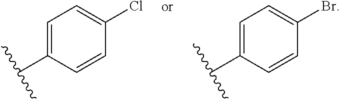 [SMILES: *c1ccc(Cl)cc1 ; "or" ; *c1ccc(Br)cc1]

In certain embodiments, the ring system:

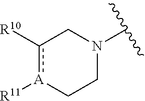

corresponds to:

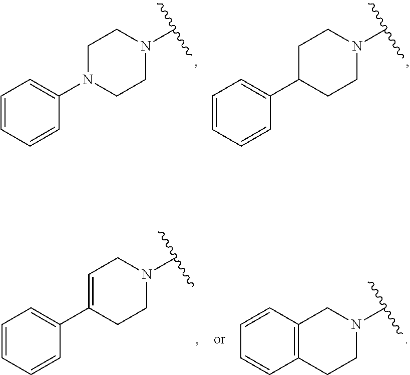 [SMILES: *N1CCN(c2ccccc2)CC1 ; *N1CCC(c2ccccc2)CC1 ; *N1CC=C(c2ccccc2)CC1 ; *N1CCc2ccccc2C1]

### Compounds of Formula (VI)

In certain embodiments, the present disclosure provides a compound of Formula (VI):

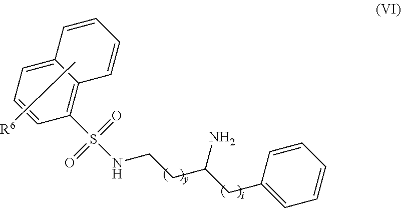

or a pharmaceutically acceptable salt thereof,
wherein:
R⁶ is hydrogen or —N(R^{6a})₂;
each R^{6a} is independently substituted or unsubstituted alkyl; and
y and i are each independently 0 or 1.

In certain embodiments, a compound of Formula (VI) is of Formula (VI-a):

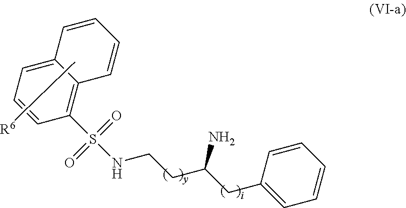

or a pharmaceutically acceptable salt thereof.

In certain embodiments, a compound of Formula (VI) is of Formula (VI-b):

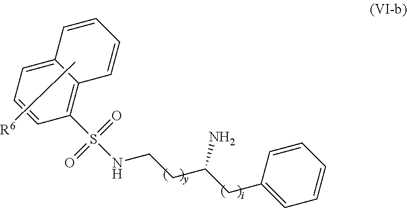

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, y is 0. In certain embodiments, y is 1. In certain embodiments, i is 0. In certain embodiments, i is 1.

In certain embodiments, y is 0; and i is 0. In certain embodiments, y is 0; and i is 1. In certain embodiments, y is 1; and i is 0. In certain embodiments, y is 1; and i is 1.

In certain embodiments, each instance of R^{6a} is independently unsubstituted alkyl, e.g., unsubstituted C₁₋₆alkyl, e.g., unsubstituted C₁alkyl, C₂alkyl, C₃alkyl, C₄alkyl, C₅alkyl, or C₆alkyl. In certain embodiments, each instance of R^{6a} is independently substituted alkyl, e.g., substituted C₁₋₆alkyl, e.g., substituted C₁alkyl, C₂alkyl, C₃alkyl, C₄alkyl, C₅alkyl, or C₆alkyl.

### Compounds of Formula (VII)

In certain embodiments, the present disclosure provides a compound of Formula (VII):

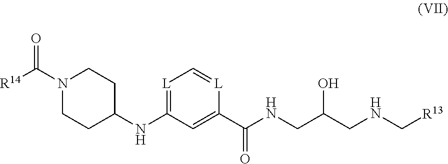

or a pharmaceutically acceptable salt thereof,
wherein:
R¹³ is substituted or unsubstituted aryl;
R¹⁴ is substituted or unsubstituted alkyl; and
L is independently CH or N, wherein at least one of L must be N.

In certain embodiments, a compound of Formula (VII) is of Formula (VII-a):

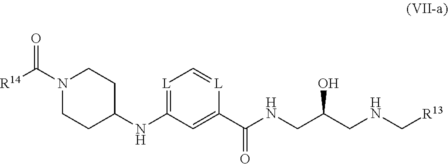

or a pharmaceutically acceptable salt thereof.

In certain embodiments, a compound of Formula (VII) is of Formula (VII-b):

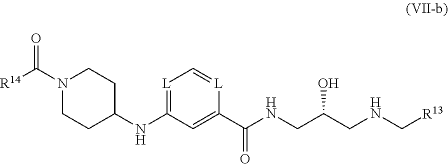

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, R¹³ is substituted or unsubstituted aryl. In certain embodiments, R¹³ is monosubstituted phenyl. In certain embodiments, R¹³ is disubstituted phenyl. In certain embodiments, R¹³ is trisubstituted phenyl. In certain embodiments, R¹³ is tetrasubstituted phenyl. In certain embodiments, R¹³ is pentasubstituted phenyl. In certain embodiments, R¹³ is unsubstituted phenyl.

In certain embodiments, R¹⁴ is substituted or unsubstituted alkyl. In certain embodiments, R¹⁴ is substituted or unsubstituted C₁₋₆alkyl. In certain embodiments, R¹⁴ is substituted or unsubstituted C₁₋₃alkyl. In certain embodiments, R¹⁴ is substituted or unsubstituted methyl. In certain embodiments, R¹⁴ is substituted or unsubstituted ethyl. In certain embodiments, R¹⁴ is substituted or unsubstituted propyl. In certain embodiments, R¹⁴ is substituted or unsubstituted isopropyl.

### Compounds of Formula (VIII)

In certain embodiments, the present disclosure provides a compound of Formula (VIII):

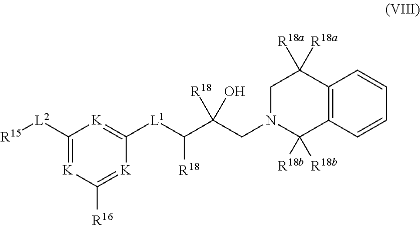

or a pharmaceutically acceptable salt thereof,
wherein:
L¹ is O, —C(═O)NH—, or —C(═O)NMe-;
L² is a bond or NH;
each R¹⁵ is independently substituted or unsubstituted cycloalkyl, substituted or unsubstituted heterocyclyl, or substituted or unsubstituted heteroaryl;
R¹⁶ is hydrogen or —NHR¹⁷;
R¹⁷ is substituted or unsubstituted heterocyclyl;
each R¹⁸ is independently hydrogen or —CH₃;
each R^{18a} is hydrogen or both instances of R^{18a} are taken together with their intervening atoms to form a substituted or unsubstituted cyclopropyl ring;
each R^{18b} is independently hydrogen or —CH₃;
or one instance of R^{18a} and one instance of R^{18b} are taken together with their intervening atoms to form a substituted or unsubstituted pyrrolidine or piperidine ring; and
each K is independently CH or N, wherein no more than two of K can be N.

In certain embodiments, a compound of Formula (VIII) is of Formula (VIII-a):

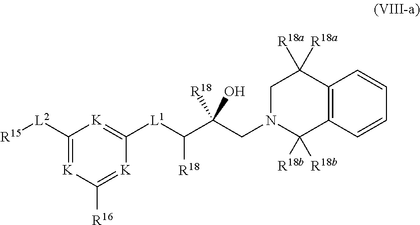

or a pharmaceutically acceptable salt thereof.

In certain embodiments, a compound of Formula (VIII) is of Formula (VIII-b):

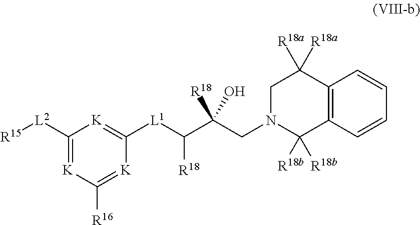

or a pharmaceutically acceptable salt thereof.

In certain embodiments, a compound of Formula (VIII) is of Formula (VIII-c):

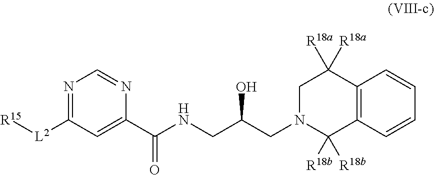

or a pharmaceutically acceptable salt thereof.

In certain embodiments, a compound of Formula (VIII) is of Formula (VIII-d):

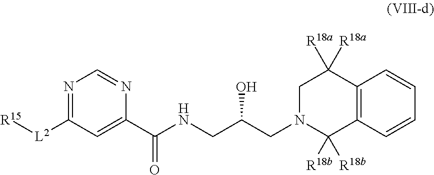

or a pharmaceutically acceptable salt thereof.

In certain embodiments, a compound of Formula (VIII) is of Formula (VIII-e):

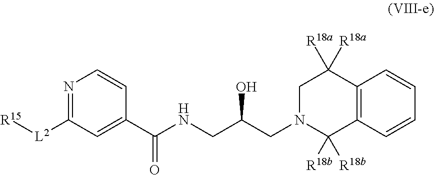

or a pharmaceutically acceptable salt thereof.

In certain embodiments, a compound of Formula (VIII) is of Formula (VIII-f):

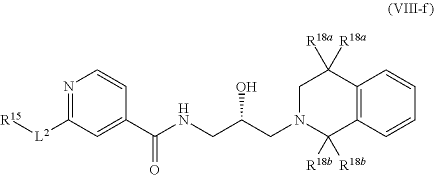

or a pharmaceutically acceptable salt thereof.

In certain embodiments, a compound of Formula (VIII) is of Formula (VIII-g):

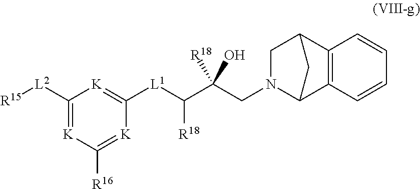

or a pharmaceutically acceptable salt thereof.

In certain embodiments, a compound of Formula (VIII) is of Formula (VIII-h):

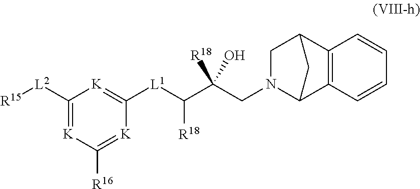

or a pharmaceutically acceptable salt thereof.

In certain embodiments, a compound of Formula (VIII) is of Formula (VIII-i):

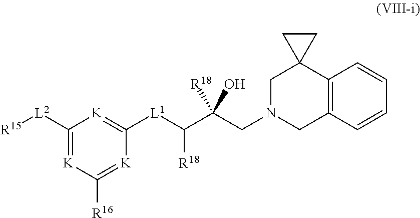

or a pharmaceutically acceptable salt thereof.

In certain embodiments, a compound of Formula (VIII) is of Formula (VIII-j):

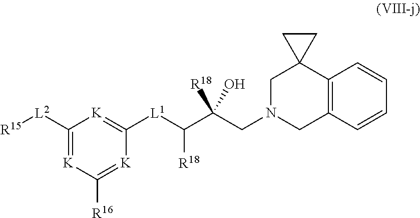

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, L¹ is O. In certain embodiments, L¹ is —C(═O)NH—. In certain embodiments, L¹ is —C(═O)NMe-.

In certain embodiments, L² is a bond. In certain embodiments, L² is NH.

In certain embodiments, L¹ is O; and L² is a bond. In certain embodiments, L¹ is O; and L² is NH. In certain embodiments, L¹ is C(═O)NH; and L² is a bond. In certain embodiments, L¹ is C(═O)NH; and L² is NH. In certain embodiments, L¹ is C(═O)NMe; and L² is a bond. In certain embodiments, L¹ is C(═O)NMe; and L² is NH.

In certain embodiments, R¹⁵ is substituted or unsubstituted cycloalkyl. In certain embodiments, R¹⁵ is substituted or unsubstituted 3-6 membered cycloalkyl. In certain embodiments, R¹⁵ is substituted or unsubstituted cyclopropyl. In certain embodiments, R¹⁵ is substituted or unsubstituted cyclobutyl. In certain embodiments, R¹⁵ is substituted or unsubstituted cyclopentyl. In certain embodiments, R¹⁵ is substituted or unsubstituted cyclohexyl.

In certain embodiments, R¹⁵ is substituted or unsubstituted heterocyclyl. In certain embodiments, R¹⁵ is substituted or unsubstituted 4-6 membered heterocyclyl. In certain embodiments, R¹⁵ is substituted or unsubstituted oxetanyl. In certain embodiments, R¹⁵ is substituted or unsubstituted azetidinyl. In certain embodiments, R¹⁵ is substituted or unsubstituted tetrahydrofuryl. In certain embodiments, R¹⁵ is substituted or unsubstituted pyrrolidinyl. In certain embodiments, R¹⁵ is substituted or unsubstituted tetrahydropyranyl. In certain embodiments, R¹⁵ is substituted or unsubstituted piperidinyl. In certain embodiments, R¹⁵ is substituted or unsubstituted piperazinyl.

In certain embodiments, R¹⁵ is of formula:

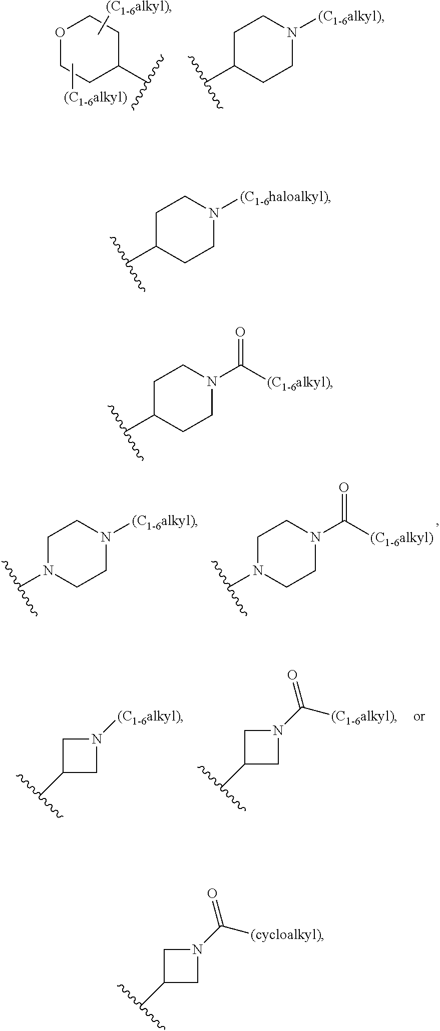

wherein the alkyl, haloalkyl, or cycloalkyl is substituted or unsubstituted.

In certain embodiments, R¹⁵ is of formula:

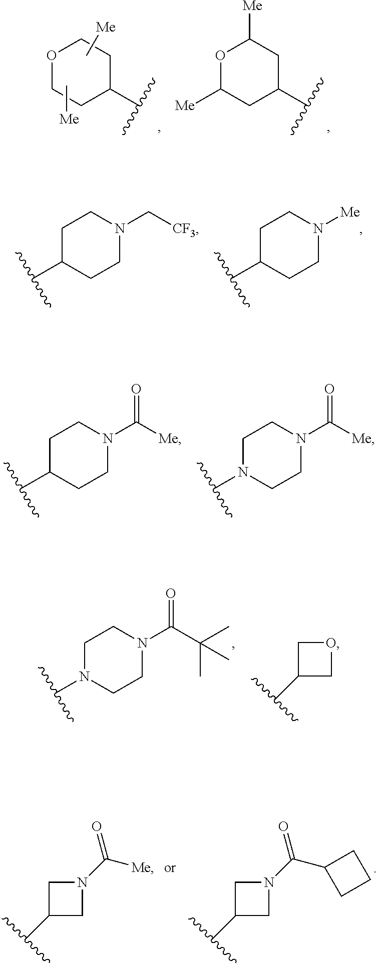

In certain embodiments, R¹⁵ is substituted or unsubstituted heteroaryl. In certain embodiments, R¹⁵ is substituted or unsubstituted 5-6 membered heteroaryl. In certain embodiments, R¹⁵ is substituted or unsubstituted imidazolyl. In certain embodiments, R¹⁵ is substituted or unsubstituted pyrazolyl. In certain embodiments, R¹⁵ is substituted or unsubstituted oxazolyl. In certain embodiments, R¹⁵ is substituted or unsubstituted thiazolyl. In certain embodiments, R¹⁵ is substituted or unsubstituted oxadiazolyl. In certain embodiments, R¹⁵ is substituted or unsubstituted thiadiazolyl. In certain embodiments, R¹⁵ is substituted or unsubstituted triazolyl. In certain embodiments, R¹⁵ is substituted or unsubstituted tetrazolyl. In certain embodiments, R¹⁵ is substituted or unsubstituted pyridyl. In certain embodiments, R¹⁵ is substituted or unsubstituted pyrazinyl. In certain embodiments, R¹⁵ is substituted or unsubstituted pyrimidinyl. In certain embodiments, R¹⁵ is substituted or unsubstituted pyridizinyl.

In certain embodiments, R¹⁵ is a substituted or unsubstituted 8- to 10-membered bicyclic heteroaryl having 1-4 heteroatoms independently selected from nitrogen, oxygen, and sulfur. In certain embodiments, R¹⁵ is a substituted or unsubstituted 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl). In certain embodiments, R¹⁵ is a substituted or unsubstituted 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl). In certain embodiments, R¹⁵ is unsubstituted benzothiazole; and L² is a bond.

In certain embodiments, R¹⁶ is hydrogen. In certain embodiments, R¹⁶ is —NHR¹⁷; and R¹ is substituted or unsubstituted heterocyclyl. In certain embodiments, R¹⁶ is —NHR¹⁷; and R¹⁷ is substituted or unsubstituted 4-6 membered heterocyclyl. In certain embodiments, R¹⁶ is —NHR¹⁷; and R¹⁷ is substituted or unsubstituted oxetanyl. In certain embodiments, R¹⁶ is —NHR¹⁷; and R¹⁷ is substituted or unsubstituted azetidinyl. In certain embodiments, R¹⁶ is —NHR¹⁷; and R¹⁷ is substituted or unsubstituted tetrahydrofuryl. In certain embodiments, R¹⁶ is —NHR¹⁷; and R¹⁷ is substituted or unsubstituted pyrrolidinyl. In certain embodiments, R¹⁶ is —NHR¹⁷; and R¹⁷ is substituted or unsubstituted tetrahydropyranyl. In certain embodiments, R¹⁶ is —NHR¹⁷; and R¹⁷ is substituted or unsubstituted piperidinyl. In certain embodiments, R¹⁶ is —NHR⁷; and R¹⁷ is substituted or unsubstituted piperazinyl.

In certain embodiments, each R⁸ is hydrogen. In certain embodiments, one instance of R¹⁸ is —CH₃. In certain embodiments, two instances of R¹⁸ is —CH₃.

In certain embodiments, each R^{18a} is hydrogen. In certain embodiments, both instances of R^{18a} are taken together with their intervening atoms to form a substituted or unsubstituted cyclopropyl ring.

In certain embodiments, each R^{18b} is hydrogen. In certain embodiments, one of R^{18b} is —CH₃. In certain embodiments, both of R^{18b} is —CH₃.

In certain embodiments, one instance of R^{18a} and one instance of R^{18b} are taken together with their intervening atoms to form a substituted or unsubstituted pyrrolidine ring. In certain embodiments, one instance of R^{18a} and one instance of R^{18b} are taken together with their intervening atoms to form a substituted or unsubstituted piperidine ring.

In certain embodiments, the ring to which R^{18a} and R^{18b} are attached is of formula:

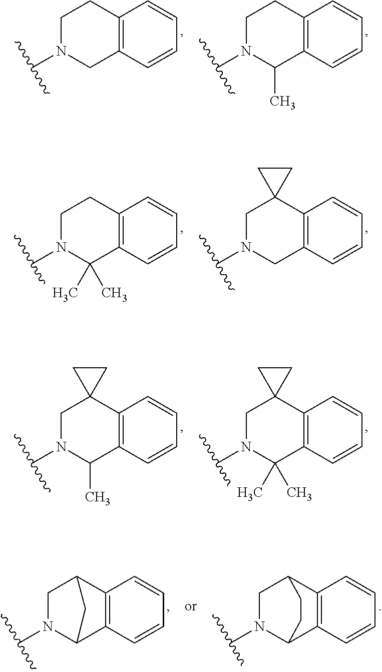

In certain embodiments, one instance of K is CH. In certain embodiments, two instances of K are CH. In certain embodiments, all three instances of K are CH. In certain embodiments, one instance of K is N. In certain embodiments, two instances of K are N.

In certain embodiments, a provided compound is not of any one of the following formulae:

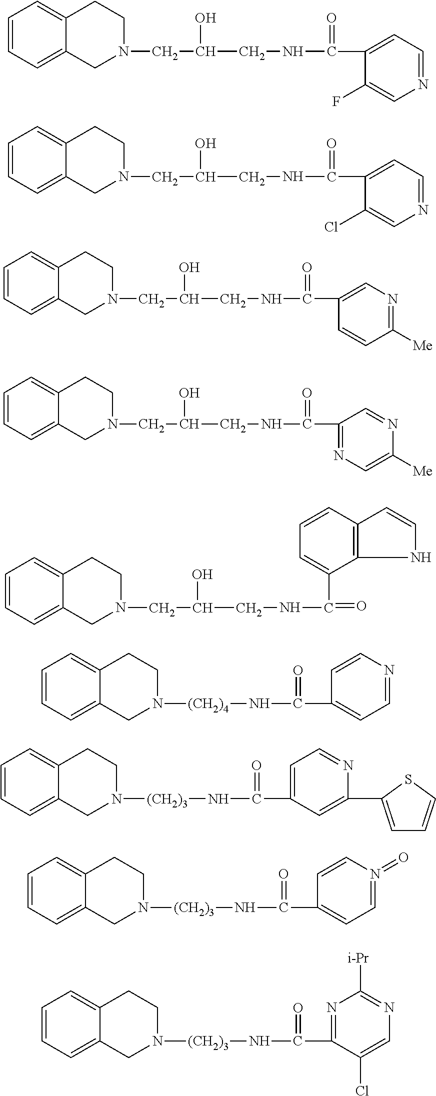

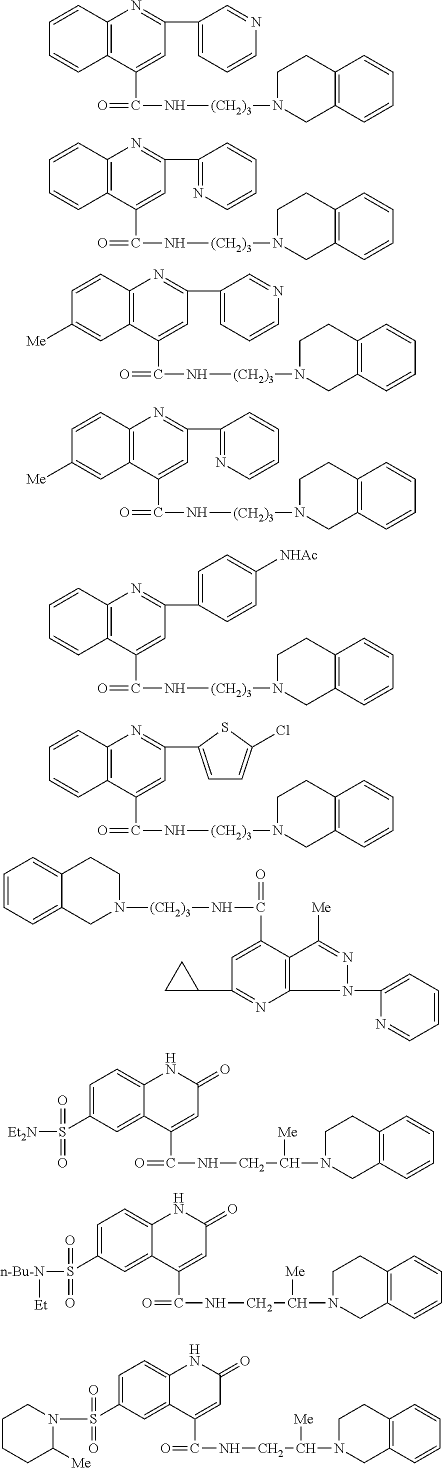

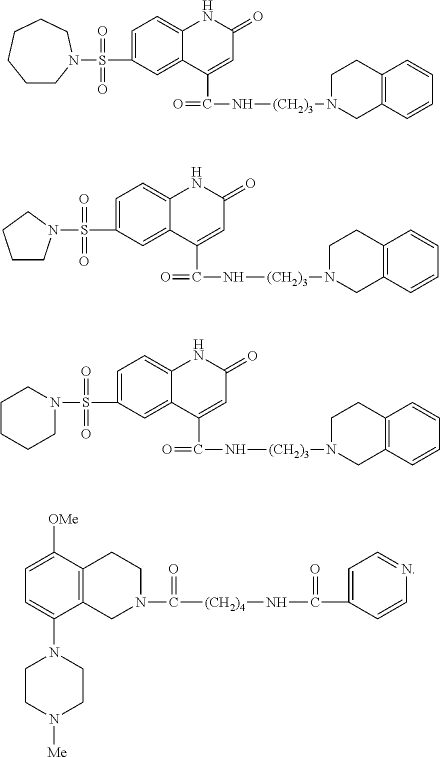

### Compounds of Formula (IX)

In certain embodiments, the present disclosure provides a compound of Formula (IX):

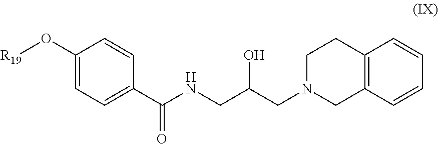

or a pharmaceutically acceptable salt thereof, wherein R¹⁹ is substituted or unsubstituted heterocyclyl.

In certain embodiments, a compound of Formula (IX) is of Formula (IX-a):

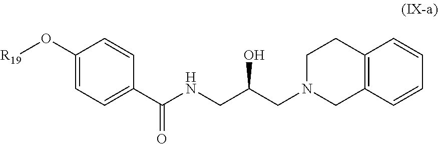

or a pharmaceutically acceptable salt thereof.

In certain embodiments, a compound of Formula (IX) is of Formula (IX-b):

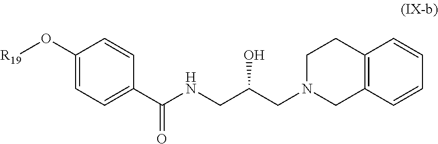

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, R¹⁹ is substituted or unsubstituted heterocyclyl. In certain embodiments, R¹⁹ is substituted or unsubstituted 4-6 membered heterocyclyl. In certain embodiments, R¹⁹ is substituted or unsubstituted oxetanyl. In certain embodiments, R¹⁹ is substituted or unsubstituted azetidinyl. In certain embodiments, R¹⁹ is substituted or unsubstituted tetrahydrofuryl. In certain embodiments, R¹⁹ is substituted or unsubstituted pyrrolidinyl. In certain embodiments, R¹⁹ is substituted or unsubstituted tetrahydropyranyl. In certain embodiments, R¹⁹ is substituted or unsubstituted piperidinyl. In certain embodiments, R¹⁹ is substituted or unsubstituted piperazinyl.

In certain embodiments, R¹⁹ is of formula:

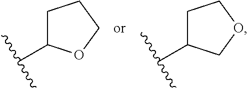 [SMILES: *C1CCCO1 ; *C1CCOC1]

### Compounds of Formula (X)

In certain embodiments, the present disclosure provides a compound of Formula (X):

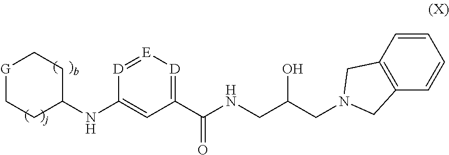

or a pharmaceutically acceptable salt thereof,
wherein:
D is CH or N;
E is C(R²¹) or N, wherein if E is C(R²¹) then at least one instance of D is N and if E is N then neither instance of D is N;
G is O or N(R²⁰);
R²⁰ is hydrogen, substituted or unsubstituted alkyl, —SO₂R^{20a}, or —C(═O)R^{20a}
R^{20a} is substituted or unsubstituted alkyl or substituted or unsubstituted cycloalkyl;
R²¹ is hydrogen or substituted or unsubstituted morpholine; and
j and b are each independently 0 or 1.

In certain embodiments, a compound of Formula (X) is of Formula (X-a):

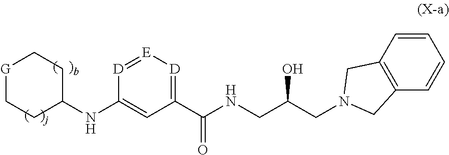

or a pharmaceutically acceptable salt thereof.

In certain embodiments, a compound of Formula (X) is of Formula (X-b):

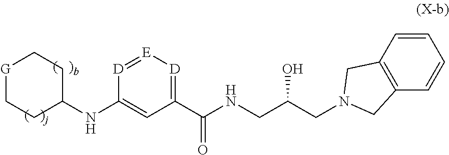

or a pharmaceutically acceptable salt thereof.

In certain embodiments, the provided compound is of a free base form. In certain embodiments, the provided compound is in the form of a pharmaceutically acceptable salt as generally defined herein.

In certain embodiments, the ring comprising D and E is of formula:

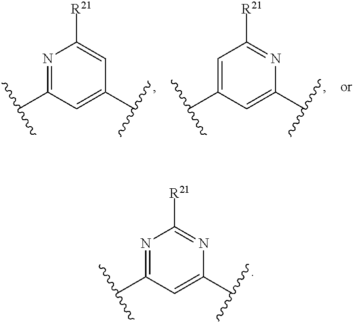

In certain embodiments, the ring comprising D and E is of formula:

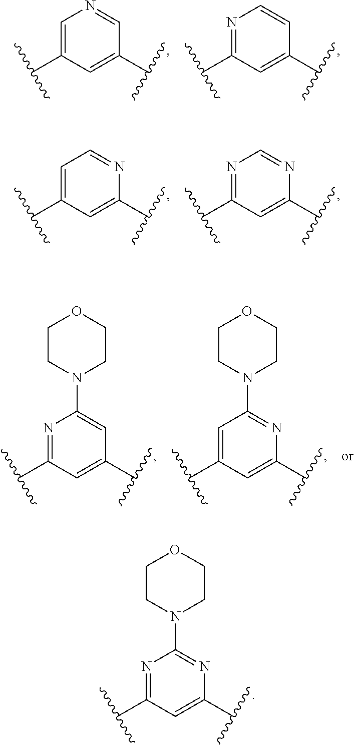

In certain embodiments, G is O. In certain embodiments, G is N(R²⁰); and R²⁰ is hydrogen. In certain embodiments, G is N(R²⁰); and R²⁰ is substituted alkyl. In certain embodiments, G is N(R²⁰); and R²⁰ is substituted or unsubstituted C₁₋₆alkyl. In certain embodiments, G is N(R²⁰); and R²⁰ is substituted or unsubstituted C₁₋₃alkyl. In certain embodiments, G is N(R²⁰); and R²⁰ is substituted or unsubstituted methyl. In certain embodiments, G is N(R²⁰); and R²⁰ is substituted or unsubstituted ethyl. In certain embodiments, G is N(R²⁰); and R²⁰ is substituted or unsubstituted propyl. In certain embodiments, G is N(R²⁰); and R²⁰ is substituted or unsubstituted isopropyl.

In certain embodiments, G is N(R²⁰); R²⁰ is —SO₂R^{20a}; and R^{20a} is substituted or unsubstituted alkyl. In certain embodiments, G is N(R²⁰); R²⁰ is —SO₂R^{20a}; and R^{20a} is substituted or unsubstituted C₁₋₆alkyl. In certain embodiments, G is N(R²⁰); R²⁰ is —SO₂R^{20a}; and R^{20a} is substituted or unsubstituted C₁₋₃alkyl. In certain embodiments, G is N(R²⁰); R²⁰ is —SO₂R^{20a}; and R^{20a} is substituted or unsubstituted methyl. In certain embodiments, G is N(R²⁰); R²⁰ is —SO₂R^{20a}; and R^{20a} is substituted or unsubstituted ethyl. In certain embodiments, G is N(R²⁰); R²⁰ is —SO₂R^{20a}; and R^{20a} is substituted or unsubstituted propyl. In certain embodiments, G is N(R²⁰); R²⁰ is —SO₂R^{20a}; and R^{20a} is substituted or unsubstituted isopropyl.

In certain embodiments, G is N(R²⁰); R²⁰ is —SO₂R^{20a}; and R^{20a} is substituted or unsubstituted cycloalkyl. In certain embodiments, G is N(R²⁰); R²⁰ is —SO₂R^{20a}; and R^{20a} is substituted or unsubstituted 3-6 membered cycloalkyl. In certain embodiments, G is N(R²⁰); R²⁰ is —SO₂R^{20a}; and R^{20a} is substituted or unsubstituted cyclopropyl. In certain embodiments, G is N(R²⁰); R²⁰ is —SO₂R^{20a}; and R^{20a} is substituted or unsubstituted cyclobutyl. In certain embodiments, G is N(R²⁰); R²⁰ is —SO₂R^{20a}; and R^{20a} is substituted or unsubstituted cyclopentyl. In certain embodiments, G is N(R²⁰); R²⁰ is —SO₂R^{20a}; and R^{20a} is substituted or unsubstituted cyclohexyl.

In certain embodiments, G is N(R²⁰); R²⁰ is —C(═O)R^{20a}; and R^{20a} is substituted or unsubstituted alkyl. In certain embodiments, G is N(R²⁰); R²⁰ is —C(═O)R^{20a}; and R^{20a} is substituted or unsubstituted C₁₋₆alkyl. In certain embodiments, G is N(R²⁰); R²⁰ is —C(═O)R^{20a}; and R^{20a} is substituted or unsubstituted C₁₋₃alkyl. In certain embodiments, G is N(R²⁰); R²⁰ is —C(═O)R^{20a}; and R^{20a} is substituted or unsubstituted methyl. In certain embodiments, G is N(R²⁰); R²⁰ is —C(═O)R^{20a}; and R^{20a} is substituted or unsubstituted ethyl. In certain embodiments, G is N(R²⁰); R²⁰ is —C(═O)R^{20a}; and R^{20a} is substituted or unsubstituted propyl. In certain embodiments, G is N(R²⁰); R²⁰ is —C(═O)R^{20a}; and R^{20a} is substituted or unsubstituted isopropyl.

In certain embodiments, G is N(R²⁰); R²⁰ is —C(═O)R^{20a}; and R^{20a} is substituted or unsubstituted cycloalkyl. In certain embodiments, G is N(R²⁰); R²⁰ is —C(═O)R^{20a}; and R^{20a} is substituted or unsubstituted 3-6 membered cycloalkyl. In certain embodiments, G is N(R²⁰); R²⁰ is —C(═O)R^{20a}; and R^{20a} is substituted or unsubstituted cyclopropyl. In certain embodiments, G is N(R²⁰); R²⁰ is —C(═O)R^{20a}; and R^{20a} is substituted or unsubstituted cyclobutyl. In certain embodiments, G is N(R²⁰); R²⁰ is —C(═O)R^{20a}; and R^{20a} is substituted or unsubstituted cyclopentyl. In certain embodiments, G is N(R²⁰); R²⁰ is —C(═O)R^{20a}; and R^{20a} is substituted or unsubstituted cyclohexyl.

In certain embodiments, j is 0; and b is 0. In certain embodiments, j is 1; and b is 0. In certain embodiments, j is 0; and b is 1. In certain embodiments, j is 1; and b is 1.

In certain embodiments, the ring comprising G is of formula:

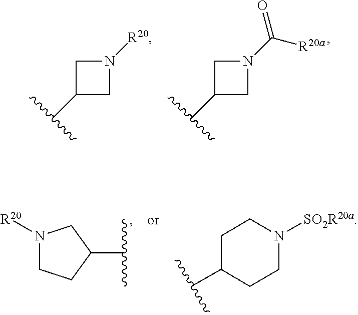

In certain embodiments, the ring comprising G is of formula:

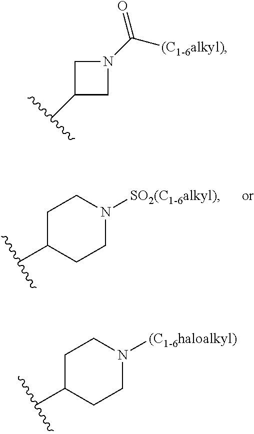

wherein the alkyl and haloalkyl are substituted or unsubstituted.

In certain embodiments, the ring comprising G is of formula:

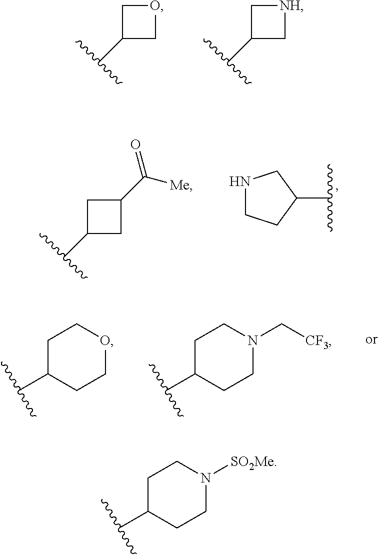

### Compounds of Formula (XI)

In certain embodiments, the present disclosure provides a compound of Formula (XI):

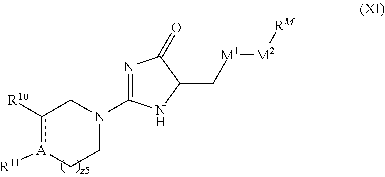

or a pharmaceutically acceptable salt thereof,
wherein:
R^{M} is substituted or unsubstituted carbocyclyl, substituted or unsubstituted heterocyclyl, or a phenyl ring substituted with 0, 1, 2, 3, 4, or 5 R⁹;
each occurrence of R⁹ is independently substituted or unsubstituted alkyl, —OR^{9a}, —C(═O)R^{9a}, —C(═O)OR^{9a}, —(C═O)NHR^{9a}, —NH(C═O)R^{9a}, —CN, or halogen, or two vicinal R⁹ are taken together with their intervening atoms to form a substituted or unsubstituted heterocyclic ring;
each occurrence of R^{9a} is independently hydrogen, independently substituted or unsubstituted alkyl, or substituted or unsubstituted aryl;
M¹ is C(═O) or C(OH);
M² is a bond, NH, or NHCH₂;
when [symbol] is a single bond, then A is N and R¹¹ is present, or A is CH or C(OH) and R¹¹ is present;
when [symbol] is a double bond, then A is N and R¹¹ is absent, or A is C and R¹¹ is present; and
R¹⁰ is hydrogen;
R¹¹ is substituted or unsubstituted alkyl, substituted or unsubstituted aralkyl, substituted or unsubstituted heteroaralkyl, substituted or unsubstituted aryl, or substituted or unsubstituted heteroaryl; or
R¹⁰ and R¹¹ are taken together with their intervening atoms to form an unsubstituted fused phenyl ring; and
z5 is 0 or 1.

In certain embodiments, the provided compound is of a free base form. In certain embodiments, the provided compound is in the form of a pharmaceutically acceptable salt as generally defined herein.

In certain embodiments, R⁹ is a halogen (e.g., fluoro, chloro, bromo, or iodo).

In certain embodiments, R⁹ is —OR^{9a}; and R^{9a} is substituted or unsubstituted alkyl. In certain embodiments, R⁹ is —OR^{9a}; and R^{9a} is substituted or unsubstituted C₁₋₆alkyl. In certain embodiments, R⁹ is —OR^{9a}; and R^{9a} is substituted or unsubstituted C₁₋₃alkyl. In certain embodiments, R⁹ is —OR^{9a}; and R^{9a} is substituted or unsubstituted methyl. In certain embodiments, R⁹ is —OR^{9a}; and R^{9a} is substituted or unsubstituted ethyl. In certain embodiments, R⁹ is —OR^{9a}; and R^{9a} is substituted or unsubstituted propyl. In certain embodiments, R⁹ is —OR^{9a}; and R^{9a} is substituted or unsubstituted isopropyl.

In certain embodiments, the phenyl ring to which R⁹ is attached is of formula:

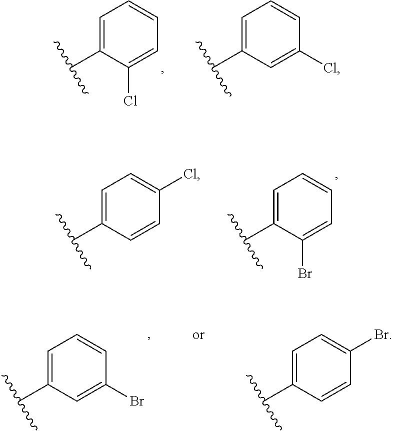

In certain embodiments, the phenyl ring to which R⁹ is attached is of formula:

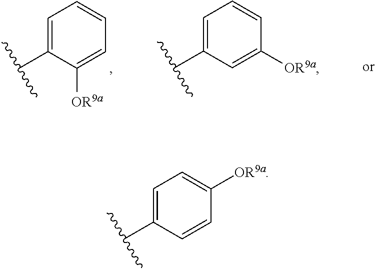

In certain embodiments, the ring system:

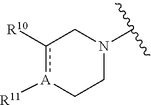

corresponds to:

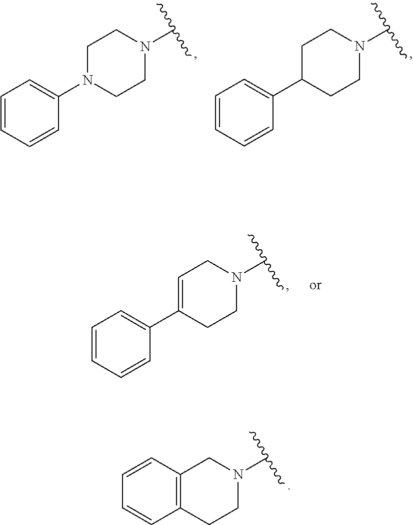 [SMILES: *N1CCN(c2ccccc2)CC1 ; *N1CCC(c2ccccc2)CC1 ; *N1CC=C(c2ccccc2)CC1 ; *N1CCc2ccccc2C1]

### Compounds of Formula (XII)

In certain embodiments, the present disclosure provides a compound of Formula (XII):

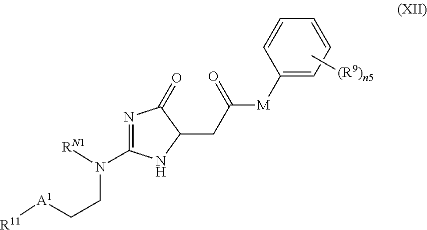

or a pharmaceutically acceptable salt thereof,
wherein:
each occurrence of R⁹ is independently substituted or unsubstituted alkyl, —OR^{9a}, —C(═O)R^{9a}, —C(═O)OR^{9a}, —(C═O)NHR^{9a}, —NH(C═O)R^{9a}, —CN, or halogen, or two vicinal R⁹ are taken together with their intervening atoms to form a substituted or unsubstituted heterocyclic ring;
each occurrence of R^{9a} is independently hydrogen, independently substituted or unsubstituted alkyl, or substituted or unsubstituted aryl;
M is a bond or NH;
A¹ is a bond or NR^{N2};
each of R^{N1} or R^{N2} is independently hydrogen, or substituted or unsubstituted alkyl;
R¹¹ is substituted or unsubstituted alkyl, substituted or unsubstituted aralkyl, substituted or unsubstituted heteroaralkyl, substituted or unsubstituted aryl, or substituted or unsubstituted heteroaryl;
n5 is 0, 1, 2, 3, 4, or 5; and
z5 is 0 or 1.

In certain embodiments, the provided compound is of a free base form. In certain embodiments, the provided compound is in the form of a pharmaceutically acceptable salt as generally defined herein.

In certain embodiments, R⁹ is a halogen (e.g., fluoro, chloro, bromo, or iodo).

In certain embodiments, R⁹ is —OR^{9a}; and R^{9a} is substituted or unsubstituted alkyl. In certain embodiments, R⁹ is —OR^{9a}; and R^{9a} is substituted or unsubstituted C₁₋₆alkyl. In certain embodiments, R⁹ is —OR^{9a}; and R^{9a} is substituted or unsubstituted C₁₋₃alkyl. In certain embodiments, R⁹ is —OR^{9a}; and R^{9a} is substituted or unsubstituted methyl. In certain embodiments, R⁹ is —OR^{9a}; and R^{9a} is substituted or unsubstituted ethyl. In certain embodiments, R⁹ is —OR^{9a}; and R^{9a} is substituted or unsubstituted propyl. In certain embodiments, R⁹ is —OR^{9a}; and R^{9a} is substituted or unsubstituted isopropyl.

In certain embodiments, the phenyl ring to which R⁹ is attached is of formula:

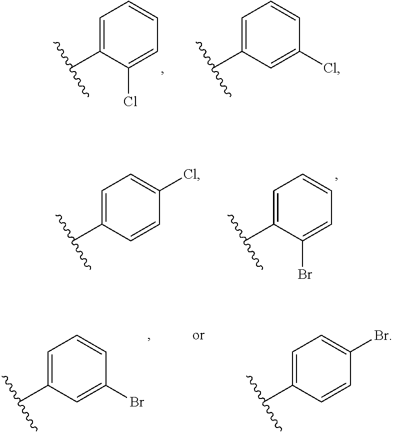

In certain embodiments, the phenyl ring to which R⁹ is attached is of formula:

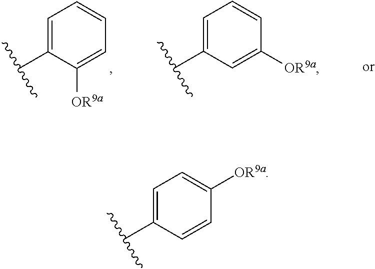

In certain embodiments, M is a bond. In certain embodiments, M is NH.

In certain embodiments, M is a bond and the phenyl ring to which R⁹ is attached is of formula:

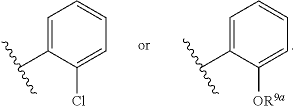

In certain embodiments, M is NH and the phenyl ring to which R⁹ is attached is of formula:

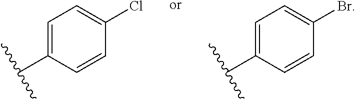 [SMILES: *c1ccc(Cl)cc1 ; "or" ; *c1ccc(Br)cc1]

### Compounds of Formula (XIII)

In certain embodiments, the present disclosure provides a compound of Formula (XIII):

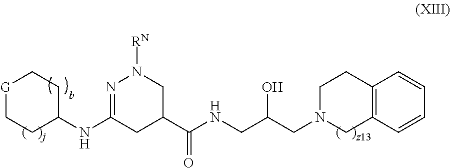

or a pharmaceutically acceptable salt thereof,
wherein:
R^{N} is hydrogen or substituted or unsubstituted alkyl;
G is O or N(R²⁰);
R²⁰ is hydrogen, substituted or unsubstituted alkyl, —SO₂R^{20a}, or —C(═O)R^{20a};
R^{20a} is substituted or unsubstituted alkyl or substituted or unsubstituted cycloalkyl;
j, b, and z13 are each independently 0 or 1.

In certain embodiments, the provided compound is of a free base form. In certain embodiments, the provided compound is in the form of a pharmaceutically acceptable salt as generally defined herein.

In certain embodiments, G is O. In certain embodiments, G is N(R²⁰); and R²⁰ is hydrogen. In certain embodiments, G is N(R²⁰); and R²⁰ is substituted alkyl. In certain embodiments, G is N(R²⁰); and R²⁰ is substituted or unsubstituted C₁₋₆alkyl. In certain embodiments, G is N(R²⁰); and R²⁰ is substituted or unsubstituted C₁₋₃alkyl. In certain embodiments, G is N(R²⁰); and R²⁰ is substituted or unsubstituted methyl. In certain embodiments, G is N(R²⁰); and R²⁰ is substituted or unsubstituted ethyl. In certain embodiments, G is N(R²⁰); and R²⁰ is substituted or unsubstituted propyl. In certain embodiments, G is N(R²⁰); and R²⁰ is substituted or unsubstituted isopropyl.

In certain embodiments, G is N(R²⁰); R²⁰ is —SO₂R^{20a}; and R^{20a} is substituted or unsubstituted alkyl. In certain embodiments, G is N(R²⁰); R²⁰ is —SO₂R^{20a}; and R^{20a} is substituted or unsubstituted C₁₋₆alkyl. In certain embodiments, G is N(R²⁰); R²⁰ is —SO₂R^{20a}; and R^{20a} is substituted or unsubstituted C₁₋₃alkyl. In certain embodiments, G is N(R²⁰); R²⁰ is —SO₂R^{20a}; and R^{20a} is substituted or unsubstituted methyl. In certain embodiments, G is N(R²⁰); R²⁰ is —SO₂R^{20a}; and R^{20a} is substituted or unsubstituted ethyl. In certain embodiments, G is N(R²⁰); R²⁰ is —SO₂R^{20a}; and R^{20a} is substituted or unsubstituted propyl. In certain embodiments, G is N(R²⁰); R²⁰ is —SO₂R^{20a}; and R^{20a} is substituted or unsubstituted isopropyl.

In certain embodiments, G is N(R²⁰); R²⁰ is —SO₂R^{20a}; and R^{20a} is substituted or unsubstituted cycloalkyl. In certain embodiments, G is N(R²⁰); R²⁰ is —SO₂R^{20a}; and R^{20a} is substituted or unsubstituted 3-6 membered cycloalkyl. In certain embodiments, G is N(R²⁰); R²⁰ is —SO₂R^{20a}; and R^{20a} is substituted or unsubstituted cyclopropyl. In certain embodiments, G is N(R²⁰); R²⁰ is —SO₂R^{20a}; and R^{20a} is substituted or unsubstituted cyclobutyl. In certain embodiments, G is N(R²⁰); R²⁰ is —SO₂R^{20a}; and R^{20a} is substituted or unsubstituted cyclopentyl. In certain embodiments, G is N(R²⁰); R²⁰ is —SO₂R^{20a}; and R^{20a} is substituted or unsubstituted cyclohexyl.

In certain embodiments, G is N(R²⁰); R²⁰ is —C(═O)R^{20a}; and R^{20a} is substituted or unsubstituted alkyl. In certain embodiments, G is N(R²⁰); R²⁰ is —C(═O)R^{20a}; and R^{20a} is substituted or unsubstituted C₁₋₆alkyl. In certain embodiments, G is N(R²⁰); R²⁰ is —C(═O)R^{20a}; and R^{20a} is substituted or unsubstituted C₁₋₃alkyl. In certain embodiments, G is N(R²⁰); R²⁰ is —C(═O)R^{20a}; and R^{20a} is substituted or unsubstituted methyl. In certain embodiments, G is N(R²⁰); R²⁰ is —C(═O)R^{20a}; and R^{20a} is substituted or unsubstituted ethyl. In certain embodiments, G is N(R²⁰); R²⁰ is —C(═O)R^{20a}; and R^{20a} is substituted or unsubstituted propyl. In certain embodiments, G is N(R²⁰); R²⁰ is —C(═O)R^{20a}; and R^{20a} is substituted or unsubstituted isopropyl.

In certain embodiments, G is N(R²⁰); R²⁰ is —C(═O)R^{20a}; and R^{20a} is substituted or unsubstituted cycloalkyl. In certain embodiments, G is N(R²⁰); R²⁰ is —C(═O)R^{20a}; and R^{20a} is substituted or unsubstituted 3-6 membered cycloalkyl. In certain embodiments, G is N(R²⁰); R²⁰ is —C(═O)R^{20a}; and R^{20a} is substituted or unsubstituted cyclopropyl. In certain embodiments, G is N(R²⁰); R²⁰ is —C(═O)R^{20a}; and R^{20a} is substituted or unsubstituted cyclobutyl. In certain embodiments, G is N(R²⁰); R²⁰ is —C(═O)R^{20a}; and R^{20a} is substituted or unsubstituted cyclopentyl. In certain embodiments, G is N(R²⁰); R²⁰ is —C(═O)R^{20a}; and R^{20a} is substituted or unsubstituted cyclohexyl.

In certain embodiments, j is 0; and b is 0. In certain embodiments, j is 1; and b is 0. In certain embodiments, j is 0; and b is 1. In certain embodiments, j is 1; and b is 1.

In certain embodiments, the ring comprising G is of formula:

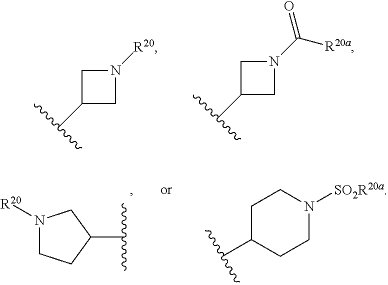

In certain embodiments, the ring comprising G is of formula:

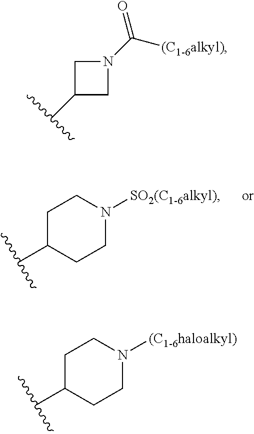

wherein the alkyl and haloalkyl are substituted or unsubstituted.

In certain embodiments, the ring comprising G is of formula:

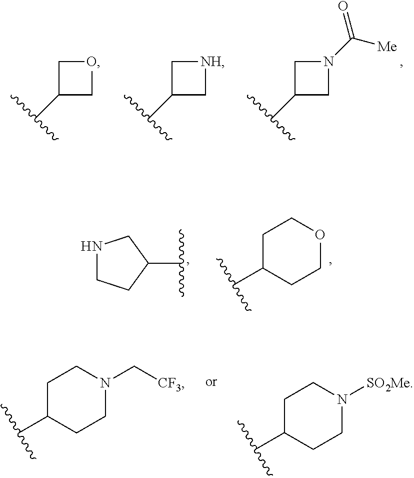

### Exemplary Compounds

In certain embodiments, a provided compound is a compound listed in Table 1A-1O, or a pharmaceutically acceptable salt thereof. In certain embodiments, a provided compound is a compound listed in Table 1A, or a pharmaceutically acceptable salt thereof.

TABLE 1A
Exemplary Compounds

| | | | LCMS |
|---|---|---|---|
| Cmpd | | Exact | m/z |
| No | Structure | Mass | (M + H) |
| 1-1 | 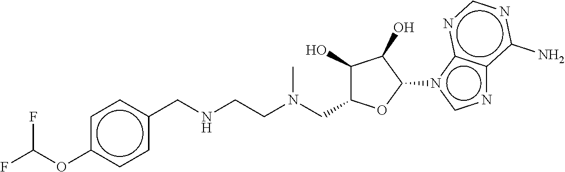 | 479.2093 | 480 |
| 2-1 | 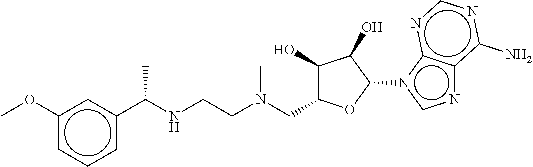 | 457.2438 | 458 |
| 3-1 | 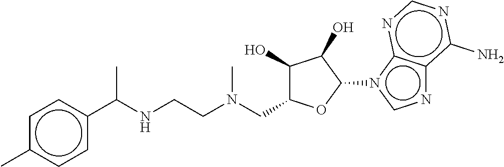 | 441.2488 | 442 |
| 4-1 | 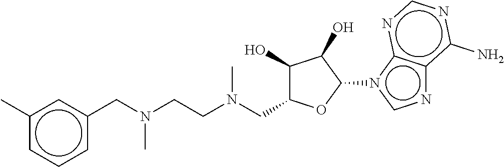 | 441.2488 | 442 |
| 5-1 | 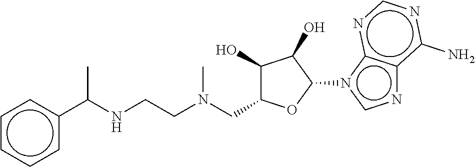 | 427.2332 | 428 |
| 6-1 | 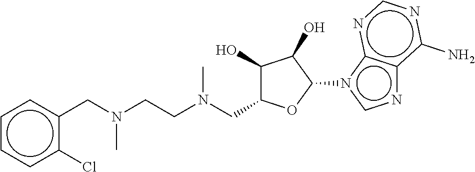 | 461.1942 | 462 |
| 7-1 | 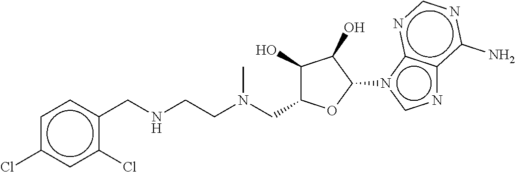 | 481.1396 | 482 |
| 8-1 | 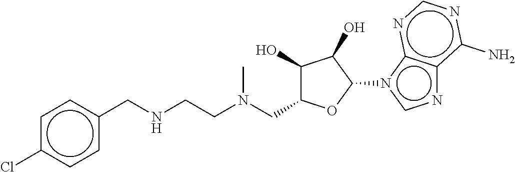 | 447.1786 | 448 |
| 9-1 | 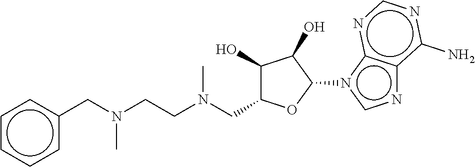 | 427.2332 | 429 |
| 10-1 | 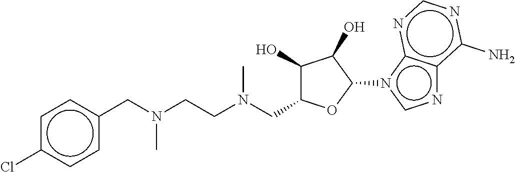 | 461.1942 | 462 |
| 11-1 | 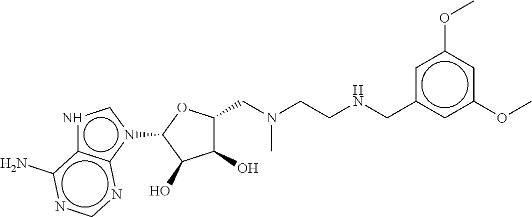 | 473.2387 | 474 |
| 12-1 | 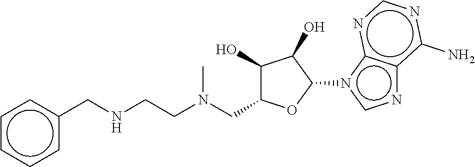 | 413.2175 | 414 |
| 13-1 | 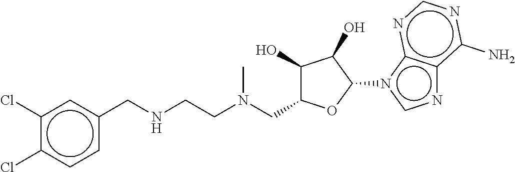 | 481.1396 | — |
| 14-1 | 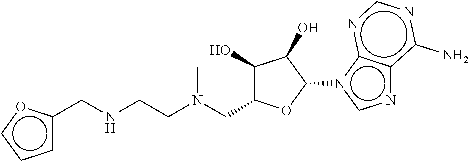 | 403.1968 | 404 |
| 15-1 | 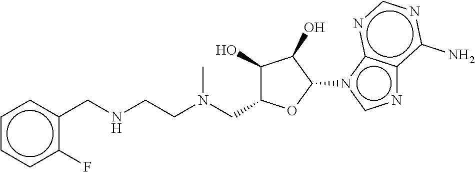 | 431.2081 | 432 |
| 16-1 | 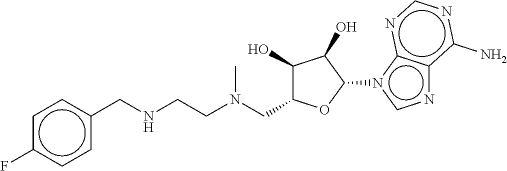 | 431.2081 | 432 |
| 17-1 | 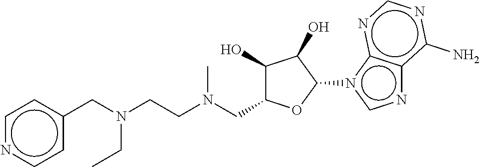 | 442.2441 | 443 |
| 18-1 | 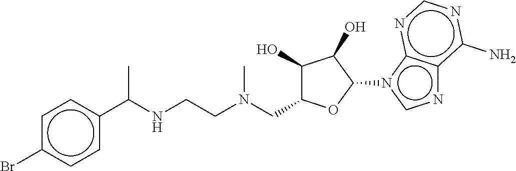 | 505.1437 | 508 |
| 19-1 | 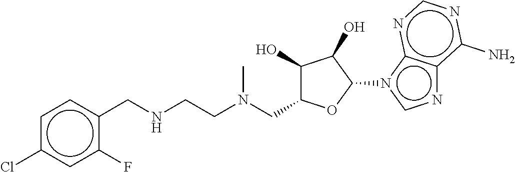 | 465.1691 | 466 |
| 20-1 | 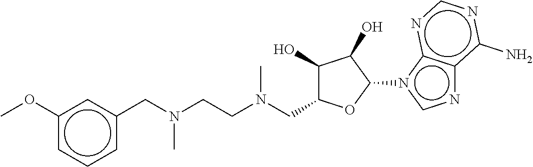 | 457.2438 | 459 |
| 21-1 | 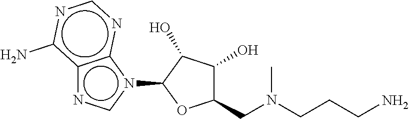 | 337.1862 | 338.3 |
| 22-1 | 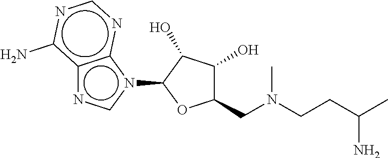 | 351.2019 | 352.1 |
| 23-1 | 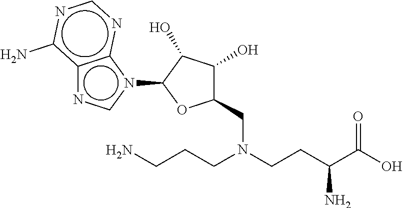 | 424.2183 | — |
| 24-1 | 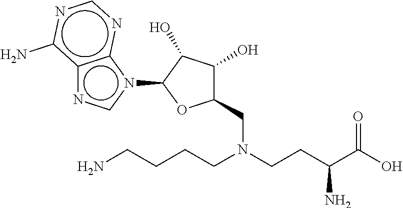 | 438.2339 | — |
| 25-1 | 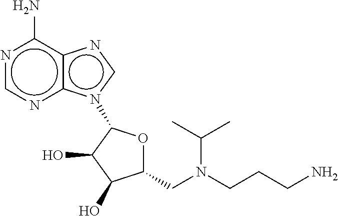 | 365.2175 | 366.3 |
| 26-1 | 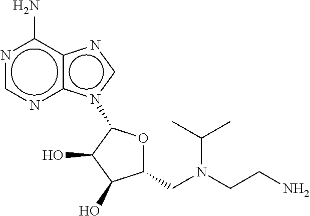 | 351.2019 | 352.3 |
| 27-1 | 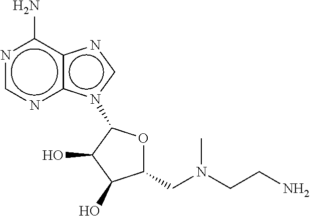 | 323.1706 | 324.2 |
| 28-1 | 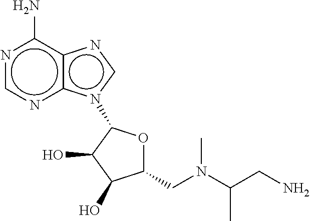 | 337.1862 | — |
| 29-1 | 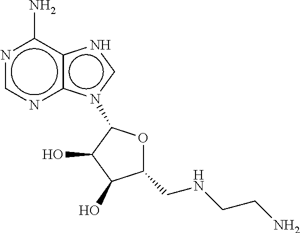 | 309.1549 | — |
| 30-1 | 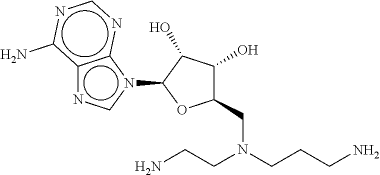 | 366.2128 | 367.1 |
| 31-1 | 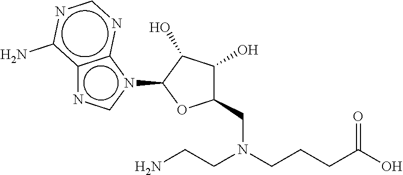 | 395.1917 | 396 |
| 32-1 | 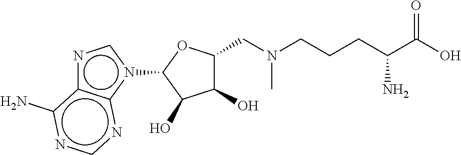 | 395.1917 | 396.2 |
| 33-1 | 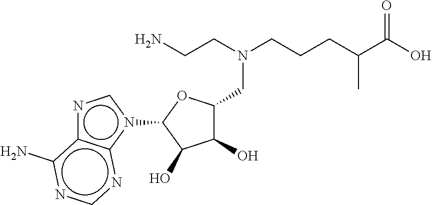 | 423.2230 | 424.2 |
| 34-1 | 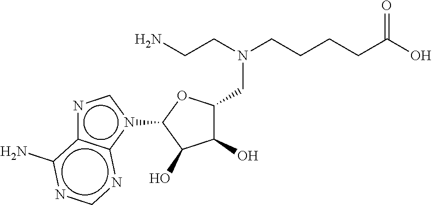 | 409.2074 | 410.2 |
| 35-1 | 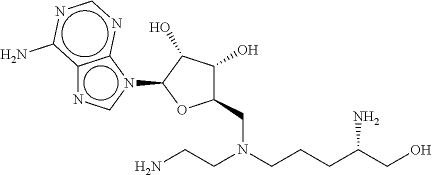 | 410.2390 | 411.2 |
| 36-1 | 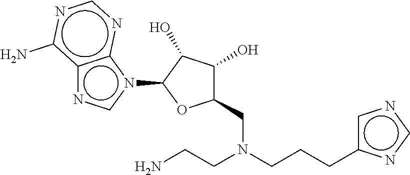 | 416.2159 | — |
| 37-1 | 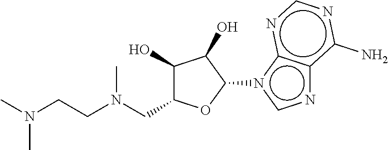 | 351.2019 | — |
| 38-1 | 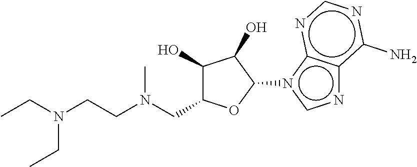 | 379.2332 | — |
| 39-1 | 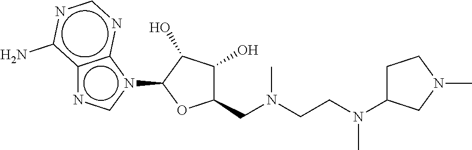 | 420.2597 | 421 |
| 40-1 | 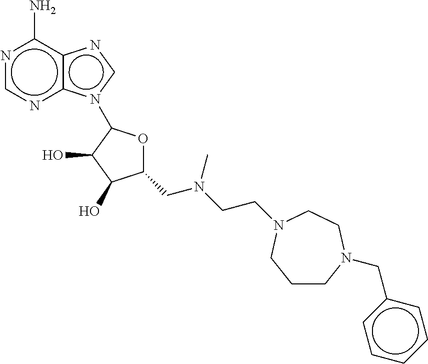 | 496.2910 | 497 |
| 41-1 | 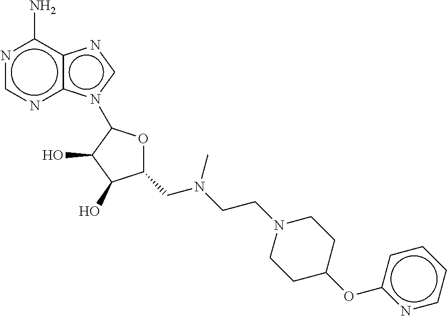 | 484.2547 | 485 |
| 42-1 | 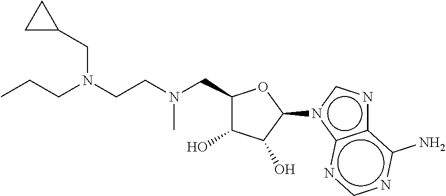 | 419.2645 | 420 |
| 43-1 | 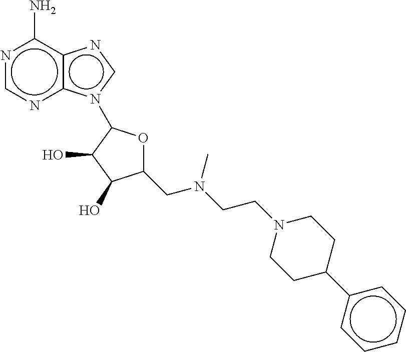 | 467.2645 | 468 |
| 44-1 | 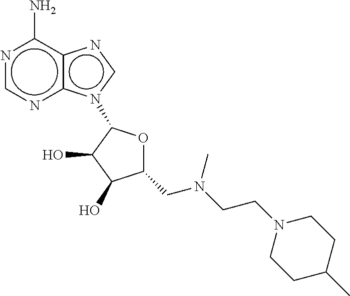 | 405.2488 | 406 |
| 45-1 | 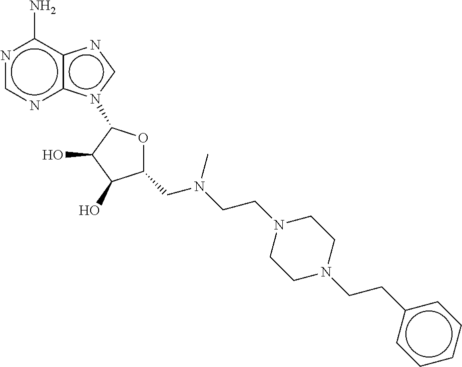 | 496.2910 | 498 |
| 46-1 | 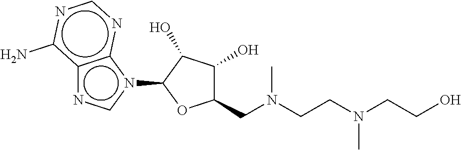 | 381.2125 | 382 |
| 47-1 | 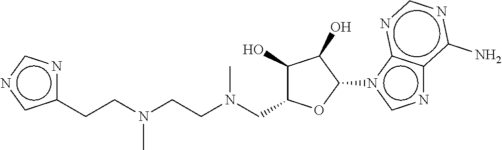 | 430.2315 | 432 |
| 48-1 | 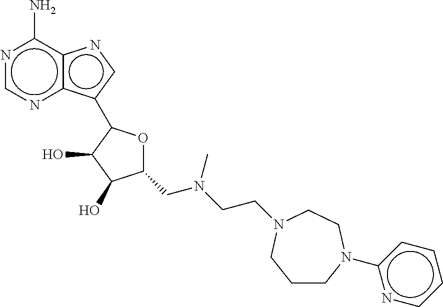 | 483.2706 | 484 |
| 49-1 | 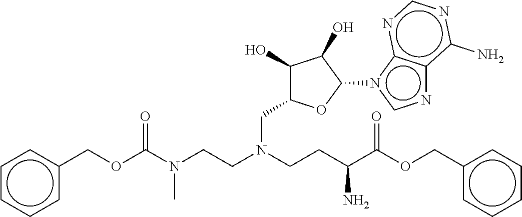 | 648.3020 | 649.3 |
| 50-1 | 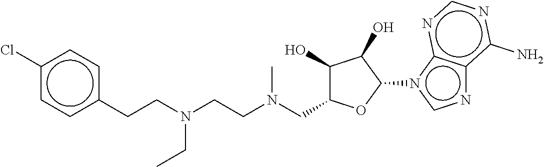 | 489.2255 | 490.2 |
| 51-1 | 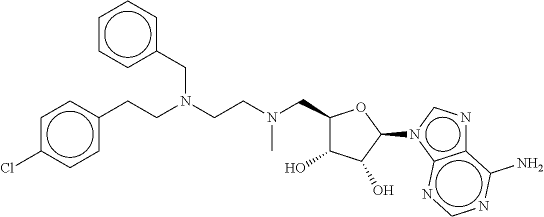 | 551.2412 | 552.3 |
| 52-1 | 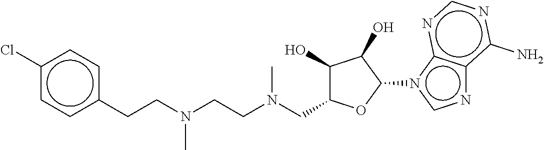 | 475.2099 | 476.2 |
| 53-1 | 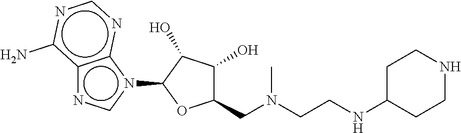 | 406.2441 | 407 |
| 54-1 | 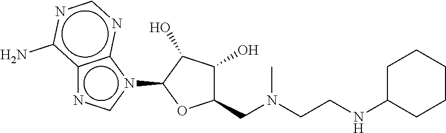 | 405.2488 | 406 |
| 55-1 | 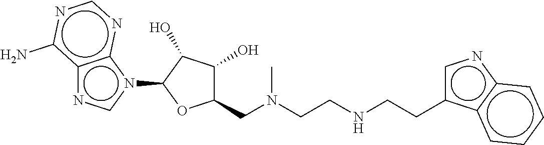 | 465.2363 | 467 |
| 56-1 | 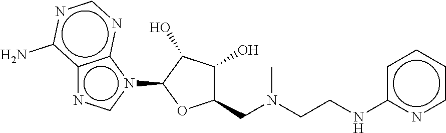 | 400.1971 | 401 |
| 57-1 | 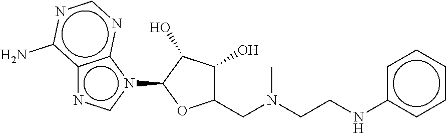 | 399.2019 | 400 |
| 58-1 | 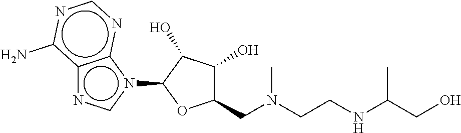 | 381.2125 | 382 |
| 59-1 | 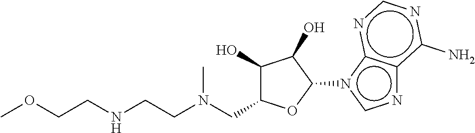 | 381.2125 | 382 |
| 60-1 | 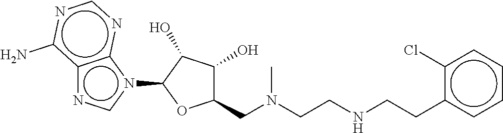 | 461.1942 | 462 |
| 61-1 | 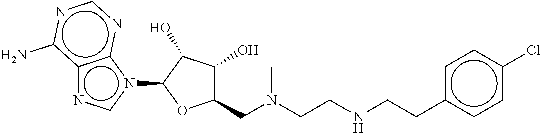 | 461.1942 | — |
| 62-1 | 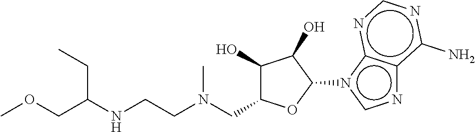 | 409.2438 | 410 |
| 63-1 | 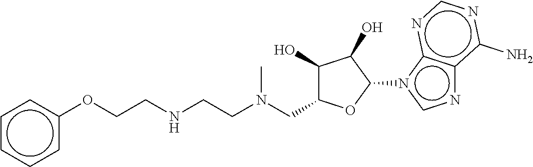 | 443.2281 | 444 |
| 64-1 | 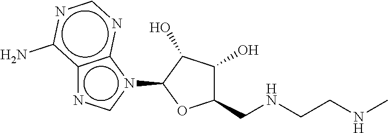 | 323.1706 | — |
| 65-1 | 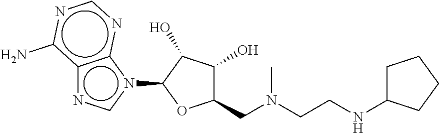 | 391.2332 | 392 |
| 66-1 | 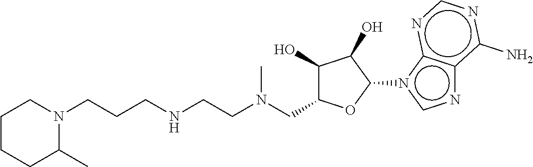 | 462.3067 | 463 |
| 67-1 | 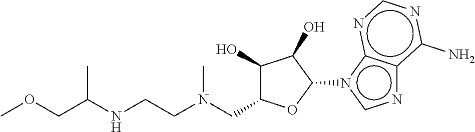 | 395.2281 | 396 |
| 68-1 | 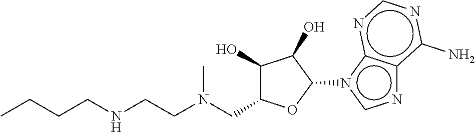 | 379.2332 | 380 |
| 69-1 | 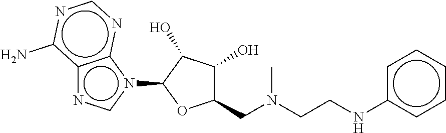 | 400.1971 | 401 |
| 70-1 | 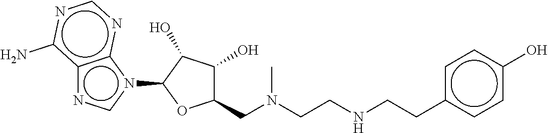 | 443.2281 | 444 |
| 71-1 | 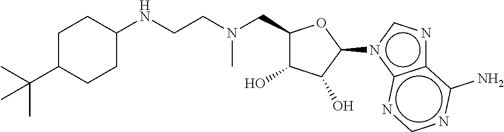 | 461.3114 | 463 |
| 72-1 | 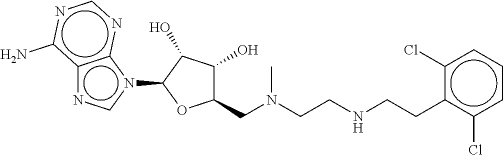 | 495.1552 | 496 |
| 73-1 | 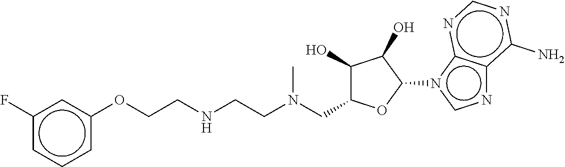 | 461.2187 | 462 |
| 74-1 | 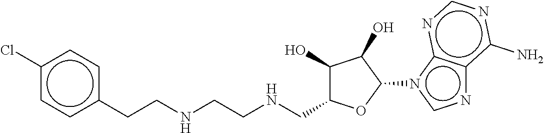 | 447.1786 | 448.2 |
| 75-1 | 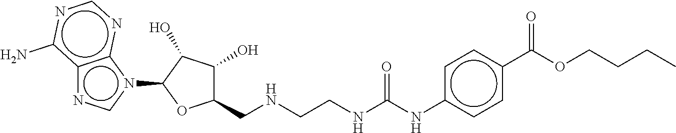 | 528.2445 | 529.3 |
| 76-1 | 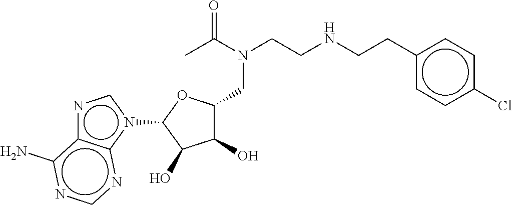 | 489.1891 | 490.1 |
| 77-1 | 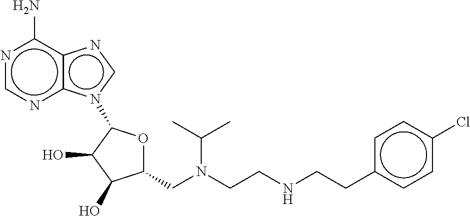 | 489.2255 | 490.2 |
| 78-1 | 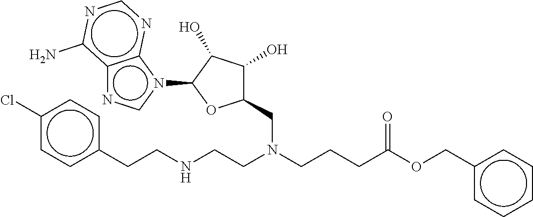 | 623.2623 | 624.2 |
| 79-1 | 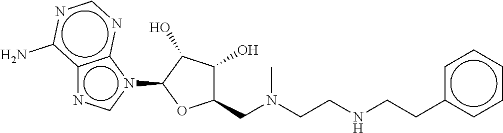 | 427.2332 | 428.2 |
| 80-1 | 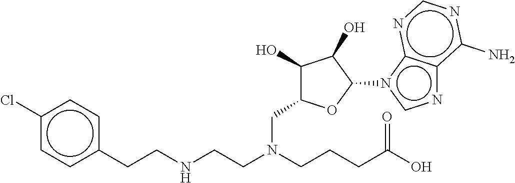 | 533.2153 | 534.2 |
| 81-1 | 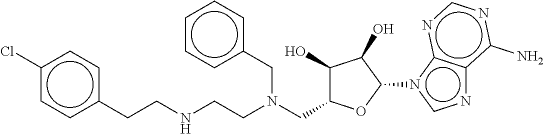 | 537.2255 | 538.2 |
| 82-1 | 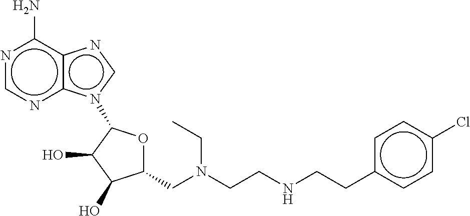 | 475.2099 | 476.1 |
| 83-1 | 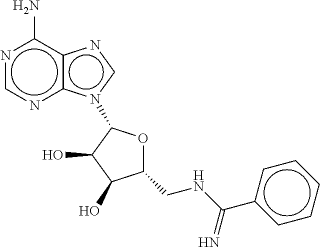 | 369.1549 | — |
| 84-1 | 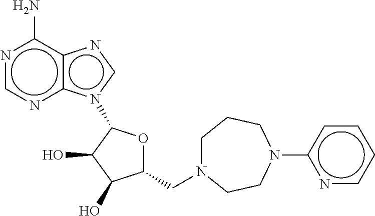 | 426.2128 | — |
| 85-1 | 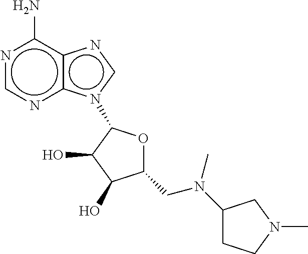 | 363.2019 | — |
| 86-1 | 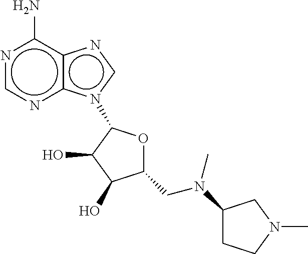 | 363.2019 | — |
| 87-1 | 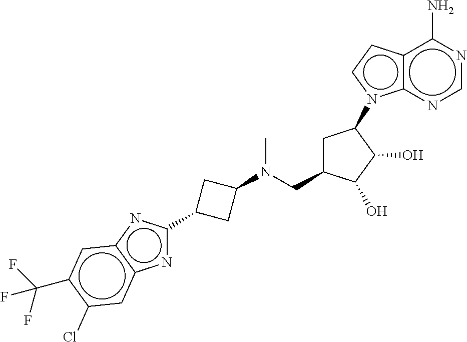 | 548.1789 | — |
| 88-1 | 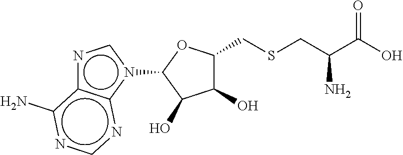 | 370.1059 | — |
| 89-1 | 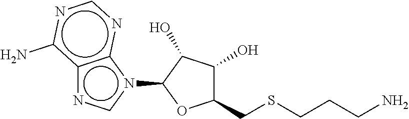 | 340.1318 | 341.6 |
| 90-1 | 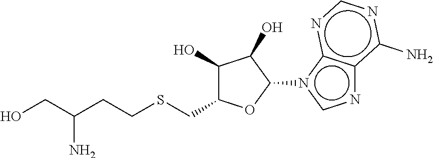 | 370.1423 | 371 |
| 91-1 | 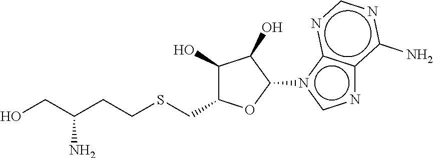 | 370.1423 | 371.1 |
| 92-1 | 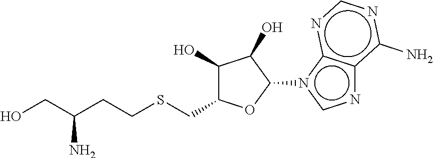 | 370.1423 | 371.1 |
| 93-1 | 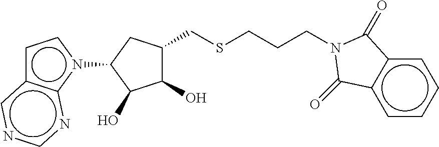 | 452.1518 | 453.2 |
| 94-1 | 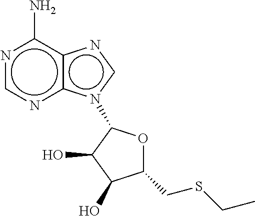 | 311.1052 | — |
| 95-1 | 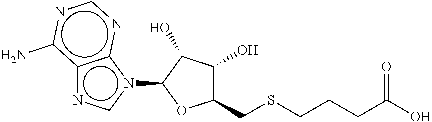 | 369.1107 | — |
| 96-1 | 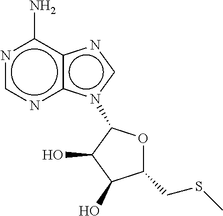 | 297.0896 | — |
| 97-1 | 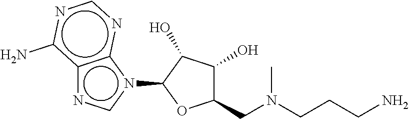 | — | — |

In certain embodiments, a provided compound is a compound listed in Table 1B, or a pharmaceutically acceptable salt thereof.

TABLE 1B
Exemplary Compounds

| | | | LCMS |
|---|---|---|---|
| Cmpd | | Exact | m/z |
| No | Structure | Mass | (M + H) |
| 2-2 | 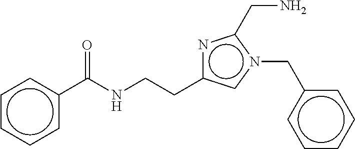 | 334.1794 | 335.3 |

In certain embodiments, a provided compound is a compound listed in Table 1C, or a pharmaceutically acceptable salt thereof.

TABLE 1C
Exemplary Compounds

| | | | LCMS |
|---|---|---|---|
| Cmpd | | Exact | m/z |
| No | Structure | Mass | (M + H) |
| 2-3 | 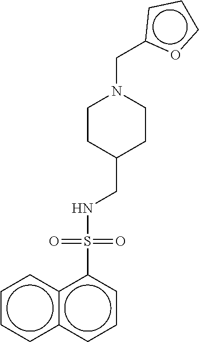 | 384.1508 | 385.2 |
| 3-3 | 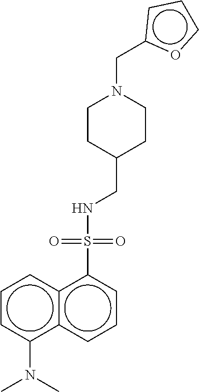 | 427.1930 | 428.3 |
| 4-3 | 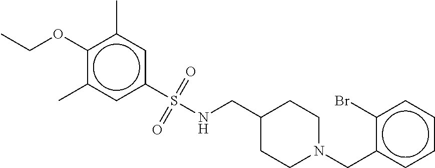 | 494.1239 | 496.2 |
| 5-3 | 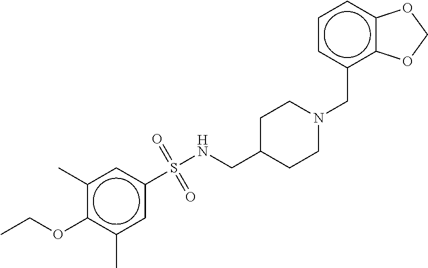 | 460.2032 | 461.3 |
| 6-3 | 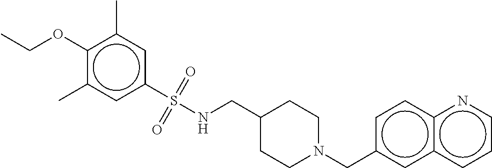 | 467.2243 | 468.3 |
| 7-3 | 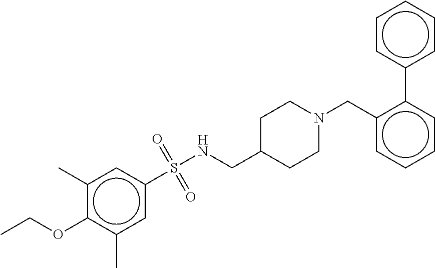 | 0492.2447 | 493.3 |
| 8-3 | 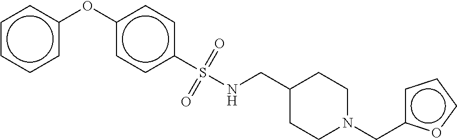 | 426.1613 | 427.1 |
| 9-3 | 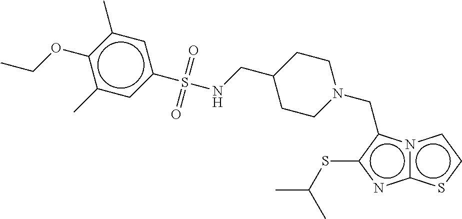 | 536.1950 | 537.3 |
| 10-3 | 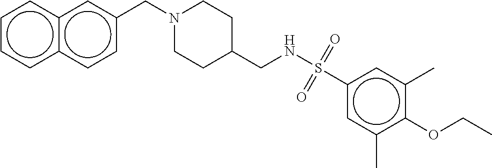 | 466.2290 | 467.3 |
| 11-3 | 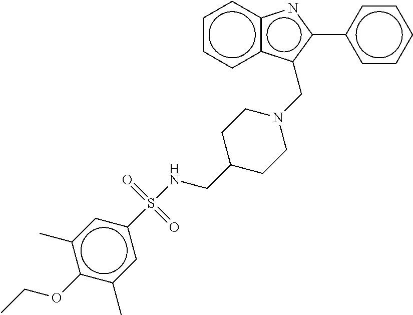 | 530.2477 | 532.2 |
| 12-3 | 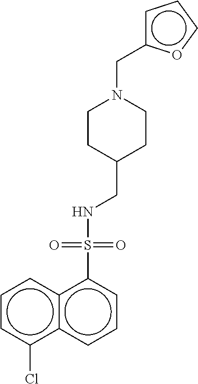 | 418.1118 | 419.1 |
| 13-3 | 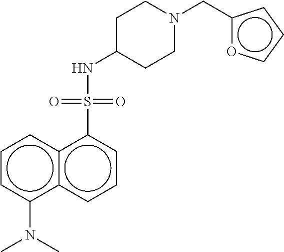 | 413.1773 | 414.2 |
| 14-3 | 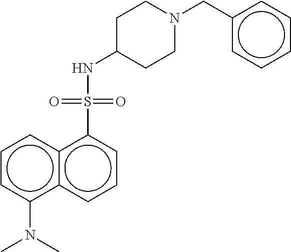 | 423.1980 | 424.2 |
| 15-3 | 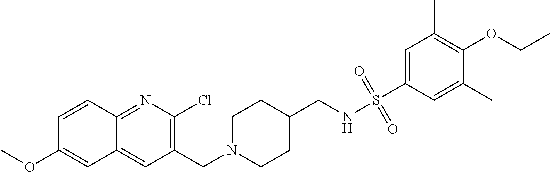 [SMILES: CCOc1c(C)cc(S(=O)(=O)NCC2CCN(Cc3cc4cc(OC)ccc4nc3Cl)CC2)cc1C] | — | — |

In certain embodiments, a provided compound is a compound listed in Table 1D, or a pharmaceutically acceptable salt thereof.

TABLE 1D
Exemplary compounds

| | | | LCMS |
|---|---|---|---|
| Cmpd | | Exact | m/z |
| No | Structure | Mass | (M + H) |
| 1-4 | 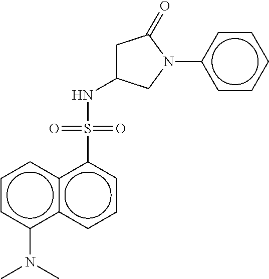 | 409.1460 | 410.2 |
| 2-4 | 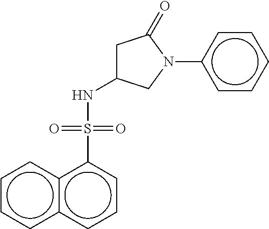 | 366.1038 | 367.1 |
| 3-4 | 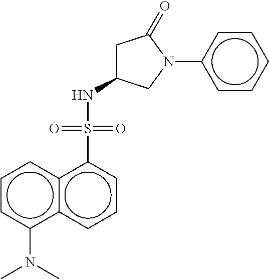 | 409.1460 | 410.2 |
| 4-4 | 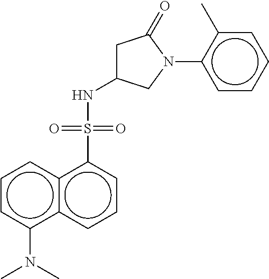 | 423.1617 | 424.1 |
| 5-4 | 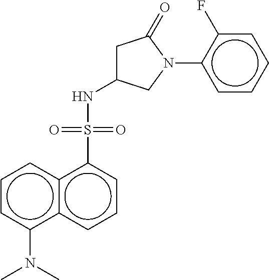 | 427.1366 | 428.1 |
| 6 -4 | 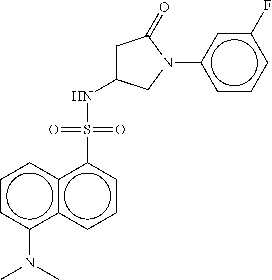 | 427.1366 | 428.1 |
| 7-4 | 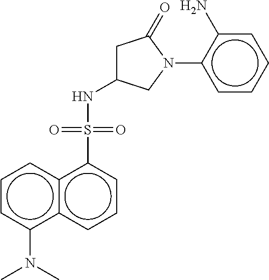 | 424.1569 | 425.1 |
| 8-4 | 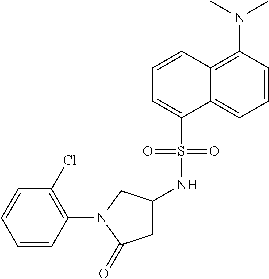 [SMILES: CN(C)c1cccc2c(S(=O)(=O)NC3CC(=O)N(c4ccccc4Cl)C3)cccc12] | — | — |

In certain embodiments, a provided compound is a compound listed in Table 1E, or a pharmaceutically acceptable salt thereof.

TABLE 1E
Exemplary Compounds

| | | | LCMS |
|---|---|---|---|
| | | | m/z |
| Cmpd | | Exact | (M + |
| No | Structure | Mass | H) |
| 1-5 | 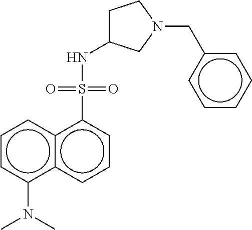 | 409.1824 | 410.2 |
| 2-5 | 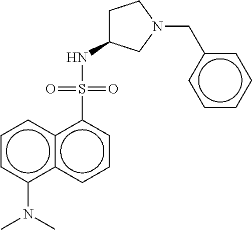 | 409.1824 | 410.2 |
| 3-5 | 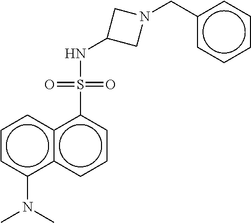 | 395.1667 | 396.1 |

In certain embodiments, a provided compound is a compound listed in Table 1F, or a pharmaceutically acceptable salt thereof.

TABLE 1F
Exemplary Compounds

| | | | LCMS |
|---|---|---|---|
| Cmpd | | Exact | m/z |
| No | Structure | Mass | (M + H) |
| 1-6 | 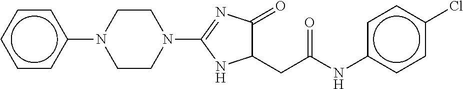 | 411.1462 | 412.3 |
| 2-6 | 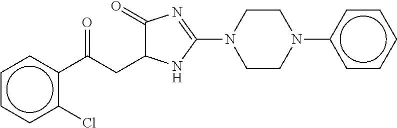 | 396.1353 | 397.3 |
| 3-6 | 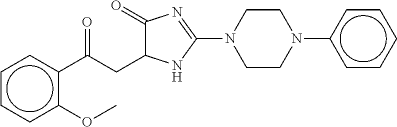 | 392.1848 | 393.2 |
| 4-6 | 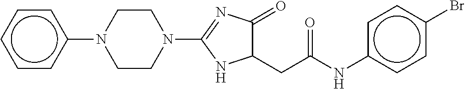 | 455.0957 | 456.4 |
| 5-6 | 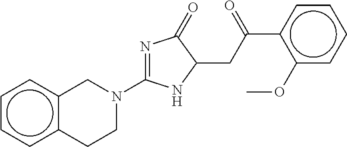 | 363.1583 | 364.2 |
| 6-6 | 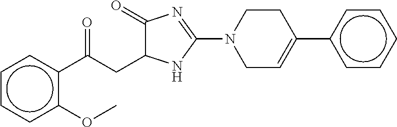 | 389.1739 | 390.3 |

In certain embodiments, a provided compound is a compound listed in Table 1G, or a pharmaceutically acceptable salt thereof.

TABLE 1G
Exemplary Compounds

| | | | LCMS |
|---|---|---|---|
| Cmpd | | Exact | m/z |
| No | Structure | Mass | (M + H) |
| 1-7 | 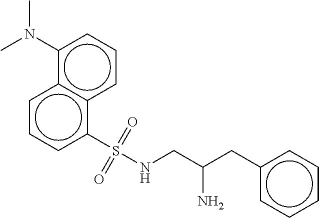 | 383.1667 | 384.2 |
| 2-7 | 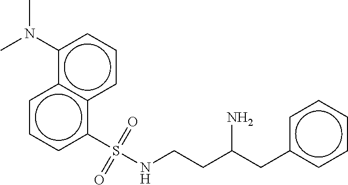 | 397.1824 | 398.2 |
| 3-7 | 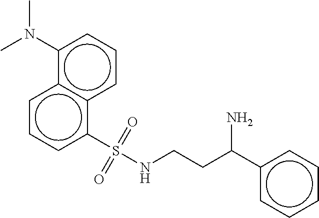 | 383.1667 | 384.2 |
| 4-7 | 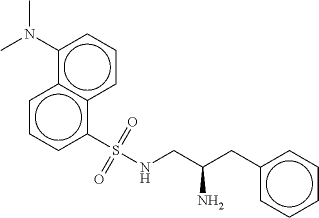 | 383.1667 | 384.2 |
| 5-7 | 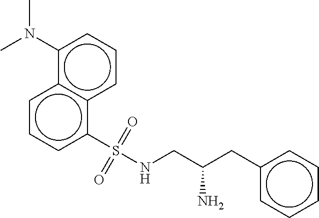 | 383.1667 | 384.2 |

In certain embodiments, a provided compound is a compound listed in Table 1H, or a pharmaceutically acceptable salt thereof.

TABLE 1H
Exemplary Compouds

| | | | LCMS |
|---|---|---|---|
| Cmpd | | Exact | m/z |
| No | Structure | Mass | (M + H) |
| 1-8 | 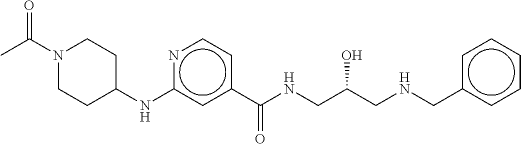 | 425.2427 | 426.3 |

In certain embodiments, a provided compound is a compound listed in Table 1I, or a pharmaceutically acceptable salt thereof.

TABLE 1I
Exemplary Compounds

| | | | LCMS |
|---|---|---|---|
| Cmpd | | Exact | m/z |
| No | Structure | Mass | (M + H) |
| 1-9 | 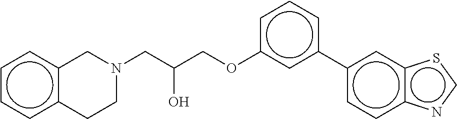 | 416.1558 | 417.2 |
| 2-9 | 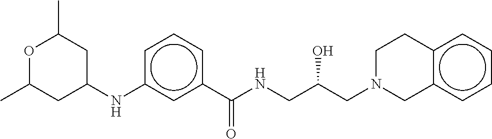 | 437.2678 | 438.3 |
| 3-9 | 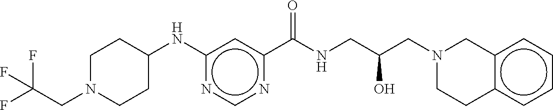 | 492.2461 | 493.3 |
| 4-9 | 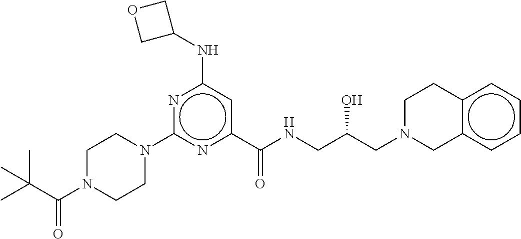 | 551.3220 | 552.3 |
| 5-9 | 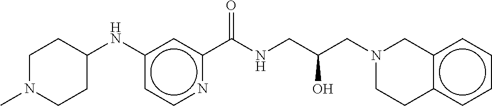 | 423.2634 | 424.2 |
| 6-9 | 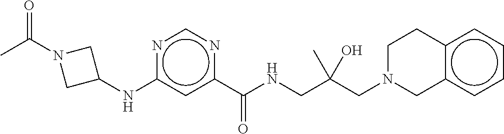 | 438.2379 | 439.2 |
| 7-9 | 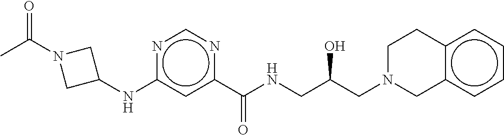 | 424.2223 | 425.1 |
| 8-9 | 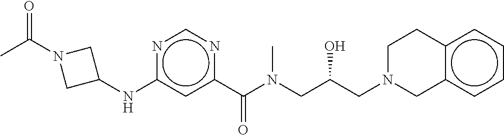 | 438.2379 | 439.2 |
| 9-9 | 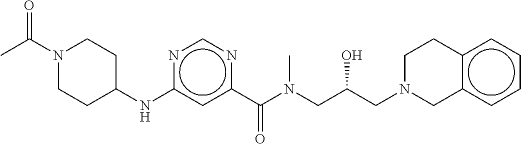 | 466.2692 | 467.3 |
| 10-9 | 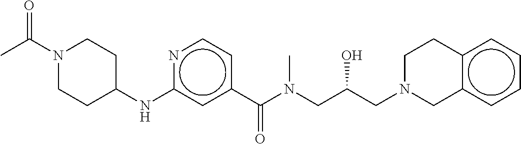 | 465.2740 | 466.3 |
| 11-9 | 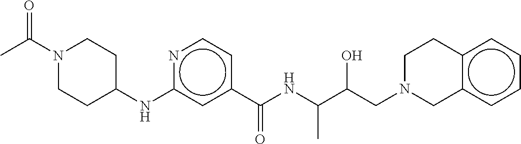 | 465.2740 | 466.2 |
| 12-9 | 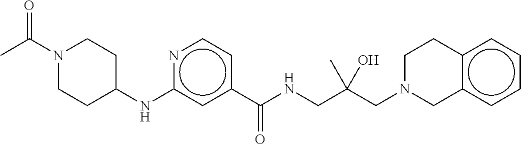 | 465.2740 | — |
| 13-9 | 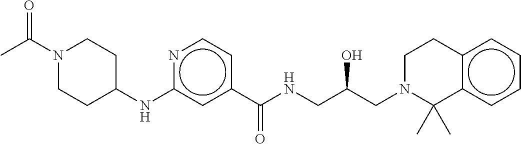 | 479.2896 | 480.1 |
| 14-9 | 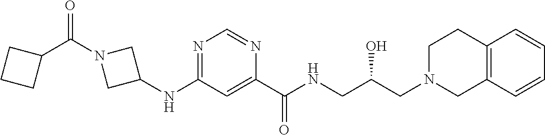 [SMILES: O=C(NC[C@H](O)CN1CCc2ccccc2C1)c1cc(NC2CN(C(=O)C3CCC3)C2)ncn1] | 464.2536 | 465.2 |
| 15-9 | 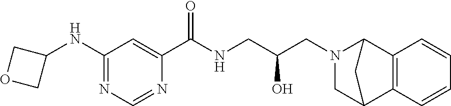 [SMILES: O=C(NC[C@H](O)CN1CC2CC1c1ccccc12)c1cc(NC2COC2)ncn1] | 395.1957 | — |
| 16-9 | 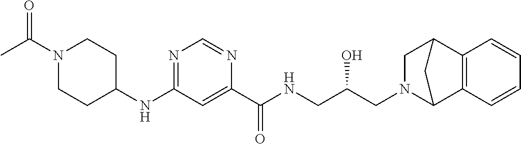 [SMILES: CC(=O)N1CCC(Nc2cc(C(=O)NC[C@H](O)CN3CC4CC3c3ccccc34)ncn2)CC1] | 464.2536 | — |
| 17-9 | 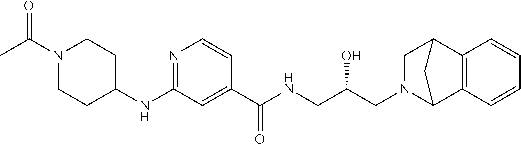 [SMILES: CC(=O)N1CCC(Nc2cc(C(=O)NC[C@H](O)CN3CC4CC3c3ccccc34)ccn2)CC1] | 463.2583 | — |
| 18-9 | 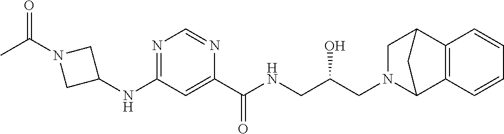 [SMILES: CC(=O)N1CC(Nc2cc(C(=O)NC[C@H](O)CN3CC4CC3c3ccccc34)ncn2)C1] | 436.2223 | — |
| 19-9 | 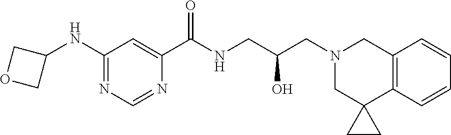 [SMILES: O=C(NC[C@H](O)CN1Cc2ccccc2C2(CC2)C1)c1cc(NC2COC2)ncn1] | 409.2114 | — |
| 20-9 | 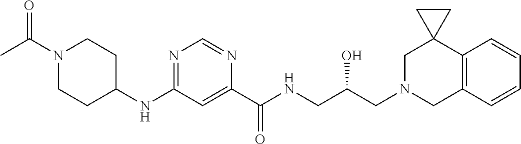 [SMILES: CC(=O)N1CCC(Nc2cc(C(=O)NC[C@H](O)CN3Cc4ccccc4C4(CC4)C3)ncn2)CC1] | 478.2692 | — |
| 21-9 | 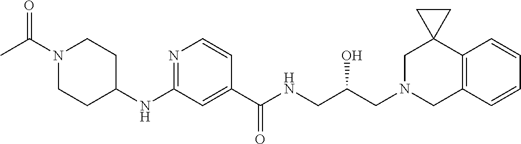 [SMILES: CC(=O)N1CCC(Nc2cc(C(=O)NC[C@H](O)CN3Cc4ccccc4C4(CC4)C3)ccn2)CC1] | 477.2740 | — |
| 22-9 | 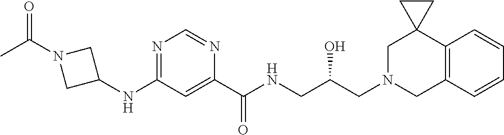 [SMILES: CC(=O)N1CC(Nc2cc(C(=O)NC[C@H](O)CN3Cc4ccccc4C4(CC4)C3)ncn2)C1] | 450.2379 | — |
| 23-9 | 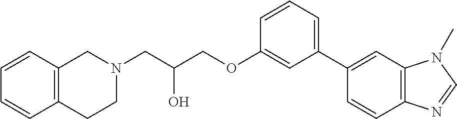 [SMILES: Cn1cnc2ccc(-c3cccc(OCC(O)CN4CCc5ccccc5C4)c3)cc21] | 413.2103 | — |
| 24-9 | 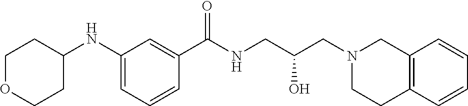 [SMILES: O=C(NC[C@@H](O)CN1CCc2ccccc2C1)c1cccc(NC2CCOCC2)c1] | 409.2365 | — |

In certain embodiments, a provided compound is a compound listed in Table 1J, or a pharmaceutically acceptable salt thereof.

TABLE 1J
Exemplary Compounds

| | | | LCMS |
|---|---|---|---|
| Cmpd | | Exact | m/z |
| No | Structure | Mass | (M + H) |
| 1-10 | 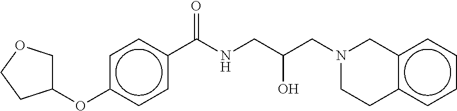 | 396.2049 | 397.2 |

In certain embodiments, a provided compound is a compound listed in Table 1K, or a pharmaceutically acceptable salt thereof.

TABLE 1K
Exemplary Compounds

| | | | LCMS |
|---|---|---|---|
| Cmpd | | Exact | m/z |
| No | Structure | Mass | (M + H) |
| 1-11 | 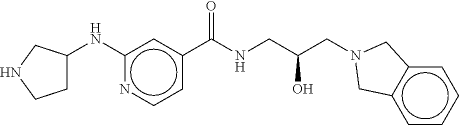 | 381.2165 | 382.1 |
| 2-11 | 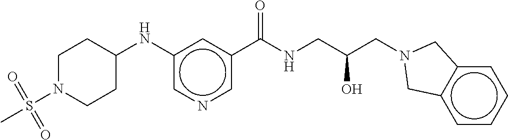 | 473.2097 | 474.3 |
| 3-11 | 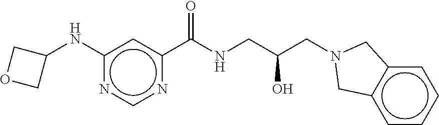 | 369.1801 | 370.1 |
| 4-11 | 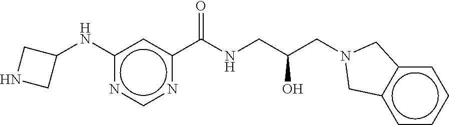 | 368.1961 | 369.1 |
| 5-11 | 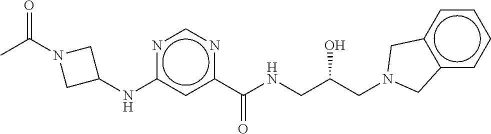 | 410.2066 | 411.3 |
| 6-11 | 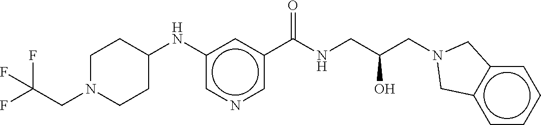 | 477.2352 | 478.1 |
| 7-11 | 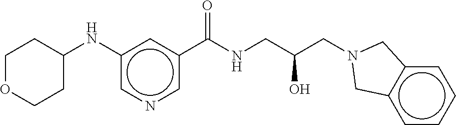 | 396.2161 | 397.1 |
| 8-11 | 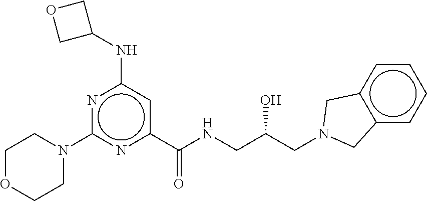 | 454.2329 | 455.1 |
| 9-11 | 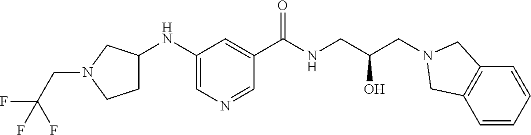 [SMILES: O=C(NC[C@H](O)CN1Cc2ccccc2C1)c1cncc(NC2CCN(CC(F)(F)F)C2)c1] | — | — |

In certain embodiments, a provided compound is a compound listed in Table 1L, or a pharmaceutically acceptable salt thereof.

TABLE 1-L
Exemplary Compounds

| | | | LCMS |
|---|---|---|---|
| Cmpd | | Exact | m/z |
| No | Structure | Mass | (M + H) |
| 1-12 | 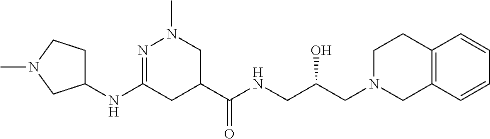 [SMILES: CN1CCC(NC2=NN(C)CC(C(=O)NC[C@H](O)CN3CCc4ccccc4C3)C2)C1] | 428.2900 | — |

In certain embodiments, a provided compound is a compound listed in Table 1M, or a pharmaceutically acceptable salt thereof.

TABLE 1M
Exemplary Compounds

| | | | LCMS |
|---|---|---|---|
| Cmpd | | Exact | m/z |
| No | Structure | Mass | (M + H) |
| 1-13 | 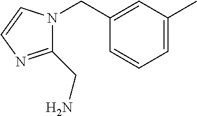 | 201.1266 | — |
| 2-13 | 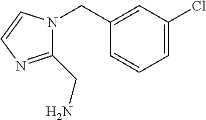 [SMILES: NCc1nccn1Cc1cccc(Cl)c1] | 221.072 | — |
| 3-13 | 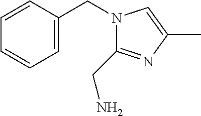 | 201.1266 | — |
| 4-13 | 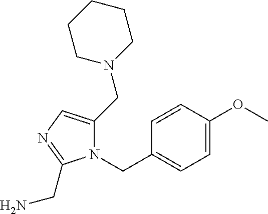 [SMILES: COc1ccc(Cn2c(CN3CCCCC3)cnc2CN)cc1] | 314.2107 | — |
| 5-13 | 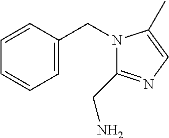 | 201.1266 | — |
| 6-13 | 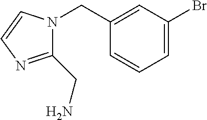 [SMILES: NCc1nccn1Cc1cccc(Br)c1] | 265.0215 | — |
| 7-13 | 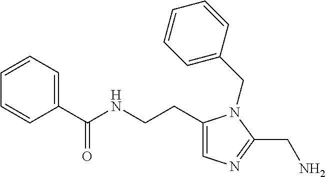 [SMILES: NCc1ncc(CCNC(=O)c2ccccc2)n1Cc1ccccc1] | 334.1794 | — |

In certain embodiments, a provided compound is a compound listed in Table 1N, or a pharmaceutically acceptable salt thereof.

TABLE 1N
Exemplary Compounds

| | | | LCMS |
|---|---|---|---|
| Cmpd | | Exact | m/z |
| No | Structure | Mass | (M + H) |
| 1-14 | 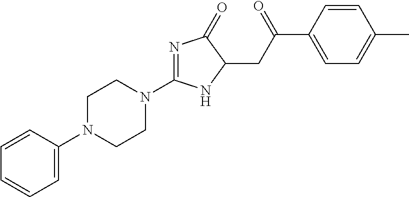 [SMILES: Cc1ccc(C(=O)CC2NC(N3CCN(c4ccccc4)CC3)=NC2=O)cc1] | 376.1899 | — |
| 2-14 | 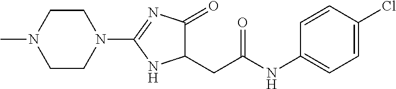 [SMILES: CN1CCN(C2=NC(=O)C(CC(=O)Nc3ccc(Cl)cc3)N2)CC1] | 349.1306 | — |
| 3-14 | 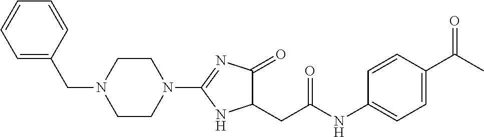 [SMILES: CC(=O)c1ccc(NC(=O)CC2NC(N3CCN(Cc4ccccc4)CC3)=NC2=O)cc1] | 433.2114 | — |
| 4-14 | 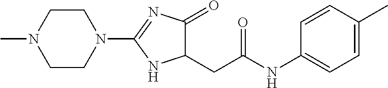 | 329.1852 | — |
| 5-14 | 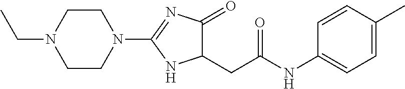 [SMILES: CCN1CCN(C2=NC(=O)C(CC(=O)Nc3ccc(C)cc3)N2)CC1] | 343.2008 | — |
| 6-14 | 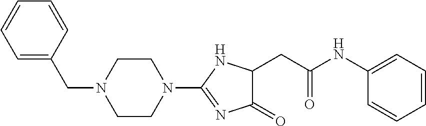 [SMILES: O=C(CC1NC(N2CCN(Cc3ccccc3)CC2)=NC1=O)Nc1ccccc1] | 391.2008 | — |
| 7-14 | 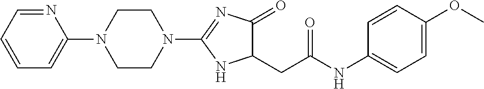 [SMILES: COc1ccc(NC(=O)CC2NC(N3CCN(c4ccccn4)CC3)=NC2=O)cc1] | 408.191 | — |
| 8-14 | 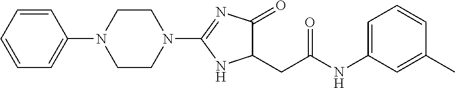 [SMILES: Cc1cccc(NC(=O)CC2NC(N3CCN(c4ccccc4)CC3)=NC2=O)c1] | 391.2008 | — |
| 9-14 | 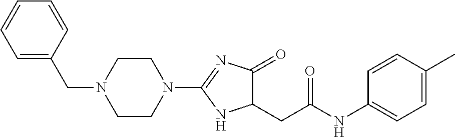 [SMILES: Cc1ccc(NC(=O)CC2NC(N3CCN(Cc4ccccc4)CC3)=NC2=O)cc1] | 405.2165 | — |
| 10-14 | 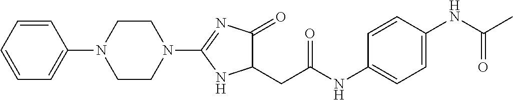 [SMILES: CC(=O)Nc1ccc(NC(=O)CC2NC(N3CCN(c4ccccc4)CC3)=NC2=O)cc1] | 434.2066 | — |
| 11-14 | 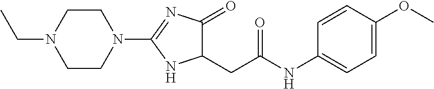 [SMILES: CCN1CCN(C2=NC(=O)C(CC(=O)Nc3ccc(OC)cc3)N2)CC1] | 359.1957 | — |
| 12-14 | 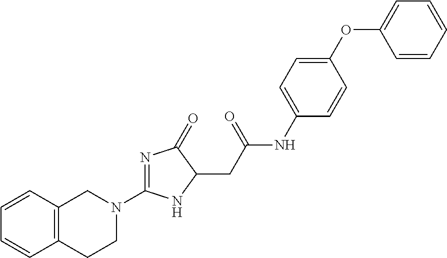 [SMILES: O=C(CC1NC(N2CCc3ccccc3C2)=NC1=O)Nc1ccc(Oc2ccccc2)cc1] | 440.1848 | — |
| 13-14 | 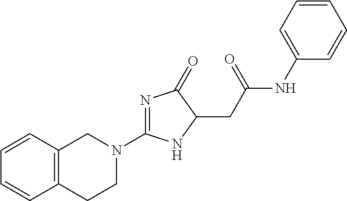 [SMILES: O=C(CC1NC(N2CCc3ccccc3C2)=NC1=O)Nc1ccccc1] | 348.1586 | — |
| 14-14 | 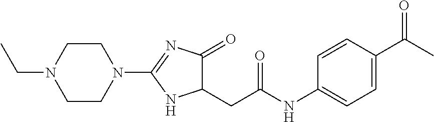 [SMILES: CCN1CCN(C2=NC(=O)C(CC(=O)Nc3ccc(C(C)=O)cc3)N2)CC1] | 371.1957 | — |
| 15-14 | 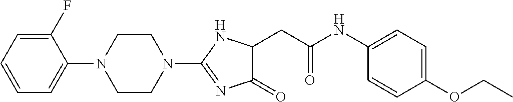 [SMILES: CCOc1ccc(NC(=O)CC2NC(N3CCN(c4ccccc4F)CC3)=NC2=O)cc1] | 439.202 | — |
| 16-14 | 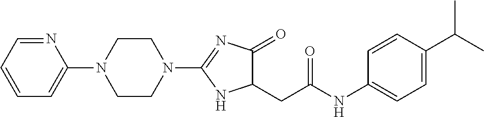 [SMILES: CC(C)c1ccc(NC(=O)CC2NC(N3CCN(c4ccccn4)CC3)=NC2=O)cc1] | 420.2274 | — |
| 17-14 | 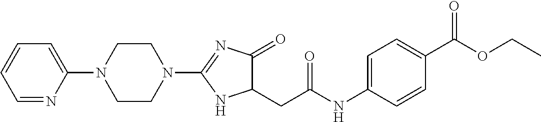 [SMILES: CCOC(=O)c1ccc(NC(=O)CC2NC(N3CCN(c4ccccn4)CC3)=NC2=O)cc1] | 450.2016 | — |
| 18-14 | 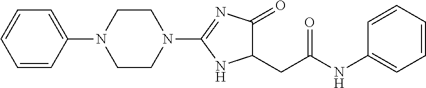 [SMILES: O=C(CC1NC(N2CCN(c3ccccc3)CC2)=NC1=O)Nc1ccccc1] | 377.1852 | — |
| 19-14 | 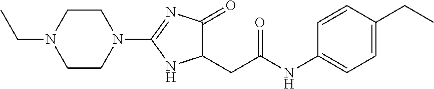 [SMILES: CCc1ccc(NC(=O)CC2NC(N3CCN(CC)CC3)=NC2=O)cc1] | 357.2165 | — |
| 20-14 | 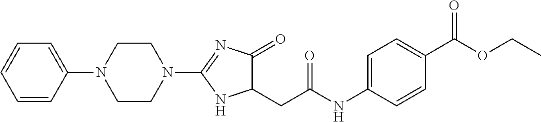 [SMILES: CCOC(=O)c1ccc(NC(=O)CC2NC(N3CCN(c4ccccc4)CC3)=NC2=O)cc1] | 449.2063 | — |
| 21-14 | 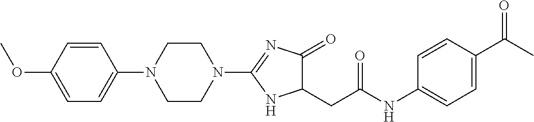 [SMILES: COc1ccc(N2CCN(C3=NC(=O)C(CC(=O)Nc4ccc(C(C)=O)cc4)N3)CC2)cc1] | 449.2063 | — |
| 22-14 | 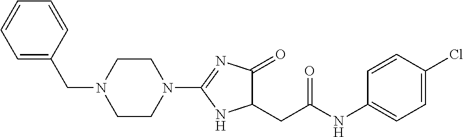 [SMILES: O=C(CC1NC(N2CCN(Cc3ccccc3)CC2)=NC1=O)Nc1ccc(Cl)cc1] | 425.1619 | — |
| 23-14 | 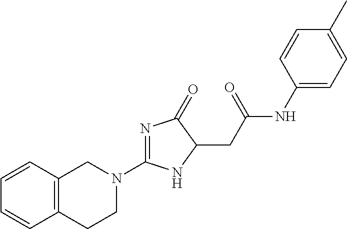 [SMILES: Cc1ccc(NC(=O)CC2NC(N3CCc4ccccc4C3)=NC2=O)cc1] | 362.1743 | — |
| 24-14 | 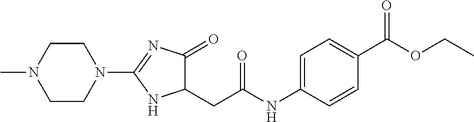 [SMILES: CCOC(=O)c1ccc(NC(=O)CC2NC(N3CCN(C)CC3)=NC2=O)cc1] | 387.1907 | — |
| 25-14 | 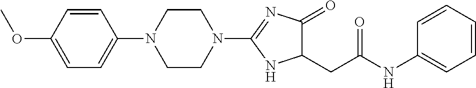 [SMILES: COc1ccc(N2CCN(C3=NC(=O)C(CC(=O)Nc4ccccc4)N3)CC2)cc1] | 407.1957 | — |
| 26-14 | 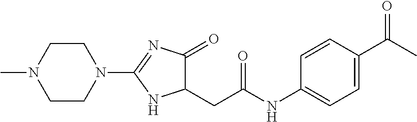 [SMILES: CC(=O)c1ccc(NC(=O)CC2NC(N3CCN(C)CC3)=NC2=O)cc1] | 357.1801 | — |
| 27-14 | 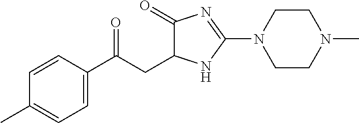 | 314.1743 | — |
| 28-14 | 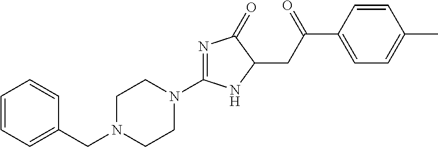 [SMILES: Cc1ccc(C(=O)CC2NC(N3CCN(Cc4ccccc4)CC3)=NC2=O)cc1] | 390.2056 | — |
| 29-14 | 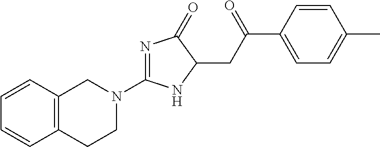 [SMILES: Cc1ccc(C(=O)CC2NC(N3CCc4ccccc4C3)=NC2=O)cc1] | 347.1634 | — |
| 30-14 | 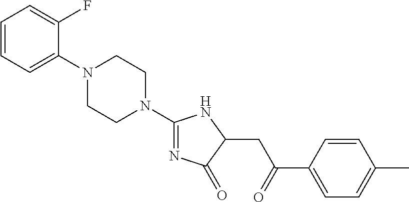 [SMILES: Cc1ccc(C(=O)CC2NC(N3CCN(c4ccccc4F)CC3)=NC2=O)cc1] | 394.1805 | — |
| 31-14 | 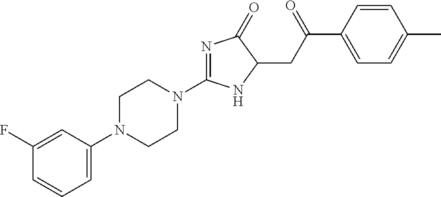 [SMILES: Cc1ccc(C(=O)CC2NC(N3CCN(c4cccc(F)c4)CC3)=NC2=O)cc1] | 394.1805 | — |
| 32-14 | 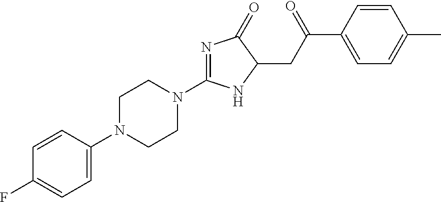 [SMILES: Cc1ccc(C(=O)CC2NC(N3CCN(c4ccc(F)cc4)CC3)=NC2=O)cc1] | 394.1805 | — |
| 33-14 | 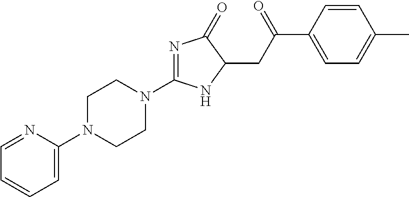 [SMILES: Cc1ccc(C(=O)CC2NC(N3CCN(c4ccccn4)CC3)=NC2=O)cc1] | 377.1852 | — |
| 34-14 | 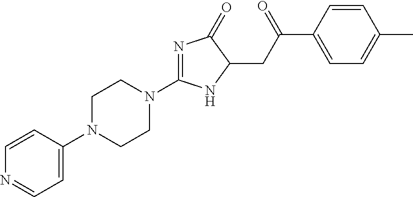 [SMILES: Cc1ccc(C(=O)CC2NC(N3CCN(c4ccncc4)CC3)=NC2=O)cc1] | 377.1852 | — |
| 35-14 | 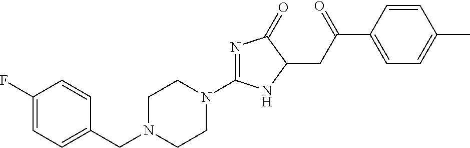 [SMILES: Cc1ccc(C(=O)CC2NC(N3CCN(Cc4ccc(F)cc4)CC3)=NC2=O)cc1] | 408.1962 | — |
| 36-14 | 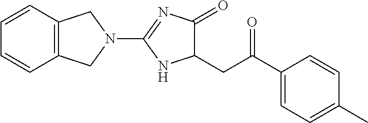 [SMILES: Cc1ccc(C(=O)CC2NC(N3Cc4ccccc4C3)=NC2=O)cc1] | 333.1477 | — |
| 37-14 | 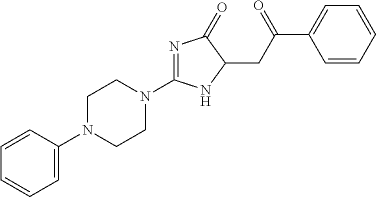 [SMILES: O=C(CC1NC(N2CCN(c3ccccc3)CC2)=NC1=O)c1ccccc1] | 362.1743 | — |
| 38-14 | 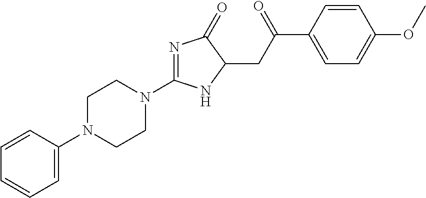 [SMILES: COc1ccc(C(=O)CC2NC(N3CCN(c4ccccc4)CC3)=NC2=O)cc1] | 392.1848 | — |
| 39-14 | 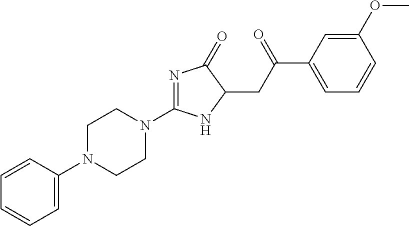 [SMILES: COc1cccc(C(=O)CC2NC(N3CCN(c4ccccc4)CC3)=NC2=O)c1] | 392.1848 | — |
| 40-14 | 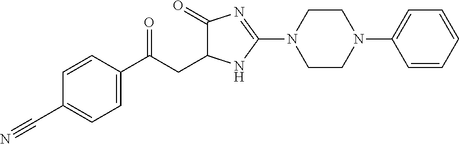 [SMILES: N#Cc1ccc(C(=O)CC2NC(N3CCN(c4ccccc4)CC3)=NC2=O)cc1] | 387.1695 | — |
| 41-14 | 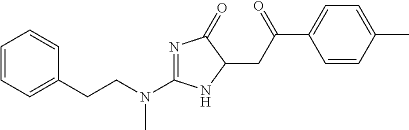 [SMILES: Cc1ccc(C(=O)CC2NC(N(C)CCc3ccccc3)=NC2=O)cc1] | 349.179 | — |
| 42-14 | 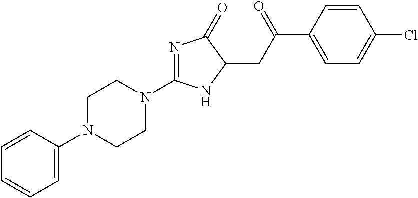 [SMILES: O=C(CC1NC(N2CCN(c3ccccc3)CC2)=NC1=O)c1ccc(Cl)cc1] | 396.1353 | — |
| 43-14 | 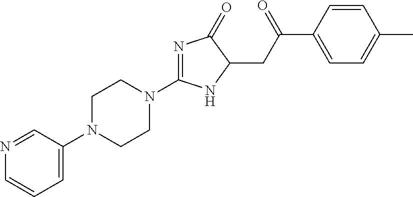 [SMILES: Cc1ccc(C(=O)CC2NC(N3CCN(c4cccnc4)CC3)=NC2=O)cc1] | 377.1852 | — |
| 44-14 | 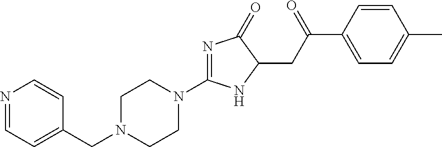 [SMILES: Cc1ccc(C(=O)CC2NC(N3CCN(Cc4ccncc4)CC3)=NC2=O)cc1] | 391.2008 | — |
| 45-14 | 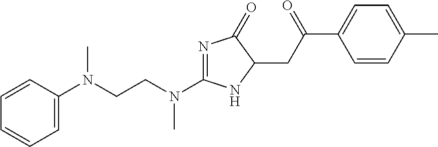 [SMILES: Cc1ccc(C(=O)CC2NC(N(C)CCN(C)c3ccccc3)=NC2=O)cc1] | 378.2056 | — |
| 46-14 | 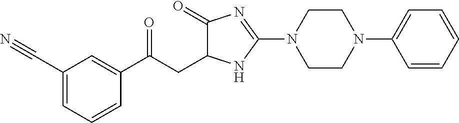 [SMILES: N#Cc1cccc(C(=O)CC2NC(N3CCN(c4ccccc4)CC3)=NC2=O)c1] | 387.1695 | — |
| 47-14 | 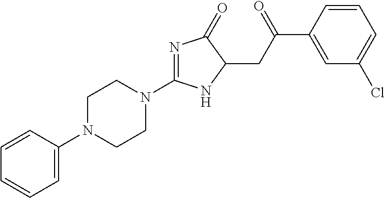 [SMILES: O=C(CC1NC(N2CCN(c3ccccc3)CC2)=NC1=O)c1cccc(Cl)c1] | 396.1353 | — |
| 48-14 | 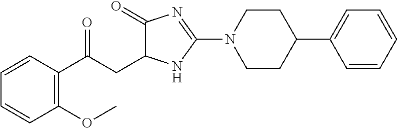 [SMILES: COc1ccccc1C(=O)CC1NC(N2CCC(c3ccccc3)CC2)=NC1=O] | 391.1896 | — |
| 49-14 | 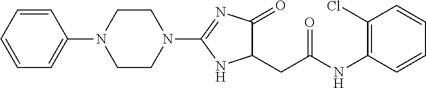 [SMILES: O=C(CC1NC(N2CCN(c3ccccc3)CC2)=NC1=O)Nc1ccccc1Cl] | 411.1462 | — |
| 50-14 | 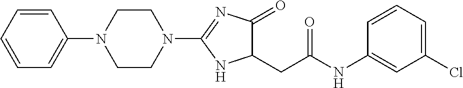 [SMILES: O=C(CC1NC(N2CCN(c3ccccc3)CC2)=NC1=O)Nc1cccc(Cl)c1] | 411.1462 | — |
| 51-14 | 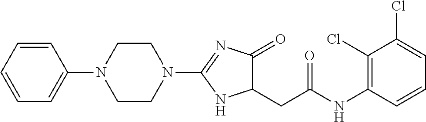 [SMILES: O=C(CC1NC(N2CCN(c3ccccc3)CC2)=NC1=O)Nc1cccc(Cl)c1Cl] | 445.1072 | — |
| 52-14 | 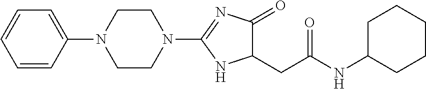 [SMILES: O=C(CC1NC(N2CCN(c3ccccc3)CC2)=NC1=O)NC1CCCCC1] | 383.2321 | — |
| 53-14 | 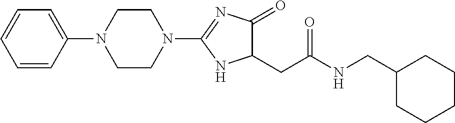 [SMILES: O=C(CC1NC(N2CCN(c3ccccc3)CC2)=NC1=O)NCC1CCCCC1] | 397.2478 | — |
| 54-14 | 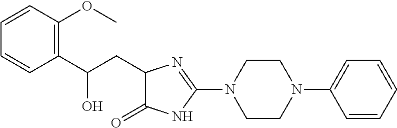 [SMILES: COc1ccccc1C(O)CC1N=C(N2CCN(c3ccccc3)CC2)NC1=O] | 394.2005 | — |
| 55-14 | 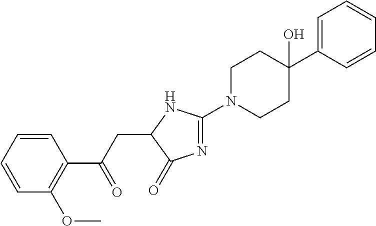 [SMILES: COc1ccccc1C(=O)CC1NC(N2CCC(O)(c3ccccc3)CC2)=NC1=O] | 407.1845 | — |
| 56-14 | 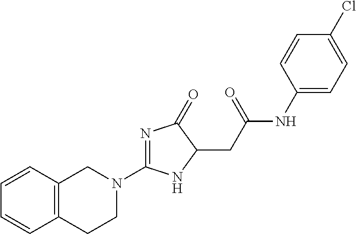 [SMILES: O=C(CC1NC(N2CCc3ccccc3C2)=NC1=O)Nc1ccc(Cl)cc1] | 382.1197 | — |
| 57-14 | 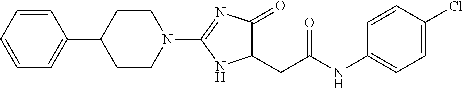 [SMILES: O=C(CC1NC(N2CCC(c3ccccc3)CC2)=NC1=O)Nc1ccc(Cl)cc1] | 410.151 | — |
| 58-14 | 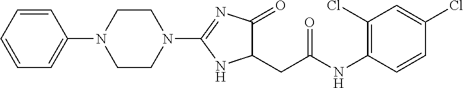 [SMILES: O=C(CC1NC(N2CCN(c3ccccc3)CC2)=NC1=O)Nc1ccc(Cl)cc1Cl] | 445.1072 | — |
| 59-14 | 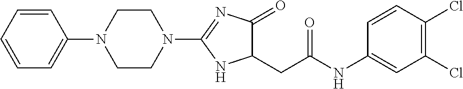 [SMILES: O=C(CC1NC(N2CCN(c3ccccc3)CC2)=NC1=O)Nc1ccc(Cl)c(Cl)c1] | 445.1072 | — |
| 60-14 | 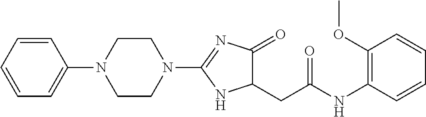 [SMILES: COc1ccccc1NC(=O)CC1NC(N2CCN(c3ccccc3)CC2)=NC1=O] | 407.1957 | — |
| 61-14 | 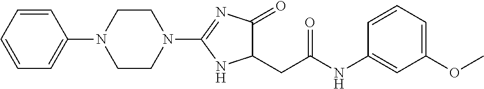 [SMILES: COc1cccc(NC(=O)CC2NC(N3CCN(c4ccccc4)CC3)=NC2=O)c1] | 407.1957 | — |
| 62-14 | 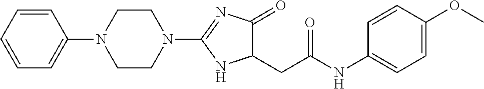 [SMILES: COc1ccc(NC(=O)CC2NC(N3CCN(c4ccccc4)CC3)=NC2=O)cc1] | 407.1957 | — |
| 63-14 | 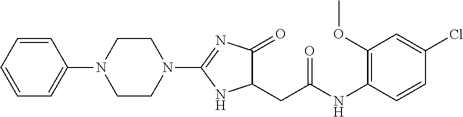 [SMILES: COc1cc(Cl)ccc1NC(=O)CC1NC(N2CCN(c3ccccc3)CC2)=NC1=O] | 441.1568 | — |
| 64-14 | 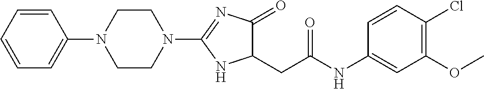 [SMILES: COc1cc(NC(=O)CC2NC(N3CCN(c4ccccc4)CC3)=NC2=O)ccc1Cl] | 441.1568 | — |
| 65-14 | 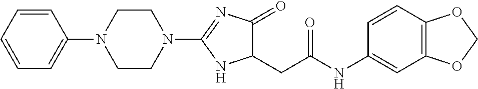 [SMILES: O=C(CC1NC(N2CCN(c3ccccc3)CC2)=NC1=O)Nc1ccc2c(c1)OCO2] | 421.175 | — |
| 66-14 | 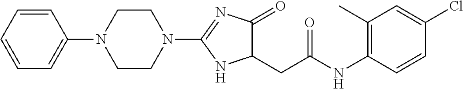 [SMILES: Cc1cc(Cl)ccc1NC(=O)CC1NC(N2CCN(c3ccccc3)CC2)=NC1=O] | 425.1619 | — |
| 67-14 | 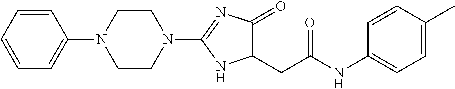 [SMILES: Cc1ccc(NC(=O)CC2NC(N3CCN(c4ccccc4)CC3)=NC2=O)cc1] | 391.2008 | — |
| 68-14 | 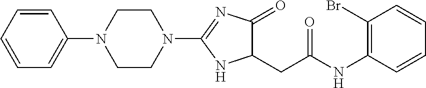 [SMILES: O=C(CC1NC(N2CCN(c3ccccc3)CC2)=NC1=O)Nc1ccccc1Br] | 455.0957 | — |
| 69-14 | 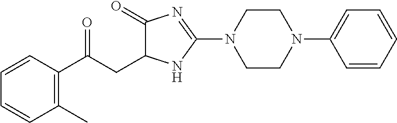 [SMILES: Cc1ccccc1C(=O)CC1NC(N2CCN(c3ccccc3)CC2)=NC1=O] | 376.1899 | — |
| 70-14 | 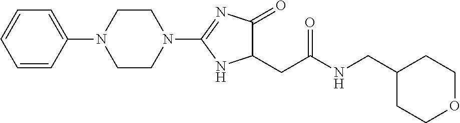 [SMILES: O=C(CC1NC(N2CCN(c3ccccc3)CC2)=NC1=O)NCC1CCOCC1] | 399.227 | — |
| 71-14 | 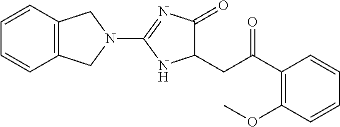 [SMILES: COc1ccccc1C(=O)CC1NC(N2Cc3ccccc3C2)=NC1=O] | 349.1426 | — |
| 72-14 | 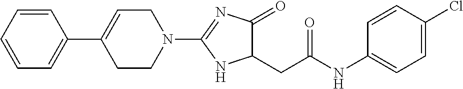 [SMILES: O=C(CC1NC(N2CC=C(c3ccccc3)CC2)=NC1=O)Nc1ccc(Cl)cc1] | 408.1353 | — |
| 73-14 | 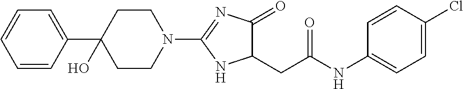 [SMILES: O=C(CC1NC(N2CCC(O)(c3ccccc3)CC2)=NC1=O)Nc1ccc(Cl)cc1] | 426.1459 | — |
| 74-14 | 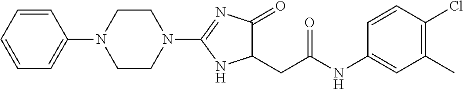 [SMILES: Cc1cc(NC(=O)CC2NC(N3CCN(c4ccccc4)CC3)=NC2=O)ccc1Cl] | 425.1619 | — |
| 75-14 | 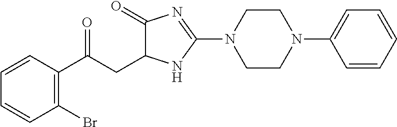 [SMILES: O=C(CC1NC(N2CCN(c3ccccc3)CC2)=NC1=O)c1ccccc1Br] | 440.0848 | — |

In certain embodiments, a provided compound is a compound listed in Table 1O, or a pharmaceutically acceptable salt thereof.

TABLE 1O
Exemplary Compounds

| | | | LCMS |
|---|---|---|---|
| Cmpd | | Exact | m/z |
| No | Structure | Mass | (M + H) |
| 1-15 | 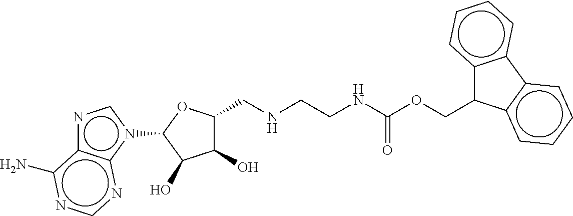 | 531.2230 | — |

In certain embodiments, a provided compound inhibits PRMT5. In certain embodiments, a provided compound inhibits wild-type PRMT5. In certain embodiments, a provided compound inhibits a mutant PRMT5. In certain embodiments, a provided compound inhibits PRMT5, e.g., as measured in an assay described herein. In certain embodiments, the PRMT5 is from a human. In certain embodiments, a provided compound inhibits PRMT5 at an IC₅₀ less than or equal to 10 μM. In certain embodiments, a provided compound inhibits PRMT5 at an IC₅₀ less than or equal to 1 μM. In certain embodiments, a provided compound inhibits PRMT5 at an IC₅₀ less than or equal to 0.1 μM. In certain embodiments, a provided compound inhibits PRMT5 in a cell at an EC₅₀ less than or equal to 10 μM. In certain embodiments, a provided compound inhibits PRMT5 in a cell at an EC50 less than or equal to 1 μM. In certain embodiments, a provided compound inhibits PRMT5 in a cell at an EC₅₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM. In certain embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective for PRMT5 relative to one or more other methyltransferases.

It will be understood by one of ordinary skill in the art that the PRMT5 can be wild-type PRMT5, or any mutant or variant of PRMT5.

In certain embodiments embodiment, the mutant or variant of PRMT5 contains one or more mutations (e.g., conservative substitutions). In certain embodiments, provided herein is a PRMT5 point mutant. In certain embodiments, the PRMT point mutant has an amino acid sequence that a degree of homology to the amino acid sequence of SEQ ID NO: 1 of at least about 80%, e.g., at least about 85%, at least about 90%, at least about 95%, or at least about 97%. Further provided is a protein that has a degree of homology to the amino acid sequence of SEQ ID NO: 2 of at least about 80%, e.g., at least about 85%, at least about 90%, at least about 95%, or at least about 97%.

In certain embodiments, the PRMT5 is isoform A (GenBank accession no. NP006100) (SEQ ID NO.:1):

MAAMAVGGAG GSRVSSGRDL NCVPEIADTL GAVAKQGFDF
LCMPVFHPRF KREFIQEPAK NRPGPQTRSD LLLSGRDWNT
LIVGKLSPWI RPDSKVEKIR RNSEAAMLQE LNFGAYLGLP
AFLLPLNQED NTNLARVLTN HIHTGHHSSM FWMRVPLVAP
EDLRDDIIEN APTTHTEEYS GEEKTWMWWH NFRTLCDYSK
RIAVALEIGA DLPSNHVIDR WLGEPIKAAI LPTSIFLTNK
KGFPVLSKMH QRLIFRLLKL EVQFIITGTN HHSEKEFCSY
LQYLEYLSQN RPPPNAYELF AKGYEDYLQS PLQPLMDNLE
SQTYEVFEKD PIKYSQYQQA IYKCLLDRVP EEEKDTNVQV
LMVLGAGRGP LVNASLRAAK QADRRIKLYA VEKNPNAVVT
LENWQFEEWG SQVTVVSSDM REWVAPEKAD IIVSELLGSF
ADNELSPECL DGAQHFLKDD GVSIPGEYTS FLAPISSSKL
YNEVRACREK DRDPEAQFEM PYVVRLHNFH QLSAPQPCFT
FSHPNRDPMI DNNRYCTLEF PVEVNTVLHG FAGYFETVLY
QDITLSIRPE THSPGMFSWF PILFPIKQPI TVREGQTICV
RFWRCSNSKK VWYEWAVTAP VCSAIHNPTG RSYTIGL

In certain embodiments, the PRMT5 is isoform B (GenBank accession no. NP001034708) (SEQ ID NO.:2)

MRGPNSGTEK GRLVIPEKQG FDFLCMPVFH PRFKREFIQE
PAKNRPGPQT RSDLLLSGRD WNTLIVGKLS PWIRPDSKVE
KIRRNSEAAM LQELNFGAYL GLPAFLLPLN QEDNTNLARV
LTNHIHTGHH SSMFWMRVPL VAPEDLRDDI IENAPTTHTE
EYSGEEKTWM WWHNFRTLCD YSKRIAVALE IGADLPSNHV
IDRWLGEPIK AAILPTSIFL TNKKGFPVLS KMHQRLIFRL
LKLEVQFIIT GTNHHSEKEF CSYLQYLEYL SQNRPPPNAY
ELFAKGYEDY LQSPLQPLMD NLESQTYEVF EKDPIKYSQY
QQAIYKCLLD RVPEEEKDTN VQVLMVLGAG RGPLVNASLR
AAKQADRRIK LYAVEKNPNA VVTLENWQFE EWGSQVTVVS
SDMREWVAPE KADIIVSELL GSFADNELSP ECLDGAQHFL
KDDGVSIPGE YTSFLAPISS SKLYNEVRAC REKDRDPEAQ
FEMPYVVRLH NFHQLSAPQP CFTFSHPNRD PMIDNNRYCT
LEFPVEVNTV LHGFAGYFET VLYQDITLSI RPETHSPGMF
SWFPILFPIK QPITVREGQT ICVRFWRCSN SKKVWYEWAV
TAPVCSAIHN PTGRSYTIGL

In certain embodiments, the PRMT5 is transcript variant 1 (GenBank accession no. NM_006109).

### Pharmaceutical Compositions

The present disclosure provides pharmaceutical compositions comprising a compound described herein, e.g., a compound of Formula (I)-(XIII), or a pharmaceutically acceptable salt thereof, as described herein, and optionally a pharmaceutically acceptable excipient. It will be understood by one of ordinary skill in the art that the compounds described herein, or salts thereof, may be present in various forms, such as amorphous, hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting PRMT5. In certain embodiments, the effective amount is an amount effective for treating a PRMT5-mediated disorder. In certain embodiments, the effective amount is a prophylactically effective amount. In certain embodiments, the effective amount is an amount effective to prevent a PRMT5-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

### Methods of Treatment

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In certain embodiments, a compound described herein is administered one or more times per day, for multiple days. In certain embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (I)-(XIII). In certain embodiments, the additional therapeutically active agent is not a compound of Formula (I)-(XIII). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In certain embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the present discosure are kits (e.g., pharmaceutical packs). The kits provided may comprise a provided pharmaceutical composition or compound and a container (e.g., a vial, ampule, bottle, syringe, and/or dispenser package, or other suitable container). In certain embodiments, provided kits may optionally further include a second container comprising a pharmaceutical excipient for dilution or suspension of a provided pharmaceutical composition or compound. In certain embodiments, a provided pharmaceutical composition or compound provided in the container and the second container are combined to form one unit dosage form. In certain embodiments, a provided kits further includes instructions for use.

Compounds and compositions described herein are generally useful for the inhibition of PRMT5. In certain embodiments, methods of treating PRMT5-mediated disorder in a subject are provided which comprise administering an effective amount of a compound described herein (e.g., a compound of Formula (I)-(XIII)), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a PRMT5-mediated disorder. In certain embodiments, the subject is susceptible to a PRMT5-mediated disorder.

As used herein, the term “PRMT5-mediated disorder” means any disease, disorder, or other pathological condition in which PRMT5 is known to play a role. Accordingly, in certain embodiments, the present disclosure relates to treating or lessening the severity of one or more diseases in which PRMT5 is known to play a role.

In certain embodiments, the present disclosure provides a method of inhibiting PRMT5 comprising contacting PRMT5 with an effective amount of a compound described herein (e.g., a compound of Formula (I)-(XIII)), or a pharmaceutically acceptable salt thereof. The PRMT5 may be purified or crude, and may be present in a cell, tissue, or subject. Thus, such methods encompass both inhibition of in vitro and in vivo PRMT5 activity. In certain embodiments, the method is an in vitro method, e.g., such as an assay method. It will be understood by one of ordinary skill in the art that inhibition of PRMT5 does not necessarily require that all of the PRMT5 be occupied by an inhibitor at once. Exemplary levels of inhibition of PRMT5 include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In certain embodiments, provided is a method of inhibiting PRMT5 activity in a subject in need thereof comprising administering to the subject an effective amount of a compound described herein (e.g., a compound of Formula (I)-(XIII)), or a pharmaceutically acceptable salt thereof, or a pharmaceutical composition thereof.

In certain embodiments, provided is a method of altering gene expression in a cell which comprises contacting a cell with an effective amount of a compound of Formula (I)-(XIII)), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, provided is a method of altering transcription in a cell which comprises contacting a cell with an effective amount of a compound of Formula (I)-(XIII), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, a method is provided of selecting a therapy for a subject having a disease associated with PRMT5-mediated disorder or mutation comprising the steps of determining the presence of PRMT5-mediated disorder or gene mutation in the PRMT5 gene or and selecting, based on the presence of PRMT5-mediated disorder a gene mutation in the PRMT5 gene a therapy that includes the administration of a provided compound. In certain embodiments, the disease is cancer.

In certain embodiments, a method of treatment is provided for a subject in need thereof comprising the steps of determining the presence of PRMT5-mediated disorder or a gene mutation in the PRMT5 gene and treating the subject in need thereof, based on the presence of a PRMT5-mediated disorder or gene mutation in the PRMT5 gene with a therapy that includes the administration of a provided compound. In certain embodiments, the subject is a cancer patient.

In certain embodiments, a provided compound is useful in treating a proliferative disorder, such as cancer, a benign neoplasm, an autoimmune disease, or an inflammatory disease. For example, while not being bound to any particular mechanism, PRMT5 has been shown to be involved in cyclin D1 dysregulated cancers. Increased PRMT5 activity mediates key events associated with cyclin DI-dependent neoplastic growth including CUL4 repression, CDT1 overexpression, and DNA re-replication. Further, human cancers harboring mutations in Fbx4, the cyclin D1 E3 ligase, exhibit nuclear cyclin D1 accumulation and increased PRMT5 activity. See, e.g., Aggarwal et al., Cancer Cell. (2010) 18(4):329-40. Additionally, PRMT5 has also been implicated in accelerating cell cycle progression through G1 phase and modulating regulators of G1; for example, PRMT5 may upregulate cyclin-dependent kinase (CDK) 4, CDK6, and cyclins D1, D2 and E1. Moreover, PRMT5 may activate phosphoinositide 3-kinase (PI3K)/AKT signaling. See, e.g., Wei et al., Cancer Sci. (2012) 103(9):1640-50. PRMT5 has been reported to play a role in apoptosis through methylation of E2F-1. See, e.g., Cho et al., EMBO J. (2012) 31:1785-1797; Zheng et al., Mol. Cell. (2013) 52:37-51. PRMT5 has been reported to be an essential regulator of splicing and affect the alternative splicing of ‘sensor’ mRNAs that can then lead to defects in downstream events such as apoptosis. See, e.g., Bezzi et al., Genes Dev. (2013) 27:1903-1916. PRMT5 has been reported to play a role in the RAS-ERK pathway. See, e.g., Andrew-Perez et al., Sci Signal. (2011) Sep. 13; 4(190)ra58 doi: 10.1126/scisignal.2001936. PRMT5 has been reported to affect C/EBPb target genes through interaction with the Mediator complex and hence affect cellular differentiation and inflammatory response. See, e.g., Tsutsui et al., J. Biol. Chem. (2013) 288:20955-20965. PRMT5 has been shown to methylate HOXA9 essential for ELAM expression during the EC inflammatory response. See, e.g., Bandyopadhyay et al., Mol. Cell. Biol. (2012) 32:1202-1203. Thus in certain embodiments, the inhibition of PRMT5 by a provided compound is useful in treating the following non-limiting list of cancers: breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous cystadenocarcinoma. See, e.g., Pal et al., EMBO J. (2007) 26:3558-3569 (mantle cell lymphoma); Wang et al., Mol. Cell Biol. (2008) 28:6262-77 (chronic lymphocytic leukemia (CLL)); and Tae et al., Nucleic Acids Res. (2011) 39:5424-5438.

In certain embodiments, the inhibition of PRMT5 by a provided compound is useful in treating prostate cancer and lung cancer, in which PRMT5 has been shown to play a role. See, e.g., Gu et al., PLoS One 2012; 7(8):e44033; Gu et al., Biochem. J. (2012) 446:235-241. In certain embodiments, a provided compound is useful to delay the onset of, slow the progression of, or ameliorate the symptoms of cancer. In certain embodiments, a provided compound is administered in combination with other compounds, drugs, or therapeutics to treat cancer.

In certain embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrim's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer, and vulvar cancer (e.g., Paget's disease of the vulva).

In certain embodiments, a provided compound is useful in treating a metabolic disorder, such as diabetes or obesity. For example, while not being bound to any particular mechanism, a role for PRMT5 has been recognized in adipogenesis. Inhibition of PRMT5 expression in multiple cell culture models for adipogenesis prevented the activation of adipogenic genes, while overexpression of PRMT5 enhanced adipogenic gene expression and differentiation. See, e.g., LeBlanc et al., Mol Endocrinol. (2012) 26:583-597. Additionally, it has been shown that adipogenesis plays a pivotal role in the etiology and progression of diabetes and obesity. See, e.g., Camp et al., Trends Mol Med. (2002) 8:442-447. Thus in certain embodiments, the inhibition of PRMT5 by a provided compound is useful in treating diabetes and/or obesity.

In certain embodiments, a provided compound is useful to delay the onset of, slow the progression of, or ameliorate the symptoms of, diabetes. In certain embodiments, the diabetes is Type 1 diabetes. In certain embodiments, the diabetes is Type 2 diabetes. In certain embodiments, a provided compound is useful to delay the onset of, slow the progression of, or ameliorate the symptoms of, obesity. In certain embodiments, a provided compound is useful to help a subject lose weight. In certain embodiments, a provided compound could be used in combination with other compounds, drugs, or therapeutics, such as metformin and insulin, to treat diabetes and/or obesity.

In certain embodiments, a provided compound is useful in treating a blood disorder, e.g., a hemoglobinopathy, such as sickle cell disease or β-thalassemia. For example, while not being bound to any particular mechanism, PRMT5 is a known repressor of γ-globin gene expression, and increased fetal γ-globin (HbF) levels in adulthood are associated with symptomatic amelioration in sickle cell disease and P-thalassemia. See, e.g., Xu et al., Haematologica. (2012) 97:1632-1640; Rank et al., Blood. (2010) 116:1585-1592. Thus in certain embodiments, the inhibition of PRMT5 by a provided compound is useful in treating a blood disorder, such as a hemoglobinopathy such as sickle cell disease or β-thalassemia.

In certain embodiments, a provided compound is useful to delay the onset of, slow the progression of, or ameliorate the symptoms of, sickle cell disease. In certain embodiments, a provided compound is useful to delay the onset of, slow the progression of, or ameliorate the symptoms of, β-thalassemia. In certain embodiments, a provided compound could be used in combination with other compounds, drugs, or therapeutics, to treat a hemoglobinopathy such as sickle cell disease or β-thalassemia.

In certain embodiments, a provided compound is useful in treating inflammatory and autoimmune disease. PRMT5 is reported to activate NFkB signaling pathway through the methylation of p65. PRMT5 is reported to interact with Death receptor 4 and Death receptor 5 contributing to TRAIL-induced activation of inhibitor or kB kinase (IKK) and nuclear factor-kB (NF-kB). See, e.g., Tanaka et al., Mol. Cancer. Res. (2009) 7:557-569; Wei et al., Proc. Nat'l. Acad. Sci. USA (2013) 110:13516-21.

The term “inflammatory disease” refers to those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory diseases include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis.

In certain embodiments, the inflammatory disease is an acute inflammatory disease (e.g., for example, inflammation resulting from infection). In certain embodiments, the inflammatory disease is a chronic inflammatory disease (e.g., conditions resulting from asthma, arthritis and inflammatory bowel disease). The compounds may also be useful in treating inflammation associated with trauma and non-inflammatory myalgia. The compounds may also be useful in treating inflammation associated with cancer.

Exemplary autoimmune diseases, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, a provided compound is useful in somatic cell reprogramming, such as reprogramming somatic cells into stem cells. See, e.g., Nagamatsu et al., J Biol Chem. (2011) 286:10641-10648. In certain embodiments, a provided compound is useful in germ cell development, and are thus envisioned useful in the areas of reproductive technology and regenerative medicine. See, e.g., Ancelin et al., Nat. Cell. Biol. (2006) 8:623-630.

### General Synthetic Routes

In certain embodiments, compounds described herein can prepared using methods shown in Scheme 1. Compounds of Formula (I) can be prepared through nucleophilic coupling of amines or thiols of formulae (Z-1) or (Z-2) under conditions S1 with a compound of formula (Z-3), wherein LG is a leaving group, followed by deprotection of the acetonide group of (Z-4) under conditions S2. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S2 comprise an organic or inorganic acid (e.g., camphorsulfonic acid or hydrochloric acid).

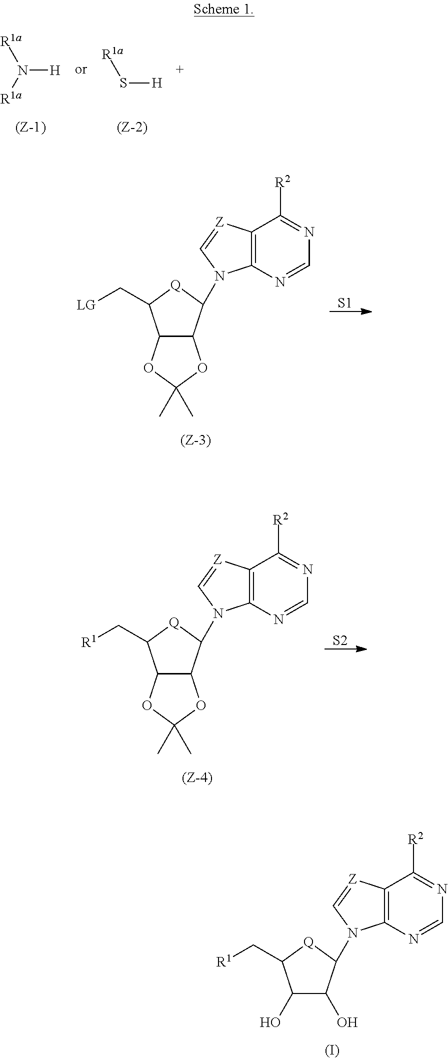

In certain embodiments, compounds described herein can prepared using methods shown in Scheme 2. Compounds of Formula (I-a) can be prepared through nucleophilic coupling of amines of formula (Z-5) under conditions S1 with a compound of formula (Z-3), wherein LG is a leaving group, followed by deprotection of the acetonide group of (Z-6) under conditions S2. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S2 comprise an organic or inorganic acid (e.g., camphorsulfonic acid or hydrochloric acid).

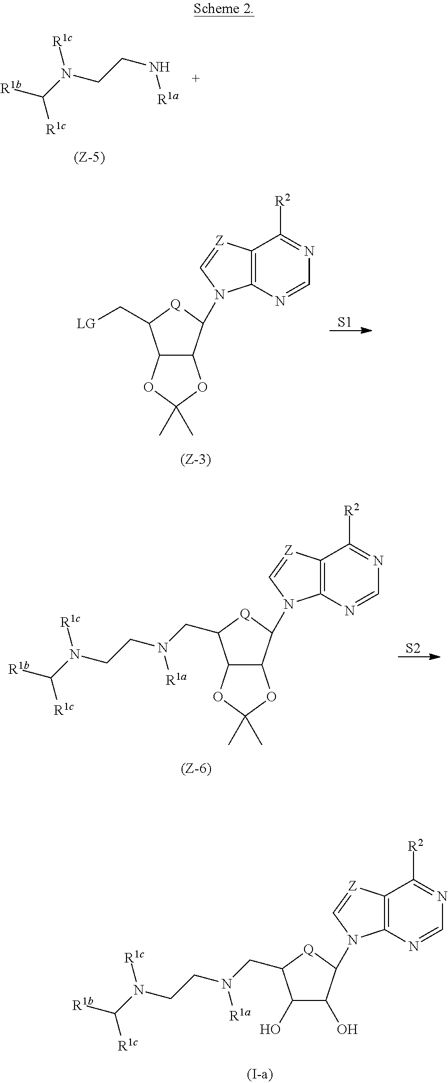

In certain embodiments, compounds described herein can prepared using methods shown in Scheme 3. Compounds of Formula (I-b) can be prepared through nucleophilic coupling of amines of formula (Z-7) under conditions S1 with a compound of formula (Z-3), wherein LG is a leaving group, followed by deprotection of the nitrogen protecting group of (Z-8) and acetonide group of (Z-9) under conditions S3 and S2, respectively. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S2 comprise an organic or inorganic acid (e.g., camphorsulfonic acid or hydrochloric acid).

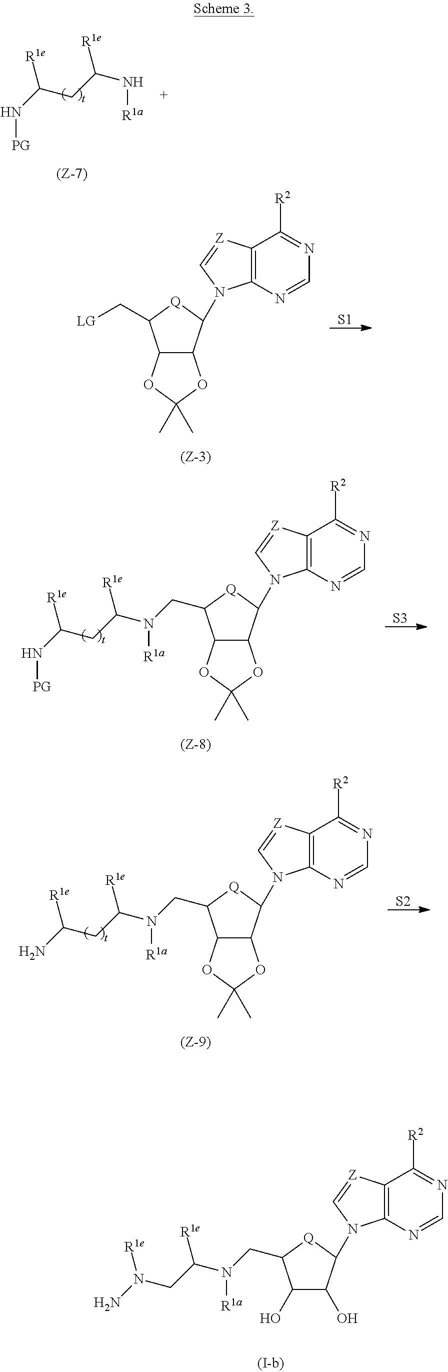

In certain embodiments, compounds described herein can prepared using methods shown in Scheme 4. Compounds of Formula (I-c) can be prepared through nucleophilic coupling of amines of formula (Z-10) under conditions S1 with a compound of formula (Z-3), wherein LG is a leaving group, followed by deprotection of the acetonide group of (Z-11) under conditions S2. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S2 comprise an organic or inorganic acid (e.g., camphorsulfonic acid or hydrochloric acid).

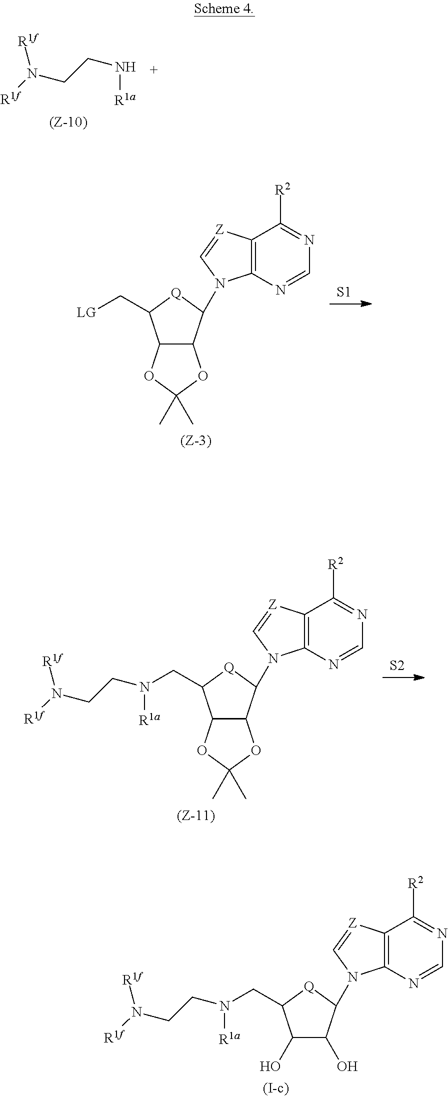

In certain embodiments, compounds described herein can prepared using methods shown in Scheme 5. Compounds of Formula (I-d) can be prepared through nucleophilic coupling of amines of formula (Z-12) under conditions S1 with a compound of formula (Z-3), wherein LG is a leaving group, followed by deprotection of the nitrogen protecting group of (Z-13) and acetonide group of (Z-14) under conditions S3 and S2, respectively. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S2 comprise an organic or inorganic acid (e.g., camphorsulfonic acid or hydrochloric acid).

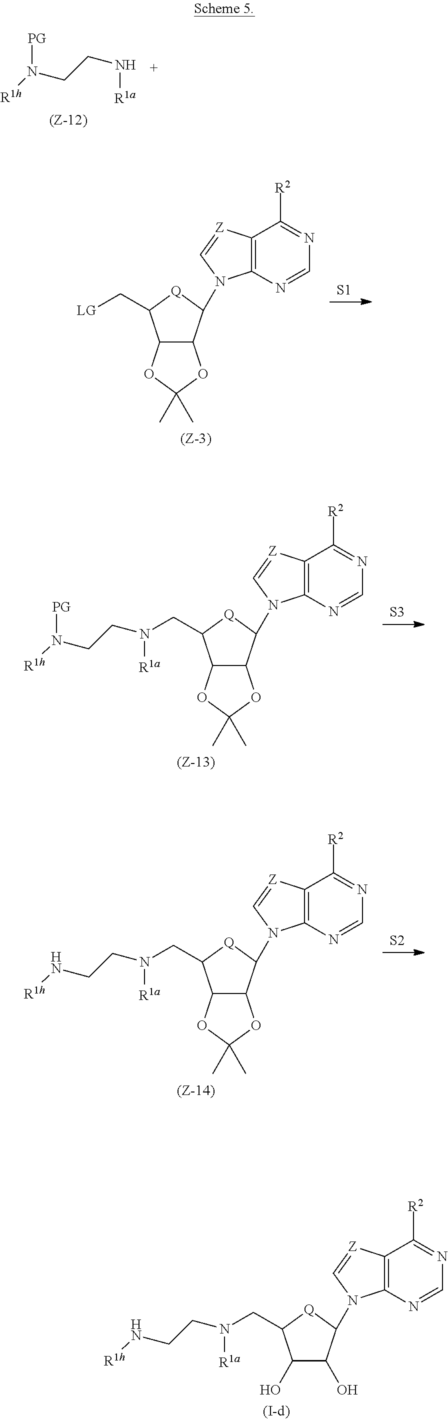

In certain embodiments, compounds described herein can prepared using methods shown in Scheme 6. Compounds of Formula (I-f) can be prepared through nucleophilic coupling of thiols of formula (Z-15) under conditions S1 with a compound of formula (Z-3), wherein LG is a leaving group, followed by deprotection of the acetonide group of (Z-16) under conditions S2. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S2 comprise an organic or inorganic acid (e.g., camphorsulfonic acid or hydrochloric acid).

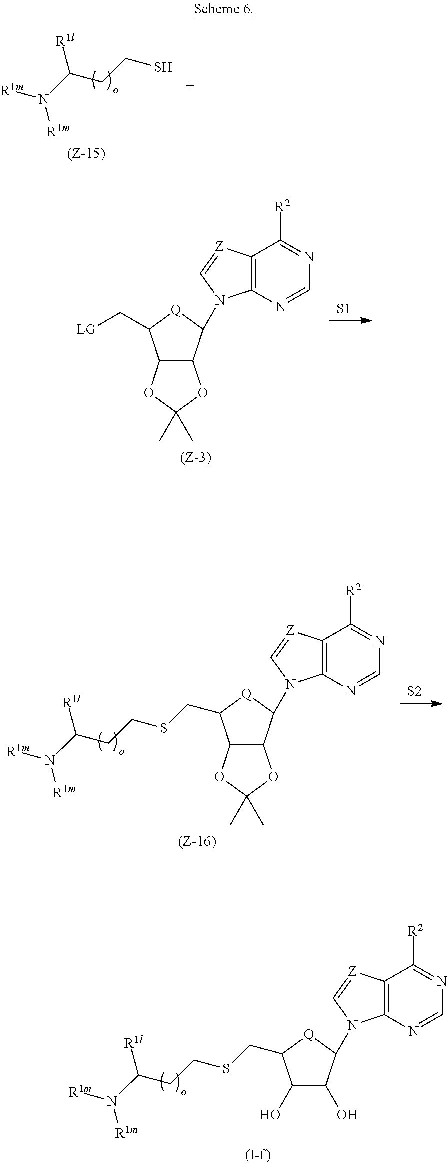

In certain embodiments, compounds described herein can prepared using methods shown in Scheme 7. Compounds of formula (Z-22) can be prepared through hydrolysis and nucleophilic coupling of thiolactones of formula (Z-18), under conditions S1 with a compound of formula (Z-3), wherein LG is a leaving group, followed by ester reduction of (Z-19), optional chiral HPLC separation under conditions S5, deprotection of the nitrogen protecting group of (Z-20) or (Z-21) under conditions S3, and deprotection of the acetonide group of (Z-20) or (Z-21) under conditions S2. In certain embodiments, conditions S1comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S2 comprise an organic or inorganic acid (e.g., camphorsulfonic acid or hydrochloric acid).

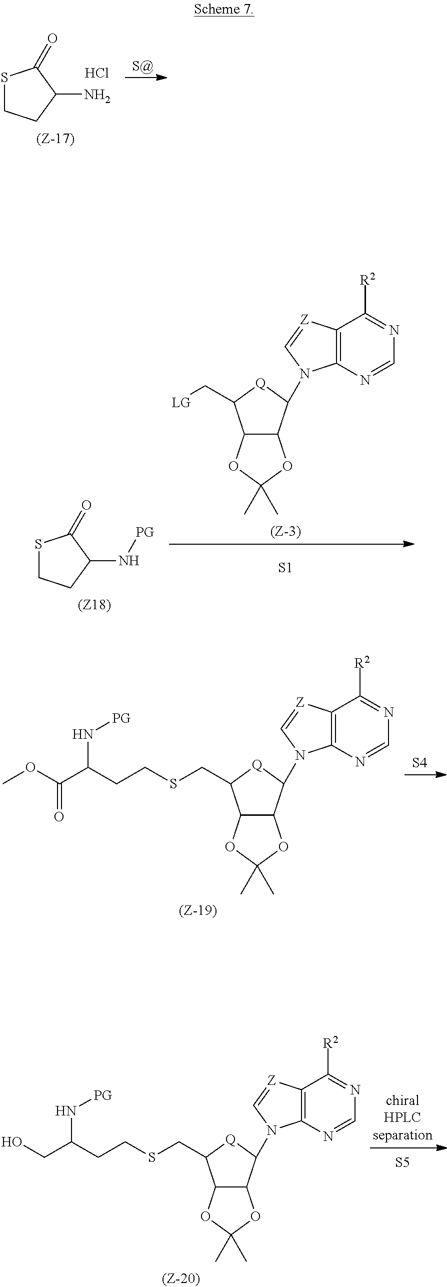

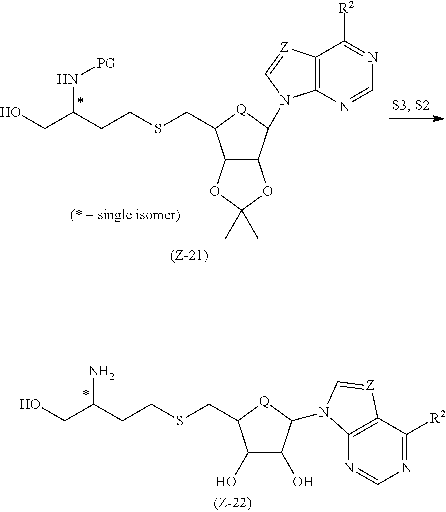

In certain embodiments, compounds described herein can prepared using methods shown in Scheme 8. Compounds of Formula (II) can be prepared through nucleophilic coupling of imidazoles of formula (Z-23) with benzylic electrophiles of formula (Z-24) under conditions S1, followed by electrophilic C-alkylation of intermediates of formula (Z-25) under conditions S6 and hydroxyl group displacement of intermediates of formula (Z-27) under conditions S8. Alternatively, the synthetic sequence can be reversed, wherein imidazoles of formula (Z-23) are first C-alkylated under conditions S6 prior to nucleophilic coupling with benzylic electrophiles of formula (Z-24) under conditions S1. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1comprise a carbonate or bicarbonate base (e.g., sodium bicarbonate or potassium carbonate). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S6 comprise addition of formaldehyde or an equivalent (e.g., paraformaldehyde). In certain embodiments, conditions S6 comprise prior N-protection of imidazoles (Z-23) or (Z-25) as a formyl hemiaminal. In certain embodiments, conditions S6 comprise an organic or inorganic acid (e.g., acetic acid or hydrochloric acid). In certain embodiments, conditions S6 comprise heating. In certain embodiments, conditions S8 comprise conversion of the free alcohol of (Z-27) into a leaving group prior to displacement with ammonia or an equivalent thereof (e.g., sodium azide followed by reduction). In certain embodiments, conditions S8 comprise prior oxidation of the free alcohol of (Z-27) into an aldehyde converted into compounds of Formula (II) under reductive amination conditions with ammonia or an equivalent thereof (e.g., hydroxylamine followed by reduction).

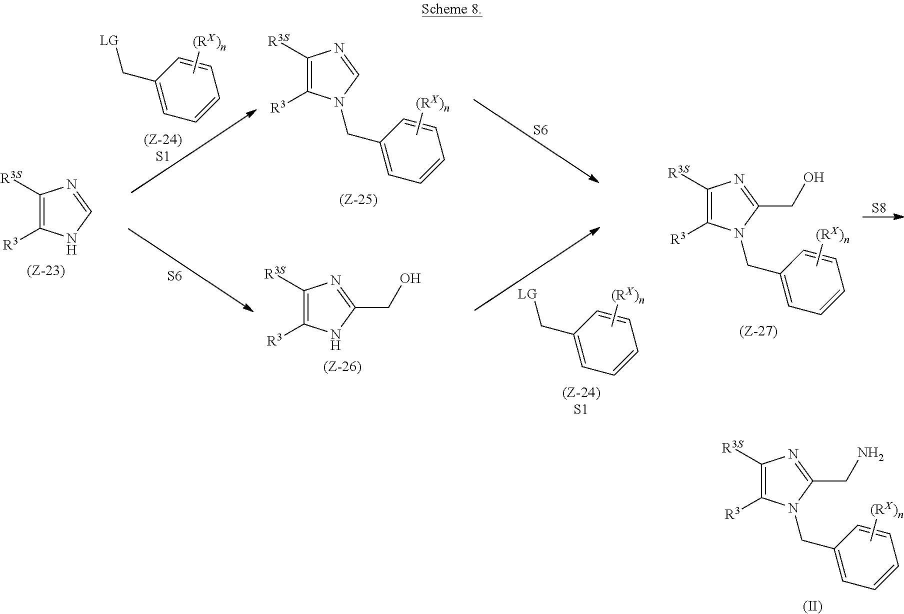

In certain embodiments, compounds described herein can prepared using methods shown in Scheme 9. Compounds of Formula (III) can be prepared through reductive amination of aldehydes of formula (Z-29) with piperidines of formula (Z-28) under conditions S9, followed by deprotection of intermediates of formula (Z-30) under conditions S3 and N-sulfonylation with compounds of formula (Z-31) under conditions S10. Alternatively, the synthetic sequence can be reversed, wherein piperidines of formula (Z-32) are first N-sulfonylated with compounds of formula (Z-31) under conditions S10 followed by deprotection of intermediates of formula (Z-33) under conditions S3 and reductive amination with aldehydes of formula (Z-29). In certain embodiments, conditions S9 comprise a borohydride reducing agent (e.g., sodium triacetoxyborohydride or sodium cyanoborohydride). In certain embodiments, the leaving group LG is a halogen (e.g., chlorine, bromine, or iodine). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S10 comprise a base (e.g., an organic base or an inorganic base). In certain embodiments, conditions S10 comprise an amine base (e.g., triethylamine or N,N-diisopropylethylamine). In certain embodiments, conditions S10 comprise an alkoxide base (e.g., sodium methoxide or potassium tert-butoxide). In certain embodiments, conditions S10 comprise a carbonate or bicarbonate base (e.g., sodium bicarbonate or potassium carbonate).

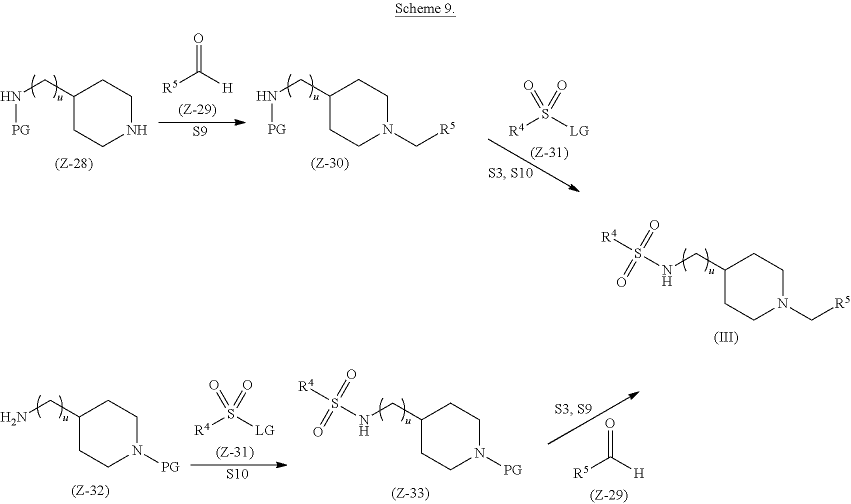

In certain embodiments, compounds described herein can prepared using methods shown in Scheme 10. Compounds of Formula (IV) can be prepared through nucleophilic coupling of amines of formula (Z-33) with benzylic or aryl electrophiles of formula (Z-34) under conditions S1, followed by deprotection of intermediates of formula (Z-35) under conditions S3 and N-sulfonylation of amines of formula (Z-36) with compounds of formula (Z-37) under conditions S10. In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, conditions S1 comprise a metal-catalyzed cross coupling reaction (e.g., a palladium catalyzed N-arylation reaction). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S10 comprise a base (e.g., an organic base or an inorganic base). In certain embodiments, conditions S10 comprise an amine base (e.g., triethylamine or N,N-diisopropylethylamine). In certain embodiments, conditions S10 comprise an alkoxide base (e.g., sodium methoxide or potassium tert-butoxide). In certain embodiments, conditions S10 comprise a carbonate or bicarbonate base (e.g., sodium bicarbonate or potassium carbonate).

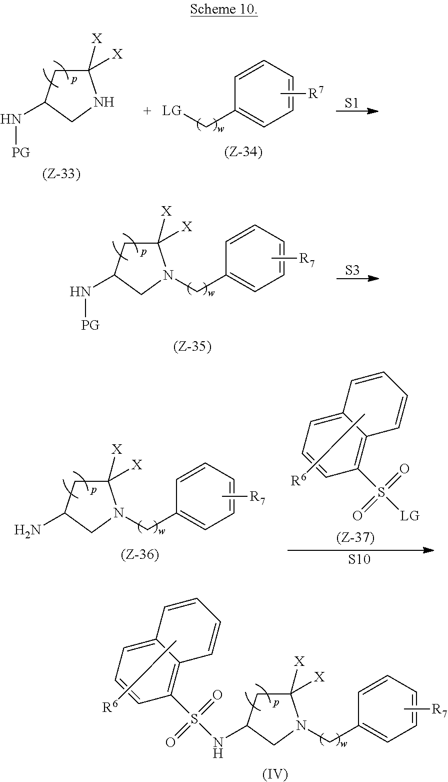

In certain embodiments, compounds described herein can prepared using methods shown in Scheme 11. Compounds of formula (Z-42) can be prepared through condensation of amines or anilines of formula (Z-38) with diacids of formula (Z-37), followed by Curtius rearrangement of acids of formula (Z-39) under conditions S12, deprotection of intermediates of formula (Z-40) under conditions S3, and N-sulfonylation of amines of formula (Z-41) with compounds of formula (Z-37) under conditions S10. In certain embodiments, conditions S11 comprise a base (e.g., an organic base or an inorganic base). In certain embodiments, conditions S11 comprise an amine base (e.g., triethylamine or N,N-diisopropylethylamine). In certain embodiments, conditions S11 comprise an alkoxide base (e.g., sodium methoxide or potassium tert-butoxide). In certain embodiments, conditions S11 comprise a carbonate or bicarbonate base (e.g., sodium bicarbonate or potassium carbonate). In certain embodiments, conditions S11 comprise an organic or inorganic acid (e.g., trifluoroacetic acid or hydrochloric acid). In certain embodiments, conditions S12 comprise use of an alkyl or aryl phosphoryl azide (e.g., diphenylphosphoryl azide). In certain embodiments, conditions S12 comprise prior conversion of the carboxylic acid of (Z-39) to an acid halide (e.g., acid chloride) and reaction with a metal azide (e.g., sodium azide). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S10 comprise a base (e.g., an organic base or an inorganic base). In certain embodiments, conditions S10 comprise an amine base (e.g., triethylamine or N,N-diisopropylethylamine). In certain embodiments, conditions S10 comprise an alkoxide base (e.g., sodium methoxide or potassium tert-butoxide). In certain embodiments, conditions S10 comprise a carbonate or bicarbonate base (e.g., sodium bicarbonate or potassium carbonate).

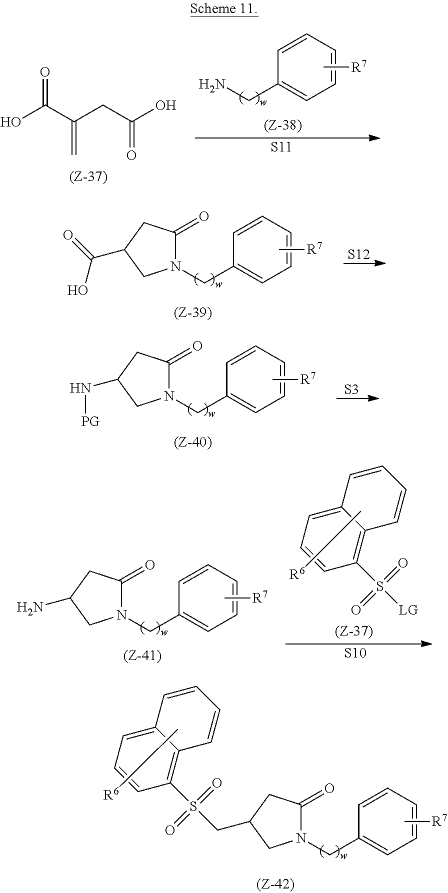

In certain embodiments, compounds described herein can prepared using methods shown in Scheme 12. Compounds of Formula (V) can be prepared through olefination of intermediate glyoxylates of formula (Z-73), wherein R^{P2} is substituted or unsubstituted alkyl or substituted or unsubstituted aryl, with phosphorous ylides of formula (Z-43a) or phosphonate esters of formula (Z-43b), wherein each R^{P1} is independently substituted or unsubstituted alkyl or substituted or unsubstituted aryl, under conditions S12, followed by condensation of unsaturated esters of formula (Z-44) with guanidines of formula (Z-45) under conditions S13. In certain embodiments, conditions S12 comprise a base (e.g., an organic base or an inorganic base). In certain embodiments, conditions S12 comprise an amine base (e.g., triethylamine or N,N-diisopropylethylamine). In certain embodiments, conditions S12 comprise an alkoxide base (e.g., sodium methoxide or potassium tert-butoxide). In certain embodiments, conditions S12 comprise a carbonate or bicarbonate base (e.g., sodium bicarbonate or potassium carbonate). In certain embodiments, conditions S13 comprise a base (e.g., an organic base or an inorganic base). In certain embodiments, conditions S13 comprise an amine base (e.g., triethylamine or N,N-diisopropylethylamine). In certain embodiments, conditions S13 comprise an alkoxide base (e.g., sodium methoxide or potassium tert-butoxide). In certain embodiments, conditions S13 comprise a carbonate or bicarbonate base (e.g., sodium bicarbonate or potassium carbonate). In certain embodiments, conditions S13 comprise heating. In certain embodiments, each R^{P1} is unsubstituted ethyl or unsubstituted phenyl. In certain embodiments, R^{P2} is unsubstituted methyl, ethyl, or tert-butyl.

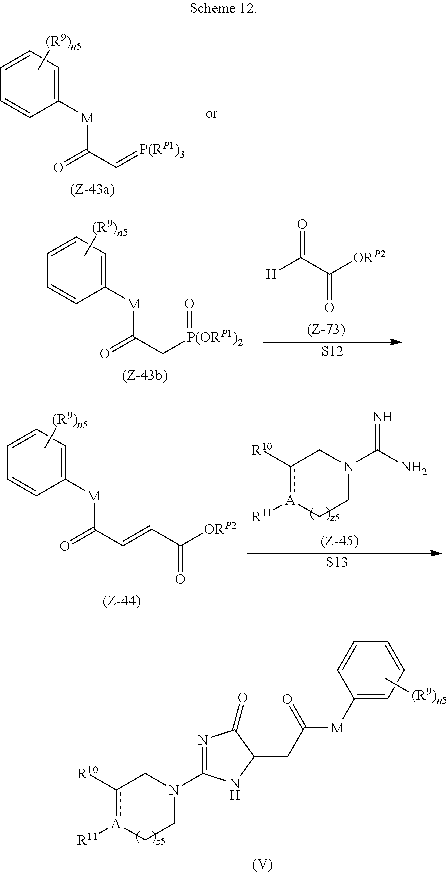

In certain embodiments, compounds described herein can prepared using methods shown in Scheme 13. Compounds of Formula (V) can be prepared through olefination of intermediate glyoxylates of formula (Z-73), wherein R^{P2} is substituted or unsubstituted alkyl or substituted or unsubstituted aryl, with phosphorous ylides of formula (Z-46a) or phosphonate esters of formula (Z-46b), wherein each R^{P1} is independently substituted or unsubstituted alkyl or substituted or unsubstituted aryl, under conditions S12, followed by condensation of unsaturated esters of formula (Z-47) with guanidines of formula (Z-45) under conditions S13, hydrolysis of esters of formula (Z-48) under conditions S14, and coupling of acids of formula (Z-49) under conditions S15. In certain embodiments, conditions S12 comprise a base (e.g., an organic base or an inorganic base). In certain embodiments, conditions S12 comprise an amine base (e.g., triethylamine or N,N-diisopropylethylamine). In certain embodiments, conditions S12 comprise an alkoxide base (e.g., sodium methoxide or potassium tert-butoxide). In certain embodiments, conditions S12 comprise a carbonate or bicarbonate base (e.g., sodium bicarbonate or potassium carbonate). In certain embodiments, conditions S13 comprise a base (e.g., an organic base or an inorganic base). In certain embodiments, conditions S13 comprise an amine base (e.g., triethylamine or N,N-diisopropylethylamine). In certain embodiments, conditions S13 comprise an alkoxide base (e.g., sodium methoxide or potassium tert-butoxide). In certain embodiments, conditions S13 comprise a carbonate or bicarbonate base (e.g., sodium bicarbonate or potassium carbonate). In certain embodiments, conditions S13 comprise heating. In certain embodiments, each R^{P1} is unsubstituted ethyl or unsubstituted phenyl. In certain embodiments, R^{P2} is unsubstituted methyl, ethyl, or tert-butyl. In certain embodiments, conditions S14 comprise an organic or inorganic hydroxide base (e.g., tetrabutylammonium or sodium hydroxide). In certain embodiments, conditions S15 comprise an amide coupling reagent (e.g., diisopropylcarbodiimide, HATU, or propylphosphonic anhydride). In certain embodiments, conditions S15 comprise prior conversion of acid (Z-49) to an intermediate aldehyde and reaction with an aryl metal species (e.g., an aryl Grignard or lithium species) followed by oxidation.

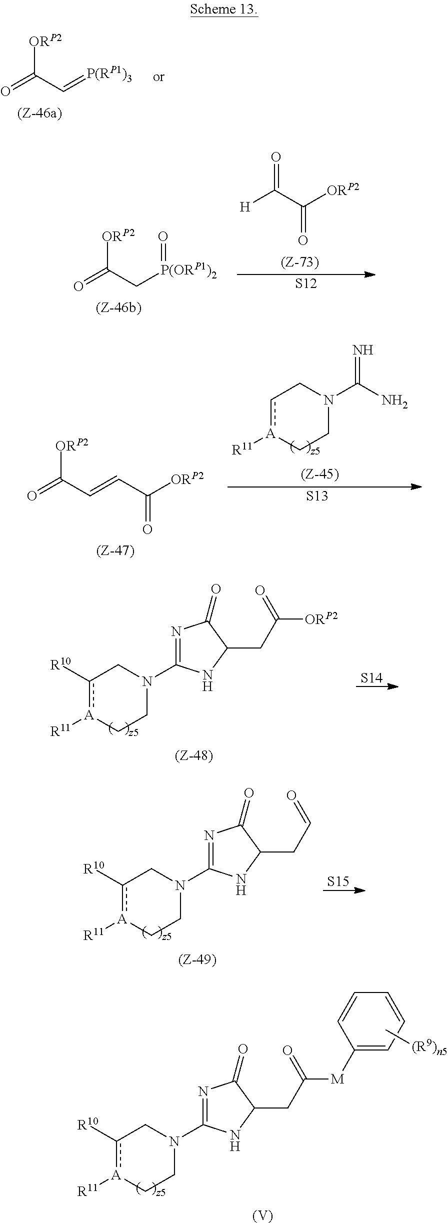

In certain embodiments, compounds described herein can prepared using methods shown in Scheme 14. Compounds of Formula (VI) can be prepared through N-sulfonylation of amines of formula (Z-50) with compounds of formula (Z-37) under conditions S10 followed by deprotection of intermediates of formula (Z-51) under conditions S3. In certain embodiments, conditions S10 comprise a base (e.g., an organic base or an inorganic base). In certain embodiments, conditions S10 comprise an amine base (e.g., triethylamine or N,N-diisopropylethylamine). In certain embodiments, conditions S10 comprise an alkoxide base (e.g., sodium methoxide or potassium tert-butoxide). In certain embodiments, conditions S10 comprise a carbonate or bicarbonate base (e.g., sodium bicarbonate or potassium carbonate). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid).

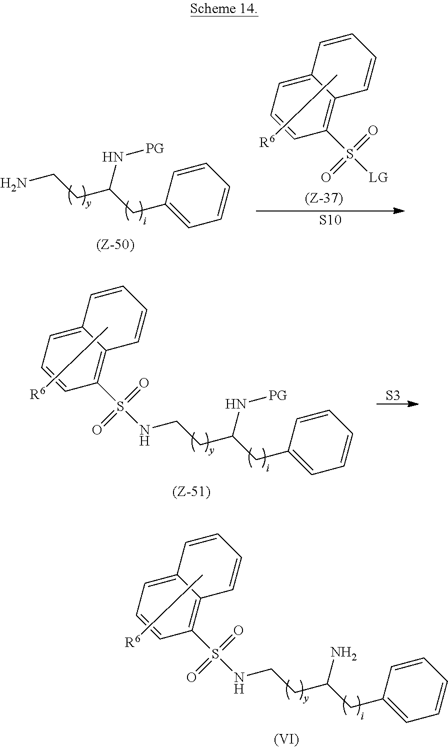

In certain embodiments, compounds described herein can prepared using methods shown in Scheme 15. Compounds of formula (Z-57) can be prepared through N-acylation of piperidines of formula (Z-52) with compounds of formula (Z-51) under conditions S15, followed by deprotection of intermediates of formula (Z-53) under conditions S3, N-arylation of amines of formula (Z-54) with compounds of formula (Z-55) under conditions S16, and hydrolysis of compounds of formula (Z-56) under conditions S14. In certain embodiments, the leaving group LG of compound (Z-51) is a halogen (e.g., chlorine, bromine, or iodine) and conditions S15 comprise a base (e.g., an organic base or an inorganic base). In certain embodiments, the leaving group LG of compound (Z-51) is a —OH and conditions S15 comprise an amide coupling reagent (e.g., diisopropylcarbodiimide, HATU, or propylphosphonic anhydride). In certain embodiments, the nitrogen protecting group PG is a carbamate protecting group (e.g., Boc, Cbz, or Fmoc). In certain embodiments, conditions S3 comprise an organic or inorganic acid (e.g., trifluoroacetic acid or hydrochloric acid). In certain embodiments, conditions S16 comprise a base (e.g., an organic base or an inorganic base). In certain embodiments, conditions S16 comprise an amine base (e.g., triethylamine or N,N-diisopropylethylamine). In certain embodiments, conditions S16 comprise an alkoxide base (e.g., sodium methoxide or potassium tert-butoxide). In certain embodiments, conditions S16 comprise a carbonate or bicarbonate base (e.g., sodium bicarbonate or potassium carbonate). In certain embodiments, conditions S16 comprise a metal-catalyzed cross coupling reaction (e.g., a palladium catalyzed N-arylation reaction). In certain embodiments, R^{P2} is unsubstituted methyl, ethyl, or tert-butyl. In certain embodiments, conditions S14 comprise an organic or inorganic hydroxide base (e.g., tetrabutylammonium or sodium hydroxide).

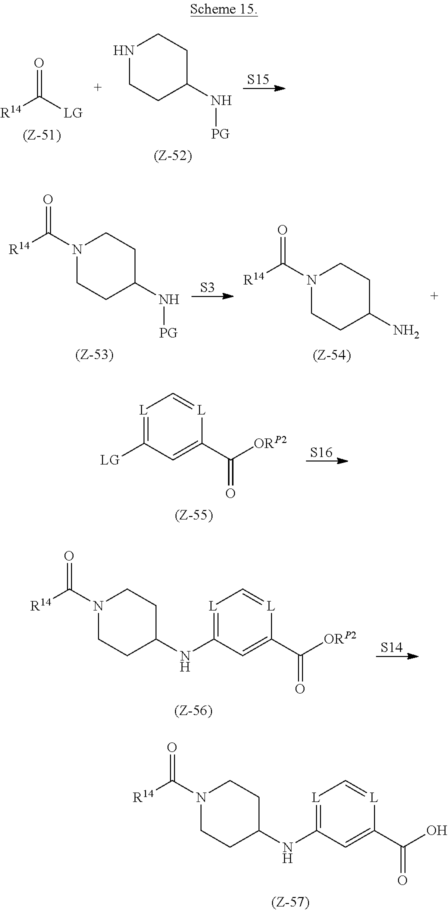

In certain embodiments, compounds described herein can prepared using methods shown in Scheme 16. Compounds of Formula (VII) can be prepared through nucleophilic coupling of amines of formula (Z-58) with electrophiles of formula (Z-59) followed by opening of epoxides of formula (Z-60) under conditions S1 with ammonia or an equivalent, coupling of amines of formula (Z-61) with acids of formula (Z-57) under conditions S15, and deprotection of intermediates of formula (Z-62) under conditions S3. In certain embodiments, the nitrogen protecting group PG is a carbamate protecting group (e.g., Boc, Cbz, or Fmoc). In certain embodiments, the leaving group LG is a halogen (e.g., chlorine, bromine, or iodine). In certain embodiments, the leaving group LG is a sulfonate ester (e.g., tosylate, mesylate, or triflate). In certain embodiments, conditions S1 comprise a base (e.g., an organic base or an inorganic base). In certain embodiments, conditions S1 comprise an amine base (e.g., triethylamine or N,N-diisopropylethylamine). In certain embodiments, conditions S1 comprise an alkoxide base (e.g., sodium methoxide or potassium tert-butoxide). In certain embodiments, conditions S1 comprise a carbonate or bicarbonate base (e.g., sodium bicarbonate or potassium carbonate). In certain embodiments, conditions S15 comprise an amide coupling reagent (e.g., diisopropylcarbodiimide, HATU, or propylphosphonic anhydride). In certain embodiments, conditions S3 comprise an organic or inorganic acid (e.g., trifluoroacetic acid or hydrochloric acid).

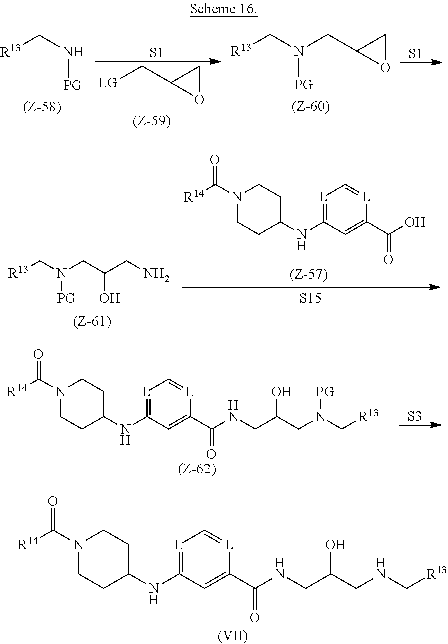

In certain embodiments, compounds described herein can prepared using methods shown in Scheme 17. Similarly to compounds of Formula (VII), compounds of Formula (VIII) can be prepared through opening of epoxides of formula (Z-60) with amines of formula (Z-64) under conditions S1. Compounds of formula (Z-63) can be prepared through routes analogous to those described herein (see Scheme 15 and 16).

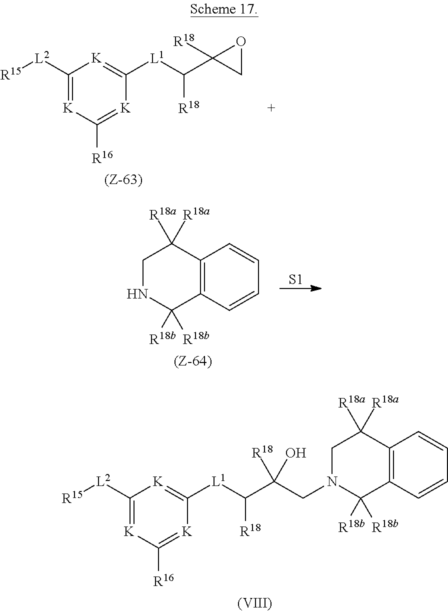

In certain embodiments, compounds described herein can prepared using methods shown in Scheme 18. Similarly to compounds of Formula (VII), compounds of Formula (IX) can be prepared through nucleophilic coupling of amine (Z-65) with electrophiles of formula (Z-59) followed by opening of epoxides of formula (Z-66) under conditions S1 with ammonia or an equivalent, and coupling of amines of formula (Z-67) with acids of formula (Z-68) under conditions S15.

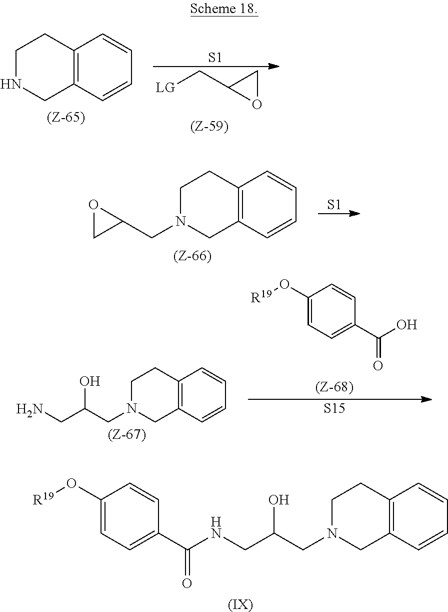

In certain embodiments, compounds described herein can prepared using methods shown in Scheme 19. Similarly to compounds of Formula (VII), compounds of Formula (X) can be prepared through nucleophilic coupling of amine (Z-69) with electrophiles of formula (Z-59) followed by opening of epoxides of formula (Z-70) under conditions S1 with ammonia or an equivalent, and coupling of amines of formula (Z-71) with acids of formula (Z-72) under conditions S15. Compounds of formula (Z-72) can be prepared through routes analagous to those described herein (see Scheme 15 and 16).

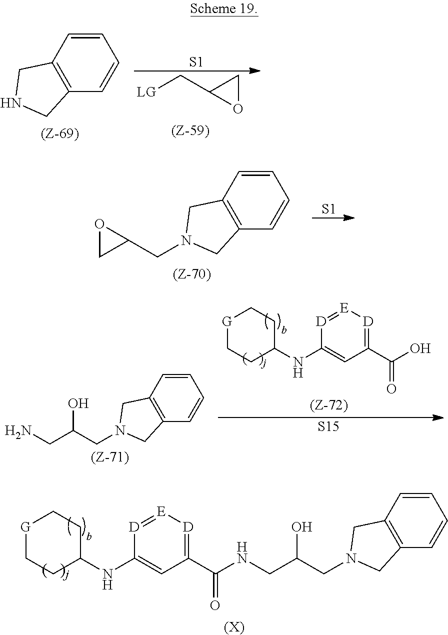

### Examples

In order that the invention described herein may be more fully understood, the following examples are set forth. It should be understood that these examples are for illustrative purposes only and are not to be construed as limiting this invention in any manner.

### A. Synthetic Methods

### Compound 91-1 & 92-1: (2S, 3S, 4R, 5R)-2-((((S)-3-amino-4-hydroxybutyl)thio)methyl)-5-(6-amino-9H-purin-9-yl)tetrahydrofuran-3,4-diol and (2S, 3S, 4R, 5R)-2-((((R)-3-amino-4-hydroxybutyl)thio)methyl)-5-(6-amino-9H-purin-9-yl)tetrahydrofuran-3,4-diol

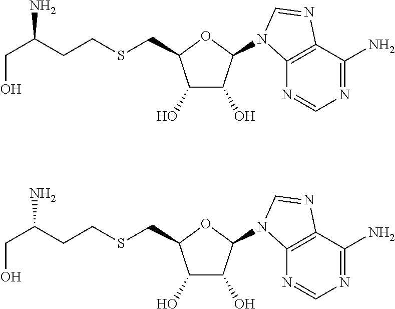 [SMILES: Nc1ncnc2c1ncn2[C@@H]1O[C@H](CSCC[C@H](N)CO)[C@@H](O)[C@H]1O ; Nc1ncnc2c1ncn2[C@@H]1O[C@H](CSCC[C@@H](N)CO)[C@@H](O)[C@H]1O]

### Step 1: tert-butyl (2-oxotetrahydrothiophen-3-yl)carbamate

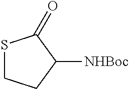 [SMILES: CC(C)(C)OC(=O)NC1CCSC1=O]

To a solution of 3-aminodihydrothiophen-2(3H)-one hydrochloride (2 g, 13.0 mmol) and triethylamine (4 mL, 28.64 mmol) in chloroform (20 mL) was added di-tert-butyl dicarbonate (2.8 g, 13.0 mmol). The mixture was stirred at 25° C. for 2 h. The mixture was washed with water (15 mL×2), 2% hydrochloric acid solution (20 mL), and brine (20 mL). The organic layers were dried over sodium sulfate, filtered and concentrated to afford tert-butyl (2-oxotetrahydrothiophen-3-yl)carbamate (2.6 g, 93% yield) as a pale yellow solid, which was used in next step without further purification. 1H NMR (500 MHz, CDCl₃): δ 5.01 (brs, 1H), 4.29-4.27 (m, 1H), 3.35-3.21 (m, 2H), 2.87-2.85 (m, 1H), 2.00-1.96 (m, 1H), 1.45 (s, 9H) ppm; ESI-MS (m/z): 162.1[M−56+1]⁺.

### Step 2: methyl S-(((3aS, 4S, 6R, 6aR)-6-(6-amino-9H-purin-9-yl)-2,2-dimethyl tetrahydrofuro[3,4-d][1,3]dioxol-4-yl)methyl)-N-(tert-butoxycarbonyl)homocysteinate

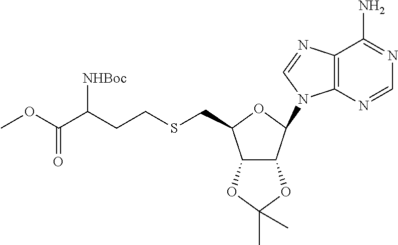 [SMILES: COC(=O)C(CCSC[C@H]1O[C@@H](n2cnc3c(N)ncnc32)[C@@H]2OC(C)(C)O[C@@H]21)NC(=O)OC(C)(C)C]

A mixture of tert-butyl (2-oxotetrahydrothiophen-3-yl)carbamate (2.2 g, 10.12 mmol) and sodium methoxide (0.55 g, 10.12 mmol) in methanol (22 mL) was stirred at ambient temperature for 5 min. 9-((3aR, 4R, 6S, 6aS)-6-(chloromethyl)-2,2-dimethyltetrahydrofuro[3,4-d][1,3]dioxol-4-yl)-9H-purin-6-amine (2.2 g, 6.71 mmol) was added and the mixture was stirred at reflux temperature for 3 h. The mixture was concentrated and the residue was purified by prep-HPLC to afford the desired product (2.5 g, 69% yield) as a white solid. 1H NMR (500 MHz, CDCl₃): δ 8.36 (s, 1H), 7.95 (s, 1H), 6.08 (d, J=3.5 Hz, 1H), 5.78 (brs, 2H), 5.52 (d, J=5.5 Hz, 1H), 5.19-5.15 (m, 1H), 5.08-5.06 (m, 1H), 4.42-4.36 (m, 2H), 3.74-3.73 (m, 3H), 2.90-2.75 (m, 2H), 2.56 (t, J=7.0 Hz, 2H), 2.10-2.00 (m, 1H), 1.90-1.80 (m, 1H), 1.62 (s, 3H), 1.44 (s, 9H), 1.41 (s, 3H) ppm; ESI-MS (m/z): 539.1 [M+1]⁺.

### Step 3: tert-butyl ((S)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate and tert-butyl ((R)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate

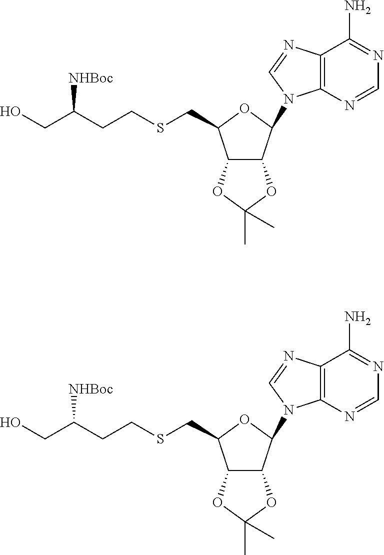 [SMILES: CC(C)(C)OC(=O)N[C@H](CO)CCSC[C@H]1O[C@@H](n2cnc3c(N)ncnc32)[C@@H]2OC(C)(C)O[C@@H]21 ; CC(C)(C)OC(=O)N[C@@H](CO)CCSC[C@H]1O[C@@H](n2cnc3c(N)ncnc32)[C@@H]2OC(C)(C)O[C@@H]21]

Under nitrogen atmosphere, to a stirred suspension of lithium aluminum hydride (353 mg, 9.28 mmol) in dry tetrahydrofuran (33 mL) at ambient temperature was added a solution of methyl S-(((3aS, 4S, 6R, 6aR)-6-(6-amino-9H-purin-9-yl)-2,2-dimethyltetrahydrofuro[3,4-d][1,3]dioxol-4-yl)methyl)-N-(tert-butoxycarbonyl)homocysteinate (2.5 g, 4.64 mmol) in dry tetrahydrofuran (11 mL) drop wise. The mixture was stirred at ambient temperature for 3 h. The mixture was treated with sat. ammonium chloride and extracted with ethyl acetate (30 mL×4). The combined organic layers were washed with brine (20 mL). The organic phase was dried over sodium sulfate, filtered, and concentrated. The residue was purified by Combi-Flash (40 g silica gel, start ethyl acetate:dichloromethane:methanol=10:10:0 to 10:10:2 by gradient, 50 mL/min, 40 min, 2.0 L total solvent volume) to afford a mixture of diastereomers (1.7 g, 72% yield). This mixture (1.2 g) was separated by chiral HPLC to afford tert-butyl ((S)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate and tert-butyl ((R)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate (530 mg, 44% yield). ¹H NMR (500 MHz, CD₃OD): δ 8.32 (s, 1H), 8.24 (s, 1H), 6.19 (d, J=2.5 Hz, 1H), 5.24-5.23 (m, 1H), 5.10-5.07 (m, 1H), 4.38-4.34 (m, 1H), 3.65-3.60 (m, 1H), 3.48-3.40 (m, 2H), 2.86-2.77 (m, 2H), 2.65-2.50 (m, 2H), 1.82-1.72 (m, 1H), 1.62-1.55 (m, 4H), 1.43 (s, 9H), 1.40 (s, 3H) ppm; ESI-MS (m/z): 511.3 [M+1]⁺. ¹H NMR (500 MHz, CD₃OD): δ 8.32 (s, 1H), 8.23 (s, 1H), 6.19 (d, J=2.5 Hz, 1H), 5.54-5.51 (m, 1H), 5.10-5.07 (m, 1H), 4.38-4.34 (m, 1H), 3.62-3.59 (m, 1H), 3.48-3.40 (m, 2H), 2.88-2.77 (m, 2H), 2.65-2.50 (m, 2H), 1.82-1.72 (m, 1H), 1.62-1.55 (m, 4H), 1.42 (s, 9H), 1.40 (s, 3H) ppm; ESI-MS (m/z): 511.2 [M+1]⁺.

### Step 4: (2S, 3S, 4R, 5R)-2-((((S)-3-amino-4-hydroxybutyl)thio)methyl)-5-(6-amino-9H-purin-9-yl)tetrahydrofuran-3,4-diol

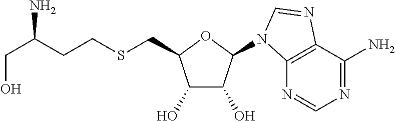 [SMILES: Nc1ncnc2c1ncn2[C@@H]1O[C@H](CSCC[C@H](N)CO)[C@@H](O)[C@H]1O]

A solution of tert-butyl ((S)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate (530 mg, 1.05 mmol) in 6N hydrochloric acid (3 mL) was stirred at ambient temperature for 30 min. The mixture was adjusted to pH=9-10 with ammonium hydroxide. The mixture was purified by prep-HPLC to afford the desired title compound (242 mg, 63% yield) as a white solid. 1H NMR (500 MHz, CD₃OD): δ 8.32 (s, 1H), 8.22 (s, 1H), 6.01 (d, J=4.5 Hz, 1H), 4.81 (t, J=5.5 Hz, 1H), 4.36 (t, J=4.5 Hz, 1H), 4.22 (q, J=5.5 Hz, 1H), 3.58 (dd, J=11.5 and 4.0 Hz, 1H), 3.39 (dd, J=10.5 and 6.5 Hz, 1H), 3.08-2.92 (m, 3H), 2.68 (t, J=7.0 Hz, 2H), 1.85-1.76 (m, 1H), 1.70-1.62 (m, 1H) ppm; ESI-MS (m/z): 371.1 [M+1]⁺. Step 5: (2S, 3S, 4R, 5R)-2-((((R)-3-amino-4-hydroxybutyl)thio)methyl)-5-(6-amino-9H-purin-9-yl)tetrahydrofuran-3,4-diol

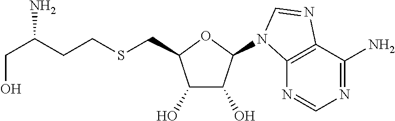 [SMILES: Nc1ncnc2c1ncn2[C@@H]1O[C@H](CSCC[C@@H](N)CO)[C@@H](O)[C@H]1O]

A solution of tert-butyl ((R)-4-((((3aS, 4S, 6R, 6aR)-6-(6-amino-9H-purin-9-yl)-2,2-dimethyltetrahydrofuro[3,4-d][1,3]dioxol-4-yl)methyl)thio)-1-hydroxybutan-2-yl)carbamate (440 mg, 0.86 mmol) in 6N hydrochloric acid (3 mL) was stirred at ambient temperature for 30 min. The mixture was adjusted to pH=9-10 with ammonium hydroxide. The mixture was purified by prep-HPLC to afford the desired title compound (202 mg, 63% yield) as a white solid. 1H NMR (500 MHz, CD₃OD): δ 8.32 (s, 1H), 8.22 (s, 1H), 6.01 (d, J=5.0 Hz, 1H), 4.81 (t, J=5.0 Hz, 1H), 4.36 (t, J=5.0 Hz, 1H), 4.22 (q, J=5.0 Hz, 1H), 3.65-3.55 (m, 1H), 3.45-3.38 (m, 1H), 3.15-2.92 (m, 3H), 2.73-2.63 (m, 2H), 1.88-1.78 (m, 1H), 1.72-1.62 (m, 1H) ppm; ESI-MS (m/z): 371.1 [M+1]⁺.

### Compound 1-8: (R)-2-((1-acetylpiperidin-4-yl)amino)-N-(3-(benzylamino)-2-hydroxypropyl)isonicotinamide

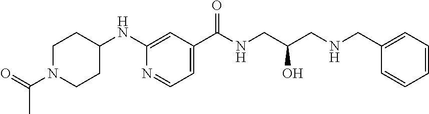 [SMILES: CC(=O)N1CCC(Nc2cc(C(=O)NC[C@H](O)CNCc3ccccc3)ccn2)CC1]

### Step 1: tert-butyl benzylcarbamate

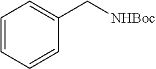 [SMILES: CC(C)(C)OC(=O)NCc1ccccc1]

To a solution of phenylmethanamine (10 g, 0.093 mol) in acetonitrile (150 mL) was added di-tert-butyl dicarbonate (24 g, 0.11 mol), the mixture was stirred at 30° C. for 18 h. Water (50 mL) was added, the mixture was extracted with ethyl acetate (2×150 mL), the organic layer was dried over sodium sulfate, filtered, concentrated to give the title compound (19.2 g, 78%).

### Step 2: (R)-tert-butyl benzyl(oxiran-2-ylmethyl)carbamate

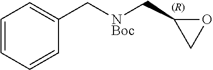

To a solution of tert-butyl benzylcarbamate (10 g, 0.048 mol) in dimethylformamide (250 mL) was added sodium hydride (2.3 g, 0.096 mol) at 0° C., the mixture was stirred at 0° C. for 1 h, (S)-oxiran-2-ylmethyl 3-nitrobenzenesulfonate (18.6 g, 0.072 mol) was added, the mixture was stirred at 30° C. for 2 h. The reaction was quenched with sat. ammonium chloride (100 mL), extracted with ethyl acetate (2×200 mL), the combined organic layer was washed with brine (100 mL), dried over sodium sulfate, filtered, and concentrated to give the title compound (12.6 g, 71%).

### Step 3: (S)-tert-butyl(3-amino-2-hydroxypropyl)(benzyl)carbamate

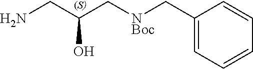

To a solution of (R)-tert-butyl benzyl(oxiran-2-ylmethyl)carbamate (15 g, 0.057 mol) in ethanol (200 mL) was added ammonium hydroxide (500 mL), the mixture was stirred at 30° C. for 16 h. The reaction mixture was quenched with sat. sodium hydroxide (500 mL), extracted with ethyl acetate (3×500 mL), the combined organic layer was concentrated and purified by silica gel chromatography (dichloromethane:methanol=10:1) to give the title compound (8 g, 90%).

### Step 4: (S)-tert-butyl(3-(2-((1-acetylpiperidin-4-yl)amino)isonicotinamido)-2-hydroxypropyl)(benzyl)carbamate

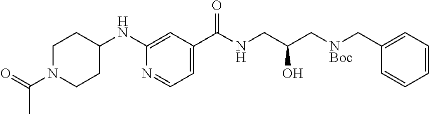 [SMILES: CC(=O)N1CCC(Nc2cc(C(=O)NC[C@H](O)CN(Cc3ccccc3)C(=O)OC(C)(C)C)ccn2)CC1]

To a solution of 2-((1-acetylpiperidin-4-yl)amino)isonicotinic acid (600 mg, 2.28 mmol) in dichloromethane (20 mL) was added (S)-tert-butyl(3-amino-2-hydroxypropyl)(benzyl)carbamate (957 mg, 3.42 mmol) and HATU (1.3 g, 3.42 mmol). The mixture was stirred at 30° C. for 16 h. Water (30 mL) was added, the mixture was extracted with dichloromethane (3×20 mL), and the organic layer was concentrated and used for next step directly without purification.

### Step 5: (R)-2-((1-acetylpiperidin-4-yl)amino)-N-(3-(benzylamino)-2-hydroxypropyl)isonicotinamide

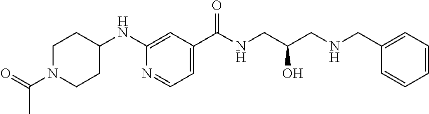 [SMILES: CC(=O)N1CCC(Nc2cc(C(=O)NC[C@H](O)CNCc3ccccc3)ccn2)CC1]

To a solution of (S)-tert-butyl(3-(2-((1-acetylpiperidin-4-yl)amino)isonicotinamido)-2-hydroxypropyl)(benzyl)carbamate (800 mg, 1.6 mmol) in dichloromethane (10 mL) was added TFA (2 mL) at 0° C., the mixture was stirred at 30° C. for 16 h, the mixture was concentrated and purified by preparative HPLC to give the title compound (120.8 mg, 20%). ¹H NMR (400 MHz, CD₃OD) ppm 1.36-1.55 (m, 2H) 1.99-2.17 (m, 5H) 2.66 (dd, J=12.30, 8.03 Hz, 1H) 2.76 (dd, J=12.30, 4.02 Hz, 1H) 2.87-2.98 (m, 1H) 3.26-3.32 (m, 1H) 3.44 (d, J=5.77 Hz, 2H) 3.77-4.07 (m, 5H) 4.44 (d, J=13.30 Hz, 1H) 6.78-6.91 (m, 2H) 7.23-7.48 (m, 5H) 8.04 (d, J=5.27 Hz, 1H); ESI-MS (m/z): 426.3 [M+1]+.

### Compound 2-2: N-(2-(2-(aminomethyl)-1-benzyl-1H-imidazol-4-yl)ethyl)benzamide

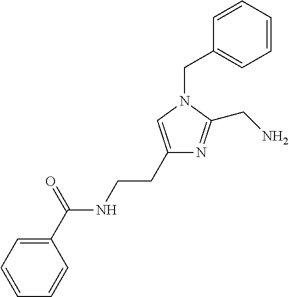 [SMILES: NCc1nc(CCNC(=O)c2ccccc2)cn1Cc1ccccc1]

### Step 1: 2-(2-(1H-imidazol-4-yl)ethyl)isoindoline-1,3-dione

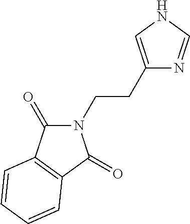 [SMILES: O=C1c2ccccc2C(=O)N1CCc1c[nH]cn1]

A mixture of 2-(1H-imidazol-4-yl)ethan-1-amine dihydrochloride (18.4 g, 0.1 mol), isobenzofuran-1,3-dione (15 g, 0.1 mol) and triethylamine (20 g, 0.2 mol) in 125 mL acetic acid was refluxed with stirring for 2 h. After cooling to ambient temperature, the mixture was evaporated under reduced pressure, the residue was diluted with 150 mL water and neutralized with sodium carbonate (pH=7-8). The precipitate was filtered off and washed with water and diethyl ether to give the title product as a white solid (22.1 g, 91.6% yield).

### Step 2: 1,3-bis(acetoxymethyl)-4-(2-(1,3-dioxoisoindolin-2-yl)ethyl)-1H-imidazol-3-ium acetate

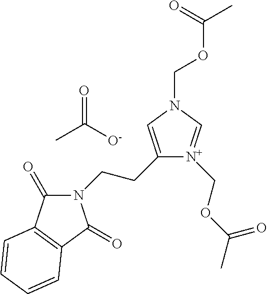

A mixture of 2-(2-(1H-imidazol-4-yl)ethyl)isoindoline-1,3-dione (14.5 g, 0.06 mol) and paraformaldehyde (5.4 g, 0.18 mol) in 80 mL acetic acid was stirred at 80° C. for 0.5 h. Acetic anhydride (22 g, 0.216 mol) was then added and the mixture was stirred for 3 h at 80° C. The mixture was then evaporated under reduced pressure to give the crude product as a mixture of title compound (˜25 g) as a pale yellow oil.

### Step 3: (4-(2-(1,3-dioxoisoindolin-2-yl)ethyl)-1H-imidazole-1,2-diyl)bis(methylene) diacetate and (5-(2-(1,3-dioxoisoindolin-2-yl)ethyl)-1H-imidazole-1,2-diyl)bis(methylene) diacetate

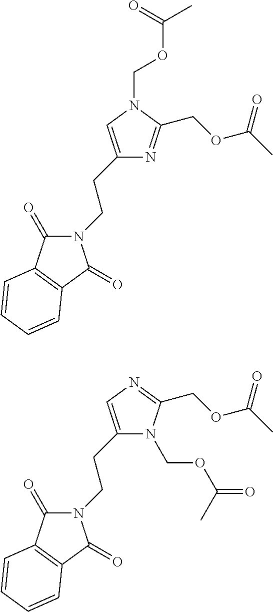 [SMILES: CC(=O)OCc1nc(CCN2C(=O)c3ccccc3C2=O)cn1COC(C)=O ; CC(=O)OCc1ncc(CCN2C(=O)c3ccccc3C2=O)n1COC(C)=O]

A mixture of 1,3-bis(acetoxymethyl)-4-(2-(1,3-dioxoisoindolin-2-yl)ethyl)-1H-imidazol-3-ium acetate (25 g, 0.056 mol) and 19 g acetic acid was heated with stirring for 7 h at 150° C. Then the reaction mixture was diluted with 100 mL water and 150 mL chloroform, the organic layer was separated, washed with aqueous sodium carbonate, dried with sodium sulfate and evaporated under reduced pressure. The residue was purified by flash-chromatography (silica gel, chloroform:ethanol=50-20:1) to give the mixture of products as a yellow oil (mixture of isomers ˜60:40, 10.2 g, 47.2% yield).

### Step 4: 1,2-bis(acetoxymethyl)-3-benzyl-5-(2-(1,3-dioxoisoindolin-2-yl)ethyl)-1H-imidazol-3-ium bromide

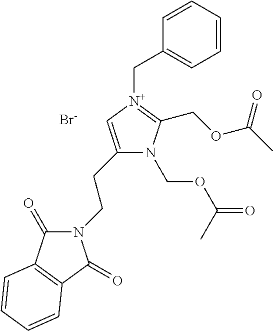 [SMILES: CC(=O)OCc1n(COC(C)=O)c(CCN2C(=O)c3ccccc3C2=O)c[n+]1Cc1ccccc1 ; [Br-]]

A solution containing (4-(2-(1,3-dioxoisoindolin-2-yl)ethyl)-1H-imidazole-1,2-diyl)bis(methylene) diacetate and (5-(2-(1,3-dioxoisoindolin-2-yl)ethyl)-1H-imidazole-1,2-diyl)bis(methylene) diacetate (mixture of isomers, 10.0 g, 0.026 mol) and benzyl bromide (4.4 g, 0.026 mol) in 50 mL chloroform was refluxed with stirring for 4 h. After cooling to ambient temperature the mixture was purified and separated by flash-chromatography (silica gel, chloroform:ethanol=10-5:1) to give the desired product as a yellow oil (3 g).

### Step 5: (4-(2-aminoethyl)-1-benzyl-1H-imidazol-2-yl)methanol

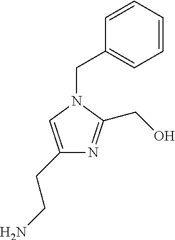 [SMILES: NCCc1cn(Cc2ccccc2)c(CO)n1]

A solution of 1,2-bis(acetoxymethyl)-3-benzyl-5-(2-(1,3-dioxoisoindolin-2-yl)ethyl)-1H-imidazol-3-ium bromide (3.0 g, 0.0054 mol) and hydrazine (1.4 g, 0.02 mol) in 50 mL ethanol was stirred at ambient temperature for 21 h. The mixture was diluted with 150 mL chloroform and the precipitate was filtered off. The filtrate was evaporated under reduced pressure to give a mixture of the desired product and MeCON₂H₃(1.1 g) as a yellow oil.

### Step 6: (4-(2-benzamidoethyl)-1-benzyl-1H-imidazol-2-yl)methyl benzoate

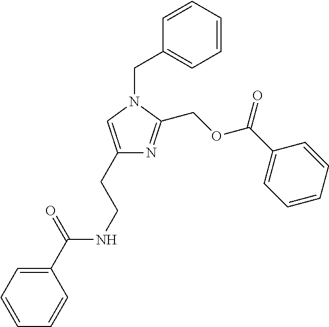 [SMILES: O=C(NCCc1cn(Cc2ccccc2)c(COC(=O)c2ccccc2)n1)c1ccccc1]

A mixture of benzoic acid (2.3 g, 0.019 mol) and carbonyldiimidazole (3.1 g, 0.019 mol) in 20 mL chloroform was refluxed for 1 h. After cooling to ambient temperature, (4-(2-aminoethyl)-1-benzyl-1H-imidazol-2-yl)methanol (1.1 g, 0.0047 mol) was added to the solution. The reaction mixture was stirred for 15 h at ambient temperature and diluted with 30 mL water. The organic layer was separated, washed with aqueous sodium carbonate, dried with sodium sulfate, and evaporated under reduced pressure. The residue was then purified by flash-chromatography (silica gel, chloroform:ethanol=10:1) to give the crude product (2.3 g) as a yellow oil.

### Step 7: N-(2-(1-benzyl-2-(hydroxymethyl)-1H-imidazol-4-yl)ethyl)benzamide

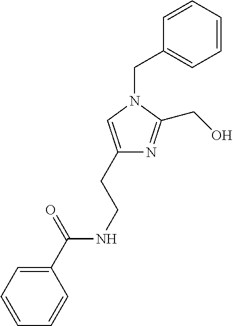 [SMILES: O=C(NCCc1cn(Cc2ccccc2)c(CO)n1)c1ccccc1]

A mixture of (4-(2-benzamidoethyl)-1-benzyl-1H-imidazol-2-yl)methyl benzoate (2.3 g, 0.0052 mol), potassium hydroxide (0.9 g, 0.016 mol), 25 mL methanol, and 50 mL water was stirred at ambient temperature for 15 h. The mixture was extracted with chloroform (2×50 mL), organic layers were combined, dried with sodium sulfate, and purified by flash-chromatography (silica gel, chloroform:ethanol=10:1) to give the title product as a white solid, (0.5 g, 28.5%).

### Step 8: N-(2-(1-benzyl-2-formyl-1H-imidazol-4-yl)ethyl)acetamide

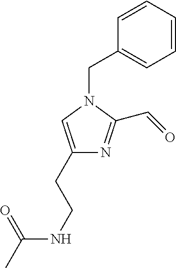 [SMILES: CC(=O)NCCc1cn(Cc2ccccc2)c(C=O)n1]

To a solution of N-(2-(1-benzyl-2-(hydroxymethyl)-1H-imidazol-4-yl)ethyl)benzamide (0.5 g, 1.5 mmol) in dichloromethane (15 mL) was added manganese (IV) oxide (1.3 g, 0.015 mol) and the mixture was stirred for 15 h at ambient temperature. The reaction mixture was filtered off and concentrated under reduced pressure to give the crude product as a colorless oil (0.6 g, 120.7%) which was used in the next step without further purification.

### Step 9: N-(2-(1-benzyl-2-((hydroxyimino)methyl)-1H-imidazol-4-yl)ethyl)acetamide

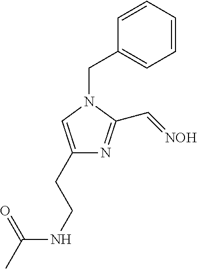

A solution of N-(2-(1-benzyl-2-formyl-1H-imidazol-4-yl)ethyl)acetamide (0.6 g, 1.8 mmol), hydroxylamine hydrochloride (0.152 g, 2.2 mmol), and sodium carbonate (0.117 g, 1.1 mmol) in a mixture of ethanol (30 mL) and water (30 mL) was stirred for 24 h at ambient temperature. The precipitate was filtered off and washed with water to give the crude desired product as a white solid (0.4 g, 63.8%) which was used in the next step without further purification.

### Step 10: N-(2-(2-(aminomethyl)-1-benzyl-1H-imidazol-4-yl)ethyl)benzamide

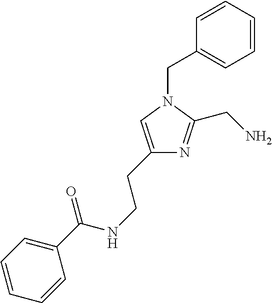 [SMILES: NCc1nc(CCNC(=O)c2ccccc2)cn1Cc1ccccc1]

A mixture of N-(2-(1-benzyl-2-((hydroxyimino)methyl)-1H-imidazol-4-yl)ethyl)acetamide (0.4 g, 1.15 mmol), Raney-Nickel (0.7 mL 50% suspension in water), ethanol (40 mL) was autoclaved and stirred at ambient temperature under pressure (40 bar of H₂) for 40 h. The precipitate was filtered off and washed with chloroform (20 mL). The combined filtrate was dried with sodium sulfate and concentrated under reduced pressure to give the product as a yellow oil (0.4 g, 104.2% yield). ¹H NMR (DMSO-d6, 400 MHz) δ (ppm): 8.57 (m, 1H), 7.89 (m, 2H), 7.54-7.42 (m, 3H), 7.36-7.17 (m, 5H), 6.88 (s, 1H), 5.17 (s, 2H), 3.64 (s, 2H), 3.47 (m, 2H), 2.68 (m, 2H); LCMS m/z=335.3 (M+H).

### Compound 2-3: 5-(2-(3-chlorophenyl)-2-oxoethyl)-2-(4-phenylpiperazin-1-yl)-1H-imidazol-4(5H)-one

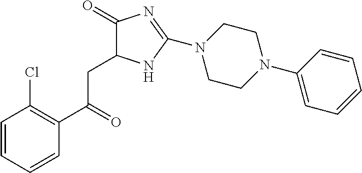 [SMILES: O=C(CC1NC(N2CCN(c3ccccc3)CC2)=NC1=O)c1ccccc1Cl]

### Step 1: (2-(2-chlorophenyl)-2-oxoethyl) triphenylphosphonium bromide

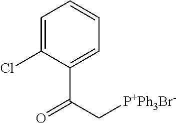

A solution of 2-bromo-3-chloroacetophenone (2.0 g, 0.0085 mol) and triphenylphosphine (2.92 g, 0.0113 mol) in chloroform (20 mL) was stirred at ambient temperature for 15 min. The reaction mixture was added to diethyl ether (70 mL) to obtained white solid. The solid was filtered off and washed with the diethyl ether and dried in vacuo to give title compound (3.5 g, quantitative yield) as a white solid.

### Step 2: 1-(2-chlorophenyl)-2-(triphenyl-5-phosphanylidene)ethan-1-one

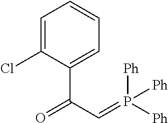 [SMILES: O=C(C=P(c1ccccc1)(c1ccccc1)c1ccccc1)c1ccccc1Cl]

A solution of (2-(3-chlorophenyl)-2-oxoethyl) triphenylphosphonium bromide (3.5 g) in 10% aqueous sodium carbonate (80 mL) was stirred at ambient temperature for overnight. To the reaction mixture was added dichloromethane and the mixture was stirred for 15 min. The organic layer was separated and evaporated in vacuo to give an oily crude product. The crude was triturated with diethyl ether to give the title compound (1.28 g, 35.02% yield) as a white solid. LCMS: 84.42% (m/z): 415.27 [M+H]+.

### Step 3: (E)-ethyl 4-(2-chlorophenyl)-4-oxobut-2-enoate

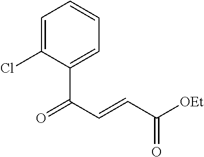 [SMILES: CCOC(=O)/C=C/C(=O)c1ccccc1Cl]

To a solution of 1-(2-chlorophenyl)-2-(triphenyl-5-phosphanylidene)ethan-1-one (1.28 g, 0.0030 mol) in tetrahydrofuran (12 mL) was added ethyl glyoxalate (0.75 g, 0.0037 mol) at ambient temperature and the reaction mixture was stirred overnight. The mixture was diluted with water and extracted with ethyl acetate and organic layer was separated, dried over sodium sulfate and evaporated in vacuo. The crude was purified by flash column chromatography and the product was eluted at 12% ethyl acetate in hexane. The evaporation of pure fractions gave title compound (0.57 g, 49.36% yield). ¹H NMR (400 MHz, CDCl₃): δ 7.56-7.47 (m, J=7.2, 8.8 Hz, 4H), 7.40-7.38 (m, 1H), 6.67 (d, J=16 Hz, 1H), 4.30 (q, 2H, J=7.2 Hz), 1.35 (t, J=7.2 Hz, 3H) ppm; LCMS: 97.88% (m/z): 239.15 [M+H]+.

### Step 4: 4-phenylpiperazine-1-carboximidamide

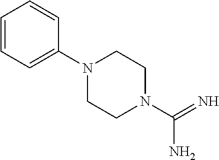 [SMILES: N=C(N)N1CCN(c2ccccc2)CC1]

To a solution of 1-phenyl piperazine (2.85 g, 0.0176 mol) in ethanol (8 mL) were added 1H-pyrazolecaroxamide hydrochloride (2.0 g, 0.0136 mol) and N, N-diisopropylethylamine (10.7 mL, 0.0612 mol) at ambient temperature. The reaction mixture was heated at 80° C. for 6 h. The reaction mixture was evaporated in vacuo and triturated with acetonitrile to give the title compound (3.1 g, 100% yield) as an off white solid. 1H NMR (400 MHz, DMSO-d6): δ 7.70 (s, 3H), 7.24 (dd, J=7.2, 8.4 Hz, 2H), 6.98 (d, J=8 Hz, 2H), 6.83 (t, J=7.2 Hz, 1H), 3.59 (t, J=5.2, 4H), 3.21 (t, J=5.2, 4H) ppm. LCMS: 91.65% (m/z)=205.28 [M+1]⁺.

### Step 5: 5-(2-(2-chlorophenyl)-2-oxoethyl)-2-(4-phenylpiperazin-1-yl)-1H-imidazol-4(5H)-one

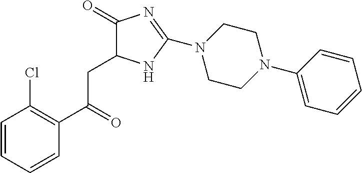 [SMILES: O=C(CC1NC(N2CCN(c3ccccc3)CC2)=NC1=O)c1ccccc1Cl]

To a solution of (E)-ethyl 4-(2-chlorophenyl)-4-oxobut-2-enoateate (0.15 g, 0.0006 mol) in acetonitrile (2.0 mL) were added 4-phenyl piperazine-1-carboximidamide (0.141 g, 0.00087 mol) and potassium carbonate (0.173 g, 0.0012 mol). The reaction was heated at 70° C. for 1 h. The reaction was filtered and washed with acetonitrile (5.0 mL) and water (2×5 mL). The solid was concentrated under reduced pressure to give 5-(2-(2-chlorophenyl)-2-oxoethyl)-2-(4-phenylpiperazin-1-yl)-1H-imidazol-4(5H)-one (0.035 g, 15.44% yield) as off white solid. 1H NMR (400 MHz, DMSO-d6): δ 8.24 (s, 1H), 7.73 (d, J=8 Hz, 1H), 7.57-7.54 (m, 2H), 7.49-7.45 (m, 1H), 7.24 (dd, J=7.6, 8.8 Hz, 2H), 6.99 (d, J=8.0 Hz, 2H), 6.82 (t, J=7.2 Hz, 1H), 4.27 (m, 1H), 3.69 (m, 4H), 3.37 (m, 1H), 3.22-3.17 (m, 5H) ppm; LCMS: 99.25% (m/z): 397.26 [M+1]+.

### Compound 12-3: 5-chloro-N-((1-(furan-2-ylmethyl)piperidin-4-yl)methyl)naphthalene-1-sulfonamide

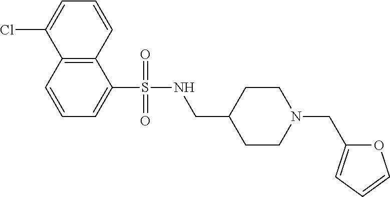 [SMILES: O=S(=O)(NCC1CCN(Cc2ccco2)CC1)c1cccc2c(Cl)cccc12]

### Step 1: tert-butyl ((1-(furan-2-ylmethyl)piperidin-4-yl)methyl)carbamate

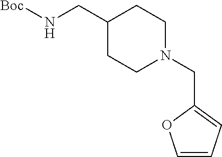 [SMILES: CC(C)(C)OC(=O)NCC1CCN(Cc2ccco2)CC1]

To a solution of tert-butyl (piperidin-4-ylmethyl)carbamate (19.9 g, 93 mmol) in dichloroethane (400 mL) was added furan-2-carbaldehyde (13.4 g, 139 mmol). The mixture was stirred for 1 h at ambient temperature, followed by addition of NaBH(OAc)₃ (29.6 g, 0.139 mol) and stirred for another 3 h. The resulting mixture was washed with saturated aqueous sodium bicarbonate solution and brine, the organic phase was dried and concentrated to give a residue, which was purified by chromatography on silica gel (eluted with petroleum ether:ethyl acetate=10:1-2:1) to afford tert-butyl ((1-(furan-2-ylmethyl)piperidin-4-yl)methyl)carbamate (22.5 g, 82% yield) as a red oil.

### Step 2: (1-(furan-2-ylmethyl)piperidin-4-yl)methanamine

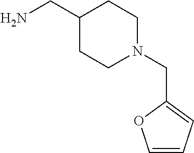 [SMILES: NCC1CCN(Cc2ccco2)CC1]

To a solution of tert-butyl ((1-(furan-2-ylmethyl)piperidin-4-yl)methyl)carbamate (22.5 g, 76 mmol) in methyl tert-butyl ether (120 mL) was added a solution of hydrochloric acid/methyl tert-butyl ether (120 mL). The mixture was stirred overnight at ambient temperature. The resulting solution was filtered. The solid was dissolved into water and basified by addition of 1 N sodium hydroxide to pH 11, then extracted 3 times with ethyl acetate/tetrahydrofuran (2:1). The organic phase was dried and concentrated under reduced pressure to afford the title compound (12 g, 81% yield) as a red oil. 1H NMR (CDCl₃, 400 MHz) δ (ppm): 7.33-7.32 (m, 1H), 6.26-6.25 (m, 1H), 6.14-6.13 (m, 1H), 3.47 (s, 2H), 2.88-2.85 (m, 2H), 2.52-2.51 (m, 2H), 1.92-1.91 (m, 2H), 1.66-1.64 (m, 2H), 1.22 (s, 1H), 1.11 (s, 2H).

### Step 3: 5-chloro-N-((1-(furan-2-ylmethyl)piperidin-4-yl)methyl)naphthalene-1-sulfonamide

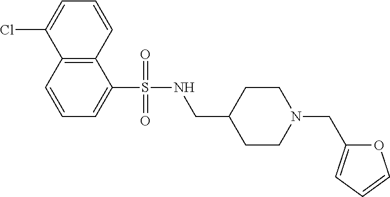 [SMILES: O=S(=O)(NCC1CCN(Cc2ccco2)CC1)c1cccc2c(Cl)cccc12]

To a solution of (1-(furan-2-ylmethyl)piperidin-4-yl)methanamine (200 mg, 1.0 mmol) in dichloromethane (7 mL) was added triethylamine (1 mL). The mixture was stirred at 0° C. for 30 min. Then 5-chloronaphthalene-1-sulfonyl chloride (230 mg, 1.0 mmol) in dichloromethane (3 mL) was added at 0° C. The mixture was stirred at ambient temperature for 2 h. The reaction mixture was extracted with dichloromethane, washed with water, dried over sodium sulfate, and concentrated to give the crude product, which was purified by prep-HPLC to give 5-chloro-N-((1-(furan-2-ylmethyl)piperidin-4-yl)methyl)naphthalene-1-sulfonamide. 1H NMR (CDCl₃, 400 MHz) δ (ppm): 8.52 (t, J=9.54, 2H), 8.18-8.27 (m, 1H), 7.55-7.66 (m, 2H), 7.43-7.53 (m, 1H), 7.27 (s, 1H), 6.17-6.27 (m, 1H), 6.08 (d, J=2.76, 1 H), 4.78 (br. s., 1H), 3.35-3.43 (m, 2H), 2.63-2.76 (m, 4H), 1.77 (t, J=10.67, 2H), 1.47 (d, J=12.55, 2H), 1.21-1.32 (m, 1H), 0.98-1.11 (m, 2H). LCMS (m/z): 419.1 [M+H]⁺.

### Compound 2-5: (S)—N-(1-benzylpyrrolidin-3-yl)-5-(dimethylamino)naphthalene-1-sulfonamide

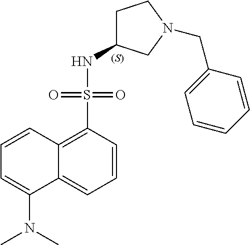

To a solution of (S)-1-benzylpyrrolidin-3-amine (60 mg, 0.34 mmol) in pyridine (5 mL) was added 5-(dimethylamino)naphthalene-1-sulfonyl chloride (92 mg, 0.34 mmol) in pyridine (2 mL) at 0° C. The mixture was stirred at 30° C. for 30 min under microwave. Water was added to the mixture, which was extracted with dichloromethane, the organic layer was dried over sodium sulfate, concentrated and purified by prep-HPLC to give the desired product (S)—N-(1-benzylpyrrolidin-3-yl)-5-(dimethylamino)naphthalene-1-sulfonamide (24.6 mg, 6 mmol). 1H NMR (CD₃OD, 400 MHz) δ: 8.56 (d, J=8.4, 1H), 8.35 (d, J=8.4, 1H), 8.22 (d, J=6.0, 1H), 7.54-7.61 (m, 2H), 7.22-7.28 (m, 4H), 7.12-7.14 (m, 2H), 3.69-3.74 (m, 1H), 3.32-3.34 (m, 2H), 2.87 (s, 6H), 2.39-2.49 (m, 3H), 2.07-2.11 (m, 1H), 1.86-1.92 (m, 1H), 1.44-1.49 (m, 1H). LCMS (m/z): 410.2 [M+H]⁺.

### Compound 3-5: N-(1-benzylazetidin-3-yl)-5-(dimethylamino)naphthalene-1-sulfonamide

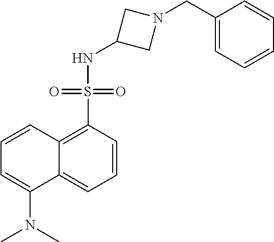 [SMILES: CN(C)c1cccc2c(S(=O)(=O)NC3CN(Cc4ccccc4)C3)cccc12]

### Step 1: tert-butyl 1-benzylazetidin-3-ylcarbamate

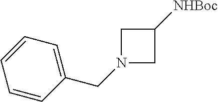 [SMILES: CC(C)(C)OC(=O)NC1CN(Cc2ccccc2)C1]

A mixture of tert-butyl azetidin-3-ylcarbamate hydrochloride (200 mg, 0.96 mmol), benzaldehyde (203 mg, 1.92 mmol), triethylamine (0.133 mL, 0.96 mmol) and acetic acid (115 mg, 1.92 mmol) in methanol (10 mL) was stirred at 24° C. for 30 min. Then NaBH₃CN (181 mg, 2.88 mmol) was added to the mixture. The resulting mixture was stirred at 24° C. for 12 h and concentrated under reduced pressure to give the crude product, which was purified by column to give tert-butyl 1-benzylazetidin-3-ylcarbamate (120 mg, 48% yield). LCMS (m/z): 262.2 [M+H]⁺.

### Step 2: 1-benzylazetidin-3-amine

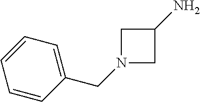 [SMILES: NC1CN(Cc2ccccc2)C1]

To a solution of tert-butyl 1-benzylazetidin-3-ylcarbamate (100 mg, 0.38 mmol) in ethyl acetate (10 mL) was added dropwise hydrochloric acid/ethyl acetate (10 mL) with ice-water bath. The mixture was stirred at 24° C. for 2 h and concentrated under reduced pressure to give 1-benzylazetidin-3-amine (74 mg, 99% yield). LCMS (m/z): 162.2 [M+H]⁺.

### Step 3: N-(1-benzylazetidin-3-yl)-5-(dimethylamino)naphthalene-1-sulfonamide

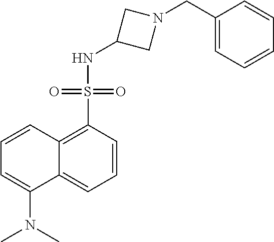 [SMILES: CN(C)c1cccc2c(S(=O)(=O)NC3CN(Cc4ccccc4)C3)cccc12]

5-(dimethylamino)naphthalene-1-sulfonyl chloride (258.9 mg, 0.96 mmol) in pyridine (5 mL) was added dropwise to a solution of 1-benzylazetidin-3-amine hydrochloride (155 mg, 0.96 mmol) in pyridine (5 mL) cooled with an ice-water bath. The mixture was stirred at 24° C. for 12 h and concentrated under reduced pressure to give the crude product, which was purified by prep-HPLC to give N-(1-benzylazetidin-3-yl)-5-(dimethylamino)naphthalene-1-sulfonamide (89.5 mg, 24% yield). 1H NMR (400 MHz, DMSO-d6): δ 8.44 (d, J=8 Hz, 2H), 8.29 (d, J=8 Hz, 1H), 8.09-8.07 (m, 1H), 7.63-7.58 (m, 2H), 7.26-7.16 (m, 4H), 7.11-7.09 (m, 2H), 3.73-3.70 (m, 1H), 3.33 (s, 2H), 3.14-3.10 (m, 2H), 2.81 (s, 6H), 2.64-2.61 (m, 2H) ppm; ESI-MS (m/z): 396.1 [M+1]⁺.

### Compound 3-4: (S)-5-(dimethylamino)-N-(5-oxo-1-phenylpyrrolidin-3-yl)naphthalene-1-sulfonamide

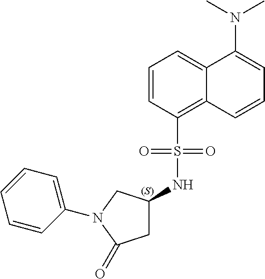

### Step 1: 5-oxo-1-phenylpyrrolidine-3-carboxylic acid

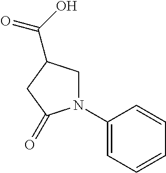 [SMILES: O=C(O)C1CC(=O)N(c2ccccc2)C1]

To a solution of 2-methylene-succinic acid (20 g, 150 mmol) in water (100 mL) was added phenylamine (14.3 g, 150 mmol). The resulting mixture was refluxed for 45 min. After cooling to 0° C., the product was filtered and the solid cake was washed with cold water and dissolved in aqueous sodium hydroxide. The aqueous phase was treated with charcoal, filtered, and acidified with diluted hydrochloric acid. The precipitate was filtered and washed with water, then dried to give compound 5-oxo-1-phenylpyrrolidine-3-carboxylic acid (20 g, 63% yield) as a light white solid. 1H NMR (CD₃OD, 400 MHz) δ (ppm): 7.58-7.56 (m, 2H), 7.40-7.36 (m, 2H), 7.21-7.17 (m, 2H), 4.17-4.08 (m, 2H), 3.46-3.38 (m, 1H), 2.88-2.86 (d, J=7.6 Hz, 2H).

### Step 2: tert-butyl (5-oxo-1-phenylpyrrolidin-3-yl)carbamate

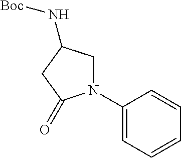 [SMILES: CC(C)(C)OC(=O)NC1CC(=O)N(c2ccccc2)C1]

To a solution of 5-oxo-1-phenyl-pyrrolidine-3-carboxylic acid (10 g, 48.8 mmol) in tert-butanol (200 mL) was added diphenylphosphoryl azide (12.8 g, 53.7 mmol) and triethylamine (7.4 g, 73.2 mmol). The mixture was refluxed overnight. The solvent was removed by reduced pressure to give a residue, which was purified by column chromatograph on silica gel to give compound the desired compound (9.8 g, 73% yield) as brown solid. 1H NMR (CDCl₃, 400 MHz) δ (ppm): 7.59-7.57 (m, 2H), 7.39-7.36 (m, 2H), 7.18-7.14 (m, 1H), 4.85 (s, 1H), 4.42 (s, 1H), 4.19-4.15 (m, 1H), 3.74-3.71 (m, 1H), 3.01-2.94 (m, 1H), 2.51-2.46 (m, 1H), 1.45 (s, 9H).

### Step 3: 4-amino-1-phenylpyrrolidin-2-one

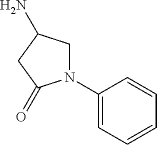 [SMILES: NC1CC(=O)N(c2ccccc2)C1]

A solution of (tert-butyl (5-oxo-1-phenylpyrrolidin-3-yl)carbamate in hydrochloric acid/ethyl acetate was stirred at ambient temperature for 2 h. The mixture was filtered to produce a white solid. The solid cake was dissolved in water. The solution was basified by addition of 1 N sodium hydroxide to pH 14 and extracted with ethyl acetate 6 times. The organic layer was dried over sodium sulfate and concentrated to give 4-amino-1-phenylpyrrolidin-2-one (5 g, 80% yield) as white solid. 1H NMR (CD₃OD, 400 MHz) δ (ppm): 7.60-7.58 (m, 2H), 7.39-7.35 (m, 2H), 7.19-7.15 (m, 1H), 4.13-4.09 (m, 1H), 3.78-3.73 (m, 1H), 3.61-3.57 (m, 1H), 2.91-2.84 (m, 1H), 2.40-2.35 (m, 1H).

### Step 4: 5-(dimethylamino)-N-(5-oxo-1-phenylpyrrolidin-3-yl)naphthalene-1-sulfonamide

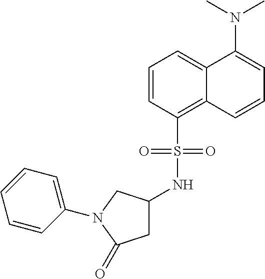 [SMILES: CN(C)c1cccc2c(S(=O)(=O)NC3CC(=O)N(c4ccccc4)C3)cccc12]

To a solution of 4-amino-1-phenylpyrrolidin-2-one (50 mg, 0.28 mmol) in pyridine (5 mL) was added 5-(dimethylamino)naphthalene-1-sulfonyl chloride (76 mg, 0.28 mmol) in pyridine (2 mL) at 0° C. The mixture was stirred at 30° C. for 30 min under microwave. Water was added to the mixture which was extracted with dichloromethane, the organic layer was concentrated, and the crude material was purified by prep-HPLC to give the compound 5-(dimethylamino)-N-(5-oxo-1-phenylpyrrolidin-3-yl)naphthalene-1-sulfonamide.

### Step 5: (S)-5-(dimethylamino)-N-(5-oxo-1-phenylpyrrolidin-3-yl)naphthalene-1-sulfonamide

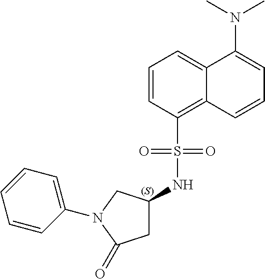

The compound 5-(dimethylamino)-N-(5-oxo-1-phenylpyrrolidin-3-yl) naphthalene-1-sulfonamide was purified by SFC to give the compound (S)-5-(dimethylamino)-N-(5-oxo-1-phenylpyrrolidin-3-yl)naphthalene-1-sulfonamide. ¹H NMR (CD₃OD, 400 MHz) δ: 8.61 (d, J=8.53 Hz, 1H), 8.34-8.42 (m, 1H), 8.29 (dd, J=7.28, 1.00 Hz, 1H), 7.57-7.69 (m, 2H), 7.26-7.38 (m, 5H), 7.10-7.20 (m, 1H), 4.00-4.12 (m, 1H), 3.88 (dd, J=10.42, 6.90 Hz, 1H), 3.52 (dd, J=10.29, 4.02 Hz, 1H), 2.91 (s, 6H), 2.66 (dd, J=17.19, 8.16 Hz, 1H), 2.31 (dd, J=17.19, 4.64 Hz, 1H). LCMS (m/z): 410.2 [M+H]+.

Compound 2-7: N-(3-amino-4-phenylbutyl)-5-(dimethylamino)naphthalene-1-sulfonamide

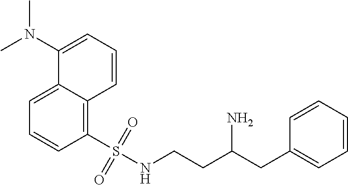 [SMILES: CN(C)c1cccc2c(S(=O)(=O)NCCC(N)Cc3ccccc3)cccc12]

### Step 1: tert-butyl 1-carbamoyl-3-phenylpropan-2-ylcarbamate

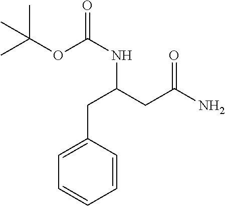 [SMILES: CC(C)(C)OC(=O)NC(CC(N)=O)Cc1ccccc1]

To a solution of 3-((tert-butoxycarbonyl)amino)-4-phenylbutanoic acid (100 mg, 0.358 mmol) in dichloromethane (10 mL) was added carbonyldiimidazole (75 mg, 0.54 mmol) in portions at 34° C. The mixture was stirred at 34° C. for 30 min then the resulting solution was added dropwise to ammonium hydroxide and stirred at 34° C. for 16 h. The mixture was extracted with dichloromethane (×3) and the organic layers was dried over sodium sulfate and concentrated under reduced pressure give tert-butyl 1-carbamoyl-3-phenylpropan-2-ylcarbamate (80 mg, 81% yield).

### Step 2: tert-butyl 4-amino-1-phenylbutan-2-ylcarbamate

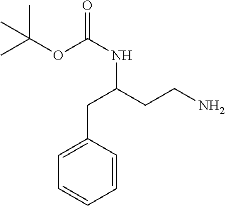 [SMILES: CC(C)(C)OC(=O)NC(CCN)Cc1ccccc1]

To a solution of tert-butyl 1-carbamoyl-3-phenylpropan-2-ylcarbamate (50 mg, 0.18 mmol) in tetrahydrofuran was added dropwise BH₃ THF (1.8 mL, 1.8 mmol) with ice-water bath. The mixture was stirred at 32° C. for 12 h then was quenched with methanol at 0° C. and concentrated under reduced pressure to give the crude product which was used without further purification. LCMS (m/z): 265.2 [M+H]⁺.

### Step 3: tert-butyl (4-(5-(dimethylamino)naphthalene-1-sulfonamido)-1-phenylbutan-2-yl)carbamate

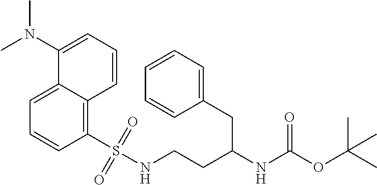 [SMILES: CN(C)c1cccc2c(S(=O)(=O)NCCC(Cc3ccccc3)NC(=O)OC(C)(C)C)cccc12]

To a solution of tert-butyl 4-amino-1-phenylbutan-2-ylcarbamate (47.6 mg, 0.18 mmol) in pyridine was added a solution of 5-(dimethylamino)naphthalene-1-sulfonyl chloride (24 mg, 0.09 mmol) in dichloromethane. The mixture was stirred at 34° C. for 12 h and concentrated in vacuo to give the crude product which was used to the next step without further purification. LCMS (m/z): 498.3 [M+H]⁺.

### Step 4: N-(3-amino-4-phenylbutyl)-5-(dimethylamino)naphthalene-1-sulfonamide

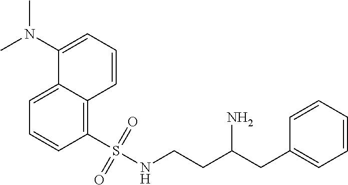 [SMILES: CN(C)c1cccc2c(S(=O)(=O)NCCC(N)Cc3ccccc3)cccc12]

To a solution of tert-butyl 2-oxo-1-phenylpiperidin-4-ylcarbamate (49.8 mg, 0.1 mmol) in ethyl acetate (10 mL) was added dropwise hydrochloric acid/ethyl acetate (10 mL) with ice-water bath. The mixture was stirred at 32° C. for 2 h and concentrated in vacuo to give N-(3-amino-4-phenylbutyl)-5-(dimethylamino)naphthalene-1-sulfonamide (9.2 mg, 24% yield). ¹H NMR (400 MHz, CD₃OD): δ (ppm) 8.69-8.47 (m, 2H), 8.33 (d, J=8.7 Hz, 1H), 8.21 (d, J=7.3 Hz, 1H), 7.61 (q, J=7.7 Hz, 2H), 7.39-7.24 (m, 4H), 7.14 (d, J=6.9 Hz, 2H), 3.49 (quin, J=6.7 Hz, 1H), 3.04-2.93 (m, 2H), 2.90 (s, 6H), 2.86-2.72 (m, 2H), 1.74 (q, J=6.7 Hz, 2H); ESI-MS (m/z): 398.2 [M+1]⁺.

### Compound 1-7: N-(3-amino-3-phenylpropyl)-5-(dimethylamino)naphthalene-1-sulfonamide

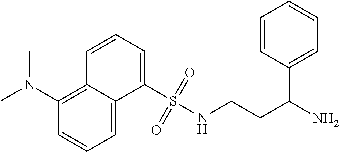 [SMILES: CN(C)c1cccc2c(S(=O)(=O)NCCC(N)c3ccccc3)cccc12]

### Step 1: 3-((tert-butoxycarbonyl)amino)-3-phenylpropanoic acid

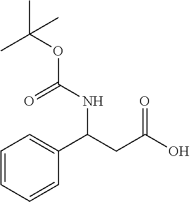 [SMILES: CC(C)(C)OC(=O)NC(CC(=O)O)c1ccccc1]

To a mixture of 3-amino-3-phenylpropanoic acid (100 mg, 0.61 mmol) and sodium carbonate (194 mg, 1.83 mmol) in dioxane (10 mL) and water (10 mL) was added dropwise di-tert-butyl dicarbonate (200 mg, 0.92 mmol) at 0° C. The mixture was stirred at 30° C. for 12 h. The resulting mixture was filtered and the filtrate was adjusted to pH 4 with aqueous hydrochloric acid. The residue was extracted with dichloromethane (20 mL×3) and the combined organic layers was dried over sodium sulfate and concentrated in vacuo to give 3-((tert-butoxycarbonyl)amino)-3-phenylpropanoic acid (140 mg, 87% yield).

### Step 2: tert-butyl (3-amino-3-oxo-1-phenylpropyl)carbamate

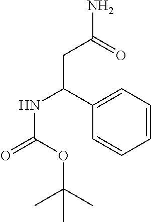 [SMILES: CC(C)(C)OC(=O)NC(CC(N)=O)c1ccccc1]

To a solution of 3-((tert-butoxycarbonyl)amino)-3-phenylpropanoic acid (26.5 mg, 0.1 mmol) in tetrahydrofuran (10 mL) was added carbonyldiimidazole (21.1 mg, 0.15 mmol) in portions at 0° C. The mixture was stirred at 30° C. for 30 min then the resulting solution was added dropwise to ammonium hydroxide (10 mL) and stirred at 32° C. for 12 h. The mixture was extracted with dichloromethane (×3) and the organic layer was dried over sodium sulfate and concentrated in vacuo give tert-butyl (3-amino-3-oxo-1-phenylpropyl)carbamate (22 mg, 83% yield).

### Step 3: tert-butyl (3-amino-1-phenylpropyl)carbamate

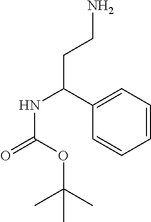 [SMILES: CC(C)(C)OC(=O)NC(CCN)c1ccccc1]

To a solution of tert-butyl (3-amino-3-oxo-1-phenylpropyl)carbamate (80 mg, 0.303 mmol) in tetrahydrofuran (10 mL) was added dropwise BH₃ THF (3.03 mL, 3.03 mmol) with ice-water bath. The mixture was stirred at 32° C. for 12 h then was quenched with methanol at 0° C. and concentrated in vacuo to give the crude product which was used to the next without further purification. LCMS (m/z): 251.2 [M+H]⁺.

### Step 4: tert-butyl (3-((5-(dimethylamino)naphthalene)-1-sulfonamido)-1-phenylpropyl)carbamate

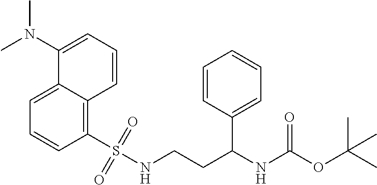 [SMILES: CN(C)c1cccc2c(S(=O)(=O)NCCC(NC(=O)OC(C)(C)C)c3ccccc3)cccc12]

To a solution of tert-butyl (3-amino-1-phenylpropyl)carbamate (57 mg, 0.23 mmol) in pyridine was added a solution of 5-(dimethylamino)naphthalene-1-sulfonyl chloride (62 mg, 0.23 mmol) in dichloromethane. The mixture was stirred at 35° C. for 12 h and concentrated in vacuo to give the crude product which was used to the next without further purification. LCMS (m/z): 484.2 [M+H]⁺.

### Step 5: N-(3-amino-3-phenylpropyl)-5-(dimethylamino)naphthalene-1-sulfonamide

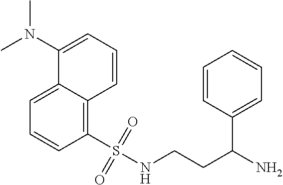 [SMILES: CN(C)c1cccc2c(S(=O)(=O)NCCC(N)c3ccccc3)cccc12]

To a solution of tert-butyl (3-((5-(dimethylamino)naphthalene)-1-sulfonamido)-1-phenylpropyl)carbamate (40 mg, 0.083 mmol) in methanol (10 mL) was added dropwise hydrochloric acid/ethyl acetate (10 mL) cooled with ice-water bath. The mixture was stirred at 32° C. for 12 h. The solvent was removed in vacuo to give the desired product (9.9 mg, 31% yield). ¹H NMR (400 MHz, CD₃OD): δ 8.66-8.48 (m, 2H), 8.33 (d, J=8.7 Hz, 1H), 8.11 (d, J=7.2 Hz, 1H), 7.62 (t, J=8.1 Hz, 1H), 7.55 (t, J=7.9 Hz, 1H), 7.43-7.34 (m, 3H), 7.31 (d, J=7.4 Hz, 1H), 7.27-7.18 (m, 2H), 4.24 (dd, J=5.4, 9.5 Hz, 1H), 2.90 (s, 6H), 2.85-2.63 (m, 3H), 2.24-2.10 (m, 1H), 2.10-1.93 (m, 1H) ppm; ESI-MS (m/z): 384.2 [M+1]⁺.

### Compound 5-7: (S)—N-(2-amino-3-phenylpropyl)-5-(dimethylamino)naphthalene-1-sulfonamide

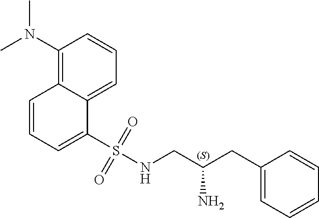

### Step 1: (S)-tert-butyl (1-amino-1-oxo-3-phenylpropan-2-yl)carbamate

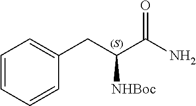

To a solution of (S)-2-amino-3-phenylpropanamide (820 mg, 5 mmol) and triethylamine (1.01 g, 10 mmol) in dichloromethane (30 mL) was added di-tert-butyl dicarbonate (1.31 g, 6 mmol) at 0° C. The mixture was allowed to warm slowly to ambient temperature and was stirred at ambient temperature for 3 h. The reaction mixture was washed with water, extracted with dichloromethane, the organic layer was dried over sodium sulfate, and concentrated to give the crude (S)-tert-butyl (1-amino-1-oxo-3-phenylpropan-2-yl)carbamate which was used in next step without further purification.

### Step 2: (S)-tert-butyl (1-amino-3-phenylpropan-2-yl)carbamate

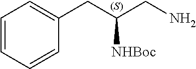

To a solution of (S)-tert-butyl (1-amino-1-oxo-3-phenylpropan-2-yl)carbamate (264 mg, 1 mmol) in tetrahydrofuran (20 mL) was added BH₃ Me₂S (1 mL) at 0° C. The mixture was reflux overnight. The reaction mixture was quenched with methanol, extracted with ethyl acetate, the organic layer was dried over sodium sulfate and concentrated to give the crude (S)-tert-butyl (1-amino-3-phenylpropan-2-yl)carbamate which was used in next step without further purification.

### Step 3: (S)-tert-butyl (1-(5-(dimethylamino)naphthalene-1-sulfonamido)-3-phenylpropan-2-yl)carbamate

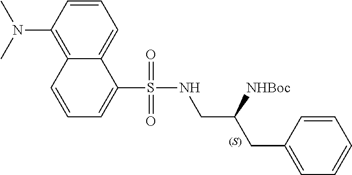

To a solution of (S)-tert-butyl (1-amino-3-phenylpropan-2-yl)carbamate (250 mg, 1 mmol) and triethylamine (101 mg, 2 mmol) in dichloromethane (7 mL) was added 5-(dimethylamino)naphthalene-1-sulfonyl chloride (269 mg, 1 mmol) in dichloromethane (3 mL) at 0° C. The mixture was allowed to warm slowly to ambient temperature overnight. The reaction mixture was washed with water, extracted with dichloromethane, the organic layer was dried over sodium sulfate and concentrated to give the crude (S)-tert-butyl (1-(5-(dimethylamino)naphthalene-1-sulfonamido)-3-phenylpropan-2-yl)carbamate which was used in next step without further purification.

### Step 4: (S)—N-(2-amino-3-phenylpropyl)-5-(dimethylamino)naphthalene-1-sulfonamide

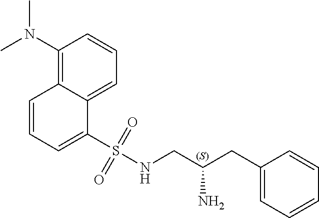

To a solution of (S)-tert-butyl (1-(5-(dimethylamino)naphthalene-1-sulfonamido)-3-phenylpropan-2-yl)carbamate (120 mg, 0.25 mmol) in ethyl acetate (7 mL) was added hydrochloric acid/ethyl acetate (7 mL) at 0° C. The mixture was stirred at 0° C. for 30 min, then allowed to warm slowly to ambient temperature and stirred at ambient temperature overnight. The reaction mixture was concentrated to give the crude which was purified by prep-HPLC to give the desired (S)—N-(2-amino-3-phenylpropyl)-5-(dimethylamino) naphthalene-1-sulfonamide. ¹H NMR (CD₃OD, 400 MHz) δ: 8.50-8.64 (m, 1H), 8.34 (d, J=8.78 Hz, 1H), 8.12 (d, J=7.03 Hz, 1H), 7.50-7.66 (m, 2H), 7.18-7.33 (m, 4H), 7.05 (d, J=6.53 Hz, 2H), 3.20-3.29 (m, 1H), 2.82-2.94 (m, 8H), 2.64-2.79 (m, 2H). LCMS (m/z): 384.2 [M+H]⁺.

### LC-MS Conditions

Method A (LCMS-B (0-60AB_ELSD_2MIN))

Experiments performed on an Agilent 1200 HPLC (with a PDA detector and a ELSD detector) with Agilent 6100 MSD mass spectrometer using ESI as ionization source using an Xtimate™-C18 30*2.1 mm column and a 0.8 ml/minute flow rate. Acquire Time: 2 min, Wavelength: UV220, Oven Temp.: 50° C. The solvent system was a gradient starting with 100% water containing 0.038% TFA (solvent A) and 0% acetonitrile containing 0.02% TFA (solvent B), followed by a gradient up to 40% solvent A and 60% solvent B over the next 0.9 minutes. This was maintained for 0.6 minutes before returning to 100% solvent A over the next 0.5 minute. Total run time was 2 min.

### Method B (LCMS-C(10-80_AB))

Experiments performed on an SHIMADZU 20A HPLC (with a PDA detector) with SHIMADZU 2010EV MSD mass spectrometer using ESI as ionization source using an Xtimate™-C18 30*2.1 mm column and a 1.2 ml/minute flow rate. The solvent system was a gradient starting with 90% water containing 0.038% TFA (solvent A) and 10% acetonitrile containing 0.02% TFA (solvent B), followed by a gradient up to 20% solvent A and 80% solvent B over the next 0.9 minutes. This was maintained for 0.6 minutes before returning to 90% solvent A and 10% solvent B over the next 0.5 minute. Total run time was 2 min.

Method C (LCMS-E(5-95AB_220&254 nm))

Experiments performed on an SHIMADZU 20A HPLC (with a PDA detector) with SHIMADZU 2010EV MSD mass spectrometer using ESI as ionization source using an Merk RP-18e 2*25 mm column and a 1.5 ml/minute flow rate. The solvent system was a gradient starting with 95% water containing 0.038% TFA (solvent A) and 5% acetonitrile containing 0.02% TFA (solvent B), followed by a gradient up to 5% solvent A and 95% solvent B over the next 0.7 minutes. This was maintained for 0.4 minutes before returning to 95% solvent A and 5% solvent B over the next 0.4 minute. Total run time was 1.5 min.

### Method D (LCMS-A(0-30_AB))

Experiments performed on an SHIMADZU 20A HPLC (with a PDA detector) with SHIMADZU 2010EV MSD mass spectrometer using ESI as ionization source using an Xtimate™-C18 30*2.1 mm column and a 1.2 ml/minute flow rate. The solvent system was a gradient starting with 100% water containing 0.038% TFA (solvent A) and 0% acetonitrile containing 0.02% TFA (solvent B), followed by a gradient up to 70% solvent A and 30% solvent B over the next 0.9 minutes. This was maintained for 0.6 minutes before returning to 100% solvent A over the next 0.5 minute. Total run time was 2 min.

### General HPLC Conditions (Acidic)

Mobile phase A: 4 L H₂O\1.5 ml TFA; Mobile phase B: 4 L ACN\0.75 ml TFA

Column: HPLC-D: Innovation C18 UPLC Column 2.1×30 mm, 2.6 um

HPLC-E: Xtimate C18 2.1*30 mm*3 um

HPLC-H: Innovation C18 UPLC Column 2.1×30 mm, 2.6 um

Column temperature: 50° C.; Wavelength: 220 nm&254 nm&215 nm

### General HPLC Conditions (Basic)

Mobile phase A: 4 L H₂O\2 ml NH₄OH; Mobile phase B: Acetonitrile

Column: HPLC-B: XBridge C18 2.1*50 mm, 5 um

HPLC-C: Xbridge shield RP18 2.1*50 mm, 5 u

Column temperature: 30° C.; Wavelength: 220 nm&254 nm&215 nm

### General HPLC Conditions (Neutral)

Mobile phase A: H₂O; Mobile phase B: Acetonitrile

Column: HPLC-B: XBridge C18 2.1*50 mm, 5 um

HPLC-C: Xbridge shield RP18 2.1*50 mm, 5 um

Column temperature: 30° C.; Wavelength: 220 nm&254 nm&215 nm

### Method A (0-30AB_6MIN)

Flow Rate: 0.8 ml/min

Gradient: 0% B to 30% B in 4.2 min, holding 30% B for 1 min, 30% B to 0% B in 0.01 min, holding 0% B for 1.09 min and then end.

### Method B (0-60AB_6MIN)

Flow Rate: 0.8 ml/min

Gradient: 0% B to 60% B in 4.2 min, holding 60% B for 1 min, 60% B to 0% B in 0.01 min, holding 0% B for 1.09 min and then end.

### Method C (10-80AB_6MIN)

Flow Rate: 0.8 ml/min

Gradient: 10% B to 80% B in 4.2 min, holding 80% B for 1 min, 80% B to 10% B in 0.01 min, holding 10% B for 1.09 min and then end.

### Chiral HPLC Conditions:

### Method A (OJ-H):

Column: Chiralcel OJ-H 250×4.6 mm I.D., 5 um

Mobile phase: A/B=90/10, A: Hexane with 0.1% DEA, B: Ethanol

Flow rate: 0.5 mL/min

Wavelength: 220 nm

### Method B (OD-H):

Column: Chiralcel OD-H 250×4.6 mm I.D., 5 um

Mobile phase: A/B=90/10, A: Hexane with 0.1% DEA, B: Ethanol

Flow rate: 0.5 mL/min

Wavelength: 220 nm

### Method C (AD-H):

Column: Chiralpak AD-H 250×4.6 mm I.D., 5 um

Mobile phase: A/B=90/10, A: Hexane with 0.1% DEA, B: Ethanol

Flow rate: 0.5 mL/min

Wavelength: 220 nm

### Method D (AS-H):

Column: Chiralpak OJ-H 250×4.6 mm I.D., 5 um

Mobile phase: A/B=90/10, A: Hexane with 0.1% DEA, B: Ethanol

Flow rate: 0.5 mL/min

Wavelength: 220 nm

### B. Biological Data

### PRMT5 Biochemical Assay

General Materials.

S-adenosylmethionine (SAM), S-adenosylhomocysteine (SAH), bicine, KCl, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 1-15 was synthesized with a C-terminal linker-affinity tag motif and a C-terminal amide cap by 21^{st} Century Biochemicals. The peptide was high high-performance liquid chromatography (HPLC) purified to greater than 95% purity and confirmed by liquid chromatography mass spectrometry (LC-MS). The sequence was Ac-SGRGKGGKGLGKGGA[K-Biot]-amide (SEQ ID NO.:3).

Molecular Biology:

Full-length human PRMT5 (NM_006109.3) transcript variant 1 clone was amplified from a fetal brain cDNA library, incorporating flanking 5′ sequence encoding a FLAG tag (MDYKDDDDK) (SEQ ID NO.:4) fused directly to Ala 2 of PRMT5. Full-length human MEP50 (NM_024102) clone was amplified from a human testis cDNA library incorporating a 5′ sequence encoding a 6-histidine tag (MHHHHHH) (SEQ ID NO.:5) fused directly to Arg 2 of MEP50. The amplified genes were sublconed into pENTR/D/TEV (Life Technologies) and subsequently transferred by Gateway™ attL×attR recombination to pDEST8 baculvirus expression vector (Life Technologies).

Protein Expression.

Recombinant baculovirus and Baculovirus-Infected Insect Cells (BIIC) were generated according to Bac-to-Bac kit instructions (Life Technologies) and Wasilko, 2006, respectively. Protein over-expression was accomplished by infecting exponentially growing Spodoptera frugiperda (SF9) cell culture at 1.2×10⁶ cell/ml with a 5000 fold dilution of BIIC stock. Infections were carried out at 27° C. for 72 hours, harvested by centrifugation, and stored at −80° C. for purification.

Protein Purification.

Expressed full-length human Flag-PRMT5/6His-MeP50 protein complex was purified from cell paste by NiNTA agarose affinity chromatography after a five hour equilibration of the resin with buffer containing 50 mM Tris-HCL, pH 8.0, 25 mM NaCl, and 1 mM TCEP at 4° C., to minimize the adsorption of tubulin impurity by the resin. Flag-PRMT5/6His-MeP50 was eluted with 300 mM Imidazole in the same buffer. The purity of recovered protein was 87%. Reference: Wasilko, D. J. and S. E. Lee: “TIPS: titerless infected-cells preservation and scale-up” Bioprocess J., 5 (2006), pp. 29-32.

Predicted Translations:

Flag-PRMT5

(SEQ ID NO.: 6)

MDYKDDDDKA AMAVGGAGGS RVSSGRDLNC VPEIADTLGA
VAKQGFDFLC MPVFHPRFKR EFIQEPAKNR PGPQTRSDLL
LSGRDWNTLI VGKLSPWIRP DSKVEKIRRN SEAAMLQELN
FGAYLGLPAF LLPLNQEDNT NLARVLTNHI HTGHHSSMFW
MRVPLVAPED LRDDIIENAP TTHTEEYSGE EKTWMWWHNF
RTLCDYSKRI AVALEIGADL PSNHVIDRWL GEPIKAAILP
TSIFLTNKKG FPVLSKMHQR LIFRLLKLEV QFIITGTNHH
SEKEFCSYLQ YLEYLSQNRP PPNAYELFAK GYEDYLQSPL
QPLMDNLESQ TYEVFEKDPI KYSQYQQAIY KCLLDRVPEE
EKDTNVQVLM VLGAGRGPLV NASLRAAKQA DRRIKLYAVE
KNPNAVVTLE NWQFEEWGSQ VTVVSSDMRE WVAPEKADII
VSELLGSFAD NELSPECLDG AQHFLKDDGV SIPGEYTSFL
APISSSKLYN EVRACREKDR DPEAQFEMPY VVRLHNFHQL
SAPQPCFTFS HPNRDPMIDN NRYCTLEFPV EVNTVLHGFA
GYFETVLYQD ITLSIRPETH SPGMFSWFPI LFPIKQPITV
REGQTICVRF WRCSNSKKVW YEWAVTAPVC SAIHNPTGRS
YTIG L
6His-MEP50

(SEQ ID NO.: 7)

MHHHHHHRKE TPPPLVPPAA REWNLPPNAP ACMERQLEAA
RYRSDGALLL GASSLSGRCW AGSLWLFKDP CAAPNEGFCS
AGVQTEAGVA DLTWVGERGI LVASDSGAVE LWELDENETL
IVSKFCKYEH DDIVSTVSVL SSGTQAVSGS KDICIKVWDL
AQQVVLSSYR AHAAQVTCVA ASPHKDSVFL SCSEDNRILL
WDTRCPKPAS QIGCSAPGYL PTSLAWHPQQ SEVFVFGDEN
GTVSLVDTKS TSCVLSSAVH SQCVTGLVFS PHSVPFLASL
SEDCSLAVLD SSLSELFRSQ AHRDFVRDAT WSPLNHSLLT
TVGWDHQVVH HVVPTEPLPA PGPASVTE

General Procedure for PRMT5/MEP50 Enzyme Assays on Peptide Substrates.

The assays were all performed in a buffer consisting of 20 mM Bicine (pH=7.6), 1 mM TCEP, 0.005% BSG, and 0.002% Tween20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT5/MEP50, was added to columns 11, 12, 23, 24, rows I-P for the minimum signal control. A cocktail (40 ul) containing the PRMT5/MEP50 enzyme and the peptide was added by Multidrop Combi (Thermo-Fisher). The compounds were allowed to incubate with PRMT5/MEP50 for 30 min at 25 degrees Celsius, then a cocktail (10 ul) containing ³H-SAM was added to initiate the reaction (final volume=51 ul). The final concentrations of the components were as follows: PRMT5/MEP50 was 4 nM, ³H-SAM was 75 nM, peptide was 40 nM, SAH in the minimum signal control wells was 100 uM, and the DMSO concentration was 1%. The assays were stopped by the addition of non-radioactive SAM (10 ul) to a final concentration of 600 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed three times with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% tnh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### Z-138 Methylation Assay

Z-138 suspension cells were purchased from ATCC (American Type Culture Collection, Manassas, Va.). RPMI/Glutamax medium, penicillin-streptomycin, heat inactivated fetal bovine serum, and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Symmetric di-methyl arginine antibody was purchased from EMD Millipore, Billerica, Mass., USA. 16% Paraformaldehyde was purchased from Electron Microscopy Sciences, Hatfield, Pa., USA.

Z-138 suspension cells were maintained in growth medium (RPMI 1640 supplemented with 10% v/v heat inactivated fetal bovine serum and 100 units/mL penicillin-streptomycin) and cultured at 37° C. under 5% CO₂.

Cell Treatment, in Cell Western (ICW) for Detection of Symmetric Di-Methyl Arginine and DNA Content.

Z-138 cells were seeded in assay medium at a concentration of 50,000 cells per mL to a 384-well cell culture plate with 50 μL per well. Compound (100 nL) from 384 well source plates was added directly to 384 well cell plate. Plates were incubated at 37° C., 5% CO₂ for 96 hours. After four days of incubation, 40 μL of cells from incubated plates were added to poly-D-lysine coated 384 well culture plates (BD Biosciences 356697). Plates were incubated at room temperature for 30 minutes then incubated at 37° C., 5% CO₂ for 5 hours. After the incubation, 40 μL per well of 8% paraformaldehyde in PBS (16% paraformaldahyde was diluted to 8% in PBS) was added to each plate and incubated for 30 minutes. Plates were transferred to a Biotek 405 plate washer and washed 5 times with 100 μL per well of wash buffer (1×PBS with 0.1% Triton X-100 (v/v)). Next 30 μL per well of Odyssey blocking buffer were added to each plate and incubated 1 hour at room temperature. Blocking buffer was removed and 20 μL per well of primary antibody was added (symmetric di-methyl arginine diluted 1:100 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQ5 (Biostatus limited) in Odyssey buffer with 0.1% Tween 20 (v/v)) and incubated for 1 hour at room temperature. The plates were washed 5 times with 100 μL per well wash buffer then 1 time with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{symmetric di \text{-} methyl Arginine 800 nm value}{DRAQ 5 \; 700 nm value} )$$

Each plate included fourteen control wells of DMSO only treatment (minimum inhibition) as well as fourteen control wells for maximum inhibition treated with 3 μM of a reference compound (Background wells). The average of the ratio values for each control type was calculated and used to determine the percent inhibition for each test well in the plate. Reference compound was serially diluted three-fold in DMSO for a total of nine test concentrations, beginning at 3 μM. Percent inhibition was determined and IC₅₀ curves were generated using triplicate wells per concentration of compound.

$$Percent Inhibition = 100 - ( ( \frac{( Individual Test Sample Ratio ) - ( Background Avg Ratio )}{( Minimum Inhibition Ratio ) - ( Background Average Ratio )} ) * 100 )$$

### Z-138 Proliferation Assay

Z-138 suspension cells were purchased from ATCC (American Type Culture Collection, Manassas, Va.). RPMI/Glutamax medium, penicillin-streptomycin, heat inactivated fetal bovine serum were purchased from Life Technologies, Grand Island, N.Y., USA. V-bottom polypropylene 384-well plates were purchased from Greiner Bio-One, Monroe, N.C., USA. Cell culture 384-well white opaque plates were purchased from Perkin Elmer, Waltham, Mass., USA. Cell-Titer Glo® was purchased from Promega Corporation, Madison, Wis., USA. SpectraMax M5 plate reader was purchased from Molecular Devices LLC, Sunnyvale, Calif., USA.

Z-138 suspension cells were maintained in growth medium (RPMI 1640 supplemented with 10% v/v heat inactivated fetal bovine serum and cultured at 37° C. under 5% CO₂. Under assay conditions, cells were incubated in assay medium (RPMI 1640 supplemented with 10% v/v heat inactivated fetal bovine serum and 100 units/mL penicillin-streptomycin) at 37° C. under 5% CO₂.

For the assessment of the effect of compounds on the proliferation of the Z-138 cell line, exponentially growing cells were plated in 384-well white opaque plates at a density of 10,000 cells/ml in a final volume of 50 μl of assay medium. A compound source plate was prepared by performing triplicate nine-point 3-fold serial dilutions in DMSO, beginning at 10 mM (final top concentration of compound in the assay was 20 μM and the DMSO was 0.2%). A 100 nL aliquot from the compound stock plate was added to its respective well in the cell plate. The 100% inhibition control consisted of cells treated with 200 nM final concentration of staurosporine and the 0% inhibition control consisted of DMSO treated cells. After addition of compounds, assay plates were incubated for 5 days at 37° C., 5% CO₂, relative humidity >90%.

Cell viability was measured by quantitation of ATP present in the cell cultures, adding 35 μl of Cell Titer Glo® reagent to the cell plates. Luminescence was read in the SpectraMax M5 microplate reader. The concentration of compound inhibiting cell viability by 50% was determined using a 4-parametric fit of the normalized dose response curves.

Results for certain compounds described herein are shown in Table 2.

TABLE 2
Biological Assay Results

| | Biochemical | ICW | Proliferation |
|---|---|---|---|
| Cmpd No | IC₅₀ | EC₅₀ | EC₅₀ |
| 1-1 | B | — | — |
| 2-1 | B | — | — |
| 3-1 | B | — | — |
| 4-1 | B | — | — |
| 5-1 | B | — | — |
| 6-1 | B | — | — |
| 7-1 | B | — | — |
| 8-1 | B | — | — |
| 9-1 | B | — | — |
| 10-1 | B | — | — |
| 11-1 | B | — | — |
| 12-1 | B | — | — |
| 13-1 | B | — | — |
| 14-1 | B | — | — |
| 15-1 | A | — | — |
| 16-1 | A | — | — |
| 17-1 | A | — | — |
| 18-1 | A | — | — |
| 19-1 | B | — | — |
| 20-1 | B | — | — |
| 21-1 | A | — | — |
| 22-1 | B | — | — |
| 23-1 | A | — | — |
| 24-1 | A | — | — |
| 25-1 | B | — | — |
| 26-1 | B | — | — |
| 27-1 | B | — | — |
| 28-1 | B | — | — |
| 29-1 | B | — | — |
| 30-1 | A | — | — |
| 31-1 | A | — | — |
| 32-1 | B | — | — |
| 33-1 | A | — | — |
| 34-1 | A | — | — |
| 35-1 | B | — | — |
| 36-1 | A | — | — |
| 37-1 | B | — | — |
| 38-1 | B | — | — |
| 39-1 | B | — | — |
| 40-1 | B | — | — |
| 41-1 | B | — | — |
| 42-1 | B | — | — |
| 43-1 | B | — | — |
| 44-1 | B | — | — |
| 45-1 | B | — | — |
| 46-1 | B | — | — |
| 47-1 | B | — | — |
| 48-1 | B | — | — |
| 49-1 | A | — | — |
| 50-1 | B | — | — |
| 51-1 | B | — | — |
| 52-1 | B | — | — |
| 53-1 | B | — | — |
| 54-1 | A | — | — |
| 55-1 | A | — | — |
| 56-1 | B | — | — |
| 57-1 | A | — | — |
| 58-1 | A | — | — |
| 59-1 | B | — | — |
| 60-1 | A | — | — |
| 61-1 | A | — | — |
| 62-1 | B | — | — |
| 63-1 | A | — | — |
| 64-1 | B | — | — |
| 65-1 | B | — | — |
| 66-1 | B | — | — |
| 67-1 | B | — | — |
| 68-1 | A | — | — |
| 69-1 | A | — | — |
| 70-1 | A | — | — |
| 71-1 | B | — | — |
| 72-1 | A | — | — |
| 73-1 | B | — | — |
| 74-1 | A | — | — |
| 75-1 | A | — | — |
| 76-1 | B | — | — |
| 77-1 | B | — | — |
| 78-1 | A | — | — |
| 79-1 | B | — | — |
| 80-1 | A | — | — |
| 81-1 | A | — | — |
| 82-1 | B | — | — |
| 83-1 | B | — | — |
| 84-1 | B | — | — |
| 85-1 | B | — | — |
| 86-1 | B | — | — |
| 87-1 | B | — | — |
| 88-1 | B | — | — |
| 89-1 | A | — | — |
| 90-1 | A | — | — |
| 91-1 | A | — | — |
| 92-1 | A | C | D |
| 93-1 | B | — | — |
| 94-1 | B | — | — |
| 95-1 | B | — | — |
| 96-1 | B | — | — |
| 97-1 | A | — | — |
| 2-2 | B | — | — |
| 2-3 | B | C | ** |
| 3-3 | B | C | D |
| 4-3 | B | — | ** |
| 5-3 | B | — | ** |
| 6-3 | B | — | ** |
| 7-3 | B | — | ** |
| 8-3 | B | — | ** |
| 9-3 | A | — | ** |
| 10-3 | B | — | ** |
| 11-3 | B | — | ** |
| 12-3 | B | D | — |
| 13-3 | B | — | ** |
| 14-3 | A | C | ** |
| 15-3 | B | ** | ** |
| 1-4 | B | B | C |
| 2-4 | B | B | D |
| 3-4 | A | A | C |
| 4-4 | B | B | D |
| 5-4 | A | B | C |
| 6-4 | B | C | — |
| 7-4 | A | A | C |
| 8-4 | B | C | D |
| 1-5 | B | C | ** |
| 2-5 | A | B | ** |
| 3-5 | A | B | D |
| 1-6 | B | B | ** |
| 2-6 | B | — | — |
| 3-6 | B | — | — |
| 4-6 | B | — | — |
| 5-6 | A | — | — |
| 6-6 | B | — | — |
| 1-7 | A | B | C |
| 2-7 | A | B | C |
| 3-7 | A | B | D |
| 4-7 | B | B | — |
| 5-7 | A | B | C |
| 1-8 | B | — | G |
| 1-9 | B | C | D |
| 2-9 | A | A | B |
| 3-9 | A | A | B |
| 4-9 | A | A | A |
| 5-9 | A | A | G |
| 6-9 | B | B | G |
| 7-9 | A | A | C |
| 8-9 | B | A | C |
| 9-9 | A | B | B |
| 10-9 | A | A | B |
| 11-9 | A | A | B |
| 12-9 | A | A | C |
| 13-9 | B | B | C |
| 14-9 | A | A | A |
| 15-9 | — | — | — |
| 16-9 | — | — | — |
| 17-9 | — | — | — |
| 18-9 | — | — | — |
| 19-9 | — | — | — |
| 20-9 | — | — | — |
| 21-9 | — | — | — |
| 22-9 | — | — | — |
| 23-9 | A | B | F |
| 24-9 | B | B | G |
| 1-10 | A | A | C |
| 1-11 | B | — | ** |
| 2-11 | A | B | D |
| 3-11 | B | B | D |
| 4-11 | B | B | F |
| 5-11 | A | B | C |
| 6-11 | B | B | D |
| 7-11 | B | B | C |
| 8-11 | A | A | C |
| 9-11 | B | B | ** |
| 1-12 | A | B | G |
| 1-13 | C | F | G |
| 2-13 | C | F | G |
| 3-13 | B | F | G |
| 4-13 | D | F | G |
| 5-13 | C | — | — |
| 6-13 | D | F | G |
| 7-13 | D | — | — |
| 1-14 | B | C | ** |
| 2-14 | C | — | — |
| 3-14 | C | ** | ** |
| 4-14 | D | — | — |
| 5-14 | C | — | — |
| 6-14 | D | — | — |
| 7-14 | C | — | — |
| 8-14 | C | — | — |
| 9-14 | C | — | — |
| 10-14 | D | — | — |
| 11-14 | C | — | — |
| 12-14 | B | ** | ** |
| 13-14 | C | — | — |
| 14-14 | C | — | — |
| 15-14 | C | — | — |
| 16-14 | C | — | — |
| 17-14 | C | — | — |
| 18-14 | C | — | — |
| 19-14 | C | — | — |
| 20-14 | B | C | ** |
| 21-14 | C | — | — |
| 22-14 | B | ** | ** |
| 23-14 | C | — | — |
| 24-14 | D | — | — |
| 25-14 | D | — | — |
| 26-14 | D | — | — |
| 27-14 | C | — | — |
| 28-14 | C | — | — |
| 29-14 | B | — | — |
| 30-14 | C | — | — |
| 31-14 | C | — | — |
| 32-14 | C | — | — |
| 33-14 | C | — | — |
| 34-14 | D | — | — |
| 35-14 | C | — | — |
| 36-14 | C | — | — |
| 37-14 | B | — | — |
| 38-14 | C | — | — |
| 39-14 | B | — | — |
| 40-14 | C | — | — |
| 41-14 | C | — | — |
| 42-14 | B | — | — |
| 43-14 | C | — | — |
| 44-14 | C | — | — |
| 45-14 | D | — | — |
| 46-14 | C | — | — |
| 47-14 | B | — | — |
| 48-14 | B | — | — |
| 49-14 | D | — | — |
| 50-14 | C | — | — |
| 51-14 | D | — | — |
| 52-14 | D | — | — |
| 53-14 | C | — | — |
| 54-14 | D | — | — |
| 55-14 | B | — | — |
| 56-14 | B | — | — |
| 57-14 | C | — | — |
| 58-14 | B | — | — |
| 59-14 | B | — | — |
| 60-14 | D | — | — |
| 61-14 | D | — | — |
| 62-14 | C | — | — |
| 63-14 | B | — | — |
| 64-14 | C | — | — |
| 65-14 | D | — | — |
| 66-14 | C | — | — |
| 67-14 | C | — | — |
| 68-14 | D | — | — |
| 69-14 | B | — | — |
| 70-14 | D | — | — |
| 71-14 | C | — | — |
| 72-14 | B | — | — |
| 73-14 | B | — | — |
| 74-14 | C | — | — |
| 75-14 | B | — | — |
| 1-15 | — | — | — |

For Table 2, “A” indicates an IC₅₀ or EC₅₀ < 0.100 μM, “B” indicates an IC₅₀ or EC₅₀ of 0.101-1.000 μM, “C” indicates an IC₅₀ or EC₅₀ of 1.001-10.000 μM, “D” indicates an IC₅₀ or EC₅₀ of 10.001-50 μM, and “E” indicates an IC₅₀ or EC₅₀ > 50 μM, “—” indicates no data, “F” indicates an IC₅₀ or EC₅₀ > 1 μM, “G” indicates an IC₅₀ or EC₅₀ > 5 μM, “*” indicates an IC₅₀ or EC₅₀ > 10 μM, “**” indicates an IC₅₀ or EC₅₀ > 20 μM.

### Other Embodiments

The foregoing has been a description of certain non-limiting embodiments of the invention. Those of ordinary skill in the art will appreciate that various changes and modifications to this description may be made without departing from the spirit or scope of the present invention, as defined in the following claims.

What is claimed is:

1. A compound of Formula (I): 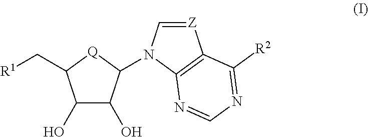
or a pharmaceutically acceptable salt thereof,
wherein
R¹ is —SR^{1a} or —N(R^{1a})₂;
each R^{1a} is independently hydrogen, substituted or unsubstituted alkyl, substituted or unsubstituted cycloalkyl, substituted or unsubstituted heterocyclyl, —C(═O)R^{Z}, —C(═NH)R^{Z}, a nitrogen protecting group when attached to nitrogen, a sulfur protecting group when attached to sulfur, or two instances of R^{1a} can be taken together with their intervening atoms to form a substituted or unsubstituted heterocyclic ring;
R^{Z} is substituted or unsubstituted alkyl or substituted or unsubstituted aryl;
R² is hydrogen or —NH₂;
Z is CH or N; and
Q is CH₂ or O.

2. The compound of claim 1, wherein the compound is of Formula (I-a): 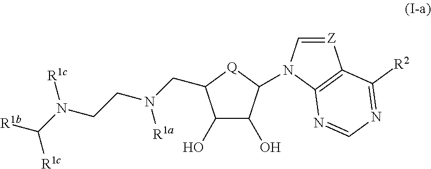
or a pharmaceutically acceptable salt thereof,
wherein:
R^{1a} is —CH₃;
R^{1b} is substituted or unsubstituted aryl or substituted or unsubstituted heteroaryl; and
each R^{1c} is independently hydrogen, —CH₃, or —CH₂CH₃.

3. The compound of claim 2, wherein the compound is of Formula (I-a1): 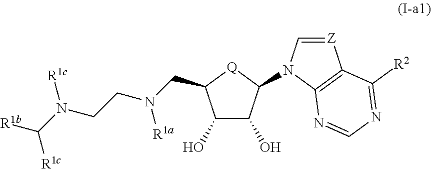
or a pharmaceutically acceptable salt thereof.

4. The compound of claim 1, wherein the compound is of Formula (I-b): 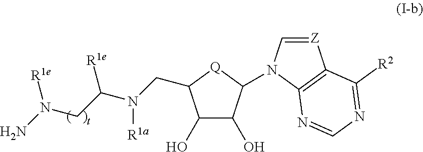
or a pharmaceutically acceptable salt thereof,
wherein:
R^{1a} is substituted or unsubstituted alkyl;
each R^{1e} is independently hydrogen, —CH₃, or —CO₂H; and
t is 0, 1, or 2.

5. The compound of claim 4, wherein the compound is of Formula (I-b1): 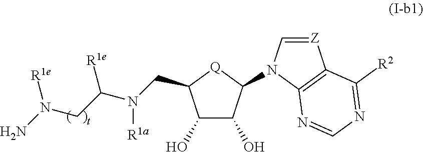
or a pharmaceutically acceptable salt thereof.

6. The compound of claim 1, wherein the compound is of Formula (I-c): 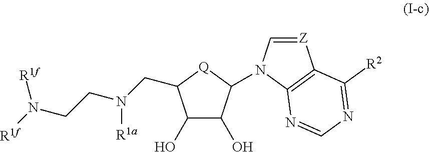
or a pharmaceutically acceptable salt thereof,
wherein:
each R^{1f} is independently substituted or unsubstituted alkyl, substituted or unsubstituted heterocyclyl, a nitrogen protecting group, or two of R^{1f} can be taken together with their intervening atoms to form a substituted or unsubstituted heterocyclic ring; and
R^{1a} is substituted or unsubstituted alkyl.

7. The compound of claim 6, wherein the compound is of Formula (I-c1): 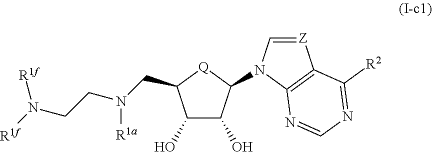
or a pharmaceutically acceptable salt thereof.

8. The compound of claim 1, wherein the compound is of Formula (I-d): 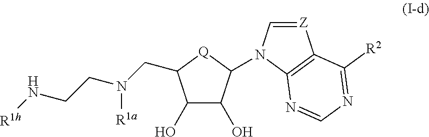
or a pharmaceutically acceptable salt thereof,
wherein:
R^{1h} is substituted or unsubstituted alkyl, substituted or unsubstituted cycloalkyl, substituted or unsubstituted heterocyclyl, substituted or unsubstituted aryl, substituted or unsubstituted heteroaryl, —C(═O)NHR^{1ha}, or a nitrogen protecting group;
R^{1ha} is substituted or unsubstituted aryl; and
R^{1a} is hydrogen, substituted or unsubstituted alkyl, or a nitrogen protecting group.

9. The compound of claim 8, wherein the compound is of Formula (I-d1): 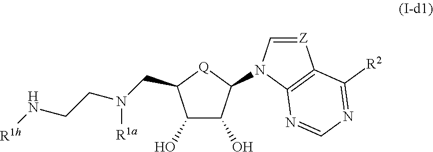
or a pharmaceutically acceptable salt thereof.

10. The compound of claim 1, wherein the compound is of Formula (I-e): 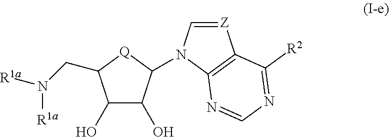
or a pharmaceutically acceptable salt thereof,
wherein:
each R^{1a} is independently —CH₃, —C(═O)R^{Z}, —C(═NH)R^{Z}, substituted or unsubstituted cycloalkyl, substituted or unsubstituted heterocyclyl, or two of R^{1a} can be taken together with their intervening atoms to form a substituted or unsubstituted heterocyclic ring; and
R^{Z} is substituted or unsubstituted aryl.

11. The compound of claim 10, wherein the compound is of Formula (I-e1): 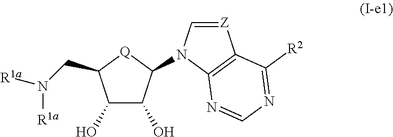
or a pharmaceutically acceptable salt thereof.

12. The compound of claim 1, wherein the compound of Formula (I-f): 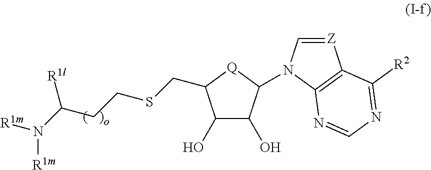
or a pharmaceutically acceptable salt thereof,
wherein:
R^{1l} is hydrogen, —CO₂H, or —CH₂OH;
each R^{1m} is independently hydrogen or two of R^{1m} can be taken together with their intervening atoms to form a substituted or unsubstituted heterocyclic ring; and
o is 0, 1, or 2.

13. The compound of claim 12, wherein the compound of Formula (I-f1): 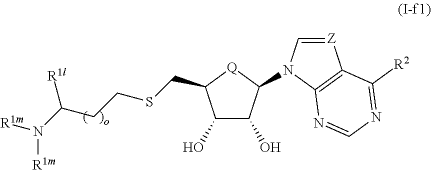
or a pharmaceutically acceptable salt thereof.

14. The compound of claim 1, wherein the compound is of Formula (I-h): 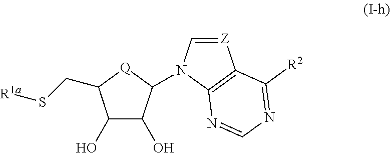
or a pharmaceutically acceptable salt thereof,
wherein:
R^{1a} is C₁₋₃ alkyl optionally substituted with one instance of —CO₂H.

15. The compound of claim 14, wherein the compound is of Formula (I-h1): 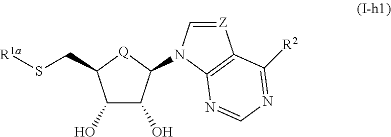
or a pharmaceutically acceptable salt thereof.

16. A compound of Formula (II): 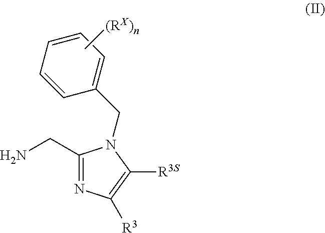
or a pharmaceutically acceptable salt thereof,
wherein:
R³ is hydrogen, methyl, ethyl, propyl, substituted or unsubstituted heteroarylalkyl, or —CH₂CH₂NHC(═O)R^{3a};
R^{3S} is hydrogen, methyl, ethyl, propyl, substituted or unsubstituted heteroarylalkyl, or —CH₂CH₂NHC(═O)R^{3a};
each occurrence of R^{3a} is independently substituted or unsubstituted alkyl, or substituted or unsubstituted aryl;
each R^{X} is independently selected from the group consisting of substituted or unsubstituted alkyl, substituted or unsubstituted cycloalkyl, substituted or unsubstituted aryl, substituted or unsubstituted heterocyclyl, substituted or unsubstituted heteroaryl, halogen, —CN, —NO₂, —OR^{X1}, —N(R^{X2})₂, —SR^{X1}, —C(═O)R^{X1}, —C(O)OR^{X1}, —C(O)SR^{X1}, —C(O)N(R^{X2})₂, —C(O)N(R^{X2})N(R^{X2})₂, —OC(O)R^{X1}, —OC(O)N(R^{X2})₂, —NR^{X2}C(O)R^{X1}, —NRC(O)N(R^{X2})₂, —NR^{X2}C(O)N(R^{X2})N(R^{X2})₂, —NR^{X2}C(O)OR^{X1}, —SC(O)R^{X1}, —C(═NR^{X2})R^{X1}, —C(═NNR^{X2})R^{X1}, —C(═NOR^{X1})R^{X1}, —C(═NR^{X2})N(R^{X2})₂, —NR^{X2}C(═NR^{X2})R^{X2}, C(═S)R^{X1}, —C(═S)N(R^{X2})₂, —NR^{X2}C(═S)R^{X1}, —S(O)R^{X1}, —OS(O)₂R^{X1}, —SO₂R^{X1}, —NRSO₂R^{X1}, or —SO₂N(R^{X2})₂;
each R^{X1} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl;
each R^{X2} is independently selected from the group consisting of hydrogen, substituted or unsubstituted alkyl, substituted or unsubstituted cycloalkyl, substituted or unsubstituted heterocyclyl, substituted or unsubstituted aryl, and substituted or unsubstituted heteroaryl, or two R^{X2} groups are taken together with their intervening atoms to form a substituted or unsubstituted heterocyclic ring; and
n is 0, 1, 2, 3, 4, or 5.

17. A compound of Formula (III): 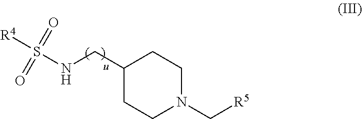
or a pharmaceutically acceptable salt thereof,
wherein:
R⁴ is substituted or unsubstituted aryl;
R⁵ is substituted or unsubstituted aryl or substituted or unsubstituted heteroaryl; and
u is 0 or 1.

18. A compound of Formula (IV): 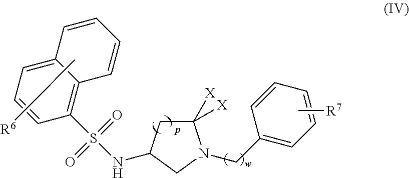
or a pharmaceutically acceptable salt thereof,
wherein:
R⁶ is hydrogen or —N(R^{6a})₂;
each R^{6a} is independently substituted or unsubstituted alkyl;
each X is hydrogen, or two X groups are joined to form ═O;
R⁷ is hydrogen, halogen, substituted or unsubstituted alkyl, or —N(R^{7a})₂;
R^{7a} is hydrogen or substituted or unsubstituted alkyl;
p is 0 or 1; and
w is 0 or 1,
wherein if p is 0, then two X groups are not joined to form ═O.

19. The compound of claim 12, wherein the compound is of Formula (IV-a): 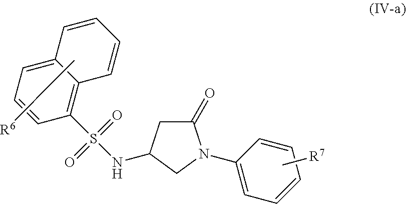
or a pharmaceutically acceptable salt thereof.

20. The compound of claim 12, wherein the compound is of Formula (IV-b): 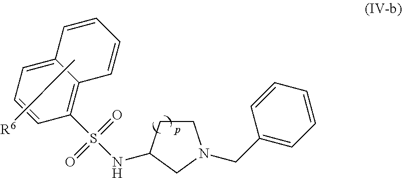
or a pharmaceutically acceptable salt thereof.

21. The compound of claim 12, wherein the compound is of Formula (IV-c): 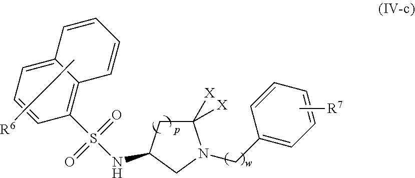
or a pharmaceutically acceptable salt thereof.

22. The compound of claim 12, wherein the compound is of Formula (IV-d): 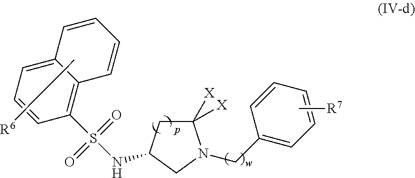
or a pharmaceutically acceptable salt thereof.

23. A compound of Formula (V): 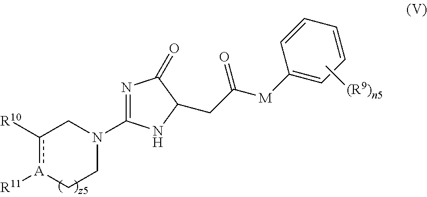
or a pharmaceutically acceptable salt thereof,
wherein:
each occurrence of R⁹ is independently substituted or unsubstituted alkyl, —OR^{9a}, —C(═O)R^{9a}, —C(═O)OR^{9a}, —(C═O)NHR^{9a}, —NH(C═O)R^{9a}, —CN, or halogen, or two vicinal R⁹ are taken together with their intervening atoms to form a substituted or unsubstituted heterocyclic ring;
each occurrence of R^{9a} is independently hydrogen, independently substituted or unsubstituted alkyl, or substituted or unsubstituted aryl;
M is a bond or NH;
when [symbol] is a single bond, then A is N and R¹¹ is present, or A is CH or C(OH) and R¹¹ is present;
when [symbol] is a double bond, then A is N and R¹¹ is absent, or A is C and R¹¹ is present; and
R¹⁰ is hydrogen;
R¹¹ is substituted or unsubstituted alkyl, substituted or unsubstituted aralkyl, substituted or unsubstituted heteroaralkyl, substituted or unsubstituted aryl, or substituted or unsubstituted heteroaryl; or
R¹⁰ and R¹¹ are taken together with their intervening atoms to form an unsubstituted fused phenyl ring; and
n5 is 0, 1, 2, 3, 4, or 5; and
z5 is 0 or 1.

24. A compound of Formula (VI): 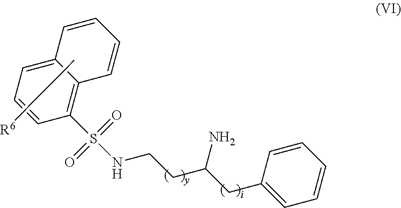
or a pharmaceutically acceptable salt thereof,
wherein:
R⁶ is hydrogen or —N(R^{6a})₂;
each R^{6a} is independently substituted or unsubstituted alkyl; and
y and i are each independently 0 or 1.

25. The compound of claim 24, wherein the compound is of Formula (VI-a): 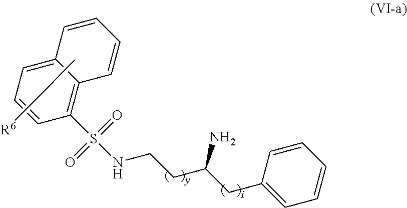
or a pharmaceutically acceptable salt thereof.

26. The compound of claim 24, wherein the compound is of Formula (VI-b): 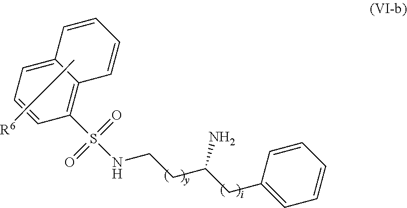
or a pharmaceutically acceptable salt thereof.

27. A compound of Formula (VII): 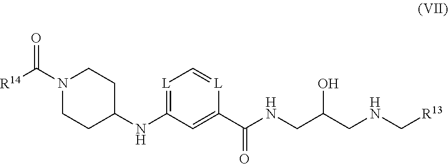
or a pharmaceutically acceptable salt thereof,
wherein:
R¹³ is substituted or unsubstituted aryl;
R¹⁴ is substituted or unsubstituted alkyl; and
L is independently CH or N, wherein at least one of L must be N.

28. The compound of claim 27, wherein the compound is of Formula (VII-a): 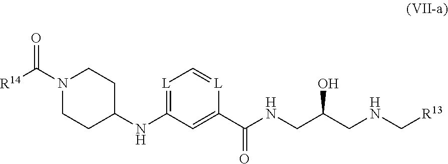
or a pharmaceutically acceptable salt thereof.

29. The compound of claim 27, wherein the compound is of Formula (VII-b): 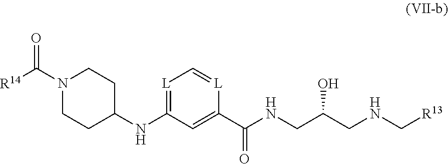
or a pharmaceutically acceptable salt thereof.

30. A compound of Formula (VIII): 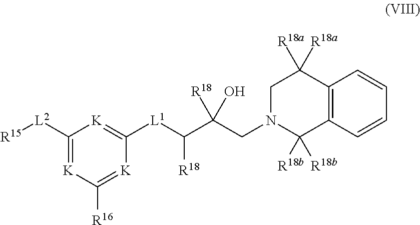
or a pharmaceutically acceptable salt thereof,
wherein:
L¹ is O, C(═O)NH, or C(═O)NMe;
L² is a bond or NH;
each R¹⁵ is independently substituted or unsubstituted cycloalkyl, substituted or unsubstituted heterocyclyl, or substituted or unsubstituted heteroaryl;
R¹⁶ is hydrogen or —NHR⁷;
R¹⁷ is substituted or unsubstituted heterocyclyl;
each R¹⁸ is independently hydrogen or —CH₃;
each R^{18a} is hydrogen or both instances of R^{18a} are taken together with their intervening atoms to form a substituted or unsubstituted cyclopropyl ring;
each R^{18b} is independently hydrogen or —CH₃;
or one instance of R^{18a} and one instance of R^{18b} are taken together with their intervening atoms to form a substituted or unsubstituted pyrrolidine or piperidine ring; and
each K is independently CH or N, wherein no more than two of K can be N.

31. The compound of claim 30, wherein the compound is of Formula (VIII-a): 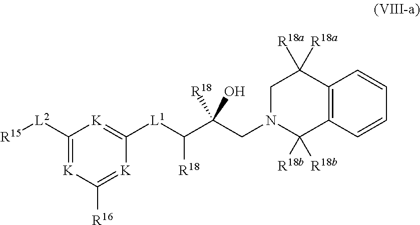
or a pharmaceutically acceptable salt thereof.

32. The compound of claim 30, wherein the compound is of Formula (VIII-b): 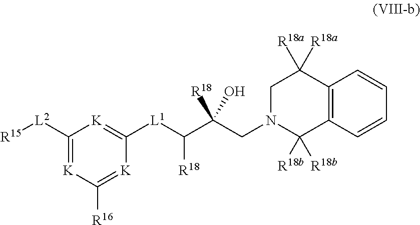
or a pharmaceutically acceptable salt thereof.

33. The compound of claim 31, wherein the compound is of Formula (VIII-c): 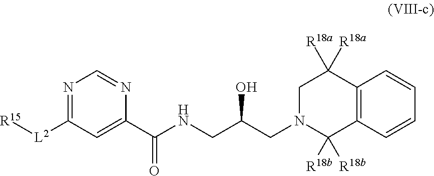
or a pharmaceutically acceptable salt thereof.

34. The compound of claim 32, wherein the compound is of Formula (VIII-d): 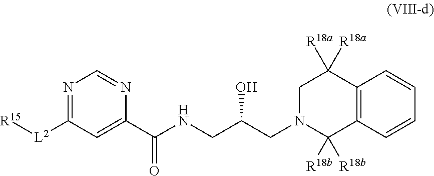
or a pharmaceutically acceptable salt thereof.

35. The compound of claim 31, wherein the compound is of Formula (VIII-e): 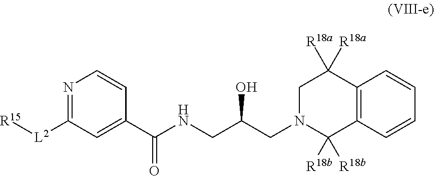
or a pharmaceutically acceptable salt thereof.

36. The compound of claim 32, wherein the compound is of Formula (VIII-f): 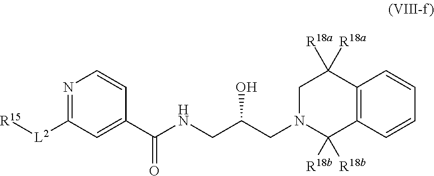
or a pharmaceutically acceptable salt thereof.

37. The compound of claim 31, wherein the compound is of Formula (VIII-g): 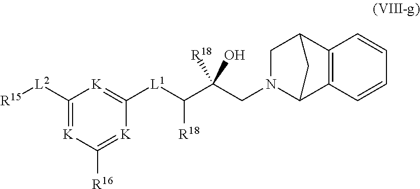
or a pharmaceutically acceptable salt thereof.

38. The compound of claim 32, wherein the compound is of Formula (VIII-h): 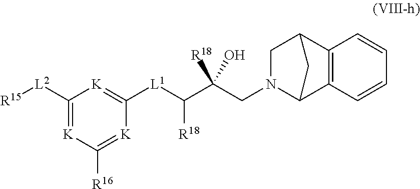
or a pharmaceutically acceptable salt thereof.

39. The compound of claim 31, wherein the compound is of Formula (VIII-i): 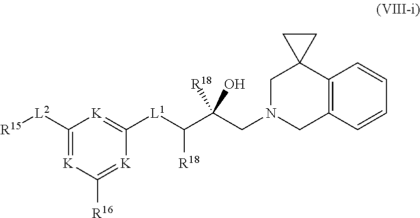
or a pharmaceutically acceptable salt thereof.

40. The compound of claim 32, wherein the compound is of Formula (VIII-j): 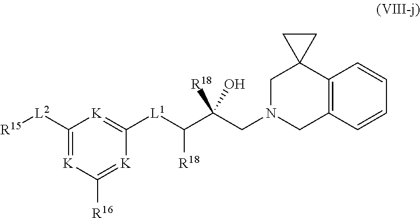
or a pharmaceutically acceptable salt thereof.

41. A compound of Formula (IX): 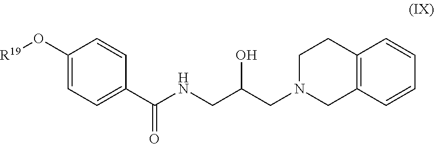
or a pharmaceutically acceptable salt thereof, wherein R¹⁹ is substituted or unsubstituted heterocyclyl.

42. The compound of claim 41, wherein the compound is of Formula (IX-a): 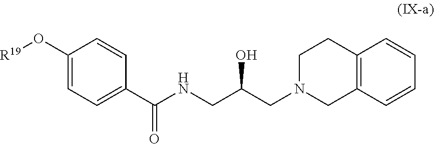
or a pharmaceutically acceptable salt thereof.

43. The compound of claim 41, wherein the compound is of Formula (IX-b): 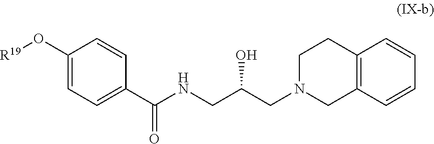
or a pharmaceutically acceptable salt thereof.

44. A compound of Formula (X): 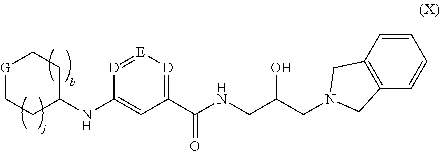
or a pharmaceutically acceptable salt thereof,
wherein:
D is CH or N;
E is C(R²¹) or N, wherein if E is C(R²¹) then at least one instance of D is N and if E is N then neither instance of D is N;
G is O or N(R²⁰);
R²⁰ is hydrogen, substituted or unsubstituted alkyl, —SO₂R^{20a}, or —C(═O)R^{20a};
R^{20a} is substituted or unsubstituted alkyl or substituted or unsubstituted cycloalkyl;
R²¹ is hydrogen or substituted or unsubstituted morpholine; and
j and b are each independently 0 or 1.

45. The compound of claim 44, wherein the compound is of Formula (X-a): 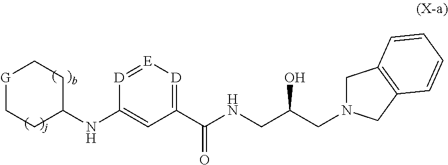
or a pharmaceutically acceptable salt thereof.

46. The compound of claim 44, wherein the compound is of Formula (X-b): 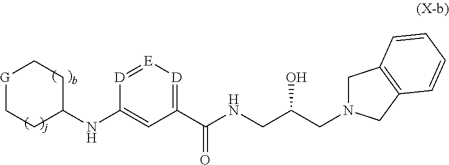
or a pharmaceutically acceptable salt thereof.

47. The compound of claim 2 or 3, wherein R^{1b} is:

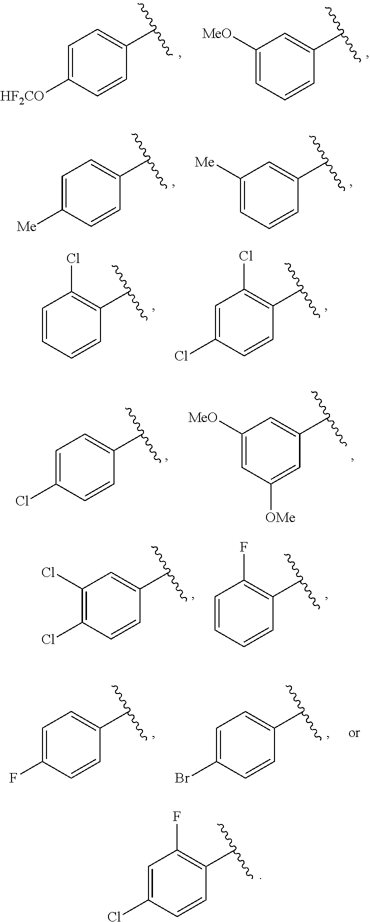

48. The compound of claim 2 or 3, wherein one R^{1c} is —CH₃ or —CH₃.

49. The compound of claim 4 or 5, wherein both instances of R^{1e} are hydrogen.

50. The compound of claim 4 or 5, wherein one instance of R^{1e} is —CO₂H.

51. The compound of claim 6 or 7, wherein one instance of R^{1f} is —CH₂(cyclopropyl) wherein, wherein cyclopropyl is substituted or unsubstituted, —CH₂CH₂OH, 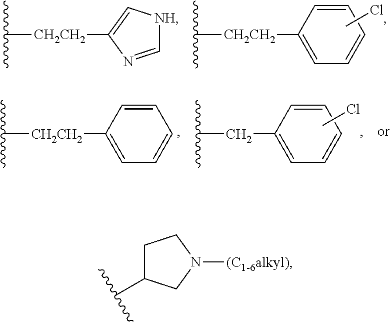
wherein C₁₋₆alkyl is substituted or unsubstituted.

52. The compound of claim 6 or 7, wherein two instances of R^{1f} are taken together to form a ring of formula:

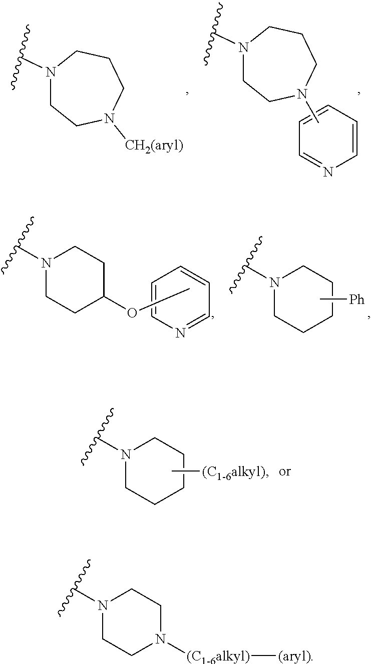

53. The compound of claim 8 or 9, wherein R^{1h} is of formula:
—CH₂CH₂OH, —CH(Me)CH₂OH, —CH₂CH₂OMe, —CH(Et)CH₂OMe, —CH(Me)CH₂OMe, —CH₂CH₂OPh, 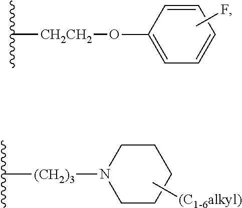
wherein C₁₋₆alkyl is substituted or unsubstituted,

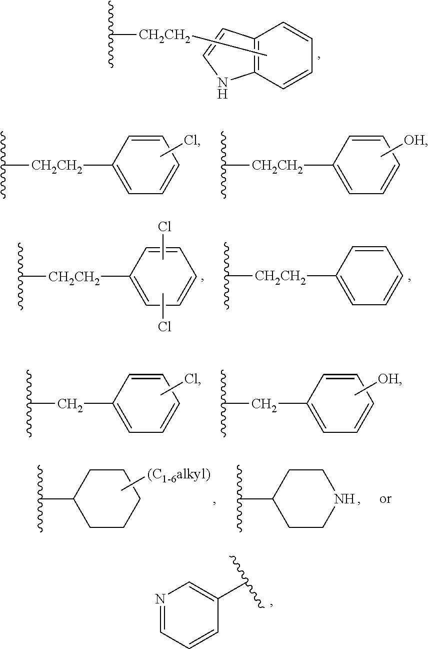

54. The compound of claim 8 or 9, wherein R^{1h} is —C(═O)NHR^{1ha}; and R^{1ha} is of formula 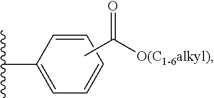
wherein C₁₋₆alkyl is substituted or unsubstituted.

55. The compound of claim 12 or 13, wherein two instances of R^{1m} are taken together to form a ring of formula

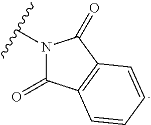 [SMILES: *N1C(=O)c2ccccc2C1=O]

56. The compound of any of claims 1-15, wherein Q is O.

57. The compound of any of claims 1-15, wherein R² is —NH₂.

58. The compound of any of claims 1-15, wherein Z is N.

59. The compound of claim 16, wherein n is 0.

60. The compound of claim 16, wherein R³ is hydrogen.

61. The compound of claim 16, wherein R³ is —CH₂CH₂NHC(═O)R^{3a}; and R^{3a} is unsubstituted phenyl.

62. The compound of claim 17, wherein R⁴ is of formula: 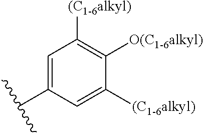
wherein C₁₋₆alkyl is substituted or unsubstituted, 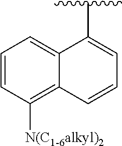
wherein C₁₋₆alkyl is substituted or unsubstituted, 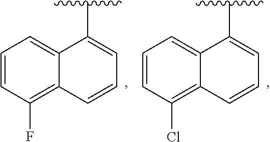
or unsubstituted naphthyl.

63. The compound of claim 17, wherein R⁵ is of formula:

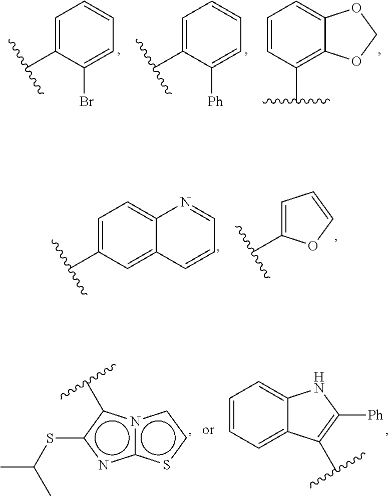

64. The compound of any of claims 19-22, wherein R⁶ is —NMe₂.

65. The compound of any of claims 19-22, wherein R⁶ is hydrogen, -Me, —F, or —NH₂.

66. The compound of claim 23, wherein the ring system: 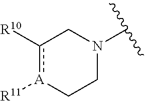
corresponds to a ring system of formula:

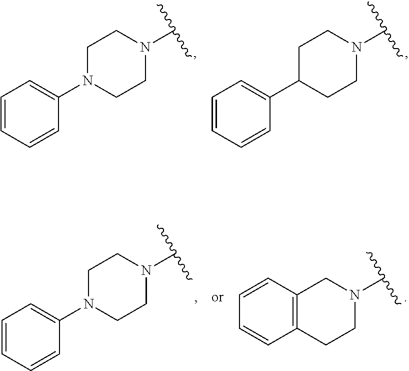 [SMILES: *N1CCN(c2ccccc2)CC1 ; *N1CCC(c2ccccc2)CC1 ; *N1CCN(c2ccccc2)CC1 ; *N1CCc2ccccc2C1]

67. The compound of claim 23, wherein M is a bond; and R⁹ is —OR^{9a}.

68. The compound of claim 23, wherein M is NH; and R⁹ is a halogen.

69. The compound of any of claims 24-26, wherein R⁶ is —NMe₂.

70. The compound of any of claims 27-29, wherein R¹³ is unsubstituted phenyl.

71. The compound of any of claims 27-29, wherein R¹⁴ is unsubstituted C₁₋₆alkyl.

72. The compound of any of claims 30-40, wherein L² is a bond.

73. The compound of any of claims 30-40, wherein L² is NH.

74. The compound of any of claims 30-40, wherein R¹⁵ is of formula: 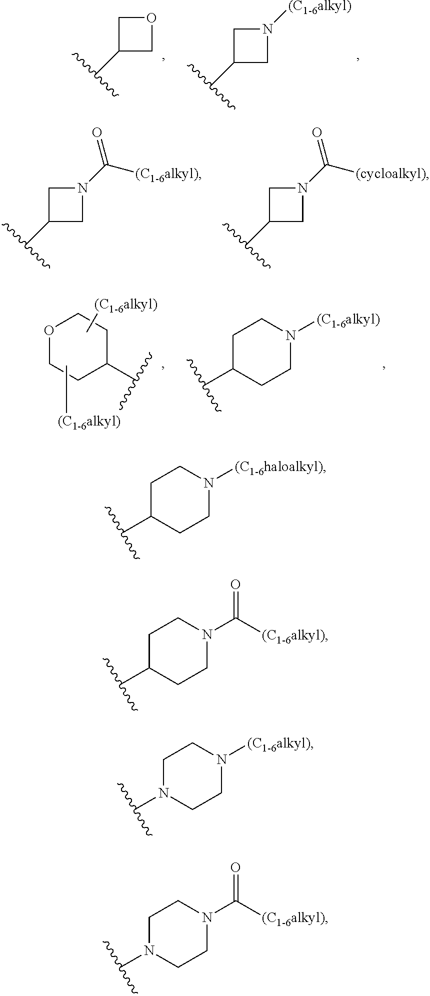
or benzothiazole,
wherein C₁₋₆alkyl or cycloalkyl is substituted or unsubstituted.

75. The compound of any of claims 41-43, wherein R¹⁹ is of formula

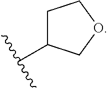 [SMILES: *C1CCOC1]

76. The compound of any of claims 44-46, wherein the ring comprising D and E is of formula:

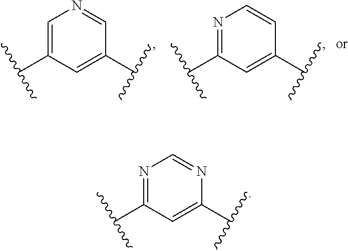 [SMILES: *c1cncc(*)c1 ; *c1ccnc(*)c1 ; *c1cc(*)ncn1]

77. The compound of any of claims 44-46, wherein the ring comprising G is of formula:

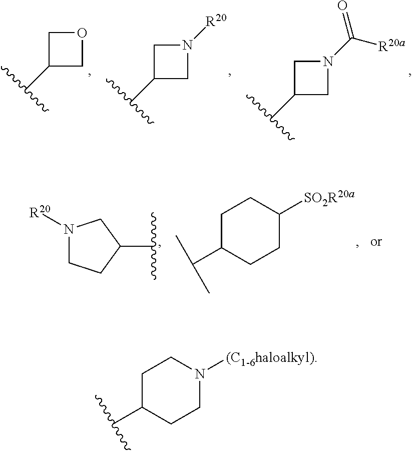

78. A compound selected from the group consisting of the compounds listed in Table 1A-Table 1O, and pharmaceutically acceptable salts thereof.

79. A pharmaceutical composition comprising a compound of any one of claims 1-78, or a pharmaceutically acceptable salt thereof, and a pharmaceutically acceptable excipient.

80. A kit or packaged pharmaceutical comprising a compound of any one of claims 1-78 and instructions for use thereof.

81. A method of inhibiting PRMT5 comprising contacting a cell with an effective amount of a compound of any one of claims 1-78 or a pharmaceutically acceptable salt thereof.

82. A method of altering gene expression comprising contacting a cell with an effective amount of a compound of any one of claims 1-78 or a pharmaceutically acceptable salt thereof.

83. A method of altering transcription comprising contacting a cell with an effective amount of a compound of any one of claims 1-78 or a pharmaceutically acceptable salt thereof.

84. The method of any one of claims 81-83, wherein the cell is in vitro.

85. The method of any one of claims 81-83, wherein the cell is in a subject.

86. A method of treating or preventing a PRMT5-mediated disorder, comprising administering to a subject in need thereof a therapeutically effective amount of a compound of any one of claims 1-78, or a pharmaceutically acceptable salt thereof, or a pharmaceutical composition of claim 79.

87. The method of claim 86, wherein the disorder is a proliferative disorder.

88. The method of claim 87, wherein the disorder is cancer.

89. The method of claim 88, wherein the cancer is hematopoietic cancer, lung cancer, prostate cancer, melanoma, or pancreatic cancer.

90. The method of claim 86, wherein the disorder is a metabolic disorder.

91. The method of claim 90, wherein the metabolic disorder is diabetes.

92. The method of claim 90, wherein the metabolic disorder is obesity.

93. The method of claim 86, wherein the disorder is a blood disorder.

94. The method of claim 93, wherein the disorder is a hemoglobinopathy.

95. The method of claim 94, wherein the disorder is sickle cell anemia.

96. The method of claim 94, wherein the disorder is β-thalessemia.


SEQUENCE LISTING (7 sequences)

SEQ ID NO: 1 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 2 - protein, 620 aa, Homo sapiens
MRGPNSGTEKGRLVIPEKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 3 - protein, 16 aa, Artificial Sequence
  Synthetic Polypeptide
SGRGKGGKGLGKGGAK

SEQ ID NO: 4 - protein, 9 aa, Artificial Sequence
  Synthetic Polypeptide
MDYKDDDDK

SEQ ID NO: 5 - protein, 7 aa, Artificial Sequence
  Synthetic Polypeptide
MHHHHHH

SEQ ID NO: 6 - protein, 645 aa, Artificial Sequence
  Synthetic Polypeptide
MDYKDDDDKAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 7 - protein, 348 aa, Artificial Sequence
  Synthetic Polypeptide
MHHHHHHRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE